(19) United States
(12) Patent Application Publication
(10) Pub. No.: US 2025/0297258 A1
(43) Pub. Date: Sep. 25, 2025

(54) OLIGONUCLEOTIDE-BASED DELIVERY VEHICLE FOR OLIGONUCLEOTIDES AGENTS AND METHODS OF USE THEREOF

(71) Applicant: Ractigen Therapeutics, Rudong Nantong, Jiangsu (CN)

(72) Inventors: Moorim KANG, Rudong Nantong, Jiangsu (CN); Longcheng LI, Rudong Nantong, Jiangsu (CN); Robert F. Place, Rudong Nantong, Jiangsu (CN)

(21) Appl. No.: 18/577,092

(22) PCT Filed: Apr. 18, 2022
(86) PCT No.: PCT/CN2022/104037

§ 371 Date: Jan. 5, 2024

(30) Foreign Application Priority Data
Jul. 7, 2021 (WO) PCT/CN2021/105081
May 6, 2022 (WO) PCT/CN2022/091076

(51) Int. Cl. C12N 15/113; A61P 25/28

(52) U.S. Cl. CPC C12N 15/1137; A61P 25/28; C12N 2310/11; C12N 2310/14; C12N 2310/314; C12N 2310/315; C12N 2310/321; C12N 2310/3231; C12N 2310/3341; C12N 2310/3519; C12Y 115/01001

(57) ABSTRACT

Provided are nucleic acids, specifically as it relates to an oligonucleotide agent comprising a double-stranded RNA (dsRNA, duplex) and a non-targeting accessory oligonucleotide (ACO) that is covalently tethered to the dsRNA and pharmaceutical use thereof.

### CROSS-REFERENCE TO RELATED APPLICATIONS

This application claims priority to the filing date of Provisional Patent Application Serial No. PCT/CN2021/105081 filed Jul. 7, 2021 and to the filing date of Provisional Patent Application Serial No. PCT/CN2022/091076 filed May 6, 2022, the disclosure of which application is herein incorporated by reference.

### FIELD OF THE INVENTION

The present application relates to the technical field of nucleic acids, specifically as it relates to an oligonucleotide agent comprising a double-stranded RNA (dsRNA, duplex) and a non-targeting accessory oligonucleotide (ACO) that is covalently tethered to the dsRNA and pharmaceutical use thereof.

### SEQUENCE LISTING

The instant application contains a Sequence Listing which has been submitted electronically in computer readable format and is hereby incorporated by reference in its entirety.

### BACKGROUND OF THE INVENTION

Oligonucleotides are an emerging class of therapeutics currently under active development for the treatment of a wide variety of diseases via a myriad of mechanisms of action (MOA). Major categories of oligonucleotide therapeutics include single-stranded antisense oligonucleotides (ASOs) and duplex (double-stranded) RNAs (dsRNAs). Single-stranded ASOs in the form of“gapmer” can be used to suppress gene expression by degrading target mRNA via an RNase H mechanism. Gapmer ASOs have a central DNA region required to support the RNase H activity and two ribonucleotide wings to increase target binding affinity of the ASOs. Another category ofASOs is steric blockers, which are typically composed uniformly of ribonucleotides and bind to pre-mRNA in the nucleus to alter mRNA splicing by blocking the binding of certain splicing factors to the mRNA.

dsRNAs can be further classified into two categories: small interfering RNA (siRNA) and small activating RNA (saRNA), both of which require Argonaute (AGO) proteins as their protein partner for function. siRNA binds to target mRNA mainly in the cytoplasm to down-regulate gene expression post-transcriptionally via the RNA interference (RNAi) mechanism. saRNA targets regulatory sequences in the nucleus such as gene promoters to upregulate gene expression at the transcriptional level via the RNAa (RNA activation) mechanism.

Almost all single gene diseases and most multi-gene diseases are caused by loss, instead of gain, of a gene's function. Loss of function could arise from epigenetic aberrations and genetic mutations, leading to transcriptional silencing and erroneous forms of the transcribed mRNA respectively One of such errors is erroneous splicing of pre-mRNA due to undesired skipping or inclusion of exons, resulting in unfunctional protein when the mRNA is translated. In such instance, ASOs have been used to correct the erroneous splicing events and ultimately to increase the gene's protein output by sterically blocking the protein-RNA binding interactions between splicing machinery components and the pre-mRNA. Several such ASO drugs have been approved by U.S. Food and Drug Administration (FDA), including an exon inclusion ASO SPINRAZA® for the treatment of spinal muscular atrophy (SMA) and 3 exon skipping ASOs for the treatment of Duchenne muscular dystrophy (DMD).

However, the therapeutic potential of such oligonucleotide modalities is limited by delivery technologies that provide access to target tissues, organs, or cell types. Thus, there is a need for improved oligonucleotide-based delivery mechanisms to address these challenges.

### SUMMARY

The present application provides a novel oligonucleotide agent comprising a double-stranded RNA (dsRNA, duplex) and a non-targeting accessory oligonucleotide (ACO or single-stranded oligonucleotide) that is covalently tethered to the dsRNA. The agent, by itself, constitutes a system termed oligonucleotide-based delivery vehicle (ODV) with “self-delivering” properties. The present inventors found, surprisingly, when ODV is applied to dsRNA (i.e., siRNA or saRNA), favorable biodistribution and activity are obtained for local administration to selected tissues and systemic delivery across several organs/tissues including liver, muscle, lung, kidney, bladder, brain, spinal cord, heart, eye, spleen, etc. The agent possesses certain benefits associated with single-stranded oligonucleotide therapeutics, for instance, unconventional nucleic acid chemistries and modification patterns conducive to delivery, biodistribution, bioavailability, stability, cellular uptake, and other pharmacological properties without concerns of compromising duplex activity.

ODV design includes an RNA duplex, such as an siRNA or saRNA, comprised of two complementary or partial complementary strands with one of the strands covalently linked to an ACO having at least 6 nucleotides with or without one or more linker moieties. The RNA duplex targets at least one nucleic acid sequence (e.g., mRNA) and optionally is chemically modified using oligonucleotide chemistry technologies (e.g., 2′fluoro, 2′-O-methyl, phosphorothioate, mesyl phosphoramidate or boranophosphate backbone, LNA, etc.) conducive to in vivo activity, stability, and safety. The ACO component is not designed to specifically target any complementary nucleic acid sequence in the subject to be administrated to. The ACO component can be chemically-modified on its backbone, nucleoside or other positions, e.g., a phosphorothioate, mesyl phosphoramidate or boranophosphate backbone, a 2′-fluoro-2′-deoxynucleoside (2′-F), a 2′-O-methyl (2′-O-Me), a 2′-O-(2-methoxyethyl) (2′-O-MOE), locked nucleic acid (LNA), bridged nucleic acid (BNA), peptide nucleic acid (PNA), 5′-(E)-vinylphosphonate moiety, 5′-methyl cytosine moiety, etc., to impart physiochemical properties conducive to improve the agent's bioavailability and delivery. Covalent linker moieties can be natural or unnatural nucleotides, ethlyglycol, carbohydrates, alkyl chains, or any other linker used to covalently connect any two oligonucleotides positioned on the 3′- or 5′-terminus of one or both of the strands within the RNA duplex.

Embodiments of the present application are based in part on the surprising discovery that an oligonucleotide agent comprising. (a) a double-stranded oligonucleotide comprising a sense strand and an antisense strand, wherein the antisense strand has complementarity to a target nucleic acid, and (b) a non-targeting single-stranded oligonucleotide, wherein the single-stranded oligonucleotide is 6-22 nucleotides in length, wherein the double-stranded oligonucleotide and the single-stranded oligonucleotide are covalently linked, with or without one or more linking components, to form the oligonucleotide agent.

In certain embodiments of the present application, the double-stranded oligonucleotide is a small interfering RNA (siRNA) or a small activating RNA (saRNA).

In certain embodiments, the single-stranded oligonucleotide comprises at least one phosphorothioate (PS) backbone substitution. In certain embodiments, the single-stranded oligonucleotide has at least 10%, at least 20%, at least 30%, at least 40%, at least 50%, at least 60%, at least 70%, at least 80%, at least 90%, at least 95%, or 100% of the phosphodiester bonds substituted with phosphorothioate (PS) bond on the backbone of the nucleotide sequence. In certain embodiments, the single-stranded oligonucleotide has 85-95% or 95-100% PS bond.

In certain embodiments, the chemical modification in the double-stranded oligonucleotide or the single-stranded oligonucleotide is an addition of a 5′-phosophate moiety at the 5′ end of the nucleotide sequence. In certain embodiments, the chemical modification is an addition of a 5′-(E)-vinvlphosphonate moiety. In certain embodiments, the chemical modification is an addition of a 5′-methyl cytosine moiety at the 5′ end of the nucleotide sequence.

In certain embodiments, the single-stranded oligonucleotide is RNA, DNA, BNA, LNA or PNA. In certain embodiments, the single-stranded oligonucleotide is 8-16 nucleotides in length. In certain embodiments, the single-stranded oligonucleotide is 10-14 nucleotides in length.

In certain embodiments of the present application, the sense strand of the double-stranded oligonucleotide in the oligonucleotide agent is at least 10 nucleotides in length. In certain embodiments of the present application, the sense strand has a nucleotide length ranging from 10-60 nucleotides. In certain embodiments, the sense strand has a nucleotide length ranging from 27-41 nucleotides.

In certain embodiments of the present application, the antisense strand has a nucleotide length ranging from 10-60 nucleotides. In certain embodiments, the antisense strand has a nucleotide length ranging from 19-25 nucleotides.

In certain embodiments, the single-stranded oligonucleotide comprises a nucleotide sequence that is at least 90% identical to the nucleotide sequence selected from SEQ ID NOs: 1-22.

The ACO may also has a specific composition of nucleotides. In some embodiments, the ACO may have a certain percentage of adenines within the nucleotide sequence of the ACO. In some embodiment, the percent composition of adenines is from about 35% to about 65%. In some embodiments, the percent composition of cytosines is from about 35% to about 72%. In some embodiments, the percent composition of guanosines is from about 35% to about 65%. In some embodiments, the percent composition of uracil is from about 35% to about 72%. In some embodiments, the percent composition of purines is from about 64% to about 78%. In some embodiments, the percent composition of pyrimidines is from about 64% to about 86%. In some embodiment, the specific combination of purines and pyrimidines is about 42-58% purines and about 42-58% pyrimidines.

In some embodiments, the nucleotide sequence of the single-stranded oligonucleotide comprises at least about 14%, at least about 28%, at least about 42%, at least about 57%, at least about 71%, at least about 85%, at least about 92%, or about 70-100% of the nucleotides having 2′Ome modification.

In some embodiments, the nucleotide sequence of the single-stranded oligonucleotide is a palindrome sequence.

In some embodiments, the single-stranded oligonucleotide comprises a chemically modified nucleotide sequence that is at least 80%, at least 85%, at least 90%, at least 95%, at least 97%, at least 99% homology, or 100% identical to a nucleotide sequence selected from the group of SEQ ID NOs: 1299-1379. In some embodiments, the single-stranded oligonucleotide comprises a chemically modified nucleotide sequence that is having 0, 1, 2 or 3 different chemical modifications than a nucleotide sequence selected from the group consisting of SEQ ID NOs: 1299-1379.

In some embodiments, the single-stranded oligonucleotide comprises a chemically modified nucleotide sequence that is at least 80%, at least 85%, at least 90%, at least 95%, at least 97%, at least 99% homology, or 100% identical to a nucleotide sequence selected from the group of SEQ ID NOs: 1299-1379. In some embodiments, the single-stranded oligonucleotide comprises a chemically modified nucleotide sequence that is having 0, 1, 2 or 3 different chemical modifications than a nucleotide sequence selected from the group consisting of SEQ ID NOs: 1299-1379.

In some embodiments, the single-stranded oligonucleotide and the double-stranded oligonucleotide is conjugated without a linking component. In some embodiments, the single-stranded oligonucleotide and the double-stranded oligonucleotide is conjugated with one or more linking components. In some embodiments, the double-stranded oligonucleotide and the single-stranded oligonucleotide are covalently conjugated by a linking component. In some embodiments, the single-stranded oligonucleotide is conjugated to a linking component. In some embodiments, the 5′ end, the 3′ end, or an internal nucleotide of the single-stranded oligonucleotide is conjugated to a linking component. In some embodiments, the double-stranded oligonucleotide comprises a sense strand and an antisense strand, and the single-stranded oligonucleotide is covalently conjugated to the sense strand, the antisense strand, or both the sense and the antisense strands of the double-stranded oligonucleotide by a linking component. In some embodiments, the single-stranded oligonucleotide is covalently conjugated to the 3′ end, the 5′ end, both the 3′ and the 5′ ends, or an internal nucleotide of the sense strand of the double-stranded oligonucleotide. In some embodiments, the single-stranded oligonucleotide is covalently conjugated to the 3′ end, the 5′ end, both the 3′ and the 5′ ends, or an internal nucleotide of the antisense strand of the double-stranded oligonucleotide. In some embodiments, the internal nucleotide in the sense or antisense strand of the double-stranded oligonucleotide is substituted by a linking component, wherein the single-stranded oligonucleotide is covalently conjugated with the linking component.

In some embodiments, more than one single-stranded oligonucleotides are covalently conjugated to the double-stranded oligonucleotide. In some embodiments, about 2-10 single-stranded oligonucleotides are covalently conjugated to the double-stranded oligonucleotide.

In some embodiments, more than one double-stranded oligonucleotides are covalently conjugated to the single-stranded oligonucleotides. In some embodiments, about 2-10 double-stranded oligonucleotides are covalently conjugated to the single-stranded oligonucleotides.

In some embodiments, the linking component conjugated with the nucleotide in the single-stranded oligonucleotide or the double-stranded oligonucleotide, or both the single-stranded oligonucleotide and the double-stranded oligonucleotide through phosphorothioate (PS) bond. In some embodiments, the substituted linking component conjugates with each or both adjacent nucleotides on the double-stranded oligonucleotide through phosphorothioate (PS) bond.

In some embodiments, the linking component comprises a direct bond, or an oxygen or sulfur atom, or a unit selected from the following group: NR1, C(O), C(O)O, C(O)NR1, SO, SO2, and SO2NH; where R1 is hydrogen, acyl, aliphatic or substituted aliphatic.

In some embodiments, the linking component is selected from the group consisting of: substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenvlheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenvlheterocyclylalkyl, alkenylheterocyclylalkenyl, alkenylheterocyclylalkynyl, alkynvlheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, wherein one or more methylenes are interrupted or terminated by O, S, S(O), SO2, N(R′)2, C(O), cleavable linking group, substituted or unsubstituted aryl, substituted or unsubstituted heteroaryl, and substituted or unsubstituted heterocyclic.

In some embodiments, the linking component is selected from one or more of an ethylene glycol chain, an alkyl chain, an alkenyl chain, an alkynyl chain, a peptide, carbohydrates, thiol linkage, a phosphodiester, a phosphorothioate, a phosphoramidate, an amide, a carbamate, a tetrazole linkage, and a benzimidazole linkage.

In some embodiments, the linking component is selected from the group consisting of.
a) L1 or S18 (spacer-18 linker) (1,1-bis(4-methoxyphenyl)-1-phenyl-2,5,8,11,14,17-hexaoxanonadecan-19-yl (2-cyanoethyl) diisopropylphosphoramidite),
b) L4 or C6 (spacer-C6 linker) (6-(bis(4-methoxyphenyl)(phenyl)methoxy)hexyl (2-cyanoethyl) diisopropylphosphoramidite);
c) L6 (1,1-bis(4-methoxyphenyl)-1-phenyl-2,5,8,11,14-pentaoxahexadecan-16-yl (2-cyanoethyl) diisopropylphosphoramidite);
d) L9 or S9 (spacer-9 linker) (2-(2-(2-(bis(4-methoxyphenyl)(phenyl)methoxy)ethoxy)ethoxy)ethyl (2-cyanoethyl) diisopropylphosphoramidite);
e) L10 or C3 (spacer-C3 linker) (3-(bis(4-methoxyphenyl)(phenyl)methoxy)propyl (2-cyanoethyl) diisopropylphosphoramidite);
f) L12(d spacer) ((2R,3S)-2-((bis(4-methoxyphenyl)(phenyl)methoxy)methyl)tetrahydrofuranpar-3-yl (2-cyanoethyl) diisopropylphosphoramidite);
g) L13 or C12 (spacer-C12 linker) (12-(bis(4-methoxyphenyl)(phenyl)methoxy)dodecyl (2-cyanoethyl) diisopropylphosphoramidite);
b) L14 (spacer-L14 linker) (((1r,4r)-4-((bis(4-methoxyphenyl)(phenyl)methoxy)methyl)cyclohexyl)methyl (2-cyanoethyl) diisopropylphosphoramidite);
i) L15 (spacer-L15 linker) (4-(2-(bis(4-methoxyphenyl)(phenyl)methoxy)ethyl)phenethyl (2-cyanoethyl) diisopropylphosphoramidite),
j) L16 (spacer-L16 linker) (2-(1-(2-(bis(4-methoxyphenyl)(phenyl)methoxy)ethyl)cyclohexyl)ethyl (2-cyanoethyl) diisopropylphosphoramidite);
k) C6x1 ((2S,3S,4S,5S)-2-((bis(4-methoxyphenyl)(phenyl)methoxy)methyl)-5-methoxy-4-(pent-4-yn-1-yloxy)tetrahydrofuran-3-yl (2-cyanoethyl) diisopropylphosphoramidite);
l) C6x2 ((2S,3S,4S,5S)-5-((bis(4-methoxyphenyl)(phenyl)methoxy)methyl)-2-methoxy-4-(pent-4-yn-1-yloxy)tetrahydrofuran-3-yl (2-cyanoethyl) diisopropylphosphoramidite),
m) C6x5 (2-((2-(bis(4-methoxyphenyl)(phenyl)methoxy)ethyl)(pent-4-yn-1-yl)amino)ethyl (2-cyanoethyl) diisopropylphosphoramidite); and
n) C6x7 ((9H-fluoren-9-yl)methyl (4-((2S,4R)-2-((bis(4-methoxyphenyl)(phenyl)methoxy)methyl)-4-((bis(diisopropylamino)phosphanyl)oxy)pyrrolidin-1-yl)-4-oxobutyl)carbamate).

In some embodiments, the oligonucleotide agent comprises a nucleotide sequence that is at least 90% identical to the nucleotide sequences selected from the group consisting of.
a) siSOD1M2-AC2(N22)-S1V3v-Qu5 (SEQ ID NO: 58) and an antisense strand having a nucleotide sequence of SEQ ID NO: 57 that has partial complementarity with the sense strand of siSOD1M2-AC2(N22)-S1V3v-Qu5 (SEQ ID NO: 58);
b) siSOD1M2-AC2(N15)-S1V3v-Qu5 (SEQ ID NO 60) and an antisense strand having a nucleotide sequence of SEQ ID NO: 57 that has partial complementarity with the sense strand of siSOD1M2-AC2(N15)-S1V3v-Qu5 (SEQ ID NO: 60);
c) siSOD1M2-AC2(N12)-S1V3v-Qu5 (SEQ ID NO: 62) and an antisense strand having a nucleotide sequence of SEQ ID NO: 57 that has partial complementarity with the sense strand of siSOD1M2-AC2(N12)-S1V3v-Qu5 (SEQ ID NO: 62); and
d) siSOD1M2-AC2(N6)-S1V3v-Qu5 (SEQ ID NO: 64) and an antisense strand having a nucleotide sequence of SEQ ID NO: 57 that has partial complementarity with the sense strand of siSOD1M2-AC2(N6)-S1V3v-Qu5 (SEQ ID NO 64).
e) The oligonucleotide agent of any one of claims 1-27, wherein the oligonucleotide agent comprises a nucleotide sequence that is at least 90% identical to the following nucleotide sequences:
f) siHTT-AC2-S1L1 (SEQ ID NO: 28) and an antisense strand having a nucleotide sequence of SEQ ID NO: 27 that has partial complementarity with the sense strand of siHTT-AC2-S1L1 (SEQ ID NO: 28).

In some embodiments, the oligonucleotide agent comprises a nucleotide sequence that is at least 90% identical to the nucleotide sequences selected from the group of
a) siApp-8-AC2(N18)-S1L1V3v (SEQ ID NO: 32) and an antisense strand having a nucleotide sequence of SEQ ID NO: 31 that has partial complementarity with the sense strand of siApp-8-AC2(N18)-S1L1V3v (SEQ ID NO: 32);
b) siApp-8-AC2(N15)-S1L1V3v (SEQ ID NO: 34) and an antisense strand having a nucleotide sequence of SEQ ID NO: 31 that has partial complementarity with the sense strand of siApp-8-AC2(N15)-S1L1V3v (SEQ ID NO: 34); and
c) siApp-8-AC2(N12)-S1L1V3v (SEQ ID NO: 36) and an antisense strand having a nucleotide sequence of SEQ ID NO: 31 that has partial complementarity with the sense strand of siApp-8-AC2(N12)-S1L1V3v (SEQ ID NO 36).

In some embodiments, the sense or antisense strand of the double-stranded oligonucleotide has a nucleotide sequence that is at least 90% identical to the nucleotide sequence selected from the group of: R6-04(20)-S1V1v(CM-4) (SEQ ID NO: 66) or R6-04(20)-S1V1v(CM-4) (SEQ ID NO: 67).

In some embodiments, the oligonucleotide agent comprises a nucleotide sequence that is at least 90% identical to the nucleotide sequences selected from the group of:
a) R6-04M1-AC2(18)-S1L1V3v (SEQ ID NO 68) and an antisense strand having a nucleotide sequence of SEQ ID NO: 67 that has partial complementarity with the sense strand of R6-04M1-AC2(18)-S1L1V3v (SEQ ID NO 68),
b) R6-04M1-AC2(16)-S1L1V3v (SEQ ID NO: 70) and an antisense strand having a nucleotide sequence of SEQ ID NO: 67 that has partial complementarity with the sense strand of R6-04M1-AC2(16)-S1L1V3v (SEQ ID NO: 70);
c) R6-04M1-AC2(15)-S1L1V3v (SEQ ID NO: 72) and an antisense strand having a nucleotide sequence of SEQ ID NO: 67 that has partial complementarity with the sense strand of R6-04M1-AC2(15)-S1L1V3v (SEQ ID NO: 72);
d) R6-04M1-AC2(14)-S1L1V3v (SEQ ID NO 74) and an antisense strand having a nucleotide sequence of SEQ ID NO: 67 that has partial complementarity with the sense strand of R6-04M1-AC2(14)-S1L V3v (SEQ ID NO 74),
e) R6-04M1-AC2(13)-S1L1V3v (SEQ ID NO: 76) and an antisense strand having a nucleotide sequence of SEQ ID NO: 67 that has partial complementarity with the sense strand of R6-04M1-AC2(13)-S1L1V3v (SEQ ID NO: 76);
f) R6-04M1-AC2(12)-S1L1V3v (SEQ ID NO: 78) and an antisense strand having a nucleotide sequence of SEQ ID NO: 67 that has partial complementarity with the sense strand of R6-04M1-AC2(12)-S1 L1V3v (SEQ ID NO: 78);
g) R6-04M1-AC2(11)-S1L1V3v (SEQ ID NO 80) and an antisense strand having a nucleotide sequence of SEQ ID NO: 67 that has partial complementarity with the sense strand of R6-04M1-AC2(11)-S1L1V3v (SEQ ID NO 80),
h) R6-04M1-AC2(10)-S1L1V3v (SEQ ID NO: 82) and an antisense strand having a nucleotide sequence of SEQ ID NO: 67 that has partial complementarity with the sense strand of R6-04M1-AC2(10)-S1L1V3v (SEQ ID NO: 82);
i) R6-04M1-AC2(9)-S1L1V3v (SEQ ID NO:84) and an antisense strand having a nucleotide sequence of SEQ ID NO: 67 that has partial complementarity with the sense strand of R6-04M1-AC2(9)-S1L1V3v (SEQ ID NO: 84); and
j) R6-04M1-AC2(8)-S1L1V3v (SEQ ID NO: 86) and an antisense strand having a nucleotide sequence of SEQ ID NO: 67 that has partial complementarity with the sense strand of R6-04M1-AC2(8)-S1L1V3v (SEQ ID NO: 86).

In some embodiments, the single-stranded oligonucleotide is conjugated to one or more conjugation groups. In some embodiments, the double-stranded oligonucleotide is conjugated to one or more conjugation groups. In some embodiments, the sense strand or the antisense strand of the double-stranded oligonucleotide is conjugated to one or more conjugation groups.

In some embodiments, the conjugation groups are selected from one or more of: a lipid, a fatty acid, a fluorophore, a ligand, a saccharide, a peptide, and an antibody. In some embodiments, the one or more conjugation groups is selected from: a cell-penetrating peptide, polyethylene glycol, an alkaloid, a tryptamine, a benzimidazole, a quinolone, an amino acid, a cholesterol, glucose and N-acetylgalactosamine.

In some embodiments, each of the sense strand and the antisense strand independently has a nucleotide length ranging from 15-35 nucleotides.

In some embodiments, the sense or antisense strand of the double-stranded oligonucleotide has a nucleotide sequence that is at least 90% identical to the nucleotide sequence is siApp-8-S1V1 (SEQ ID NO: 28) or siApp-8-S1V1 (SEQ ID NO: 27).

In some embodiments, the oligonucleotide agent comprises a small interfering RNA (siRNA), wherein the siRNA comprises a sense strand and an antisense strand to form a duplex structure, wherein the antisense strand comprises a nucleotide sequence comprising at least 10 contiguous nucleotides, with 0, 1, 2 or 3 mismatches, and having at least 85% nucleotide sequence complementarity or homology to a portion of the nucleotide sequence of SEQ ID NO: 895, wherein the oligonucleotide agent is capable of inhibiting expression of superoxide dismutase 1 (SOD1) in a cell.

In some embodiments, the sense strand of the double-stranded oligonucleotide has a nucleotide sequence that is at least 90% identical to the nucleotide sequence selected from the group consisting of: siSOD1-5 (SEQ ID NO: 357), siSOD1-8 (SEQ ID NO: 358), siSOD1-10 (SEQ ID NO: 359), siSOD1-11 (SEQ ID NO: 360), siSOD-17 (SEQ ID NO: 357), siSOD1-35 (SEQ ID NO: 362), and siSOD1-37 through siSOD1-447 (SEQ ID NOs: 363-624).

In some embodiments, the antisense strand of the double-stranded oligonucleotide has a nucleotide sequence that is at least 90% identical to the nucleotide sequence selected from the group consisting of: siSOD1-5 (SEQ ID NO: 626), siSOD1-8 (SEQ ID NO: 627), siSOD1-10 (SEQ ID NO: 628), siSOD1-11 (SEQ ID NO: 629), siSOD-17 (SEQ ID NO: 630), siSOD1-35 (SEQ ID NO: 631), and siSOD1-37- through siSOD1-447 (SEQ ID NOs: 632-893).

In some embodiments, the sense strand of the double-stranded oligonucleotide has a nucleotide sequence that is at least 90% identical to the nucleotide sequence selected from the group of: siSOD1-231-E (SEQ ID NO: 38); siSOD1-23 I-TT (SEQ ID NO: 40); siSOD1-231-M1 (SEQ ID NO: 42); siSOD1-231-S2 (SEQ ID NO: 44); siSOD1-388-E (SEQ ID NO: 46); siSOD1-388-TT (SEQ ID NO: 48), siSOD1-388-M1 (SEQ ID NO: 50), siSOD1-388-S2 (SEQ ID NO: 52); siSOD1M2-L1 (SEQ ID NO: 54); and siSOD1M2-S1V5 (SEQ ID NO. 56).

In some embodiments, the antisense strand of the double-stranded oligonucleotide has a nucleotide sequence that is at least 90% identical to the nucleotide sequence selected from the group of siSOD1-231-E (SEQ ID NO: 39), siSOD1-231-TT (SEQ ID NO: 41), siSOD1-231-M1 (SEQ ID NO: 43); siSOD1-231-S2 (SEQ ID NO: 45); siSOD1-388-E (SEQ ID NO 47), siSOD1-388-TT (SEQ ID NO: 49); siSOD1-388-M1 (SEQ ID NO: 51); siSOD1-388-S2 (SEQ ID NO: 53), siSOD1M2-L1 (SEQ ID NO: 47); and siSOD1M2-S1V1v-Qu5 (SEQ ID NO: 57).

In some embodiments, the sense strand of the siRNA has a nucleotide sequence that has at least 85% homology to the nucleotide sequence selected from the group consisting of: DS17-0001 (SEQ ID NO: 384), DS17-0002 (SEQ ID NO: 372), DS117-0003 (SEQ ID NO: 409), DS17-0004 (SEQ ID NO: 357), DS17-0005 (SEQ ID NO: 486), DS17-0029 (SEQ ID NO: 588), DS17-01N3 (SEQ ID NO: 912), DS17-02N3 (SEQ ID NO: 914), DS17-03N3 (SEQ ID NO: 916), DS17-04N3 (SEQ ID NO: 918), DS17-05N3 (SEQ ID NO: 920) and any of SEQ ID NOs: 976-1021.

In some embodiments, the antisense strand of the siRNA has a nucleotide sequence that has at least 85% homology to the nucleotide sequence selected from the group consisting of: DS17-0001 (SEQ ID NO: 653), DS17-0002 (SEQ ID NO: 641), DS17-0003 (SEQ ID NO: 678), DS17-0004 (SEQ ID NO: 626), DS17-0005 (SEQ ID NO: 755), DS17-0029 (SEQ ID NO: 857), DS17-01N3 (SEQ ID NO: 913), DS17-02N3 (SEQ ID NO: 915), DS17-03N3 (SEQ ID NO: 917), DS17-04N3 (SEQ ID NO: 919), DS17-05N3 (SEQ ID NO 921), and SEQ ID NOs: 1022-1067.

In some embodiments, the sense strand and the antisense strand of the siRNA have nucleotide sequences that are independently at least 85% homologous to the nucleotide sequence pairs selected from the following groups.

a)
(SEQ ID NO: 384 and SEQ ID NO: 653)
DS17-0001,
b)
(SEQ ID NO: 372 and SEQ ID NO: 641)
DS17-0002,
c)
(SEQ ID NO: 409 and SEQ ID NO: 678)
DS17-0003,
d)
(SEQ ID NO: 357 and SEQ ID NO: 626)
DS17-0004,
e)
(SEQ ID NO: 486 and SEQ ID NO: 755)
DS17-0005,
f)
(SEQ ID NO: 588 and SEQ ID NO: 857)
DS17-0029,
g)
(SEQ ID NO: 912 and SEQ ID NO: 913)
DS17-01N3,
h)
(SEQ ID NO: 914 and SEQ ID NO: 915)
DS17-02N3,
i)
(SEQ ID NO: 916 and SEQ ID NO: 917)
DS17-03N3,
j)
(SEQ ID NO: 918 and SEQ ID NO: 919)
DS17-04N3,
and
k)
(SEQ ID NO: 920 and SEQ ID NO: 921)
DS17-05N3.

In some embodiments, the sense strand and the antisense strand of the siRNA have nucleotide sequences that is independently at least 85% homologous to the nucleotide sequence pairs selected from the following groups.

a)
(SEQ ID NO: 922 and SEQ ID NO: 923)
DS17-01M3,
b)
(SEQ ID NO: 924 and SEQ ID NO: 925)
DS17-02M3,
c)
(SEQ ID NO: 926 and SEQ ID NO: 927)
DS17-03M3,
d)
(SEQ ID NO: 928 and SEQ ID NO: 929)
DS17-04M3,
and
e)
(SEQ ID NO: 930 and SEQ ID NO: 931)
DS17-05M3.

In some embodiments, the oligonucleotide agent comprising a siRNA and a non-targeting ACO, wherein the ACO comprises a nucleotide sequence that is at least 90%, at least 95%, or 100% identical to SEQ ID NO 954, and the oligonucleotide agent is capable of inhibiting the expression of superoxide dismutase 1 (SOD1) in a cell.

In some embodiments, the sense strand and the antisense strand of the siRNA have nucleotide sequences that independently share at least 85% homologous to the nucleotide sequence pairs selected from the following groups.

a)
(SEQ ID NO: 922 and SEQ ID NO: 923)
DS17-01M3,
b)
(SEQ ID NO: 924 and SEQ ID NO: 925)
DS17-02M3,
c)
(SEQ ID NO: 926 and SEQ ID NO: 927)
DS17-03M3,
d)
(SEQ ID NO: 928 and SEQ ID NO: 929)
DS17-04M3,
e)
(SEQ ID NO: 930 and SEQ ID NO: 931)
DS17-05M3,
f)
(SEQ ID NO: 932 and SEQ ID NO: 933)
DS17-01M3-AC1 (me14)-L9V3,
g)
(SEQ ID NO: 934 and SEQ ID NO: 935)
DS17-02M3-AC1 (me14)-L9V3,
h)
(SEQ ID NO: 936 and SEQ ID NO: 937)
DS17-03M3-AC1 (mel4)-L9V3,
i)
(SEQ ID NO: 938 and SEQ ID NO: 939)
DS17-04M3-AC1 (mel4)-L9V3,
j)
(SEQ ID NO: 940 and SEQ ID NO: 941)
DS17-05M3-AC1 (me14)-L9V3,
k)
(SEQ ID NO: 942 and SEQ ID NO: 47)
DS17-29M2-AC1 (me14)-L9V3,
l)
(SEQ ID NO: 932 and SEQ ID NO: 47)
DS17-01M3v-AC1 (me14)-L9V3,
m)
(SEQ ID NO: 934 and SEQ ID NO: 943)
DS17-02M3v-AC1 (mel4)-L9V3,
n)
(SEQ ID NO: 936 and SEQ ID NO: 944)
DS17-03M3v-AC1 (mel4)-L9V3,
o)
(SEQ ID NO: 938 and SEQ ID NO: 950)
DS17-04M3v-AC1 (mel4)-L9V3,
p)
(SEQ ID NO: 940 and SEQ ID NO: 951)
DS17-05M3v-AC1 (me14)-L9V3,
and
q)
(SEQ ID NO: 952 and SEQ ID NO: 939)
DS17-04M3-asSOD1-1-L9V3.

In some embodiments, the oligonucleotide agent comprises a non-targeting ACO conjugated sense strand of a siRNA and an antisense strand of the siRNA, wherein the non-targeting ACO conjugated sense strand comprises a linking component covalently conjugating the ACO and the sense strand, wherein the antisense strand comprises a nucleotide sequence that is at least 90%, at least 95% homology, or 100% identical to SEQ ID NO: 57. In some embodiments, the non-targeting ACO conjugated sense strand comprises a nucleotide sequence that is at least 90%, at least 95%, or 100% identical to a nucleotide sequences selected from the group consisting of SEQ ID NOs: 1197-1288 and SEQ ID NOs: 1291-1298.

In some embodiments, the linking component is selected from the linking component group listed in SEQ ID NOs: 1197-1288 in Table 28 and SEQ ID NOs: 1291-1298 in Table 30.

In some embodiments, the single-stranded oligonucleotide of the oligonucleotide agent improves the stability, bioavailability, biodistribution, and/or cellular uptake of the double-stranded oligonucleotide as compared to an oligonucleotide agent without the single-stranded oligonucleotide.

In some embodiments, the single-stranded oligonucleotide of the oligonucleotide agent increases the biodistribution of double-stranded oligonucleotide within one or more target tissues as compared to an oligonucleotide agent without the single-stranded oligonucleotide.

In some embodiments, the one or more target tissues is selected from tissues of brain, spinal cord, muscle, spleen, lung, heart, liver, bladder, and kidney. In some embodiments, the one or more target tissues is selected from the group consisting of: prefrontal cortex, cerebellum, and rest of brain, cervical, thoracic and lumbar in spinal cord; heart, forelimb, hindlimb, nape, and gluteus.

At least part of the present disclosure provides a siRNA comprising an oligonucleotide sequence having a length ranging from 16 to 35 consecutive nucleotides, wherein the oligonucleotide sequence comprises a nucleotide sequence having at least 75%, at least 80%, at least 85%, at least 90%, at least 95% or 100% homology or complementarity to an equal length portion of SEQ ID NO:59, wherein the siRNA inhibits the mRNA transcript of SOD1 gene by at least 80% as compared to the baseline of SOD1 mRNA level.

In certain embodiments, the nucleotide sequence has at least 75%, at least 80%, at least 85%, at least 90%, at least 95% or 100% homology or complementarity to an equal length portion of SEQ ID NO: 61 or 63, wherein the oligonucleotide agent inhibits the mRNA transcript of SOD1 gene by at least 80% as compared to the baseline of SOD1 mRNA level.

Certain embodiments of the present application relate to a hotspot in the 3′ UTR of the SOD1 gene, wherein the hotspot has a nucleic acid sequence selected from SEQ ID NO: 61 (H1) and SEQ ID NO: 63 (H2).

Certain embodiments of the present application relate to a siRNA target sequence in the mRNA transcript from the 3′-UTR of the SOD1 gene, wherein the target sequence in the mRNA transcript has at least 75%, at least 80%, at least 85%, at least 90%, at least 95% or 100% homology to a sequence selected from SEQ ID NOs:1068-1113.

Certain embodiments of the present application also relate to a siRNA target sequence in the mRNA transcript from the SOD1 gene, wherein the target sequence in the mRNA transcript has at least 75%, at least 80%, at least 85%, at least 90%, at least 95% or 100% homology to a sequence selected from SEQ ID NOs:88-355.

Certain embodiments of the present application also relate to a si RNA comprising a sense strand and an antisense strand, wherein the sense strand of the siRNA has a nucleotide sequence that is at least 85%, at least 90%, at least 95% or 100% homology to the nucleotide sequence selected from the group of SEQ ID NOs 976-1021, wherein the siRNA inhibits the mRNA transcript of SOD1 gene by at least 80% as compared to the baseline of SOD1 mRNA level.

The present disclosure further provides a siRNA comprising a sense strand and an antisense strand, wherein the antisense strand of the siRNA has a nucleotide sequence that is at least 85%, at least 90%, at least 95% or 100% homology to the nucleotide sequence selected from the group of SEQ ID NOs: 1022-1067, wherein the siRNA inhibits the mRNA transcript of SOD1 gene by at least 80% as compared to the baseline of SOD1 mRNA level.

In some embodiments, the sense strand and the antisense strand of the siRNA have nucleotide sequences that is independently at least 85%, at least 90%, at least 95% or 100% homology to the nucleotide sequence pairs selected from the group of siSOD1-547 through siSOD1-694 (sense strand of SEQ ID NOs: 976-1021, and antisense strand of SEQ ID NOs: 1022-1067, respectively, in Table 22).

At least part of the present disclosure also relates an antisense oligonucleotide (ASO) comprising an oligonucleotide sequence having a length ranging from 12 to 30 consecutive nucleotides, wherein the oligonucleotide sequence comprises a nucleotide sequence having at least 75%, at least 80%, at least 85%, at least 90%, at least 95% or 100% homology or complementarity to an equal length portion of SEQ ID NO:59, wherein the ASO inhibits the mRNA transcript of SOD1 gene by at least 60% as compared to the baseline of SOD1 mRNA level.

In some embodiments, the ASO has at least 75%, at least 80%, at least 85%, at least 90%, at least 95% or 100% complementarity to an equal length portion of SEQ ID NO: 65, wherein the ASO inhibits the mRNA transcript of SOD1 gene by at least 60% as compared to the baseline of SOD1 mRNA level.

Certain embodiments of the present application relate to a hotspot in the 3′ UTR of the SOD1 gene, wherein the hotspot has a nucleic acid sequence of SEQ ID NO: 65 (H3).

Certain embodiments of the present application relate to an ASO target sequence in the mRNA transcript from the 3′-UTR of the SOD1 gene, wherein the target sequence in the mRNA transcript has at least 75%, at least 80%, at least 85%, at least 90%, at least 95%, at least 97%, at least 99% or 100% homology to the nucleotide sequence selected from group consisting of SEQ ID NOs: 1114-1154.

Certain embodiments of the present application also relate to an ASO comprising a single-stranded oligonucleotide sequence that is at least 75%, at least 80%, at least 85%, at least 90%, at least 95%, at least 97%, at least 99% homology or 100% identical to the nucleotide sequence selected from group consisting of chemically modified SEQ ID NOs 1155-1195 and their unmodified naked sequences SEQ ID NOs: 1114-1154, wherein the ASO inhibits the mRNA transcript of SOD1 gene by at least 60% as compared to the baseline of SOD1 mRNA level.

Also provided here are vectors and cells comprising the oligonucleotide agents of the present disclosure. In some embodiments, the cell is a mammalian cell and is optionally a human cell. In some embodiments, the cell is a host cell. In some embodiments, the cell is in vitro. In some embodiments, the cell exists in a mammalian body.

Certain embodiments of the present application relate to a pharmaceutical composition comprising the oligonucleotide agent comprising (a) a double-stranded oligonucleotide comprising a sense strand and an antisense strand, wherein the antisense strand has complementarity to a target nucleic acid; and (b) a non-targeting single-stranded oligonucleotide, wherein the single-stranded oligonucleotide is 6-22 nucleotides in length, wherein the double-stranded oligonucleotide and the single-stranded oligonucleotide are covalently linked, with or without one or more linking components, to form the oligonucleotide agent. The target nucleic acids can be any target nucleic acid. The target nucleic acid includes, without limitation, a SOD1 gene, a HTT gene, an App gene, a SMN2 gene, etc.

In certain embodiments of the present application, the pharmaceutical composition comprises at least one pharmaceutically acceptable carrier selected from an aqueous carrier, liposome or LNP, polymer, micelle, colloid, metal nanoparticle, non-metallic nanoparticle, bioconjugates, and polypeptide.

In certain embodiments, the pharmaceutical composition decreases or silences the transcription of the SOD1 gene or SOD1 protein.

In certain embodiments, the pharmaceutical composition increases or activates the expression of the HTT, App or SMN2 gene or HTT, App or SMN2 protein.

Also provided herein are kits comprising the oligonucleotide agents or the pharmaceutical compositions of the present disclosure.

Certain embodiments relate to kits comprising a pharmaceutical composition of the present disclosure.

Certain embodiments relate to a method of decreasing or silencing the transcription of a SOD1 gene or protein, comprising administering to a subject a pharmaceutical composition of the present disclosure.

Certain embodiments relate to a method for treating or delaying the onset or progression of Amyotrophic lateral sclerosis (ALS) in a subject, the method comprising: administering to a subject a pharmaceutical composition of the present disclosure. In certain embodiments, the subject has sporadic ALS (sALS). In certain embodiments, the subject has familial ALS (fALS).

Certain embodiments of the present application relate to a method for increasing or activating expression of an HTT gene or huntington protein, comprising administering to a subject the pharmaceutical composition.

Certain embodiments of the present application relate to a method for treating or delaying the onset or progression of Huntington's disease in a subject, the method comprising: administering to the subject the pharmaceutical composition.

Certain embodiments of the present application relate to a method for treating or delaying the onset or progression spinal muscular atrophy (SMA) in a subject, the method comprising: administering to the subject the pharmaceutical composition.

Certain embodiments of the present application relate to a method for increasing or activating expression of an App gene or amyloid precursor protein (APP), comprising administering to a subject the pharmaceutical composition.

Certain embodiments of the present application relate to a method for treating or delaying the onset or progression of APP associated diseases including Cerebral Amyloid Angiopathy App-related (CAA-APP) and Alzheimer Disease (AD) in a subject, the method comprising: administering to the subject the pharmaceutical composition.

Certain embodiments of the present application relate to a method for increasing or activating expression of SMN2 gene, comprising administering to a subject the pharmaceutical composition.

Certain embodiments of the present application relate to a method for treating or delaying the onset or progression of spinal muscular atrophy (SMA) in a subject, the method comprising: administering to a subject a pharmaceutical composition of the present disclosure.

In certain embodiments, the pharmaceutical composition decreases or silences the expression of the SOD1 gene or protein.

In certain embodiments, the single-stranded oligonucleotide of the oligonucleotide agent improves the stability, bioavailability, biodistribution, and/or cellular uptake of the double-stranded oligonucleotide as compared to an oligonucleotide agent without the single-stranded oligonucleotide.

In certain embodiments, the single-stranded oligonucleotide of the oligonucleotide agent increases the biodistribution of double-stranded oligonucleotide within one or more target tissues as compared to an oligonucleotide agent without the single-stranded oligonucleotide.

In certain embodiments, the single-stranded oligonucleotide of the oligonucleotide agent increases the biodistribution of double-stranded oligonucleotide within two or more target cell types in a tissue as compared to an oligonucleotide agent without the single-stranded oligonucleotide. In certain embodiments, the one or more target tissues is selected from the tissues from brain, spinal cord, muscle, spleen, lung, heart, liver, bladder, and kidney. In certain embodiments, the one or more target tissues is selected from the group of: prefrontal cortex, cerebellum, and rest of brain; cervical, thoracic and lumbar in spinal cord; heart, forelimb, hindlimb, nape, and gluteus.

Certain embodiments of the present application relate to a use of the oligonucleotide agent of the present disclosure, in manufacturing a medicament for treating or delaying the onset or progression of Amyotrophic lateral sclerosis (ALS).

Certain embodiments of the present application relate to a use of the pharmaceutical composition of the present disclosure in manufacturing a medicament for treating or delaying the onset or progression of Amyotrophic lateral sclerosis (ALS). In certain embodiments, the ALS comprises sporadic ALS (sALS) and/or familial ALS (fALS).

Certain embodiments of the present application relate to the oligonucleotide agent of the present disclosure for use in treating or delaying the onset or progression of Amyotrophic lateral sclerosis (ALS), optionally, the ALS comprises sporadic ALS (sALS) and/or familial ALS (fALS).

Certain embodiments of the present application also relate to the pharmaceutical composition of the present disclosure for use in treating or delaying the onset or progression of Amyotrophic lateral sclerosis (ALS), optionally, the ALS comprises sporadic ALS (sALS) and/or familial ALS (fALS).

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are employed, and the accompanying drawings (also “figure” and “FIG.” herein), of which

FIG. 1A-1C is a visual illustration of example ODV structures. Double-stranded RNA (dsRNA) duplex (siRNA or saRNA) is linked to single-stranded accessory oligonucleotides (ACOs) at the 3′- (FIG. 1A), 5′-terminus (FIG. 1B) or an internal position (FIG. 1C) of their passenger (P) strand. Also labeled are the guide (G) strand and linker (L) bridging the ACO to each dsRNA.

FIG. 2 shows the ESI mass spectrograms and RP-HPLC profiles of purified ODV compounds siSOD1M2-AC2(N22)-S1V3v and siSOD1M2-AC2(N6)-S1V3v in comparison to siRNA duplexes with (siSOD1M2-L1) or without (siSOD1-388-E) linker. All duplexes were conjugated to Quasar 570 (Qu5) dye at the 5′-terminus of passenger strand. RP-HPLC analytics via reverse phase chromatography was performed using an acetonitrile gradient at a flow rate of 1.0 mL/min and detection wavelength set to 260 nm.

FIG. 3A-3B shows the in vivo knockdown activity of ODV-optimized siRNA (siHTT-AC2-S1L1) on Htt mRNA expression in the brain and spinal cord of C57BL/6 pup mice (PND4). siRNAs were injected via ICV administration at the indicated doses. Saline was injected as a negative control. siHTT-S1V1 lacked ODV composition and served as comparison for siHTT-AC2-S1L1 activity. Mice were sacrificed 3 days after treatment. Brain (FIG. 3A) and spinal cord (FIG. 3B) tissue samples were collected for analysis by RT-qPCR. Htt mRNA levels are mean values of two animals/group (n=2) relative to saline treatment after normalizing to Tbp reference levels.

FIG. 4 shows in vitro knockdown activity of App mRNA by ODV-siRNAs with ACOs at different lengths. NSC-34 cells were treated with 1 or 10 nM of the indicated siRNAs for 24 hours. Mock treatments were transfected in absence oligonucleotide. dsCon2 served as a non-specific control duplex. App mRNA levels were quantified by RT-qPCR using gene specific primer sets. Tbp was amplified as an internal reference. Shown are the mean expression values of App mRNA relative to Mock treatment after normalizing to Tbp reference levels.

FIG. 5 shows ACO) impact on in vivo knockdown activity of ODV-siRNAs in the CNS. All siRNAs were administered via ICV injection into C57BL/6 pup mice (PND4) at a 40 mg/kg dose. Saline was injected as a negative control. Mice were sacrificed 3 days after treatment. Brain and spinal cord tissue samples were collected for analysis by RT-qPCR. App mRNA levels are mean values of three animals/group (n=3) relative to saline treatment after normalizing to Tbp reference levels.

FIG. 6A-6B shows the upregulation of SMN2 mRNA transcripts via ODV-optimized saRNAs in human primary cells. An example saRNA duplex, termed R6-04(20)-S1V1v(CM-4), is an activator of human SMN2 gene expression. Several ODV variants of R6-04(20)-S1V1v(CM-4) were synthesized with ACOs at lengths ranging from 8 to 18 nucleotides. Primary human fibroblasts derived from SMA Type-2 (GM03813 cells) and SMA Type-1 (GM09677 cells) patients were transfected with each ODV-saRNA variant at 25 nM for 3 days. Mock treatments were transfected in absence oligonucleotide. dsCon2 served as a non-specific control duplex. Both full length (SMN2FL) and Δ7 (SUN2Δ7) splicing variants of SMN2 were quantified by RT-qPCR using isoform specific primer sets TBP was amplified as an internal reference. Shown are the mean expression values of SMN2FL and SMN237 transcripts relative to Mock treatment after normalizing to TBP reference levels in GM03813 (FIG. 6A) and GM09677 (FIG. 6B) cells.

FIG. 7A-7B shows the associated upregulation of SMN2 protein by ODV-saRNA in human primary cells. R6-04(20)-S1V1v(CM-4) and its ODV-saRNA variants were transfected at 25 nM for 3 days into GM03813 and GM09677 patient-derived cells. Mock treatments were transfected in absence oligonucleotide. dsCon2 served as a non-specific control duplex. Whole cell protein extracts were harvested for immunoblot analysis. Total SMN protein levels were detected using an indiscriminate monoclonal antibody that recognized both SMN1 and SMN2 gene product. Immunodetection of α/β-Tubulin served as a protein loading control. Scanning optical densitometry was used to quantify protein band intensity from example immunoblot images (not shown). Shown are relative changes in total SMN protein levels relative to Mock treatment after normalizing to α/β-Tubulin band intensity in GM03813 (FIG. 7A) and GM09677 (FIG. 7B) cells.

FIG. 8 shows the upregulation of SMN2 mRNA transcripts via ODV-saRNAs in primary mouse hepatocytes (PMH) carrying human SMN2 transgene R6-04(20)-S1V1v(CM-4) and its ODV-saRNA variants were transfected at 25 nM for 3 days in PMH cells derived from a liver harvested from the SMA-like mouse model. Mock treatments were transfected in absence oligonucleotide. dsCon2 served as a non-specific control duplex. Full length (SMN2FL) and Δ7 (SMN23Δ7) splicing variants of the human SMN2 transgene were quantified by RT-qPCR using isoform specific primer sets. Mouse Tbp (nTbp) was amplified as an internal reference Shown are the mean expression values of SMN2FL and SMN2Δ7 transcripts relative to Mock treatment after normalizing to mTbp reference levels FIG. 9 shows high throughput screening data comparing knockdown activity of 268 siRNAs on human SOD1 mRNA. HEK293A cells were transfected with each siRNA duplex at 0.1 and 10 nM for 24 hours Mock treatments were transfected in absence oligonucleotide SOD1 expression levels were quantified by RT-qPCR using gene specific primer sets. TBP was amplified as an internal reference. Shown are the mean expression values of SOD1 mRNA relative to Mock treatment after normalizing to TBP reference levels at both treatment concentrations.

FIG. 10 shows a general absence of cytotoxicity for the top 30 SOD1 si RNAs in HEK293 A cells. Dose escalating concentrations representing approximate multiples of IC₅₀ (half maximal inhibitory concentration) values were transfected into HEK293A cells. Mock treatments were transfected in absence oligonucleotide. Both mRNA expression levels and cytotoxicity were quantified for each siRNA at every dose by RT-qPCR and PI staining, respectively Plotted is optical density (OD) of PI staining in comparison to SOD1 knockdown relative to Mock treatments.

FIG. 11 shows SOD1 knockdown by 6 lead siRNAs in a human neuroblastoma cell line SH-SY5Y cells were treated with SOD1 siRNAs (i.e., siSOD1-63, siSOD1-47, siSOD1-104, siSOD1-5, siSOD1-231 and siSOD-388) at 1 and 10 nM for 24 hours. Mock treatments were transfected in absence oligonucleotide. dsCon2 served as a non-specific control duplex SOD1 mRNA levels were quantified by RT-qPCR using gene specific primer sets. TBP was amplified as an internal reference. Shown are the mean expression values of SOD1 relative to Mock treatment after normalizing to TBP.

FIG. 12A-12B shows Sod1 knockdown of 4 lead siRNAs targeting conserved sequence in mouse motor neuron-like cell lines. NSC-34 and N-2a cells were treated with SOD1 siRNAs (i.e., siSOD1-231, siSOD1-229, siSOD1-388, and siSOD1-387) at 1 and 10 nM for 24 hours Mock treatments were transfected in absence oligonucleotide. dsCon2 served as a non-specific control duplex. Mouse Sod1 levels were quantified by RT-qPCR using gene specific primer sets Mouse Tbp was amplified as an internal reference. Shown are the mean expression values of Sod1 transcript relative to Mock treatment after normalizing to nTbp reference levels in NSC-34 (FIG. 12A) and N-2a (FIG. 12B) cells.

FIG. 13A-13C shows impact of medicinal chemistry on siSOD1-231 and siSOD1-388 knockdown activity. Chemical modification patterns and duplex structures used to test knockdown activity are depicted in FIG. 13A using siSOD1-388 as model sequence. Modification symbols: Upper case in bold, 2′Ome; lower case, 2′F; chevron (\hat{ }), PS; T, deoxythymidine. Human 239A and mouse NE-4C (neuroepithelial) cells were treated with chemically modified versions of siSOD1-231 (i.e., siSOD1-231-E, siSOD1-231-TT, siSOD1-231-M1, or siSOD1-231-S2) and siSOD1-388 (i.e., siSOD1-388-E, siSOD1-388-TT, siSOD1-388-M1, or siSOD1-388-S2) at 1 and 10 nM for 24 hours. Mock treatments were transfected in absence oligonucleotide. dsCon2 served as a non-specific control duplex. Expression levels of SOD1/Sod1 was quantified by RT-qPCR using species-specific gene primer sets. TBP/Tbp was amplified as an internal reference. Shown are the mean expression values of SOD1/Sod1 transcript relative to Mock treatments after normalizing to internal reference levels in HEK293A (FIG. 13A) and NE-2C (FIG. 13B) cells FIG. 14 shows in vitro knockdown activity of Sod1 mRNA by ODV-optimized siSOD1-388-E [siSOD1M2-AC2(N15)-S1V3v-Qu5]. NSC-34 cells were treated with 0.1 or 1 nM of the indicated siRNAs for 3 days. Mock treatments were transfected in absence oligonucleotide. dsCon2 served as a non-specific control duplex. Sod1 mRNA levels were quantified by RT-qPCR using gene specific primer sets. Tbp was amplified as an internal reference Shown are the mean expression values of Sod1 mRNA relative to Mock treatment after normalizing to Tbp reference levels.

FIG. 15A-15C shows biodistribution and in vivo knockdown activity of siSOD1M2-AC2(N15)-S1V3v-Qu5 via ICV injection in the organs of pup mice (PND4). Qu5-labeled ODV-siRNA (siSOD1M2-AC2(N15)-S1V3v-Qu5) was administered via ICV injection into C57BL/6 pup mice (PND4) at a 40 mg/kg dose. Injection of a Qu5-labeled siRNA variant siSOD11M2-S1V1v-Qu5, which lacked any accessory oligonucleotide, served as a comparative control. Mice were sacrificed 3 days following treatment and whole organ fluorescence was quantified on an IVIS Imaging System using 520 nm excitation and 570 nm emission filters. FIG. 15A is an example IVIS image depicting siSOD1M2-AC2(N15)-S1V3v-Qu5 biodistribution via Qu5 signal in all major organs comparative to siSOD1M2-S1V1v-Qu5 following ICV injection. FIG. 15B quantifies fluorescence intensity of siSOD1M2-AC2(N15)-S1V3v-Qu5 emitted by each organ. Sod1 mRNA knockdown was quantified in organ tissue via RT-qPCR using gene specific primer sets. Tbp was amplified as an internal reference. FIG. 15C shows Sod1 knockdown in each organ relative to mRNA levels from a non-treated animal after normalizing to Tbp.

FIG. 16A-16B shows the biodistribution and in vivo knockdown activity of siSOD1M2-AC2(N12)-S1V3v-Qu5 via ICV injection in the organs of adult mice. Qu5-labeled ODV-siRNA (siSOD1M2-AC2(N12)-S1V3v-Qu5) was administered via bilateral ICV injection into adult C57BL/6 mice at a 10 mg/kg total dose. Injection of a Qu5-labeled siRNA variant siSOD1M2-S1V1v-Qu5, which lacked any accessory oligonucleotide, served as a comparative control. Mice were sacrificed 5 days after the injection and whole organ fluorescence was quantified on an IVIS Imaging System using 520 nm excitation and 570 nm emission filters. FIG. 16A quantifies Qu5 signal intensity of siSOD1M2-AC2(N12)-S1V3v-Qu5 emitted from each major organ comparative to siSOD1M2-S1V1v-Qu5 following ICV injection. Sod1 mRNA knockdown was quantified within tissues of the CNS and select peripheral tissues (i.e., muscle and kidney) via RT-qPCR using gene specific primer sets. Tbp was amplified as an internal reference. FIG. 16B shows mean knockdown levels of Sod1 in each of the indicated adult tissues relative to mRNA levels in saline treated animals after normalizing to Tbp.

FIG. 17 demonstrates ACO length impacts distribution of ODV-siRNA knockdown activity in CNS tissues in vivo. Sod1 ODV-siRNA variants with ACOs at lengths of either 22 nucleotides (siSOD1M2-AC2(N22)-S1V3v-Qu5) or 6 nucleotides (siSOD1M2-AC2(N6)-S1V3v-Qu5) were administered to adult C573L/6 mice via bilateral ICV injection at a 10 mg/kg total dose. Mice were sacrificed 10 days following treatment and Sod1 mRNA knockdown was quantified within different tissues of the CNS and liver via RT-qPCR using gene specific primer sets. Tbp was amplified as an internal reference. Mean expression levels from 2 animals (n=2) are shown in each of the indicated adult tissues relative to mRNA levels in non-treated animals after normalizing to Tbp.

FIG. 18 shows the inhibiting potency of 6 lead siRNAs on SOD1 mRNA level in SH-SY5Y human neuroblastoma cells. SH-SY5Y cells were treated with SOD1 siRNAs (i.e., DS17-0001, DS17-0002, DS17-0003, DS17-0004, DS17-0005 and DS17-0029) at 0.1 and 1 nM for 24 hours. Mock treatments were transfected in absence of oligonucleotide. dsCon2 served as a non-specific duplex control SOD1 mRNA levels were quantified by RT-qPCR using gene specific primer sets. TBP was amplified as an internal reference. Shown are the mean values of SOD1 mRNA relative to Mock treatment after normalizing to TBP.

FIG. 19 shows the inhibiting potency of 7 siRNAs on the SOD1 mRNA level in HEK293A cells. HEK293A cells were treated with DS17-04N3 siRNA and 6 prior art siRNAs (DS17-Vo149, DS17-Vo149(c), DS17-Vo153, and DS17-Vo153(c) from U.S. Pat. No. 10,570,395B2; DS17-Al289 and DS17-Al102 from WO2006066203A2) at 0.004, 0.016, 0.063, 0.250, 1.000 and 4.000 nM for 24 hours, respectively. Mock treatments were transfected in absence of oligonucleotide. dsCon2 served as a non-specific duplex control Mock and dsCon2 data are not shown. SOD1 mRNA levels were quantified by RT-qPCR using gene specific primer sets. TBP was amplified as an internal reference. Shown are the mean values of SOD1 mRNA relative to Mock treatment after normalizing to TBP.

FIG. 20A-20B show the inhibiting potency of 9 siRNAs on the SOD1 mRNA levels in HeLa and HEK293A cells. HeLa and HEK293A cells were treated with DS17-02N3 siRNA and 8 prior art siRNAs (DS17-Vo195&60 and DS17-Vo195 (D-2763) from US20170314028A1, and DS17-Al148, DS17-Al194, DS17-Al290, DS17-Al405, DS17-Al447 and DS17-Al600 from WO2006066203A2) at 0.004, 0.016, 0.063, 0.250, 1.000 and 4.000 nM for 24 hours. respectively FIG. 20A shows the SOD1 mRNA level in HeLa cells. FIG. 20B shows the SOD1 mRNA level in HEK293A cells. Mock treatments were transfected in absence of oligonucleotide. dsCon2 served as a non-specific duplex control. Mock and dsCon2 data are not shown. SOD1 mRNA levels were quantified by RT-qPCR using gene specific primer sets. TBP was amplified as an internal reference. Shown are the mean values of SOD1 relative to Mock treatment after normalizing to TBP.

FIG. 21A-21B show the inhibiting potency of 5 lead siRNAs with chemical modification on the SOD1 mRNA levels in T98G and HEK293A cells T98G and HEK293A cells were treated with SOD1 siRNAs (i.e., DS17-01M3, DS17-02M3, DS17-03M3, DS17-04M3 and DS17-05M3) at 0.002, 0.005, 0.015, 0.046, 0.137, 0.412, 1.235, 3.704, 11.111, 33.333 and 100 nM for 24 hours, respectively. FIG. 21A shows the SOD1 mRNA level in T98G cells. FIG. 21B shows the SOD1 mRNA level in HEK293A cells. Mock treatments were transfected in absence of oligonucleotide. dsCon2 served as a non-specific duplex control. Mock and dsCon2 data are not shown. SOD1 mRNA levels were quantified by RT-qPCR using gene specific primer sets. TBP was amplified as an internal reference. Shown are the mean values of SOD1 relative to Mock treatment after normalizing to TBP.

FIG. 22A-22B show the inhibiting potency of 4 lead ODV-siRNAs with chemical modification on the SOD1 mRNA level in HEK293A cells. Cells were treated with 4 SOD1 ODV-siRNAs (i.e., DS17-02M3-AC1(me14)-L9V3, DS17-03M3-AC1(me14)-L9V3, DS17-04M3-AC1(me14)-L9V3 and DS17-29M2-AC1(me14)-L9V3) at 0.00002, 0.00009, 0.00037, 0.0015, 0.0059, 0.023, 0.094, 0.375, 1.5 and 6 nM for 24 hours, respectively. FIG. 22A shows the SOD1 mRNA level in HEK293A cells. FIG. 22B shows the caspase 3/7 activity in HEK293A cells Mock treatments were transfected in absence of oligonucleotide. dsCon2 served as a non-specific duplex control. Mock and dsCon2 data are not shown. SOD1 mRNA levels were quantified by RT-qPCR using gene specific primer sets TBP was amplified as an internal reference. Shown are the mean values of SOD1 relative to Mock treatment after normalizing to TBP. Caspase 3/7 activity was detected using caspase 3/7 activity kit.

FIG. 23A-23B show the in vivo inhibiting potency of ODV-siRNA leads on the SOD1 mRNA level via ICV injection in CNS of adult SOD1^{G93A} mice. 3 ODV-siRNA s (i.e., DS17-01M3-AC1(me14)-L9V3, DS17-04M3-AC1(me14)-L9V3 and DS17-05M3-AC1(me14)-L9V3) were administered via unilateral ICV injection into adult SOD1^{G39A} mice on postnatal day (PND) 46 at a 0.4 mg total dose. An ODV-duplex dsCon2M3-AC1(me14)-L9V3 served as a non-specific duplex control, asSOD1-1 (Tofersen) was also injected at the same dose as a positive control. Mice were sacrificed 7 days after the treatment and SOD1 mRNA knockdown was quantified in different tissues of the CNS via RT-qPCR using gene specific primer sets. FIG. 23A shows the SOD1 mRNA level in different brain tissues. FIG. 23B shows the SOD1 mRNA level in different spinal cord tissues. Tbp was amplified as an internal reference. Mean mRNA levels of 4 animals (n=4) each treatment group are shown by indicated tissues relative to non-treated group after normalizing to Tbp.

FIG. 24A-24B show the latency and body weight change of adult SOD1^{G93A} mice after ICV injection with siRNA. ODV-siRNA (DS17-04M3-AC1(me14)-L9V3) was administered via unilateral ICV injection into adult SOD1^{G93A} mice on PND 46 at a 20 nmole total dose. FIG. 24A shows the efficacy by rotarod behavior test after PND 68. FIG. 24B shows the body weight fluctuation after the injection at PND 46 (normalized as day 0). Tofersen was injected at a 20 nmole dose as a positive control. Artificial CSF (aCSF) was injected as a non-treated negative control. Mean SOD1 mRNA levels of 4 animals (n=4) are shown in each treatment.

FIG. 25 shows the siRNA concentration in adult SOD1^{G93A} mouse brain after ICV injection. An ODV-siRNA (DS17-04M3-AC1(me14)-L9V3) was administered via unilateral ICV injection into adult SOD1^{G93A} mice on PND 46 at a 0.4 mg total dose. The mice were sacrificed at 1-, 7-, 28- and 56-days following treatment and the concentration of DS17-04M3-AC1(me14)-L9V3 was quantified in different brain tissues via stem-loop RT-qPCR using gene specific primer sets. Mean values of siRNA concentrations from 3 animals (n=3) are shown in each treatment.

FIG. 26 shows the in vivo inhibiting potency of an ODV-siRNA lead on the SOD1 mRNA levels in different CNS tissues via ICV injection in adult SOD1^{G93A} mice. An ODV-siRNA (DS17-04M3-AC1(me14)-L9V3) was administered on PND 46 days via unilateral ICV injection at 0.1-, 0.4-, 1- and 1.6-mg total dose, aCSF was injected as a non-treated negative control. Mice were sacrificed at 14 days following treatment and SOD1 mRNA knockdown was quantified in different CNS and liver tissues via RT-qPCR using gene specific primer sets. Tbp was amplified as an internal reference. Mean SOD1 mRNA levels of 1-3 animals (n=1˜3) are shown in each of the indicated tissues relative to mRNA levels in non-treated group after normalizing to Tbp.

FIG. 27 shows the in vitro inhibiting potency of ODV-siRNA leads on the SOD1 mRNA level in T98G cells. T98G cells were treated with 5 SOD1 ODV-siRNAs (i.e., DS17-01M3v-AC1(me14)-L9V3, DS17-02M3v-AC1(me14)-L9V3, DS117-03M3v-AC1(me14)-L9V3, DS17-04M3v-AC1(me14)-L9V3 and DS17-05M3v-AC1(me14)-L9V3) at 0.0003, 0.0011, 0.0044, 0.0176, 0.0703, 0.2813, 1.1250, 4.5 and 18 nM for 24 hours, respectively. Mock treatments were transfected in absence of oligonucleotide. dsCon2 served as a non-specific duplex control. Mock and dsCon2 data are not shown SOD1 mRNA levels were quantified by RT-qPCR using gene specific primer sets. TBP was amplified as an internal reference. Shown are the mean SOD1 mRNA values relative to Mock treatment after normalizing to TBP

FIG. 28 shows the in vivo inhibiting potency of ODV-siRNA leads on the SOD1 mRNA level via ICV injection in adult SOD1^{G93A} mice. 5 ODV-siRNA s (i.e., DS17-01M3v-AC1(me14)-L9V3, DS117-02M3v-AC1(me14)-L9V3, DS117-03M3v-AC1(me14)-L9V3, DS17-04M3v-AC1(me14)-L9V3 and DS17-05M3v-AC1(me14)-L9V3) were administered via unilateral ICV injection into adult SOD1^{G93A} mice on PND 46 at a 0.2 mg total dose, aCSF was injected as a non-treated negative control DS17-04M3(Scr)-AC1(me14)-L9V3 was served as a non-specific duplex control. Mice were sacrificed 14 days following treatment and SOD1 mRNA knockdown was quantified in different CNS and liver tissues via RT-qPCR using gene specific primer sets. Tbp was amplified as an internal reference. Mean SOD1 mRNA levels from 2˜4 animals (n=2˜4) are shown in each of the indicated tissues relative to mRNA levels in non-treated group after normalizing to Tbp.

FIG. 29A-29C show the in vivo inhibiting potency of different ODV-siRNA leads on the SOD1 mRNA level via ICV injection in adult SOD1^{G93A} mice. Two ODV-siRNAs (DS17-04M3-AC1(me14)-L9V3 and DS17-04M3v-AC1(me14)-L9V3) were administered on PND 46 via unilateral ICV injection, aCSF was injected as a non-treated negative control. FIG. 29A shows the SOD1 mRNA transcript level in different tissues at a low dose (0.2 mg) after treatment for 14 days. FIG. 29B shows the SOD1 mRNA transcript level in different tissues at a high dose (0.4 mg) after treatment for 14 days. FIG. 29C shows the SOD1 mRNA transcript level in different tissues at a high dose (0.4 mg) after treatment for 56 days. Mice were sacrificed at 14- and 56-days following treatment and SOD1 mRNA knockdown was quantified in different CNS and liver tissues via RT-qPCR using gene specific primer sets. Tbp was amplified as an internal reference Mean SOD1 mRNA levels from 2˜4 animals (n=2˜4) are shown in each of the indicated tissues relative to mRNA levels in non-treated group after normalizing to Tbp.

FIG. 30A-30G show the in vivo inhibiting potency of ODV-siRNA leads on the SOD) mRNA transcript level via ICV injection in adult SOD1^{G93A} mice ODV-siRNAs (DS17-04M3-AC1(me14)-L9V3 and DS17-04M3v-AC1(me14)-L9V3) were administered on PND 46 via unilateral ICV injection at a at 20 nmole total dose. Tofersen was injected at same dose as a positive control, aCSF was injected as a non-treated negative control FIG. 30A, FIG. 30B, FIG. 30C, FIG. 30D, FIG. 30E, FIG. 30F and FIG. 30G show the SOD1 mRNA levels in Brain-frontal cortex, Brain-cerebellum, Rest of brain, Spinal cord-cervical, Spinal cord-thoracic, Spinal cord-lumbar and liver tissues, respectively. Mice were sacrificed at 2- and 8-weeks following treatment and SOD1 mRNA knockdown was quantified in different CNS and liver tissues via RT-qPCR using gene specific primer sets. Tbp was amplified as an internal reference. Mean SOD1 mRNA levels of 4 animals (n=4) are shown in each of the indicated tissues relative to mRNA levels in non-treated group after normalizing to Tbp.

FIG. 31A-31C show the immunostimulatory activity of SOD1 ODV-siRNA in ICR mice. ICR mice were treated with DS17-04M3, AC1-L9V3 and DS17-04M3-AC1(me14)-L9V3 at 10.18 nmole (low dose) and 40.71 nmole (high dose) by SC injection, respectively. Treatment with saline alone served as a vehicle control to establish baseline levels. Mice were sacrificed 8 hours after treatment in serum were harvested to detect the IL-1β (FIG. 31A), IFN-γ (FIG. 31B) and TNF-α (FIG. 31C) protein levels by ELISA assay as described in ELISA assay of Materials and Methods.

FIG. 32A-32C show levels of ALT (32A), AST (32B) and CREA (32C) in ICR mouse after SC injection of DS17-04M3, AC1-L9V3 and DS17-04M3-AC1(me14)-L9v3. The indicated test compounds were administered via SC injection into ICR mice at 10.18 nmole and 40.71 nmole, respectively. Serum levels of ALT (FIG. 32A), AST (FIG. 32B) and CREA (FIG. 32C) was detected by using their specific detection kits as described in Materials and Methods FIG. 33A-333 show screening data comparing knockdown activity of 46 siRNAs targeting human SOD1 3′UTR HEK293A cells were transfected with each siRNA duplex at 0.1 and 1 nM for 24 hours. FIG. 33A shows SOD1 mRNA expression levels of 46 siRNAs sorted by location in HEK293A cells. FIG. 33B shows SOD1 mRNA levels of 46 si RNAs sorted by activity on 3′UTR in HEK293A cells. Mock treatments were transfected in absence of oligonucleotide. dsCon2 served as a non-specific duplex control. Mock and dsCon2 data are not shown. SOD1 mRNA levels were quantified by RT-qPCR using gene specific primer sets. TBP and HPRT were amplified as internal reference. Shown are the mean expression values of SOD1 mRNA relative to Mock treatment after normalizing to TBP and HPRT1 reference levels at both treatment concentrations. The value (y-axis) shows the fold changes in SOD1 mRNA expression levels by each of the siRNAs relative to Mock treatment after normalized to TBP and HPRT1 siRNAs are sorted on x-axis by their location on the 3′UTR from 540 bp and 700 bp downstream of SOD1 transcription start site (TSS). Locations of the 2 siRNA hotspot regions were marked as H1 to H2 in rectangular dotted boxes.

FIG. 34. shows the dose dependent characterization for the candidates of SOD1 in HEK293A cells. siRNAs were transfected into HEK293A cells in dose escalated concentrations representing approximate multiples of IC₅₀ (half maximal inhibitory concentration) values Mock treatments were transfected in absence of oligonucleotide (not shown). SOD1 mRNA expression levels were quantified by RT-qPCR using gene specific primer sets. TBP and HPRT1 were amplified as internal reference Shown are the mean expression values of SOD1 mRNA relative to Mock treatment after normalizing to TBP and HPRT1 reference levels at both treatment concentrations.

FIG. 35A-35B show screening data comparing knockdown activity of ASOs targeting human SOD1 3′UTR. HEK293A cells were transfected with 16 ASOs at 5 nM and 50 nM, and 33 ASOs at 3.125, 12.5, 50 and 200 nM for 24 hours. FIG. 35A show SOD1 mRNA levels of 16 ASOs in HEK293A cells FIG. 35B show SOD1 mRNA levels of 33 ASOs in HEK293A cells. Mock treatments were transfected in absence of oligonucleotide. dsCon2 served as a non-specific duplex control. Mock and dsCon2 data are not shown. SOD1 mRNA levels were quantified by RT-qPCR using gene specific primer sets TBP and HPRT1 were amplified as internal reference. Shown are the mean expression values of SOD1 mRNA relative to Mock treatment after normalizing to TBP and HPRT1 reference levels at both treatment concentrations. The value (y-axis, log 2) shows the fold changes in SOD1 mRNA expression levels by each of the ASOs relative to Mock treatment after normalized to IBP and HPRT1. ASOs are sorted on x-axis by their location on the 3′UTR from 544 bp and 576 bp downstream of SOD1 transcription start site (TSS). Locations of the 1 ASO hotspot region was marked as H3 in rectangular dotted boxes.

FIG. 36A-36C. show the dose-dependent characterization for the candidates of SOD1 ASOs in HEK293A cells. ASOs were transfected into HEK293A cells in dose escalated concentrations representing approximate multiples of IC50 (half maximal inhibitory concentration) values. Mock treatments were transfected in absence of oligonucleotide (not shown). FIG. 36A show the mRNA expression levels, FIG. 36B show the apoptosis and FIG. 36C show the cytotoxicity were quantified for each ASO at every dose by RT-qPCR, Caspase 3/7 kit (G8092, Promega) and CCK8 kit (CKO4, Dojindo, Japan) in HEK293A cells, respectively.

FIG. 37A-37B show screening data for knockdown activity and cytotoxicity of 90 ODV-siRNA designs by free uptake in PMH cells. The indicated ODV-siRNAs were added to PMH cell culture media at 0.1 μM and 1 μM final concentration. The cells were then incubated for 3 days. RD-12556 and RD-12559 served as duplex control and ODV control, respectively Mock was treated in absence of oligonucleotide and not shown. FIG. 37A shows Sod1 mRNA levels by each of the ODV-siRNAs in PMH cells. Sod1 mRNA levels were quantified by RT-qPCR using gene specific primer sets. Tbp and Hprt1 were amplified as internal reference. The value (y-axis) shows the mean expression values of Sod1 mRNA relative to Mock treatment after normalizing to Tbp and Hprt1 reference levels at both treatment concentrations. FIG. 37B shows cytotoxicity levels of 90 ODV-siRNAs in PMH cells by PI staining. A microplate reader system (Infinite M2000 Pro) was used to detect the optical density (OD) of PI staining at 535 nm excitation and 615 nm emission wavelengths. The value (y-axis) shows the mean values of PI staining by each of the ODV-siRNAs at both concentrations relative to Mock. Total 90 ODV-siRNAs are marked as (A) to (L) in 12 different rectangular dotted boxes. The rectangular dotted boxes stand for various design groups with different linkers (A), Palindromic AC1 sequence variants (B), varying numbers of PS modification (C), varying numbers of 2′Ome (D), varying ACO sizes (E), adenine-rich sequence composition (F), cytosine-rich sequence composition (G), guanine-rich sequence composition (H), uracil-rich sequence composition (1), purines-rich sequence composition (J), pyrimidines-rich sequence composition (K) and balanced purines/pyrimidine composition (L).

FIG. 38 shows the effect of various ODV linkers used in ODV-siRNAs, as indicated in group (A) of FIG. 37, on in vitro knockdown activity in PMH cells. Group A (Linker group) included 10 compounds (i.e., RD-12941, RD-12942, RD-12943, RD-12944, RD-12945, RD-12947, RD-12948, RD-12949, RD-12950 and RD-12951) RD-12556 and RD-12559 served as duplex control and ODV control, respectively. FIG. 38 shows the Sod1 mRNA levels in PMH cells after free uptake treatments of ODV-siRNAs at 0.1 μM and 1 μM for 3 days. Mock was treated in absence of oligonucleotide and not shown. Sod1 mRNA levels were quantified by RT-qPCR using gene specific primer sets. Tbp and Hprt1 were amplified as internal reference. The value (y-axis) shows the mean expression values of Sod1 mRNA relative to Mock treatment after normalizing to T bp and Hprt1 reference levels at both treatment concentrations.

FIG. 39 shows the effect of different palindromic AC1 sequence used in ODV-siRNAs, as indicated in group (B) of FIG. 37, on in vitro knockdown activity in PMH cells. Group 13 (palindromic AC1 sequence group) included 15 compounds containing sequence derivatives based on the exemplary ACO called AC1 (i.e., RD-12952, RD-12953, RD-12954, RD-12955, RD-12956, RD-12957, RD-12958, RD-12959, RD-12960, RD-12961, RD-12962, RD-12963, RD-12964, RD-12965, and RD-12966). RD-12556 and RD-12559 served as duplex control and ODV control, respectively FIG. 39 shows the Sod) mRNA levels in PMH cells after free uptake treatments of ODV-siRNAs at 0.1 μM and 1 μM for 3 days Mock was treated in absence of oligonucleotide and not shown. Sod) mRNA levels were quantified by RT-qPCR using gene specific primer sets. Tbp and Hprt1 were amplified as internal reference. The value (y-axis) shows the mean expression values of Sod1 mRNA relative to Mock treatment after normalizing to Tbp and Hprt1 reference levels at both treatment concentrations.

FIG. 40 shows the effect of PS modification used in ODV-siRNAs, as indicated in group (C) of FIG. 37, on in vitro knockdown activity in PMH cells Group C (PS modification group) included 6 compounds (i.e., RD-12967, RD-12968, RD-12969, RD-12970, RD-12971 and RD-12972) each with the same 14-nt ACO containing either 2, 4, 6, 8, 10 or 12 PS modifications, respectively RD-12556 and RD-12559 served as duplex control and ODV control, respectively. FIG. 40 shows the Sod1 mRNA levels in PMH cells after free uptake treatments of ODV-siRNAs at 0.1 μM and 1 μM for 3 days. Mock was treated in absence of oligonucleotide and not shown. Sod1 mRNA levels were quantified by RT-qPCR using gene specific primer sets. Tbp and Hprt1 were amplified as internal reference. The value (y-axis) shows the mean expression values of Sod1 mRNA relative to Mock treatment after normalizing to Tbp and Hprt1 reference levels at both treatment concentrations.

FIG. 41 shows the effect of 2′Ome modification used in ODV-siRNAs, as indicated in group (D) of FIG. 37, on in vitro knockdown activity in PMH cells. Group D (2′Ome modification group) included 7 compounds (i.e., RD-12973, RD-12974, RD-12975, RD-12976, RD-12977, RD-12978 and RD-12979) each with the same 14-nt ACO containing either 2, 4, 6, 8, 10 or 12 substitutions of 2′MOE chemistry for 2′Ome, respectively. RD-12556 and RD-12559 served as duplex control and ODV control, respectively. FIG. 41 shows the Sod) mRNA levels in PMH cells after free uptake treatments of ODV-siRNAs at 0.1 μM and 1 μM for 3 days. Mock was treated in absence of oligonucleotide and not shown. Sod1 mRNA levels were quantified by RT-qPCR using gene specific primer sets. Tbp and Hprt1 were amplified as internal reference. The value (y-axis) shows the mean expression values of Sod1 mRNA relative to Mock treatment after normalizing to Tbp and Hprt1 reference levels at both treatment concentrations.

FIG. 42 shows the effect of various ACO size used in ODV-siRNAs, as indicated in group (E) of FIG. 37, on in vitro knockdown activity in PMH cells. Group E (ACO size group) included 8 compounds (i.e., RD-12980, RD-12981, RD-12982, RD-12983, RD-12984, RD-12985, RD-12986 and RD-12987) derived by truncating the 14-nt ACO in RD-12559 to 13, 12, 11, 10, 9, 8, 7, or 6 nucleotides in length, respectively. RD-12556 and RD-12559 served as duplex control and ODV control, respectively FIG. 42 shows the Sod) mRNA levels in PMH cells after free uptake treatments of ODV-siRNAs at 0.1 μM and 1 μM for 3 days. Mock was treated in absence of oligonucleotide and not shown. Sod1 mRNA levels were quantified by RT-qPCR using gene specific primer sets. Tbp and Hprt1 were amplified as internal reference. The value (y-axis) shows the mean expression values of Sod1 mRNA relative to Mock treatment after normalizing to Tbp and Hprt1 reference levels at both treatment concentrations.

FIG. 43 shows the effect of Adenine rich used in ODV-siRNAs, as indicated in group (F) of FIG. 37, on in vitro knockdown activity in PMH cells. Group F (Adenine rich group) included 5 compounds (i.e., RD-12988, RD-12989, RD-12990, RD-12991, and RD-12992) each with a different 14-nt ACO sequence containing either 5, 6, 7, 8, or 9 total adenine nucleotides, respectively. RD-12556 and RD-12559 served as duplex control and ODV control, respectively. FIG. 43 shows the Sod1 mRNA levels in PMH cells after free uptake treatments of ODV-siRNAs at 0.1 μM and 1 μM for 3 days. Mock was treated in absence of oligonucleotide and not shown. Sod1 mRNA levels were quantified by RT-qPCR using gene specific primer sets. Tbp and Hprt1 were amplified as internal reference. The value (y-axis) shows the mean expression values of Sod1 mRNA relative to Mock treatment after normalizing to Tbp and Hprt1 reference levels at both treatment concentrations.

FIG. 44 shows the effect of Cytosine rich used in ODV-siRNAs, as indicated in group (G) of FIG. 37, on in vitro knockdown activity in PMH cells. Group G (Cytosine rich group) included 6 compounds (i.e., RD-12993, RD-12994, RD-12995, RD-12996, RD-12997, and RD-12997) each with a different 14-nt ACO sequence containing either 5, 6, 7, 8, 9, or 10 total cytosine nucleotides, respectively RD-12556 and RD-12559 served as duplex control and ODV control, respectively. FIG. 44 shows the Sod) mRNA levels in PMH cells after free uptake treatments of ODV-siRNAs at 0.1 μM and 1 μM for 3 days. Mock was treated in absence of oligonucleotide and not shown. Sod) mRNA levels were quantified by RT-qPCR using gene specific primer sets. Tbp and Hprt1 were amplified as internal reference. The value (y-axis) shows the mean expression values of Sod1 mRNA relative to Mock treatment after normalizing to Tbp and Hprt1 reference levels at both treatment concentrations.

FIG. 45 shows the effect of Guanine rich used in ODV-siRNAs, as indicated in group (H) of FIG. 37, on in vitro knockdown activity in PMH cells. Group H (Guanine rich group) included 5 compounds (i.e., RD-12999, RD-13000, RD-13001, RD-13002, and RD-13003) each with a different 14-nt ACO sequence containing either 5, 6, 7, 8, or 9 total guanine nucleotides, respectively. RD-12556 and RD-12559 served as duplex control and ODV control, respectively. FIG. 45 shows the Sod1 mRNA levels in PMH cells after free uptake treatments of ODV-siRNAs at 0.1 μM and 1 μM for 3 days. Mock was treated in absence of oligonucleotide and not shown. Sod1 mRNA levels were quantified by RT-qPCR using gene specific primer sets. Tbp and Hprt1 were amplified as internal reference. The value (y-axis) shows the mean expression values of Sod. mRNA relative to Mock treatment after normalizing to Tbp and Hprt1 reference levels at both treatment concentrations.

FIG. 46 shows the effect of Uracil rich used in ODV-siRNAs, as indicated in group (I) of FIG. 37, on in vitro knockdown activity in PMH cells. Group I (Uracil rich group) included 6 compounds (i.e., RD-13004, RD-13005, RD-13006, RD-13007, RD-13008, and RD-13009) each with a different 14-nt ACO sequence containing either 5, 6, 7, 8, 9, or 10 total uracil nucleotides, respectively RD-12556 and RD-12559 served as duplex control and ODV control, respectively. FIG. 46 shows the Sod1 mRNA levels in PMH cells after free uptake treatments of ODV-siRNAs at 0.1 μM and 1 μM for 3 days. Mock was treated in absence of oligonucleotide and not shown. Sod1 mRNA levels were quantified by RT-qPCR using gene specific primer sets. Tbp and Hprt1 were amplified as internal reference. The value (y-axis) shows the mean expression values of Sod1 mRNA relative to Mock treatment after normalizing to Tbp and Hprt1 reference levels at both treatment concentrations.

FIG. 47 shows the effect of Purine rich used in ODV-siRNAs, as indicated in group (J) of FIG. 37, on in vitro knockdown activity in PMH cells. Group J (Purine rich group) included 8 compounds each with a different 14-nt ACO sequence containing 9 purines (i.e., RD-13010, RD-13011, and RD-13012), 10 purines (i.e, RD-13013, RD-13014, and RD-13015), or 11 purines (i.e., RD-13016 and RD-13017) comprised of different amounts of adenosine and guanine. RD-12556 and RD-12559 served as duplex control and ODV control, respectively. FIG. 47 shows the Sod1 mRNA levels in PMH cells after free uptake treatments of ODV-siRNAs at 0.1 μM and 1 μM for 3 days. Mock was treated in absence of oligonucleotide and not shown. Sod1 mRNA levels were quantified by RT-qPCR using gene specific primer sets. Tbp and Hprt1 were amplified as internal reference. The value (y-axis) shows the mean expression values of Sod1 mRNA relative to Mock treatment after normalizing to Tbp and Hprt1 reference levels at both treatment concentrations.

FIG. 48 shows the effect of Pyrimidine rich used in ODV-siRNAs, as indicated in group (K) of FIG. 37, on in vitro knockdown activity in PMH cells. Group K (Pyrimidine rich group) included 8 compounds each with a different 14-nt ACO sequence containing 9 pyrimidines (i.e., RD-13018, RD-13019, and RD-13020), 10 pyrimidines (i.e., RD-13021 and RD-13022), 11 pyrimidines (i.e., RD-13023 and RD-13024), or 12 pyrimidines (i.e., RD-13025) comprised of different amounts of cytosine and uracil. FIG. 48 shows the Sod1 mRNA levels in PMH cells after free uptake treatments of ODV-siRNAs at 0.1 μM and 1 μM for 3 days. Mock was treated in absence of oligonucleotide and not shown. Sod1 mRNA levels were quantified by RT-qPCR using gene specific primer sets. Tbp and Hprt1 were amplified as internal reference. The value (y-axis) shows the mean expression values of Sod1 m RNA relative to Mock treatment after normalizing to Tbp and Hprt1 reference levels at both treatment concentrations.

FIG. 49 shows the effect of Balanced pur:pyr used in ODV-siRNAs, as indicated in group (L) of FIG. 37, on in vitro knockdown activity in PMH cells. Group L (Balanced pur:pyr group) included 6 compounds (i.e., RD-13026, RD-13027, RD-13028, RD-13029, RD-13030, and RD-13031) each with a different 14-nt ACO sequence containing a fixed 1:1 ratio of purines to pyrimidines, respectively FIG. 49 shows the Sod1 mRNA levels in PMH cells after free uptake treatments of ODV-siRNAs at 0.1 μM and 1 μM for 3 days. Mock was treated in absence of oligonucleotide and not shown. Sod) mRNA levels were quantified by RT-qPCR using gene specific primer sets. Tbp and Hprt1 were amplified as internal reference. The value (y-axis) shows the mean expression values of Sod1 mRNA relative to Mock treatment after normalizing to Tbp and Hprt1 reference levels at both treatment concentrations.

FIG. 50A-50C show the in vivo potency of ODV-siRNAs on knocking down Sod1 mRNA levels in lung tissue via intratracheal instillation (ITI) administration in adult C57BL/6J mice. The indicated oligonucleotides (i.e., RD-12401, RD-12402, RD-12403, RD-12557, RD-12929 and RD-12559) were administered via ITI at 0.6 m dose in FIG. 50A. The indicated oligonucleotides (RD-12556, RD-12929 and RD-12559) were administered via ITI at 0.1 mg (FIG. 50B) and 0.6 mg (FIG. 50C) dose. Saline was administrated as a non-treated negative control RD-12404 was a non-specific duplex used as negative control. Mice were sacrificed on day 7 and day 21 post-dosing and Sod1 mRNA knockdown was quantified in lung tissue via RT-qPCR using gene specific primer sets after RNA isolation and RT reaction. Tbp was amplified as an internal reference. Mean Sod1 mRNA levels in lung tissue of 2-7 animals (n=2-7) are shown relative to mRNA levels in non-treated group after normalizing to Tbp.

FIG. 51A-51B show the in vivo potency of ODV-siRNA on knocking down SOD1 mRNA levels in muscle tissue via intravenous (IV) and subcutaneous (SC) injection in adult SOD1^{G93A} mice. The indicated siRNA RD-12293 was administered via IV and SC injection at 20 mg/kg and 50 mg/kg, respectively. Saline was injected as a non-treated negative control. Mice were sacrificed at day 14 post-dosing and SOD1 mRNA levels were quantified in muscle and liver tissues via RT-qPCR using gene specific primer sets after RNA isolation and RT reaction. Tbp was amplified as an internal reference. Mean SOD1 mRNA levels in muscle tissue of 2˜4 animals (n=2˜4) are shown relative to mRNA levels in non-treated group after normalizing to Tbp.

FIG. 52 shows in vivo potency of different ODV-siRNA leads on knocking down Sod) mRNA expression via IV injection in adult C57BL/6J mice. The indicated ODV-siRNAs (i.e., RD-12559, RD-12556, RD-12967, RD-13180, RD-12941, RD-12942, RD-12952, RD-12982, RD-12983, RD-12979, RD-13015, RD-13006 and RD-12998) were administered via IV injection at 20 mg/kg. Saline was injected as a non-treated negative control. RD-12556 served as non-ODV duplex control. Mice were sacrificed at day 7 following treatment and Sod1 mRNA levels were quantified in different types of muscle tissue (i.e., Forelimb, Hindlimb, Nape and Gluteus) via RT-qPCR using gene specific primer sets after RNA isolation and RT reaction. Tbp was amplified as an internal reference for RNA loading. Mean Sod1 mRNA levels in muscle tissue of 3˜6 animals (n=3˜6) are shown relative to mRNA levels in non-treated group after normalizing to Tbp.

FIG. 53A-53C show in vivo potency of different ODV-siRNA leads on knocking down Sod) mRNA expression via ICV injection in adult C57BL/6J mice. The indicated siRNAs or ODV-siRNAs (i.e., RD-12559, RD-12556, RD-13334, RD-12967, RD-13180, RD-12941, RD-12942, RD-12952, RD-12982, RD-12983, RD-12979, RD-13015, RD-13006 and RD-12998) were administered via unilateral ICV injection. Saline was injected as a non-treated negative control. RD-12556 and RD-13334 served as non-ODV duplex control. FIG. 53A shows the Sod) mRNA transcript level in brain-frontal cortex, rest of brain (other than frontal cortex and cerebellum) and brain-cerebellum tissues at 0.2 mg. FIG. 53B shows the Sod1 mRNA transcript level in spinal cord-cervical, spinal cord-thoracic and spinal cord-lumbar tissues at 0.2 mg. FIG. 53C shows the Sod) mRNA transcript level in liver tissue at 0.2 mg. Mice were sacrificed at day 7 following treatment and Sod1 mRNA knockdown was quantified in CNS (i.e., brain-frontal cortex, rest of brain, brain-cerebellum, spinal cord-cervical, spinal cord-thoracic and spinal cord-lumbar) and select peripheral tissues (i.e., liver) via RT-qPCR using gene specific primer sets after RNA isolation and RT reaction. Tbp was amplified as an internal reference. Mean Sod) mRNA levels from 3˜6 animals (n=3˜6) are shown in each of the indicated tissues relative to mRNA levels in non-treated group after normalizing to Tbp.

FIG. 54A-54B show in vivo potency of different ODV-siRNA leads on knocking down Sod) mRNA expression via IV injection in adult C57BL/6J mice. The indicated siRNAs or ODV-siRNAs (i.e., RD-12559, RD-12556, RD-13334, RD-12967, RD-13180, RD-12941, RD-12942, RD-12952, RD-12982, RD-12983, RD-12979, RD-13015, RD-13006 and RD-12998) were administered via IV injection at 20 mg/kg. Saline was injected as a non-treated negative control RD-12556 and RD-13334 served as non-ODV duplex control FIG. 54A shows the Sod1 mRNA transcript level in heart, liver and spleen tissues at 20 mg/kg FIG. 54B shows the Sod1 mRNA transcript level in lung, kidney and bladder tissues at 20 mg/kg. Mice were sacrificed on day 7 following treatment and Sod1 mRNA knockdown was quantified in select peripheral tissues (i.e., heart, liver, spleen, lung, kidney and bladder) via RT-qPCR using gene specific primer sets after RNA isolation and RT reaction. Tbp was amplified as an internal reference. Mean Sod1 mRNA levels from 2˜6 animals (n=2˜6) are shown in each of the indicated tissues relative to mRNA levels in non-treated group after normalizing to Tbp.

FIG. 55A-55D show dose-dependent characterization for internal conjugated ODV (iODV)-siRNA in PMH cells. The indicated siRNAs or ODV-siRNAs (i.e., RD-12559, RD-12556 and RD-13351) were transfected into PMH cells using RNAiMAX in dose escalated concentrations representing approximate multiples of IC₅₀ values (FIG. 55A and FIG. 55B). The indicated siRNAs (i.e., RD-12559, RD-13180 and RD-13351) were added into PMH cell culture medium in dose escalated concentrations representing approximate multiples of IC₅₀ values (FIG. 55C and FIG. 55D). RD-12556 served as non-ODV duplex control. RD-12559 served as ODV-siRNA positive control. Mock treatments were transfected in absence of oligonucleotide (not shown). FIG. 55A and FIG. 55C show Sod1 mRNA levels and FIG. 55B and FIG. 55D show cell viability quantified for each oligonucleotide at every dose by RT-qPCR and CCK8 assay respectively.

FIG. 56 interprets some of the in vivo data of siSOD1 with ACO variants in Example 28. The mean average knockdown data (Mean % Reduction) in CNS tissues (brain and spinal cord, compared to liver) are listed in the table. ODV-siSOD1 variants that had possible better and worse retention with regards to local and periphery tissues knockdown activity are exemplified.

FIG. 57 shows the design of ACO sequence variants derived from the exemplary AC1. The variants include 15 sequences in which the siRNA, linker (L9), 2′MOE modification and PS backbone are fixed, but ACO sequence and length are modified relative to the AC1 sequence. The palindromes, deletions, purine/pyrimidine switches which may impact the delivery efficiency are exemplified.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Before the present invention described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the exemplary methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a sample” includes a plurality of such samples and reference to “the molecule” includes reference to one or more molecules and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention.

Further, the dates of publication provided may be different from the actual publication dates which may need to be independently confirmed.

### Definitions

The terms “Amyotrophic lateral sclerosis” or “ALS” include, but are not limited to, familial ALS (fALS), sporadic ALS (sALS), Lou Gehrig's disease, diseases associated with mutant genes Chromosome 9 Open Reading Frame 72 gene (C9orf72; 40%), superoxide dismutase 1 (SOD1; 20%), transactive response DNA-binding protein 43 (TDP43; 4%) and fused in sarcoma/translocated in liposarcoma (FUS/TLS; 4%).

The term “oligonucleotide agent” or “oligonucleotide” can be used interchangeably, and refers to polymers of nucleotides, and includes, but is not limited to, single-stranded or double-stranded nucleic acid molecules of DNA, RNA, or DNA/RNA hybrid, oligonucleotide strands containing regularly and irregularly alternating deoxyribosyl portions and ribosyl portions, as well as modified and naturally or unnaturally existing frameworks for such oligonucleotides. Specifically, the oligonucleotide agent for inhibiting mRNA transcript level of target gene described herein is a small inhibiting nucleic acid molecule (siRNA), an antisense oligonucleotide molecule (ASO), or an oligonucleotide delivery vehicle (ODV) conjugated siRNA molecule (siRNA-ACO). Also specifically, the oligonucleotide agent for activating transcription of target gene described herein is a small activating nucleic acid molecule (saRNA), or an oligonucleotide delivery vehicle (ODV) conjugated saRNA molecule (saRNA-ACO).

As used herein, the terms “subject” and “individual” are used interchangeably herein to mean any living organism that may be treated with agents of the present application. The term “patient” means a human subject or individual, including disclosure infants, children and adults.

A “therapeutically effective amount” of a composition is an amount sufficient to achieve a desired therapeutic effect, and therefore does not require cure or complete remission. In embodiments of the present application, therapeutic efficacy is an improvement in any of the disease indicators, and a therapeutically effective amount is sufficient to cause an improvement in a clinically significant condition/symptom in the treated individual. The phrases “therapeutically effective amount” and “effective amount” are used herein to mean an amount sufficient to reduce by at least about 15 percent, preferably by at least 50 percent, more preferably by at least 90 percent, or to increase at least about 50 percent, at least about 100 percent, at least about 200 percent, more preferable at least about 500 percent and most preferably prevent, a clinically significant deficit in the activity, function and response of the individual being treated.

The effective amount may vary depending on such factors as the size and weight of the subject, the type of illness, or the particular agents of the application. For example, the choice of the agent of the application could affect what constitutes an “effective amount.” One of ordinary skill in the art would be able to study the factors contained herein and make the determination regarding the effective amount of the agents of the application without undue experimentation.

The regime of administration may affect what constitutes an effective amount. The agent of the application can be administered to the subject either prior to or after the disease diagnosis or condition. Further, several divided dosages, as well as staggered dosages, can be administered daily or sequentially, or the dose can be continuously infused, or can be a bolus injection. Further, the dosages of the agent(s) of the application could be proportionally increased or decreased as indicated by the exigencies of the therapeutic or prophylactic situation.

The terms “treat.” “treated,” “treating”, or “treatment” as used herein have the meanings commonly understood in the medical arts, and therefore do not require cure or complete remission, and include any beneficial or desired clinical results. Non-limiting examples of such beneficial or desired clinical results are prolonging survival as compared to expected survival without treatment, reduced symptoms including one or more of the followings: weakness and atrophy of proximal skeletal muscles, inability to sit or walk independently, difficulties in swallowing, breathing, etc.

As used herein, “preventing” or “delaying” a disease refers to inhibiting the full development of a disease.

The term “biological sample” refers to any tissue, cell, fluid, or other material derived from an organism (e.g., human subject). In certain embodiments, the biological sample is serum or blood.

As used herein, the term “sequence identity” or “sequence homology” means that one oligonucleotide strand (sense or antisense) of, for example, an saRNA or siRNA has at least 80% similarity with a region on the coding strand or template strand of the promotor, or the sequence of a target gene.

In embodiments of the present application, one target gene is SOD1 By “target sequence” is meant a sequence fragment to which the sense strand or antisense oligonucleotide of the siRNA or saRNA is homologous or complementary. For example, in certain embodiments, a SOD1 siRNA is homologous or complementary to a target select sequence within human SOD1 transcript.

As used herein, the term “non-targeting” means that the referenced accessory oligonucleotide (ACO) which conjugates with the targeting oligonucleotide (e.g., siRNA, saRNA, and etc.) does not specifically complement to the target sequence which the targeting oligonucleotide functions, and/or that the referenced oligonucleotide (i.e., ACO) does not share the same target sequence which the targeting oligonucleotide (e.g., siRNA, saRNA, and etc.) specifically attends to function to. The targeting oligonucleotide disclosed herein is a nucleic acid sequence that specifically complements to the target sequence or the region thereof. In some embodiments, the term “non-targeting oligonucleotide” may comprise any referenced oligonucleotide except the “targeting sequence”. In some case, the “specifically complementary” may mean that the complementarity between the targeting oligonucleotide and the target sequence or the region thereof is at least about 95%. The non-targeting oligonucleotide (i.e., accessory oligonucleotide, or “ACO” used interchangeably) is not to elicit biological activity via any known mechanism, nor intended to elicit activities indicative of ASO (i.e., “mixmer” or “gapmer”) function onto a complementary nucleic acid sequence (i.e., mRNA) in a certain subject, an organ of the subject, a tissue of the subject, or a cell of the subject, when the oligonucleotide is administered. The non-targeting oligonucleotide (i.e., ACO) is to facilitate the introduction of the targeting oligonucleotide (e.g., siRNA, saRNA, and etc.) it conjugates into a certain subject, an organ of the subject, a tissue of the subject, a cell of the subject, or a cell nucleus of the subject, when the oligonucleotide conjugate is administered.

As used herein, the term “gapmer” refers to a short DNA antisense oligonucleotide (ASO) structure with modified RNA segments on both sides of the central DNA structure. In some embodiments, at least one of the modified RNA segments comprises one or more of modified nucleotides selected from locked nucleic acids (LNA), and 2′-OMe or 2′-F modified nucleotides to increase affinity to the target, increase nuclease resistance, reduce immunogenicity, and/or decrease toxicity. In some embodiments, a gapmer comprises at least one nucleotide modified with a phosphorothioate (PS) group. In some embodiments, the gamper is designed to hybridize to a target piece of RNA and silence the gene transcript through the induction of RNase H cleavage. As an example, the ASO drug “Toferson” is a gapmer that knockdowns SOD1 mRNA for treatment of ALS A possible example of a dual-action oligonucleotide (DAO) with a gapmer ASO disclosed in the present application could be “siSOD1-Toferson”.

As used herein, the term “mixmer” refers to an antisense oligonucleotide (ASO) characterized as a mixture of DNA and chemically modified nucleic acid analogs in structure. Optionally, a mixmer is composed of fully modified nucleotides or nucleic acid analogs. In some embodiments, a mixmer is designed to bind and mask complementary RNA sequence to sterically block proteins, factors, or other RNAs from interacting with targeted RNA. In some embodiments, a mixmers is designed to alter pre-mRNA splicing by displacing the spliceosome. In some embodiments, a mixmer is designed to bind and sequester microRNAs (miRNAs) in which it is adopt yet another name called an “antagomir” or an “anti-miR”.

As used herein, the terms “sense strand” or “passenger strand” are interchangeable. The sense strand of dsRNA (e.g., siRNA, saRNA) molecule can include, for example, a first nucleic acid strand of siRNA comprising a fragment of the mRNA sequence of a target gene.

As used herein, the terms “antisense strand” or “guide strand” are interchangeable. The antisense strand of an dsRNA molecule can include, for example, a second nucleic acid strand in a duplex of saRNA or siRNA that is complementary to the sense strand.

As used herein, the term “first oligonucleotide strand” can be a sense strand or an antisense strand. For example, the sense strand of a saRNA refers to an oligonucleotide strand having homology with the coding strand of the promoter DNA sequence of the target gene of the saRNA. The sense strand of a siRNA refers to an oligonucleotide strand having homology with the mRNA sequence of the target gene of the siRNA. The antisense strand refers to an oligonucleotide strand complementary with the sense strand in the dsRNA.

As used herein, the term “second oligonucleotide strand” can also be a sense strand or an antisense strand if the first oligonucleotide strand is a sense strand, the second oligonucleotide strand is an antisense strand; and if the first oligonucleotide strand is an antisense strand, the second oligonucleotide strand is a sense strand.

The term “promoter” as used herein refers to a nucleic acid sequence, which encodes no proteins and plays a regulatory role for the transcription of a protein-coding or RNA-coding nucleic acid sequence by associating with them spatially. Generally, a eukaryotic promoter contains 100 to 5,000 base pairs, although this length range is not intended to limit the term of “promoter” as used herein. Despite that the promoter sequence is generally located at the 5′ terminus of a protein-coding or RNA-coding sequence, it also exists in exon and intron sequences.

As used herein, the term “coding strand” refers to the DNA strand in the target gene that cannot be transcribed, the nucleotide sequence of which is identical to the sequence of the RNA produced by transcription (in RNA the T in DNA is replaced by U). The coding strand of the double-stranded DNA sequence of the target gene promoter described in the present disclosure refers to the promoter sequence on the same DNA strand as the DNA coding strand of the target gene.

As used herein, the term “template strand” refers to another strand of double-stranded DNA of a target gene that is complementary to the coding strand and that can be transcribed as a template into RNA that is complementary to the transcribed RNA base (A-U, G-C). During transcription, RNA polymerase binds to the template strand and moves along the 3′→5′ direction of the template strand, catalyzing RNA synthesis in the 5′→3′ direction. The template strand of the double-stranded DNA sequence of the target gene promoter described in the present disclosure refers to the promoter sequence on the same DNA strand as the DNA template strand of the target gene.

As used herein, the term “transcription start site” or TSS refers to a nucleotide that marks the initiation of transcription on the template strand of a gene. The transcription start site may be present on the template strand of the promoter region. A gene may have more than one transcription start site.

As used herein, the term “overhang” refers to an oligonucleotide strand end (5′ or 3′) with non-base paired nucleotide(s) resulting from another strand extending beyond one of the strands within the double stranded oligonucleotide. Single stranded regions extending beyond the 3′ and/or 5′ ends of the duplexes are referred to as overhangs. In certain embodiments, the overhang is from 0 to 6 nucleotides in length. It is understood that an overhang of 0 nucleotides means that there is no overhang.

As used herein, the terms “gene activation”, “activating gene expression”, “gene upregulation” and “upregulating gene expression” can be used interchangeably, and means an increase or upregulation in transcription, translation, expression or activity of a certain nucleic acid sequence as determined by measuring the transcription level, mRNA level, protein level, enzymatic activity, methylation state, chromatin state or configuration, translation level or the activity or state in a cell or biological system of a gene. These activities or states can be determined directly or indirectly. In addition, “gene activation” or “activating gene expression” refers to an increase in activity associated with a nucleic acid sequence, regardless the mechanism of such activation. For example, gene activation occurs at the transcriptional level to increase transcription into RNA and the RNA is translated into a protein, thereby increasing the expression of the protein.

As used herein, the terms “gene silencing”, “knockdown of gene expression”, “gene downregulation” and “downregulating gene expression” can be used interchangeably, and means a decrease or downregulation in transcription, translation, expression or activity of a certain nucleic acid sequence as determined by measuring the transcription level, mRNA level, protein level, enzymatic activity, methylation state, chromatin state or configuration, translation level or the activity or state in a cell or biological system of a gene. These activities or states can be determined directly or indirectly. In addition, “gene downregulation” or “downregulating gene expression” refers to a decrease in activity associated with a nucleic acid sequence, regardless the mechanism of such downregulation. For example, gene downregulation occurs at the transcriptional level to decrease or silence transcription into RNA and the RNA is not translated into a protein, thereby decreasing or silencing the expression of the protein.

As used herein, the terms “short interfering RNA”, “siRNA” and “silencing RNA” can be used interchangeably and refer to a ribonucleic acid molecule that can downregulate, knockdown, or silence target gene expression. It can be a double-stranded nucleic acid molecule. It interferes with the expression of specific genes with complementary nucleotide sequences by degrading mRNA after transcription, preventing translation siRNA binds to target mRNA mainly in the cytoplasm to down-regulate gene expression post-transcriptionally via the RNA interference (RNAi) mechanism. siRNAs may be designed to target a gene's mRNA sequence to silence its expression via the RNAi mechanism, such as SOD1, for maximizing treatment outcomes, e.g., for ALS patients. siRNAs are molecules having endogenous RNA bases or chemically modified nucleotides. The modifications do not abolish cellular activity, but rather impart increased stability and/or increased cellular potency Examples of chemical modifications include phosphorothioate groups, 2′-deoxynucleotide, 2′-OCH₃-containing ribonucleotides, 2′-F-ribonucleotides, 2′-methoxyethyl ribonucleotides, combinations thereof and the like. The siRNA can have varying lengths (e.g., 10-200 bps) and structures (e.g., hairpins, single/double strands, bulges, nicks/gaps, mismatches) and are processed in cells to provide active gene silencing. A double-stranded siRNA can have the same number of nucleotides on each strand (blunt ends) or asymmetric ends (overhangs). An overhang of 1-2 nucleotides, for example, can be present on the sense and/or the antisense strand, as well as present on the 5′- and/or the 3′-ends of a given strand. The length of the siRNA molecule is typically about 10 to about 60, about 10 to about 50, about 15 to about 30, about 17 to about 29, about 18 to about 28, about 19 to about 27, about 20 to about 26, about 21 to about 25, and about 22 to about 24 base pairs, and typically about 15, about 16, about 17, about 18, about 19, about 20, about 21, about 23, about 25, about 30, about 40, or about 50 base pairs. In addition, the terms “small interfering RNA”, “silencing RNA” and “siRNA” also contain nucleic acids other than the ribonucleotide, including, but not limited to, modified nucleotides or analogues.

As used herein, the terms “inhibition of gene expression” or “inhibiting gene expression” and “gene downregulation” or “down-regulating gene expression” can be used interchangeably, and mean an decrease in transcription, translation, expression or activity of a certain nucleic acid as determined by measuring the transcriptional level, mRNA level, protein level, enzymatic activity, methylation state, chromatin state or configuration, translation level or the activity or state in a cell or biological system of a gene. These activities or states can be determined directly or indirectly. In addition, “inhibition of gene expression”. “inhibiting gene expression”, “gene down-regulation” or “down-regulating gene expression” refers to a decrease in activity associated with a nucleic acid sequence, regardless of the mechanism of such activation. For example, inhibition of gene expression occurs at the transcriptional level to decrease transcription into RNA and the RNA is translated into a protein, thereby decreasing the expression of the protein.

As used herein, the terms “small activating RNA”, “saRNA” and “small activating ribonucleic acid” can be used interchangeably and refer to a ribonucleic acid molecule that can upregulate target gene expression. It can be a double-stranded nucleic acid molecule composed of a first nucleic acid strand containing a ribonucleotide sequence with sequence homology with the non-coding nucleic acid sequence (such as a promoter and an enhancer) of a target gene and a second nucleic acid strand containing a nucleotide sequence complementary with the first strand. The saRNA can also be comprised of a synthesized or vector-expressed single-stranded RNA molecule that prone to form a hairpin structure by two complementary regions within the molecule, wherein the first region contains a ribonucleotide sequence having sequence homology with the target sequence of a promoter of a gene, and a ribonucleotide sequence contained in the second region is complementary with the first region. The length of the duplex region of the saRNA molecule is typically about 10 to about 60, about 10 to about 50, about 10 to about 40, about 12 to about 30, about 14 to about 28, about 16 to about 26, about 18 to about 24, and about 20 to about 22 base pairs, and typically about 10, about 13, about 15, about 17, about 18, about 19, about 20, about 21, about 22, about 25, about 30, about 40, about 50, or about 60 base pairs. In addition, the terms “small activating RNA”, “saRNA” and “small activating ribonucleic acid” also contain nucleic acids other than the ribonucleotide, including, but not limited to, modified nucleotides or analogues.

As used herein, the term “ASO” and “antisense oligonucleotide” can be used interchangeably and refer to single-stranded oligonucleotides which binds to complementary mRNA to elicit RNase H-dependent knockdown or the mRNA or alter protein binding of the mRNA via steric hindrance.

As used herein, the term “hotspot” of siRNAs or ASOs refers to a nucleic acid region of at least 12 bp in length where functional siRNAs/ASOs are enriched, i.e., at least 80%, e.g., about 85%, about 90%, about 95% or about 100% of the siRNAs/ASOs designed to target this region is functional and potent of inhibiting in the mRNA transcript level of the target gene. A “hotspot” herein is defined by a nucleic acid region on the target sequence of the siRNAs/ASOs, where the 5′-ends of the functional siRNAs or ASOs are located. In a non-limiting example, a siRNA/ASO is designed according to the following criteria: (1) having a GC content between 35% and 70%, (2) with less than 5 consecutive identical nucleotides, (3) with 3 or less dinucleotide repeats; and (4) with 3 or less trinucleotide repeats. In a non-limiting example, each of the hotspot sequences disclosed herein comprises at least 4, at least 5, or at least 6 (5′-ends of) functional siRNAs or ASOs.

As used herein, the term “functional siRNA” refers to a siRNA, at a 1 nM treatment concentration free uptake by a cell, inhibiting the mRNA transcript level of its intended target gene by at least 80%, as compared to a baseline level of SOD1 mRNA. The term “non-functional siRNA” refers to a siRNA, at a 1 nM treatment concentration free uptake by a cell, not capable of inhibiting the mRNA transcript level by 80%, as compared to a baseline level of SOD1 mRNA.

As used herein, the term “functional ASO” refers to an ASO, at a 200 nM treatment concentration free uptake by a cell, inhibiting the mRNA transcript level of its intended target gene by at least 60%, as compared to a baseline level of SOD1 mRNA. The term “non-functional siRNA” refers to an ASO, at a 200 nM treatment concentration free uptake by a cell, not capable of inhibiting the mRNA transcript level by 60%, as compared to a baseline level of SOD1 mRNA.

As used herein, the term “isolated target site”, “target site” and “isolated polynucleotide” can be used interchangeably, and herein means a nucleic acid target site to which a siRNA has complementarity or hybridizes to. For example, an isolated nucleic acid sequence of a target site can include a nucleic acid sequence to which a region of siRNAs has complementarity or hybridize to.

As used herein, the term “complementary” refers to the capability of forming base pairs between two oligonucleotide strands. The base pairs are generally formed through hydrogen bonds between nucleotides in the antiparallel oligonucleotide strands. The bases of the complementary oligonucleotide strands can be paired in the Watson-Crick manner (such as A to T, A to U, and C to G) or in any other manner allowing the formation of a duplex (such as Hoogsteen or reverse Hoogsteen base pairing).

Complementarity includes complete complementarity and incomplete complementarity. “Complete complementarity” or “100% complementarity” means that each nucleotide from the first oligonucleotide strand can form a hydrogen bond with a nucleotide at a corresponding position in the second oligonucleotide strand in the double-stranded region of the siRNA molecule, with no base pair being “mispaired”. “Incomplete complementarity”, “partial complementarity”, or “mismatch” means that not all the nucleotide units of the two strands are bound with each other by hydrogen bonds. For example, for two oligonucleotide strands each of 20 nucleotides in length in the double-stranded region, if only two base pairs in this double-stranded region can be formed through hydrogen bonds, the oligonucleotide strands have a complementarity of 10%. In the same example, if 18 base pairs in this double-stranded region can be formed through hydrogen bonds, the oligonucleotide strands have a complementarity of 90%. Substantial complementarity refers to at least about 75%, about 79%, about 80%, about 85%, about 90%, about 95% or 99% complementarity.

As used herein, “3′ untranslated region” and “3′ UTR” can be used interchangeably, referring to a fragment in 3′ region of a messenger RNA (mRNA) to regulate mRNA-based processes, such as mRNA localization, mRNA stability, and translation. In addition, 3′ UTR may establish 3′ UTR-mediated protein-protein interactions (PPIs), and thus can transmit genetic information encoded in 3′ UTR to protein.

As used herein, “ODV” and “oligonucleotide delivery vehicle” are used interchangeably, which refer to an oligonucleotide molecule comprising a duplex or double-stranded RNA (e.g., siRNA or saRNA) and an ACO which is covalently linked to the duplex RNA via a linker as described in more detail below.

As used herein, “covalent linker”, “linker” and “linking component” are used interchangeably, which refer to a molecule for covalently joining two molecules, e.g., a single-stranded oligonucleotide (e.g. ACO) and a dsRNA (e.g. siRNA or saRNA), two dsRNAs, etc. As described in more detail below, the term can include, e.g., a nucleic acid linker, a peptide linker, and the like and also, includes disulfide linkers.

As used herein, the term “synthetic” refers to the manner in which oligonucleotides are synthesized, including any means capable of synthesizing or chemically modifying RNA, such as chemical synthesis, in vitro transcription, vector expression, and the like.

As used herein, the term “LNA” refers to a locked nucleic acid in which the 2′-oxygen and 4′-carbon atoms are joined by an extra bridge. As used herein, the term “BNA” refers to a 2′-O and 4′-aminoethylene bridged nucleic acid that can contain a five-membered or six-membered bridged structure with an N—O linkage. As used herein, the term “PNA” refers to a nucleic acid mimic with a pseudopeptide backbone composed of N-(2-aminoethyl) glycine units with the nucleobases attached to the glycine nitrogen via carbonyl methylene linkers.

As used herein, the upper cased “SOD1” or “SOD1 gene” refers to a gene.

As used herein, the term “SOD1 mRNA” refers to a message RNA (mRNA) generated from the expression of SOD1 gene, or the transcription of SOD1 gene.

As used herein, the terms “SOD1 protein” refers to a protein generated from the expression of SOD1 gene, or translation of the SOD1 mRNA.

Unless otherwise defined, all the technological and scientific terms used therein have the same meanings as those generally understood by those of ordinary skill in the art covering the present application.

### Overview

Aspects of the present application include an oligonucleotide agent comprising oligonucleotide-based delivery vehicle (ODV) to provide improvements in efficient targeting one or more genes associated with a disease or condition, and improvements in the delivery, chemistry, biodistribution, bioavailability, and other pharmacological properties without compromising oligonucleotide activity.

The present application is based on investigations related to compositions and methods that, a targeting oligonucleotide (siRNA, saRNA, etc.), in combination with an ACO, can activate/upregulate a gene expression and increase the amount of expression of full-length gene or protein, or knockout/silence a gene expression and decrease the amount of expression of full-length gene or protein, in order to improve therapeutic effects for genetic conditions.

The term “oligonucleotide-delivery vehicle (ODV)” refers to a structure by conjugating an “accessory oligonucleotide (ACO)” to a chemical entity or moiety, e.g., a duplex oligonucleotide, to facilitate the introduction of the molecule into or uptake by a cell, a tissue, or an organ of an individual.

The present inventors found that the ODV did not interfere with siRNA knockdown activity (Example 3) and saRNA-induced gene activation (Example 5). The present inventors also found that the length, nucleotide compositions, modifications (e.g., 2′-Ome), linking components, palindromes of the sequence, numbers of phosphorothioate (PS) backbone substitution of the ACO had effects on dsRNA activity in vivo. When administered in either brain or spinal cord tissue as compared to a dsRNA without an ODV, ODV-dsRNA shows an improved in vivo activity in the CNS via local injection.

Additional aspects of the present application include methods of treating Amyotrophic lateral sclerosis (ALS) by administering an effective amount of an oligonucleotide agent comprising a SOD1-targeting siRNA. The siRNA inhibits the expression of SOD1 gene through the RNAi silencing mechanism. The present inventors have developed SOD1 siRNAs with potent inhibitory effect, for use in the treatment of ALS.

Amyotrophic lateral sclerosis (ALS), also referred to as Lou Gehrig's disease, is an adult-onset, lethal, paralytic disorder caused by the degeneration of motor neurons. ALS is characterized by progressive, adult-onset degeneration of cranial, brainstem and spinal motor neurons, leading to death by respiratory failure within 3-5 years of diagnosis. ALS presents as a familial or a sporadic form, depending on whether or not there is a family history of the disease, with sporadic ALS (sALS) accounts for 90% of the ALS patients. The most common mutant genes account for ˜75% of ALS in the United States: Chromosome 9 Open Reading Frame 72 gene (C9orf72; 40%), superoxide dismutase 1 (SOD1; 20%), transactive response DNA-binding protein 43 (TDP43; 4%) and fused in sarcoma/translocated in liposarcoma (FUS/TLS; 4%). Mutations in the C9orf72 and SOD1 genes also account for ˜5-8% and ˜2-3% of apparently sALS, respectively. To date, most of the studies of gene silencing have been undertaken in the context of the SOD1 gene models; the high copy number SOD1^{G93A} transgenic mouse model is still a cornerstone of ALS research.

Up to now, only two disease-modifying agents, Riluzole and Edaravone, are approved by U.S. Food and Drug Administration (FDA) for ALS treatment. Riluzole inhibits glutamate release from presynaptic terminals and blocks the post-synaptic N-methyl-D-aspartate (NMDA) receptors, which have been shown to increase survival by three to six months on average. Edaravone is a free radical scavenger that lowers the neuronal damage, eliminating the lipid peroxide hydroxyl radicals and transfers the electrons to edaravone (the radical) to ameliorate the oxidative damage. The two drugs only modestly improve survival and disease progression and can't cure the ALS. To date, there is currently no effective therapy available for ALS and new therapies are needed to treat this disease.

Among the known genes underlining ALS, SOD1 gene still remains a major cause of fALS and has been considered to be an important ALS drug target. The human SOD1 gene is located on chromosome 21q22.11 and located from base pair 33,031,935 to base pair 33,041,241 with a genomic size of 9307 bp. SOD1 gene codes for the monomeric SOD1 protein (153 amino acids, molecular weight 16 kDa), and also encodes for the detoxifying copper/zinc binding SOD1 enzyme, which has been found to be localized mainly in the cytosol, as well as in the nucleus, peroxisomes, and mitochondria (Tafuri et al. 2015). The first description of the ALS disease dates back to at least 1824 by Charles Bell, however, SOD1 as the first risk gene of ALS was discovered in 1993. When the first SOD1 transgenic mouse model (SOD1^{G93A}) was established in 1994, indicating that the research on ALS entered a new era. All these lines of evidence indicate that SOD1 mutants cause disease most probably via a gain-of-function, and reducing its levels may be beneficial. The excessive oxidation of wild-type SOD1 induces toxic conformational changes SOD1 silencing significantly attenuated astrocyte-mediated toxicity towards motor neurons. Therefore, silencing SOD1 expression is an important strategy for the treatment of ALS.

### Oligonucleotide Agents

Aspects of the present application include an oligonucleotide agent that includes a single-stranded oligonucleotide (e.g., ACO) having at least 6 nucleotides in length and a targeting double-strand oligonucleotide that are covalently linked, wherein the single-stranded oligonucleotide is a non-targeting oligonucleotide.

Aspects of the present application include an oligonucleotide agent that includes a single-stranded oligonucleotide (e.g., ACO) having at least 6 nucleotides in length and a targeting double-strand oligonucleotide that are covalently linked, wherein at least one phosphodiester bond between two adjacent nucleotides in the single-stranded oligonucleotide sequence is substituted by a phosphorothioate (PS), mesyl phosphoramidate or boranophosphate bond.

Aspects of the present application include an oligonucleotide agent that includes a single-stranded oligonucleotide having at least 6 nucleotides in length and a targeting double-strand oligonucleotide that are covalently linked, wherein the single stranded-oligonucleotide comprises a palindrome sequence.

Aspects of the present application include an oligonucleotide agent that includes a single-stranded oligonucleotide having at least 6 nucleotides in length and a targeting double-strand oligonucleotide that are covalently linked, wherein at least about 14%, at least about 28%, at least about 42%, at least about 57%, at least about 71%, at least about 85%, at least about 92%, or about 100% of the nucleotides of the single-stranded oligonucleotide have a 2′-Ome modification.

Aspects of the present application include an oligonucleotide agent that includes a single-stranded oligonucleotide having at least 6 nucleotides in length and a targeting double-strand oligonucleotide that are covalently linked, wherein the single-stranded oligonucleotide comprises no more than 72% or no more than 64% of cytosines.

Aspects of the present application include an oligonucleotide agent that includes a single-stranded oligonucleotide having 6-22 nucleotides in length and a targeting double-strand oligonucleotide that are covalently linked, wherein the single-stranded oligonucleotide is capable of facilitating delivery of the double-stranded oligonucleotide in the central nervous system (CNS).

In some embodiments, the oligonucleotide agent comprises a double-stranded oligonucleotide, wherein the double-stranded oligonucleotide comprises a sense strand and an antisense strand, wherein the antisense strand has complementarity to a target nucleic acid; and a non-targeting single-stranded oligonucleotide, wherein the single-stranded oligonucleotide is 6-22 nucleotides in length. The double stranded oligonucleotide and the single-stranded oligonucleotide are covalently linked, with or without one or more linking components, to form the oligonucleotide agent.

In some embodiments, the sense strand of the double stranded targeting oligonucleotide is covalently linked to a non-targeting single-stranded oligonucleotide (NTO). In some embodiments, the antisense strand of the double stranded targeting oligonucleotide is covalently linked to the non-targeting single-stranded oligonucleotide. In certain embodiments, the NTO does not have complementarity to the target nucleic acid of the double stranded oligonucleotide. In certain embodiments, the NTO does not have complementarity to the target gene or target mRNA transcript of the double stranded oligonucleotide. In certain embodiments, the NTO does not have complementarity to a nucleic acid of a subject possessing the target nucleic acid. In some embodiments, the target nucleic acid is a mammalian nucleic acid, e.g., from human.

In some embodiments, the oligonucleotide agent has a compound formula of:

O₁-L-O₂ (Formula Ia) or

O₂-L-O₂ (Formula Ib), wherein:

O₁ is a double-stranded oligonucleotide comprising a sense strand and an antisense strand, wherein the antisense strand has complementarity to a target nucleic acid (e.g., mammalian target nucleic acid); O₂ is a non-targeting single-stranded oligonucleotide in some embodiments, the single-stranded oligonucleotide is at least 6 nucleotides in length. L is a linker for covalently linking the double stranded oligonucleotide and the single-stranded oligonucleotide.

In some embodiments, the oligonucleotide agent has a compound formula of.

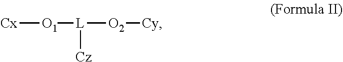

wherein

O₁ is a double stranded oligonucleotide comprising a sense strand and an antisense strand, wherein the antisense strand has complementarity to a target nucleic acid; O₂ is a non-targeting single-stranded oligonucleotide. In some embodiments, the single-stranded oligonucleotide is 6-22 nucleotides in length; L is a linker for covalently linking the double stranded oligonucleotide and the single-stranded oligonucleotide; and optional components Cx, Cy, and Cz, wherein Cx, Cy, and Cz are independently absence, or conjugation groups selected from one or more of a lipid, a fatty acid, a fluorophore, a ligand, a saccharide, a peptide, an antibody and any other commonly used conjugation groups. In some embodiments, the compound of Formula II comprises 1 conjugation group. In some embodiments, the compound of Formula II comprises 2 conjugation groups. In some embodiments, the compound of Formula II comprises 3 conjugation groups.

In some embodiments, the double-stranded oligonucleotide is a siRNA In some embodiments, the double-stranded oligonucleotide is a saRNA.

In some embodiments, the 5′ end, the 3′ end, or an internal nucleotide of the single-stranded oligonucleotide is conjugated to a linking component. In some embodiments, the internal nucleotide in the sense or antisense strand of the double-stranded oligonucleotide is substituted by a linking component, wherein the single-stranded oligonucleotide is covalently conjugated with the linking component. In some embodiments, the single-stranded oligonucleotide is covalently conjugated to the sense strand, the antisense strand, or both the sense and antisense strands of the second oligonucleotide by a linking component.

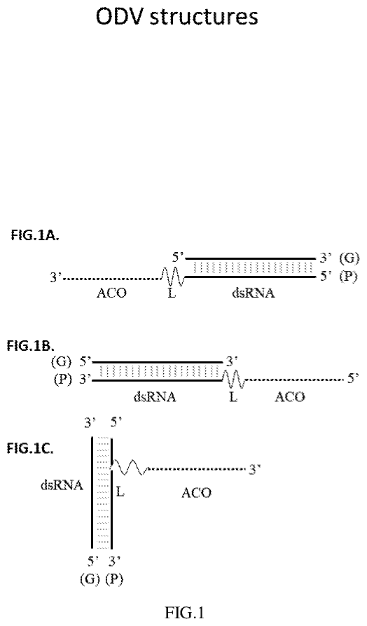

In some embodiments, the single-stranded oligonucleotide is covalently conjugated to the 3′ end, or the 5′ end, or both the 3′ and 5′ ends, or an internal nucleotide of the sense strand of the double-stranded oligonucleotide, as depicted in FIG. 1A, 1B or 1C. In some embodiments, the single-stranded oligonucleotide is covalently conjugated to the 3′ end, or the 5′ end, or both the 3′ and 5′ ends, or an internal nucleotide of the antisense strand of the double-stranded oligonucleotide.

In some embodiments, the double stranded targeting oligonucleotides and the single-stranded oligonucleotide that are covalently linked have a total nucleotide length ranging from 10 nucleotides to 500 nucleotides (e.g., 10 nucleotides to 100 nucleotides, 50 nucleotides to 100 nucleotides, 50 nucleotides to 200 nucleotides, 20 nucleotides to 100 nucleotides, 20 nucleotides to 200 nucleotides, 20 nucleotides to 300 nucleotides, 50 nucleotides to 300 nucleotides, 20 nucleotides to 80 nucleotides, 100 nucleotides to 300 nucleotides, 300 nucleotides to 500 nucleotides).

### Accessory Oligonucleotides (ACO)

Although some prior research proved that siRNAs capable of inhibiting SOD1 mRNA and decreasing SOD1 protein expression can be used to treat SOD1 protein-related diseases, e.g., for amyotrophic lateral sclerosis (ALS) patients, the inventors found there are two unsolved issues: 1) a lack of potency of SOD1 siRNA molecules and 2) a lack of efficient delivery method to deliver the siRNA molecule to cells of a target organ or tissue.

Surprisingly, the current invention found, when the dsRNA agent, e.g. an siRNA, is conjugated to a non-targeting single-stranded accessory oligonucleotide (ACO) as disclosed, bioavailability, biodistribution, and/or cellular uptake and in vivo potency of the dsRNA was significantly improved as compared to an oligonucleotide agent without the ACO. Especially in some in vivo examples in the present application, the ACO of the oligonucleotide agent increased the biodistribution of dsRNA within one, or two, or more target tissues as compared to an oligonucleotide agent without the ACO.

“Delivering into a cell,” when referring to a targeting double-strand oligonucleotide, e.g., a double-stranded RNA agent (dsRNA) such as a siRNA, a saRNA, or the like, means efficient uptake or absorption by the cell, as is understood by those skilled in the art. Absorption or uptake of an dsRNA can occur through unaided diffusive or active cellular processes, or by auxiliary agents or devices. The meaning of this term is not limited to cells in vitro; an dsRNA can also be “introduced into a cell,” wherein the cell is part of a living organism. In such an instance, introduction into the cell will include the delivery to the organism. For example, for in vivo delivery, dsRNA can be injected into a tissue site or administered systemically. In vitro introduction into a cell includes methods known in the art such as electroporation, free uptake, and lipofection. Further approaches are described herein below which is not known in the art.

The ACO of the oligonucleotide agent is a single-stranded oligonucleotide that is in favor of the oligonucleotide agent by its delivery properties. Therefore, the ACO does not target a nucleic acid in a subject which the dsRNA is targeting, or a “natural” nucleic acid from the subject, such as a target nucleic acid of the dsRNA. In some embodiments, the ACO) does not target a nucleic acid in a subject which the dsRNA is targeting. In some embodiments, the ACO does not have complementarity with a nucleic acid which the dsRNA is targeting. In some embodiments, the ACO does not have complementarity with a gene sequence or its mRNA transcript which the dsRNA is targeting.

In some embodiments, the length of the ACO comprises a nucleotide length ranging from 6 to 22 nucleotides, such as 6 nucleotides or more, 7 nucleotides or more, 8 nucleotides or more, 9 nucleotides or more, 10 nucleotides or more, 11 nucleotides or more, 12 nucleotides or more, 13 nucleotides or more, 14 nucleotides or more, 15 nucleotides or more, 16 nucleotides or more, 17 nucleotides or more, 18 nucleotides or more, 19 nucleotides or more, 20 nucleotides or more, 21 nucleotides or more, 22 nucleotides or more. In some embodiments, the length of the ACO is 6 to 18 contiguous oligonucleotides. In some embodiments, the length of the ACO is 10-14 nucleotides in length.

In some embodiments, the length of the ACO can modulate the activity and/or biodistribution of the oligonucleotide agent within the target tissue or cell of interest. For example, the present inventors found that shorter ACOs demonstrated activity of the oligonucleotide agent throughout the central nervous system, while longer ACOs demonstrated activity of the oligonucleotide agent only in particular regions of the brain, such as the cerebellum. In some embodiments, activity of the oligonucleotide agent throughout the entire central nervous system is desired. In some embodiments, activity of the oligonucleotide agent in particular regions of the brain is desired.

The ACO may comprise a sequence that is modified in order to further increase the capacity of the oligonucleotide agent to deliver a second oligonucleotide. In some embodiments, the sequence of the ACO comprises one or more of a chemically modified nucleotide, or at least one phosphodiester bond between two adjacent nucleotides in the oligonucleotide sequence is substituted by a phosphorothioate or boranophosphate bond. The chemical modifications of the ACO includes, without limitation, modification of the 2′-OH of the ribose in the nucleotide, the modification or the absence of a base in the nucleotide, the locking or bridging of a nucleic acid, a nucleotide being a peptide nucleic acid, a nucleotide being a deoxyribonucleotide (DNA), a nucleotide having a 5′-phosophate moiety, a nucleotide having a 5′-(E)-vinylphosphonate moiety, a nucleotide having a 5-methyl cytosine moiety, etc. Chemical modifications that may be found in ACOs are also further described below.

The ACO may comprise at least one phosphodiester bond substituted with phosphorothioate (PS) bond on the backbone of the nucleotide sequence. In some embodiments, the ACO comprises multiple PS backbone modifications, e.g., at least 2 PS, at least 3 PS, at least 4 PS, at least 5 PS, at least 6 PS, or greater than 6 PS backbone modifications. In some embodiments, the ACO comprises at least 10%, at least 20%, at least 30%, at least 40%, at least 50%, at least 60%, at least 70%, at least 80%, at least 90%, at least 95%, or 100% phosphodiester bond substituted with phosphorothioate (PS) bond on the backbone of the nucleotide sequence. As a non-limiting example, a 14 nt ACO may comprise 1, 2, 3, 4, 5, 6, 7, 8, 9, 10, 11, 12, or 13 PS backbone modifications. In some embodiments, the ACO having N nucleotides in length comprises N-1 PS modifications.

The ACO may also has a specific composition of nucleotides. In some embodiments, the ACO may have a certain percentage of adenines within the nucleotide sequence of the ACO. The ACO may have any percent composition of adenines. Percent compositions of adenines that find use in the present disclosure include without limitation, 30-35%, 35-40%, 40-45%, 45-50%, 50-55%, 55-60%, 60-65%, 65-70%, greater than 70%, etc. In a preferred embodiment, the percent composition of adenines is from about 35% to about 65%.

In some embodiments, the ACO may have a certain percentage of cytosines within the nucleotide sequence of the ACO. The ACO may have any percent composition of cytosines. Percent compositions of cytosines that find use in the present disclosure include without limitation, 30-35%, 35-40%, 40-45%, 45-50%, 50-55%, 55-60%, 60-65%, 65-70%, 70-75%, 75-80%, greater than 80%, etc. In a preferred embodiment, the percent composition of cytosines is from about 35% to about 72%.

In some embodiments, the ACO may have a certain percentage of guanosines within the nucleotide sequence of the ACO. The ACO may have any percent composition of guanosines. Percent compositions of guanosines that find use in the present disclosure include without limitation, 30-35%, 35-40%, 40-45%, 45-50%, 50-55%, 55-60%, 60-65%, 65-70%, greater than 70%, etc. In a preferred embodiment, the percent composition of guanosines is from about 35% to about 65%.

In some embodiments, the ACO may have a certain percentage of uracil within the nucleotide sequence of the ACO. The ACO may have any percent composition of uracil. Percent compositions of uracil that find use in the present disclosure include without limitation, 30-35%, 35-40%, 40-45%, 45-50%, 50-55%, 55-60%, 60-65%, 65-70%, 70-75%, 75-80%, greater than 80%, etc. In a preferred embodiment, the percent composition of uracil is from about 35% to about 72%.

In some embodiments, the ACO may have a certain percentage of purines within the nucleotide sequence of the ACO. The ACO may have any percent composition of purines. Percent compositions of purines that find use in the present disclosure include without limitation, 35-40%, 40-45%, 45-50%, 50-55%, 55-60%, 60-65%, 65-70%, 70-75%, 75-80%, greater than 80%, etc. In a preferred embodiment, the percent composition of purines is from about 65% to about 72%.

In some embodiments, the ACO may have a certain percentage of pyrimidines within the nucleotide sequence of the ACO. The ACO may have any percent composition of pyrimidines. Percent compositions of pyrimidines that find use in the present disclosure include without limitation, 35-40%, 40-45%, 45-50%, 50-55%, 55-60%, 60-65%, 65-70%, 70-75%, 75-86%, greater than 80%, etc. In a preferred embodiment, the percent composition of pyrimidines is from about 42% to about 58%.

In some embodiments, the ACO may have a specific combination of purines and pyrimidines. The specific combination of purines and pyrimidines may be any combination desired. Specific combinations of purines and pyrimidines that find use in the present disclosure include without limitation, about 30% purines about 70% pyrimidines, about 40% purines about 60% pyrimidines, about 50% purines about 50% pyrimidines, about 60% purines about 40% pyrimidines, about 70% purines about 30% pyrimidines, etc. In a preferred embodiment, the specific combination of purines and pyrimidines is about 42% purines and about 58% pyrimidines.

In some embodiments, the nucleotide sequence of the single-stranded oligonucleotide comprises at least 40%, at least 50%, at least 60%, or at least 70% of the nucleotides have 2′-Ome modification. In some embodiments, 70-100% of the nucleotide in ACO have 2′-Ome modification.

In some embodiments, the nucleotide sequence of the single-stranded oligonucleotide is a palindrome sequence. The term “palindromic sequence” used herein means a nucleic acid sequence in a double-stranded DNA or RNA molecule whereby reading in a certain direction (e.g., 5′ to 3′) on one strand is identical to the sequence in the same direction (e.g., 5′ to 3′) on the complementary strand. In some embodiments, the single-stranded oligonucleotide with a palindromic sequence enhances activity the double-stranded oligonucleotide or delivery of the oligonucleotide agent. In some embodiments, palindrome ACOs enhanced the protein binding capacity and knockdown activity of ODV-siRNAs on Sod1 mRNA. In some embodiments, the single-stranded oligonucleotide with palindrome sequence is selected from the group of SEQ ID NOs: 1300-1314.

Therefore, aspects of the present application further relate to an oligonucleotide agent capable of inhibiting the expression of superoxide dismutase 1 (SOD1) comprising a small interfering RNA (siRNA), and an ACO.

In some embodiments, the oligonucleotide agent comprising one or more conjugated ACOs to enhance the biodistribution of the oligonucleotide agent in particular tissues of the oligonucleotide agent, and increase permeability of the oligonucleotide agent and passage through membranes, such as the blood brain barrier.

In some embodiments, the ACO is an oligonucleotide comprising a 5′ end and a 3′ end.

In some embodiments, the dsRNA and the ACO are covalently linked, with or without one or more linking components, to form the oligonucleotide agent.

In another aspect of the present application, an oligonucleotide agent comprising a siRNA and a non-targeting ACO is provided. The ACO comprises a single-stranded oligonucleotide sequence comprising a nucleotide sequence that is at least 60% homology (e.g., at least 65%, at least 70%, at least 75%, at least 80%, at least 85%, at least 90%, at least 95%, at least 97%, at least 99% homology or 100% identical) to the nucleotide sequence selected from SEQ ID NOs: 953-954. In some embodiments, wherein the ACO comprises a nucleotide sequence that is at least 90% identical to the nucleotide sequence selected from SEQ ID NOs: 953-954.

In some embodiments, the oligonucleotide agent comprises a single-stranded oligonucleotide (ACO) tethered to a dsRNA. In some embodiments, the dsRNA is a natural nucleic acid. In some embodiments, the natural nucleic acid is a target nucleic acid. In certain embodiments, the natural nucleic acid is an intracellular nucleic acid.

In some embodiments, the ACO is a non-targeting oligonucleotide (NTO). In some embodiments, the ACO is a synthetic non-targeting oligonucleotide. In some embodiments, the ACO is a random non-targeting oligonucleotide. In some embodiments, the ACO comprises RNA, DNA, BNA, LNA PNA, or combinations thereof.

In some embodiments, the ACO interacts with one or more of: proteins in the plasma membrane, plasma proteins, peptides, ligands, lipids, fatty acids, saccharides, proteoglycan and zwitterionic phosphocholines. Such interaction of the ACO provides for increased biodistribution and enrichment of the targeting double stranded oligonucleotide of the oligonucleotide agent for local delivery to various target issues and cells of interest. Additionally, such interaction reduces or eliminates cytotoxicity of the oligonucleotide agent, confirming strong ‘on-target’ activity without overt effects on cell viability.

In certain embodiments, the protein interacting with ACO is selected from one or more of: serum albumin, IgG, Apolipoprotein A-1, Apolipoprotein A-II, Complement factor C3, Transferrin, α-1 Antitrypsin, Haptoglobin, Hemopexin, Fibrinogen, α-2-Macroglobulin, Prealbumin/TTR, Antithrombin III, α-1-Antichymotrypsin, β-2-Glycoprotein, Ceruloplasmin, α-1 Acid glycoprotein, Complement component C1q, Complement factor C4, Histidine-rich glycoprotein, Plasminogen, Fibronectin, ApoB100, Factor H, Apolipoprotein E. and Factor V.

In yet other embodiments, the protein interacting with ACO is selected from one or more of: ASGPR, EGFR, LDLR, M6PR, TLR, Stabilin, SRB, Nucleolin, AP2M1, EEA1, Rab5C, Rab7a, STX5, P115, COPH, M6PR, GCC2, ANXA2, TCP1, ALIX, TSGI01, VPS28, GLP-1, and HSP-90.

In certain embodiments, the interaction of the ACO is through direct binding or mediated by one or more conjugated ligands which is covalently linked to the ACO or the double stranded oligonucleotide, or both. In some embodiments, the one or more conjugated ligands comprise a lipid, a fatty acid, a fluorophore, a saccharide, a peptide, an antibody and any other commonly used conjugation ligands.

In certain embodiments, the conjugating ligands is selected from one or more of a cell-penetrating peptide, polyethylene glycol, an alkaloid, a tryptamine, a benzimidazole, a quinolone, an amino acid, a cholesterol, glucose and N-acetylgalactosamine. In certain embodiments, the one or more conjugation ligands is a fatty acid.

In some embodiments, the oligonucleotide agent comprising the one or more conjugation ligands enhances the biodistribution of the oligonucleotide agent in particular tissues, reduce or eliminate cytotoxicity of the oligonucleotide agent, and increase permeability of the oligonucleotide agent and passage through membranes, such as the blood brain barrier.

In some embodiments, the ACO comprises a single-stranded oligonucleotide sequence comprising a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequence selected from SEQ ID NOs: 1-22.

In some embodiments, the oligonucleotide agent comprises an ACO. In certain embodiments, the ACO comprises a nucleotide sequence that is at least 60% (e.g., at least 65%, at least 70%, at least 75%, at least 80%, at least 85%, at least 90%, at least 95%, at least 97%, at least 99% or 100%) identical to a nucleotide sequence selected from the group of AC2(N22) (SEQ ID NO: 1), AC2(N20) (SEQ ID NO: 2), AC2(N18) (SEQ ID NO: 3), AC2(N16) (SEQ ID NO 4), AC2(N15) (SEQ ID NO: 5), AC2(N14) (SEQ ID NO 6), AC2(N12) (SEQ ID NO. 7) AC2(N12) (SEQ ID NO: 7), AC2(N10) (SEQ ID NO: 8), AC2(N8) (SEQ ID NO: 9), AC2(N6) (SEQ ID NO: 10), AC2(22) (SEQ ID NO: 11), AC2(18) (SEQ ID NO: 12), AC2(16) (SEQ ID NO: 13), AC2(15) (SEQ ID NO: 14), AC2(14) (SEQ ID NO: 15), AC2(13) (SEQ ID NO: 16), AC2(12) (SEQ ID NO: 17), AC2(11) (SEQ ID NO: 18), AC2(10) (SEQ ID NO: 19), AC2(9) (SEQ ID NO: 20), AC2(8) (SEQ ID NO: 21) and AC2(6) (SEQ ID NO: 22).

In certain embodiments, the ACO comprises a chemically modified nucleotide sequence that is at least 80% (e.g., at least 85%, at least 90%, at least 95%, at least 97%, at least 99% or 100%) identical to a nucleotide sequence selected from the group of SEQ ID NOs: 1299-1379 In certain embodiments, the ACO has a chemically modified nucleotide sequence selected from the group of SEQ ID NOs: 1299-1379. In certain embodiments, the ACO has a chemically modified nucleotide sequence and a linker selected from the group of SEQ ID NOs: 1299-1379. In certain embodiments, the ACO has a chemically modified nucleotide sequence that is with 0, 1, 2 or 3 chemical modification differences to a nucleotide sequence selected from the group of SEQ ID NOs: 1299-1379.

In some embodiments, the oligonucleotide agent of the present application comprises more than one ACO, for example, 2, 3, 4, 5, 6, 7, 9, 10 ACOs, covalently linked to a dsRNA, with or without one or more linkers in between the ACOs and dsRNA. The amount of ACO, upon need, can vary from 2 to 10, or 2 to 100, or 2 to 1,000, or 2 to 10,000, linked to dsRNA via a multivalent linker, for example, a polymeric linker, in branch or liner form. In some embodiments, multiple ACOs are covalently linked to 2 or more dsRNAs, for example, 2, 3, 4, 5, 6, 7, 9, 10 or more dsRNAs, including saRNAs and/or siRNAs, in one agent.

In some embodiments, the oligonucleotide agent of the present application comprises one ACO and multiple dsRNAs, for example, 2, 3, 4, 5, 6, 7, 9, 10 dsRNAs, linked with or without one or more linkers in between the ACO and dsRNAs. The amount of dsRNA, including saRNAs and/or siRNAs, upon need, can vary from 2 to 10, or 2 to 100, or 2 to 1,000, or 2 to 10,000, linked to ACO via a multivalent linker, for example, a polymeric linker, in branch or liner form.

### Targeting Oligonucleotide

In some embodiments, the targeting oligonucleotide comprises a double stranded oligonucleotide. In some embodiments, the double-stranded oligonucleotide is a double-stranded RNA (dsRNA). The dsRNA may be any dsRNA deemed useful. dsRNAs that find use in the present disclosure include, without limitation, siRNA, saRNA, etc.

In some embodiments, the double-stranded oligonucleotide comprises a sense strand and an antisense strand, the antisense strand having complementarity to a target nucleic acid. In some embodiments, the antisense strand having complementarity to a target nucleic acid is located in a promotor sequence. In some embodiments, the antisense strand having complementarity to a target nucleic acid is located in a coding or template sequence of a gene. In some embodiments, one of the sense or antisense strands has complementarity to a target nucleic acid which is a gene transcript, e.g., a mRNA or a pre-mRNA

In some embodiments, the dsRNA comprises a sense strand that is at least 17 contiguous nucleotides. In some embodiments, the dsRNA comprises a sense strand that is at least 18 contiguous nucleotides. In some embodiments, the dsRNA comprises a sense strand that is at most 60 contiguous nucleotides.

In some embodiments, the sense strand has a length ranging from about 10 nucleotides or more, about 15 nucleotides or more, about 20 nucleotides or more, about 25 nucleotides or more, about 30 nucleotides or more, about 35 nucleotides or more, about 40 nucleotides or more, about 45 nucleotides or more, about 50 nucleotides or more, about 55 nucleotides or more, or about 60 nucleotides or more. In some embodiments, the sense strand is 10-100 nucleotides in length (e.g., 10-20 nucleotides, 10-50 nucleotides, 10-90 nucleotides, 20-95 nucleotides, 30-70 nucleotides, 40-80 nucleotides, 50-100 nucleotides, 10-40 nucleotides, 10-30 nucleotides). In some embodiments, the sense strand is 10-60 nucleotides in length (e.g., 10-20 nucleotides, 10-50 nucleotides, 10-40 nucleotides, 10-30 nucleotides) In some embodiments, the sense strand has a nucleotide length ranging from 27-41 nucleotides.

In some embodiments, the antisense strand has a length ranging from about 10 nucleotides or more, about 15 nucleotides or more, about 20 nucleotides or more, about 25 nucleotides or more, about 30 nucleotides or more, about 35 nucleotides or more, about 40 nucleotides or more, about 45 nucleotides or more, about 50 nucleotides or more, about 55 nucleotides or more, or about 60 nucleotides or more. In some embodiments, the antisense strand is 10-100 nucleotides in length (e.g., 10-20 nucleotides, 10-50 nucleotides, 10-90 nucleotides, 20-95 nucleotides, 30-70 nucleotides, 40-80 nucleotides, 50-100 nucleotides, 10-40 nucleotides, 10-30 nucleotides). In some embodiments, the antisense strand is 19-30 nucleotides in length. In some embodiments, the antisense strand is 18-26 nucleotides in length.

The double-stranded oligonucleotide may comprise a sequence that is modified in order to further increase the stability and/or the ability of the double-stranded oligonucleotide to modulate gene expression. In some embodiments, the sequence of the double-stranded oligonucleotide comprises one or more of a chemically modified nucleotide, or at least one phosphodiester bond between two adjacent nucleotides in the oligonucleotide sequence is substituted by a phosphorothioate or boranophosphate bond. The chemical modifications of the double-stranded oligonucleotide includes, without limitation, modification of the 2′-OH of the ribose in the nucleotide, the modification or the absence of a base in the nucleotide, the locking or bridging of a nucleic acid, a nucleotide being a peptide nucleic acid, a nucleotide being a deoxyribonucleotide (DNA), a nucleotide having a 5′-phosophate moiety, a nucleotide having a 5′-(E)-vinylphosphonate moiety, a nucleotide having a 5′-methyl cytosine moiety, etc. Chemical modifications that may be found in double-stranded oligonucleotides are also further described below.

Short Interfering RNA (siRNA)

Embodiments of the present application are based in part on the surprising discovery that an oligonucleotide agent (for example, siRNA, also referred to as “SOD1 gene siRNA”, “SOD1 siRNA”, or “siSOD1” herein) is capable of inhibiting or downregulating the expression of a SOD1 gene in a cell. The decrease in functional SOD1 gene transcript following administration with an oligonucleotide agent of the present application can achieve a significant decrease or downregulation in the levels of SOD1 mRNA and SOD1 protein in a cell or a mammal.

In particular, the inventors discovered that the functional oligonucleotide agents capable of inhibiting expression of superoxide dismutase 1 (SOD1) comprising a siRNA, wherein the siRNA comprises a sense strand and an antisense strand forming a double strand, wherein the antisense strand comprises a nucleotide sequence comprising at least 10 contiguous nucleotides, with 0, 1, 2 or 3 mismatches, having at least 85% nucleotide sequence complementarity or homology to a portion of the nucleotide sequence of SOD1 mRNA. As a beneficial consequence, a target sequence (e.g., an isolated nucleic acid sequence comprising the target sequence), upon interacting with the siRNA, can inhibit/downregulate the SOD1 mRNA transcript by at least 10% as compared to a baseline level of SOD1 mRNA.

Based at least in part on these discoveries, the present application features siRNA, compositions, and pharmaceutical compositions for inhibiting/downregulating the SOD) mRNA transcript by at least 10% as compared to baseline levels of SOD1 mRNA. In some embodiments, the siRNA inhibits or downregulates the SOD1 mRNA more than 10%. For instance, the siRNA inhibits or downregulates SOD1 mRNA by at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, or greater than 100% as compared to baseline levels of SOD1 mRNA.

Also provided herein are methods for preventing or treating a disease or condition induced by over-expression of SOD1 protein, a SOD1 gene mutation, and/or high or abnormal SOD) level in an individual comprising administering to the individual any of the siRNA, compositions, and/or pharmaceutical compositions described herein.

Embodiments of the present application are also based in part on the discovery that the SOD1 mRNA inhibitory oligonucleotide agents comprises a siRNA having a sense strand that is at least 85%, at least 90%, or at least 95% homology to the nucleotide sequence selected from the group of DS17-0001 (SEQ ID NO: 384), DS17-0002 (SEQ ID NO: 372), DS17-0003 (SEQ ID NO: 409), DS17-0004 (SEQ ID NO: 357), DS17-0005 (SEQ ID NO: 486), DS17-0029 (SEQ ID NO: 588), DS17-01N3 (SEQ ID NO: 912), DS17-02N3 (SEQ ID NO: 914), DS17-03N3 (SEQ ID NO: 916), DS17-04N3 (SEQ ID NO 918), and DS17-05N3 (SEQ ID NO: 920).

In some other embodiments, the SOD1 mRNA inhibitory oligonucleotide agents comprises a siRNA having an antisense strand that is at least 85%, at least 90%, or at least 95% homology to the nucleotide sequence selected from the group of DS17-0001 (SEQ ID NO: 653), DS17-0002 (SEQ ID NO 641), DS17-0003 (SEQ ID NO: 678), DS17-0004 (SEQ ID NO: 626), DS17-0005 (SEQ ID NO: 755), DS17-0029 (SEQ ID NO: 857), DS17-01N3 (SEQ ID NO. 913), DS17-02N3 (SEQ ID NO: 915), DS17-03N3 (SEQ ID NO: 917), DS17-04N3 (SEQ ID NO 919), and DS17-05N3 (SEQ ID NO: 921).

In some embodiments, the SOD1 mRNA inhibitory oligonucleotide agent comprises a siRNA, wherein the sense strand and the antisense strand of the siRNA have nucleotide sequences that is independently at least 85%, at least 90%, or at least 95% homology to the nucleotide sequence pairs selected from the group of: DS17-0001 (SEQ ID NO: 384 and SEQ ID NO: 653), DS17-0002 (SEQ ID NO: 372 and SEQ ID NO: 641), DS17-0003 (SEQ ID NO. 409 and SEQ ID NO: 678), DS17-0004 (SEQ ID NO: 357 and SEQ ID NO: 626), DS17-0005 (SEQ ID NO: 486 and SEQ ID NO: 755), DS117-0029 (SEQ ID NO: 588 and SEQ ID NO: 857), DS17-01N3 (SEQ ID NO: 912 and SEQ ID NO: 913), DS17-02N3 (SEQ ID NO: 914 and SEQ ID NO: 915), DS17-03N3 (SEQ ID NO: 916 and SEQ ID NO: 917), DS17-04N3 (SEQ ID NO: 918 and SEQ ID NO: 919), and DS17-05N3 (SEQ ID NO: 920 and SEQ ID NO 921).

A siRNA targeting 3′UTR of the SOD1 gene to inhibit SOD1 mRNA transcript level in a cell is also provided by the present disclosure. The siRNA comprises an oligonucleotide sequence having a length ranging from 16 to 35 consecutive nucleotides, wherein the continuous oligonucleotide sequence comprises a nucleotide sequence having at least 75%, at least 80%, at least 85%, at least 90%, at least 95% or 100% homology or complementarity to an equal length portion of SEQ ID NO:59, wherein the siRNA inhibits the mRNA transcript of SOD) gene by at least 80% as compared to the baseline of SOD1 mRNA level.

Surprisingly, the inventors discovered that the functional siRNAs capable of inhibiting the SOD1 mRNA transcript level were not randomly distributed on the SOD1 gene or particularly 3′-UTR of the SOD1 gene but were clustered in certain specific hotspot regions. Only some regions on the 3′-UTR of SOD1 gene are in favor of the siRNAs' function of inhibiting, for example, the regions 549 to 562 (H1; SEQ ID NO:61) and 568 to 580 (H2; SEQ ID NO 63) of SOD1 gene. Likewise, merely one region on the 3′-UTR of SOD1 gene are in favor of the ASOs' function of inhibiting, that is, the regions 552 to 566 (H3; SEQ ID NO:65). The regions disclosed herein so called “hotspots”.

The inventors also discovered that optimal target sequences/sense strand of an siRNA within the SOD1 gene or particularly 3′-UTR of the SOD1 gene include sequences having: (1) a GC content between 35% and 65%; (2) less than 5 consecutive identical nucleotides; (3) 3 or less dinucleotide repeats; and (4) 3 or less trinucleotide repeats. As a beneficial consequence, a target sequence (e.g., an isolated nucleic acid sequence comprising the target sequence), upon interacting with the siRNA, can inhibit the SOD1 mRNA transcript level by at least 80% as compared to a baseline level of SOD1 mRNA. Based at least in part on these discoveries, the present disclosure features siRNA, compositions, and pharmaceutical compositions for inhibiting the SOD1 mRNA transcript level by at least 80% as compared to baseline levels of SOD1 mRNA. Also provided herein are methods for preventing or treating a disease or condition induced by elevated level of SOD1 protein in a cell in an individual comprising administering any of the siRNA, compositions, and/or pharmaceutical compositions described herein.

Therefore, the present application discloses a hotspot of siRNA in the 3′-UTR of the SOD1 gene, wherein the oligonucleotide agent disclosed in the present application having at least 75%, at least 80%, at least 85%, at least 90%, at least 95% or 100% homology or complementarity to an equal length portion of the hotspot, wherein the oligonucleotide agent inhibits the mRNA transcript of SOD1 gene by at least 80% as compared to the baseline of SOD1 mRNA level.

The present application further discloses an isolated target site of siRNA in the 3′-UTR of the SOD1 gene, wherein the isolated target site has a nucleic acid sequence having at least 75%, at least 80%, at least 85%, at least 90%, at least 95% or 100% homology to a sequence selected from SEQ ID NOs:1068-1113.

In some embodiments, the sense strand of the siRNA has a nucleotide sequence that is at least 75%, at least 80%, at least 85%, at least 90%, at least 95%, at least 97%, at least 99% or 100% identical to the nucleotide sequence selected from SEQ ID NOs 976-1021. In some embodiments, the antisense strand of the siRNA has a nucleotide sequence that is at least 75%, at least 80%, at least 85%, at least 90%, at least 95%, at least 97%, at least 99% or 100% identical to the nucleotide sequence selected from SEQ ID NOs: 1022-1067 In certain embodiments, the select target region of the SOD1 gene comprises a nucleotide sequence that is at least 75%, at least 80%, at least 85%, at least 90%, at least 95%, at least 97%, at least 99% or 100% identical to the nucleotide sequence selected from group of: SEQ ID NOs: 1068-1113.

In some embodiments, the SOD1 mRNA inhibitory oligonucleotide agent comprises a siRNA, wherein the siRNA comprises a sense strand and an antisense strand having nucleotide sequences that is independently at least 85%, at least 90%, or at least 95% homology to the nucleotide sequence pairs selected from the group of DS17-01M3 (SEQ ID NO: 922 and SEQ ID NO: 923), DS17-02M3 (SEQ ID NO: 924 and SEQ ID NO: 925), DS17-03M3 (SEQ ID NO: 926 and SEQ ID NO: 927), DS17-04M3 (SEQ ID NO: 928 and SEQ ID NO: 929), and DS17-05M3 (SEQ ID NO: 930 and SEQ ID NO: 931).

In addition, the siRNAs provided in Tables 3 and 22 identify a site in a SOD1 transcript that is susceptible to RISC-mediated cleavage. As such, the present invention further features siRNAs that target within one of such sequences. As used herein, a siRNA is said to target within a particular site of an RNA transcript if the siRNA promotes cleavage of the transcript anywhere within that particular site. Such a siRNA will generally include at least 15 contiguous nucleotides from one of the sequences provided in Tables 3 or 22 coupled to additional nucleotide sequences taken from the region contiguous to the selected sequence in a SOD1 gene.

The siRNAs of the oligonucleotide agent described herein include an RNA strand (the antisense strand) having a region which is 60 nucleotides or less in length, i.e., 15-40 nucleotides in length, generally 19-25 nucleotides in length, which region is substantially complementary to at least part of a mRNA transcript of a SOD1 gene. The use of these siRNAs enables the targeted degradation of mRNAs of genes that are implicated in pathologies associated with SOD1 expression in mammals. Exceptionally low dosages of SOD1 siRNAs in particular can specifically and efficiently mediate RNAi, resulting in significant inhibition of expression of a SOD1 gene Using cell-based assays, the present inventors have demonstrated that siRNAs targeting SOD1 can specifically and efficiently mediate RNAi, resulting in significant inhibition of expression of a SOD1 gene. Thus, methods and oligonucleotide agents including these siRNAs are useful for treating pathological processes that can be mediated by down regulating SOD), such as in the treatment of a disorder that causes elevated SOD1 levels, e.g., amyotrophic lateral sclerosis (ALS). The following detailed description discloses how to make and use oligonucleotide agents containing siRNAs to inhibit the expression of a SOD1 gene, as well as oligonucleotide agents and methods for treating diseases and disorders caused by the expression of this gene.

In some embodiments, the continuous oligonucleotide sequence of the siRNA has five or less, i.e., 5, 4, 3, 2, 1, or 0 nucleotide differences or mismatches relative to the equal length portion of SOD1 mRNA. In some embodiments, the continuous oligonucleotide sequence of the sense strand of siRNA has three or less, i.e., 3, 2, 1, or 0 nucleotide differences or mismatches relative to the equal length portion of SOD mRNA. In some embodiments, the continuous oligonucleotide sequence of the antisense strand of siRNA has three or less, i.e., 3, 2, 1, or 0 nucleotide differences or mismatches relative to the equal length portion of SOD1 mRNA.

In some embodiments, the SOD1 mRNA disclosed herein does not contain a nucleotide mutation. In some embodiments, the SOD1 mRNA disclosed herein contains at least one nucleotide mutation. In some embodiments, the SOD1 mRNA disclosed herein contains at least one nucleotide mutation on the targeting site of the siRNA. In some embodiments, the SOD1 mRNA disclosed herein contains at least one nucleotide mutation upper stream and/or downstream the targeting site of the siRNA.

In some embodiments, the differences or mismatches locate in the middle or 3′ terminus of the oligonucleotide sequence of the siRNA. Methods and principles of siRNA molecule design are well known to those skilled in the art and are described in detail in, for example, Place et. al., Molecular Therapy Nucleic Acids (2012) 1, e15; and L1 et. al., PNAS, 2006, vol. 103, no. 46, 17337-17342, which are herein incorporated by reference in their entireties.

In one aspect, an RNA interference agent includes a single-stranded RNA that interacts with a target RNA sequence to direct the cleavage of the target RNA. Without wishing to be bound by theory, long double-stranded RNA introduced into plants and invertebrate cells is broken down into siRNA by a Type III endonuclease known as Dicer (Sharp et al., Genes Dev. 2001, 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleaves the target to induce silencing (Elbashir, et al., (2001) Genes Dev. 15:188). Thus, in one aspect the invention relates to a single-stranded RNA that promotes the formation of a RISC complex to effect silencing of the target gene.

In some embodiments, the siRNA disclosed herein comprises a sense strand and an antisense strand. The sense strand and the antisense strand comprise complementary regions capable of forming a double-stranded nucleic acid structure that inhibits the SOD1 transcription in a cell via the RNAi mechanism. The RNAi mechanism (also known as RNA interference) used herein refers to a mechanism that a double-strand nucleic acid structure is capable of downregulating target genes in a sequence-specific manner at the transcriptional level. The sense strand and the antisense strand of the siRNA can exist either on two different nucleic acid strands or on one nucleic acid strand (e.g., a contiguous nucleic acid sequence). When the sense strand and the antisense strand are located on two different strands, at least one strand of the siRNA has a 3′ overhang of 0 to 6 nucleotides in length, such that the overhangs of 0, 1, 2, 3, 4, 5 or 6 nucleotides in length, and in some cases, both strands have a 3′ overhang of 2 or 3 nucleotides in length.

The nucleotide of the overhang is, in some cases thymine deoxyribonucleotide (dT), or in some cases, natural overhangs which are nucleotides selected from or complementary to the corresponding position on the DNA target. When the sense strand and the antisense strand are located on one nucleic acid strand, in some cases, the siRNA is a hairpin single-stranded nucleic acid molecule, where the complementary regions of the sense strand and the antisense strand form a double-stranded nucleic acid structure with each other. In the siRNA disclosed herein, in some embodiments, the sense strand has a length ranging from 10 to 60 nucleotides. For example, in some embodiments, the sense strand and the antisense strand, independently comprises a length of 16, 17, 18, 19, 20, 21, 22, 23, 24, 25, 26, 27, 28, 29, 30, 31, 32, 33, 34 or 35 nucleotides. In some embodiments, the antisense strand has a length ranging from 10 to 60 nucleotides. For example, in some embodiments, the sense strand and the antisense strand, independently comprises a length of 16, 17, 18, 19, 20, 21, 22, 23, 24, 25, 26, 27, 28, 29, 30, 31, 32, 33, 34 or 35 nucleotides.

In some embodiments, one strand of the siRNA has at least 75% (e.g., at least about 79%, about 80%, about 85%, about 90%, about 95% or about 99%) sequence homology or complementarity to a nucleotide sequence fragment of a SOD1 gene transcript Specifically, the sense strand of the siRNA disclosed herein has at least 75% (e.g., at least about 79%, about 80%, about 85%, about 90%, about 95% or about 99%) sequence homology to a nucleotide sequence fragment of a SOD1 gene transcript, and the antisense strand of the siRNA disclosed herein has at least 75% (e.g., at least about 79%, about 80%, about 85%, about 90%, about 95% or about 99%) sequence homology to a nucleotide sequence fragment of a SOD1 gene transcript.

In some embodiments, the sense strand of the siRNA disclosed herein has at least 75% (e.g., at least about 79%, about 80%, about 85%, about 90%, about 95% or about 99%) sequence homology to a nucleotide sequence selected from group of SEQ ID NOs: 976-1021. In some embodiments, the antisense strand of the siRNA disclosed herein has at least 75% (e.g., at least about 79%, about 80%, about 85%, about 90%, about 95% or about 99%) sequence homology to a nucleotide sequence selected from the group of SEQ ID NOs 1022-1067.

In certain embodiments, one strand of the siRNA can have five or less, i.e., 5, 4, 3, 2, 1, or 0 nucleotide differences or mismatches relative to a nucleotide sequence of any portion of SEQ ID NO: 59 Specifically, the sense strand of the si RNA disclosed herein can have three or less, i.e., 3, 2, 1, or 0 nucleotide differences relative to the nucleotide sequence selected from the group of SEQ ID NOs: 976-1021, and the antisense strand of the siRNA disclosed herein can have three or less, i.e., 3, 2, 1, or 0 nucleotide differences relative to the nucleotide sequence selected from the group of SEQ ID NOs: 1022-1067. In some embodiments, the differences or mismatches locate in the middle or 3′ terminus of the sense or antisense strand of the siRNA.

In some embodiments, the antisense strand disclosed herein is capable of interacting with a target nucleic acid sequence of a mRNA of a SOD1 gene in a sequence specific manner, meaning that the antisense strand is capable of undergoing hybridization to a target nucleic acid through hydrogen bonding. In some embodiments, an antisense strand has a nucleotide sequence that, when written in the 5′ to 3′ direction, comprises the reverse complement of the target portion of a target nucleic acid to which it is targeted. In certain such embodiments, an antisense strand has a nucleotide sequence that, when written in the 5′ to 3′ direction, comprises the reverse complement of the target portion in a fragment of a SOD1 gene transcript.

### Antisense Oligonucleotide (ASO)

The present disclosure also provides antisense oligonucleotides (ASOs) capable of inhibit SOD1 mRNA level in a cell. The ASOs modulates a RNAi-SOD1 mRNA-SOD1 protein pathway by a RNase H-dependent mechanism of action and thus can be used to treat SOD1 protein-related disease, e.g., for amyotrophic lateral sclerosis (ALS) patients.

Disclosed in the present application is an oligonucleotide agent comprising an antisense oligonucleotide (ASO), which comprises an oligonucleotide sequence having a length ranging from 12 to 30 consecutive nucleotides, wherein the continuous oligonucleotide sequence comprises a nucleotide sequence having at least 75%, at least 80%, at least 85%, at least 90%, at least 95% or 100% homology or complementarity to an equal length portion of SEQ ID NO:59, wherein the ASO inhibits the mRNA transcript of SOD1 gene by at least 60% as compared to the baseline of SOD1 mRNA level. In some embodiments, the ASO inhibits the mRNA transcript of SOD1 gene by greater 60% as compared to the baseline of SOD1 mRNA level. For instance, the ASO inhibits the mRNA transcript of SOD1 gene by at least about 65%, at least about 70%, at least about 75%, at least about 80%, at least about 85%, at least about 90%, at least about 95%, at least about 100%, or greater than 100% as compared to the baseline of SOD1 mRNA level.

Further discloses in the present application is that the ASOs in the 3′-UTR of the SOD1 gene capable of inhibiting the SOD1 mRNA transcript level were not randomly distributed on the SOD1 gene or particularly 3′-UTR of the SOD1 gene but were clustered in certain specific hotspot regions. Only some regions on the 3′-UTR of SOD1 gene are in favor of the ASOs' function of inhibiting, for example, the region SEQ ID NO:65 (H3) of SOD1 gene. In some embodiments, the ASO disclosed herein has at least 75%, at least 80%, at least 85%, at least 90%, at least 95% or 100% complementarity to an equal length portion of the hotspot SEQ ID NO:65 (H3). In some embodiments, the ASO inhibits the mRNA transcript of SOD1 gene by at least 60% as compared to the baseline of SOD1 mRNA level. In some embodiments, the ASO comprising a single-stranded oligonucleotide sequence that is at least 75%, at least 80%, at least 85%, at least 90%, at least 95%, at least 97%, at least 99% or 100% identical to the nucleotide sequence selected from chemically modified SEQ ID NOs: 1155-1195. In some embodiments, the ASO comprising a single-stranded oligonucleotide sequence that is at least 75%, at least 80%, at least 85%, at least 90%, at least 95%, at least 97%, at least 99% or 100% identical to the nucleotide sequence selected from the unmodified naked sequences of SEQ ID NOs: 1155-1195.

In some embodiments, the ASO is a single-stranded oligonucleotide comprising a 5′ end and a 3′ end.

In some embodiments, the length of the antisense oligonucleotide comprises a nucleotide length ranging from 12 to 30 nucleotides, such as 13 nucleotides or more, 14 nucleotides or more, 15 nucleotides or more, 16 nucleotides or more, 17 nucleotides or more, 18 nucleotides or more, 19 nucleotides or more, 20 nucleotides or more, 21 nucleotides or more, 22 nucleotides or more, 23 nucleotides or more, 24 nucleotides or more, 25 nucleotides or more. In some embodiments, the length of the ASO is 15 to 25 contiguous oligonucleotides.

In some embodiments, the ASO comprises a nucleotide sequence selected from the nucleotide sequence group of SEQ ID NOs: 1155-1195.

### Chemical Modifications

All nucleotides of the oligonucleotides described herein may be natural, i.e., non-chemically modified, nucleotides or at least one nucleotide may be a chemically modified nucleotide. Non-limiting examples of the chemical modification include one or more of a combination of the following: a) modification of a phosphodiester bond of nucleotides in the oligonucleotide sequence; b) modification of 2′-OH of the ribose in the nucleotide, c) modification of a base in the nucleotide, d) at least one nucleotide in the oligonucleotide sequence being a locked nucleic acid, and e) at least one nucleotide in the oligonucleotide sequence being a deoxyribonucleotide (DNA).

In some embodiments, the nucleotides or oligonucleotides of the present application is chemically modified to enhance stability or other beneficial characteristics. The nucleic acids featured in the present application may be synthesized and/or modified by conventional methods, such as those described in “Current protocols in nucleic acid chemistry,” Beaucage, S L. et al. (Edrs.), John Wiley & Sons, Inc, New York, N.Y., USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages Specific examples of siRNA molecules that can be used in this present application include but are not limited to RNAs containing modified backbones or no natural internucleoside linkages. In some embodiments, RNAs having modified backbones include, among others, those that do not have a phosphorus atom in the backbone. In some embodiments, modified RNAs that do not have a phosphorus atom in their internucleoside backbone can also be oligonucleosides. In some embodiments, the modified oligonucleotide will have a phosphorus atom in its internucleoside backbone.

Chemical modifications of nucleotides or oligonucleotides in the present disclosure are well known to those skilled in the art, and modifications of the phosphodiester bond refer to modifications of oxygen in the phosphodiester bond, including phosphorothioate modifications and boronated phosphate modifications. The modifications disclosed herein stabilize an oligonucleotide structure, maintaining high specificity and high affinity for base pairing. The modifications disclosed herein also stabilize an ACO structure and maintain its delivering accessory properties including bioavailability, biodistribution, and/or cellular uptake of the oligonucleotide agent in various tissues prefrontal cortex, cerebellum, spinal cord (e.g., cervical, thoracic, lumber), muscle, liver, and kidney.

In some embodiments, the chemical modification is to substitute the phosphodiester bond with phosphorothioate (PS) bond on the backbone of the nucleotide sequence of the oligonucleotide agent disclosed herein. In some embodiments, the oligonucleotide agent disclosed herein comprises at least one PS backbone modification. In some embodiments, the ACO comprises at least one PS backbone modification. In some embodiments, the oligonucleotide agent comprises at least 2 PS, at least 3 PS, at least 4 PS, at least 5 PS, at least 6 PS, or greater than 6 PS backbone modifications. In some embodiments, about 90% to about 95% of the phosphodiester backbone bond of ACO are substituted with phosphorothioate (PS) bond. In some embodiments, the oligonucleotide agent comprises at least one PS backbone modification on 5′ end, 3′ end or internal site of the sense strand of the dsRNA. In some embodiments, the oligonucleotide agent comprises at least one PS backbone modification on 5′ end, 3′ end or internal site of the antisense strand of the dsRNA. In some embodiments, the oligonucleotide agent comprises at least one PS backbone modification on 5′ end, 3′ end or internal site of the single strand of the ACO.

In some embodiments, the nucleotides or oligonucleotides of the present application includes at least one chemically modified nucleotide which is modified at 2′-OH in pentose of a nucleotide, i.e., the introduction of certain substituents at the hydroxyl position of the ribose, such as 2′-fluoro modification, 2′-oxymethyl modification, 2′-oxyethylidene methoxy modification, 2,4′-dinitrophenol modification, locked nucleic acid (LNA), 2′-amino modification or 2′-deoxy modification, e.g., a 2′-deoxy-2′-fluoro modified nucleotide, a 2′-deoxy-modified nucleotide.

In some embodiments, the nucleotides or oligonucleotides of the present application includes at least one chemically modified nucleotide which is modified at the base of the nucleotide, e.g., 5′-bromouracil modification, 5′-iodouracil modification, N-methyluracil modification, or 2.6-diaminopurine modification.

In some embodiments, the chemical modification of the nucleotides or oligonucleotides in the present application is an addition of a (E)-vinylphosphonate moiety at the 5′ end of the sense or antisense sequence. In some embodiments, the chemical modification of the at least one chemically modified nucleotide is an addition of a 5′-methyl cytosine moiety at the 5′ end of the sense or antisense sequence.

In some embodiments, the nucleotides or oligonucleotides in the present application are modified at the base of the nucleotide, e.g., 5′-bromouracil modification, 5′-iodouracil modification, N-methyluracil modification, or 2,6-diaminopurine modification. In some embodiments, at least one oligonucleotide in the oligonucleotide agent includes at least one modified nucleotide, e.g., a 2′-O-methyl modified nucleotide, a nucleotide comprising a 5′-phosphorothioate group, a terminal nucleotide linked to a cholesteryl derivative or dodecanoic acid bisdecylamide group, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, and a non-natural base comprising nucleotide. In some embodiments, the first and second dsRNAs include “endo-light” modification with 2′-O-methyl modified nucleotides and nucleotides comprising a 5′-phosphorothioate group.

Modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included.

Non-limiting examples of preparation of the phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677, 5,476,925; 5,519,126; 5,536,821; 5,541,316, 5,550,111; 5,563,253; 5,571,799; 5,587,361; 5,625,050; 6,028,188; 6,124,445; 6,160,109; 6,169,170; 6,172,209, 6,239,265; 6,277,603; 6,326,199; 6,346,614, 6,444,423; 6,531,590; 6,534,639; 6,608,035, 6,683,167; 6,858,715; 6,867,294; 6,878,805, 7,015,315; 7,041,816; 7,273,933, 7,321,029; and U.S. Pat. RE39464, which are herein specifically each incorporated by reference in their entireties.

In some embodiments, the nucleotides or oligonucleotides comprise one or more of RNA, DNA, BNA, LNA or peptide nucleic acid (PNA).

The RNA of a siRNA or saRNA can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12) 3185-3193). Representative U.S. patents that teach the preparation of locked nucleic acid nucleotides include, but are not limited to, the following: U.S. Pat. Nos. 6,268,490; 6,670,461; 6,794,499; 6,998,484; 7,053,207; 7,084,125; and 7,399,845, each of which is herein incorporated by reference in its entirety.

In other RNA mimetics suitable or contemplated for use in siRNAs, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone.

Representative U.S. patents that teach the preparation of PNA compounds include, but are not limited to, U.S. Pat. Nos. 5,539,082, 5,714,331; and 5,719,262, each of which is herein incorporated by reference. Further teaching of PNA compounds can be found, for example, in Nielsen et al., Science, 1991, 254, 1497-1500.

In some embodiments, at least about 50%, at least about 60%, at least about 70%, at least about 80%, at least about 90%, or least about 95%, or about 100% nucleotides of the single-stranded oligonucleotide are chemically modified nucleotides.

In some embodiments, the sense strand and the antisense strand of the oligonucleotide agent independently comprise at least about 10%, at least about 20%, at least about 30%, at least about 40%, at least about 50%, at least about 60%, at least about 70%, at least about 80%, at least about 90%, at least about 95%, or about 100% nucleotides which are chemically modified nucleotides.

These modifications may increase the bioavailability of the oligonucleotides, increase affinity for the target sequence, and enhance resistance to nuclease hydrolysis in a cell.

In addition, to facilitate entry of the oligonucleotides into a cell, lipophilic groups such as cholesterol may be introduced at the ends of the sense or antisense strands of the oligonucleotides on the basis of the above modifications to facilitate action through a cell membrane composed of lipid bilayers and gene promoter regions within the nuclear membrane and nucleus.

In some embodiments, the oligonucleotides of the present application which, upon contact with a cell, are effective in deactivating or downregulating the expression of one or more genes in the cell, preferably by at least 10% (e.g., at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%).

In some embodiments, the oligonucleotides of the present application which, upon contact with a cell, are effective in activating or upregulating the expression of one or more genes in the cell, preferably by at least 10% (e.g., at least 20%, at least 30%, at least 40%, at least 50%, at least 60%, at least 70%, at least 80%, at least 90%, at least 100%, at least 150%, at least 200%, at least 300%, at least 500%, at least 800%, at least 1000%, at least 2000%, or at least 5000%).

One aspect of the application provides a cell comprising an oligonucleotide agent of the present application or a nucleic acid encoding the oligonucleotide agent of the present application. In one embodiment, the cell is a mammalian cell, preferably a human cell. Such cells may be ex vivo, such as cell lines or cell lines, and the like, or may be present in mammalian bodies, such as humans, including infants, children or adults.

In some embodiments, the at least one chemical modified oligonucleotide is the non-targeting single-stranded oligonucleotide. In some embodiments, the at least one chemical modified oligonucleotide is the targeting double stranded oligonucleotide. In certain embodiments, the at least one chemical modified oligonucleotide is the non-targeting single-stranded oligonucleotide and the targeting double stranded oligonucleotide.

### Covalent Linkage

Aspects of the present application include an oligonucleotide agent comprising a double-stranded targeting oligonucleotide and a non-targeting single-stranded oligonucleotide, e.g., an ACO, that are covalently linked.

In some embodiments, a double-stranded targeting oligonucleotide and a non-targeting single-stranded oligonucleotide are covalently linked by a linking component.

In some embodiments, the double-stranded oligonucleotide and the non-targeting single-stranded oligonucleotide are linked with a covalent linker. In some embodiments, the linker is a disulfide linker. Various combinations of strands can be linked, e.g., the first and second dsRNA sense strands are covalently linked or, e.g., the first and second dsRNA antisense strands are covalently linked.

In some embodiments, the sense strand of the double stranded targeting oligonucleotide is covalently linked to the single-stranded oligonucleotide. In some embodiments, the antisense strand of the double stranded targeting oligonucleotide is covalently linked to the single-stranded oligonucleotide.

In some embodiments, any of the oligonucleotides in the oligonucleotide agent of the present application includes a linking component.

Linkers typically comprise a direct bond or an atom such as oxygen or sulfur, a unit such as NR¹, C(O), C(O)O, C(O)NR¹, SO, SO₂, SO₂NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO₂, N(R′)₂, C(O), cleavable linking group, substituted or unsubstituted aryl, substituted or unsubstituted heteroaryl, substituted or unsubstituted heterocyclic; where R¹ is hydrogen, acyl, aliphatic or substituted aliphatic.

Without limitations, various types of linker functionality can be included in the subject conjugates, including but not limited to cleavable linkers, and non-cleavable linkers, as well as reversible linkers and irreversible linkers.

In some embodiments, the linker is a cleavable linker. Cleavable linkers are those that rely on processes inside a target cell to liberate the two parts the linker is holding together, e.g., the ACO and the dsRNA, as reduction in the cytoplasm, exposure to acidic conditions in a lysosome or endosome, or cleavage by specific enzymes (e.g., proteases) within the cell. As such, cleavable linkers allow the dsRNA to be released in its original form after the conjugate has been internalized and processed inside a target cell. Cleavable linkers include, but are not limited to, those whose bonds can be cleaved by enzymes (e.g., peptide linkers); reducing conditions (e.g., disulfide linkers); or acidic conditions (e.g., hydrazones and carbonates).

In some embodiments, the linking component is selected from one or more of ethylene glycol chain, an alkyl chain, a peptide, nucleic acid, carbohydrates, thiol linkage, a phosphodiester, a phosphorothioate, a phosphoramidate, an amide, and a carbamate. In some embodiments, the linking component includes, but is not limited to:
a) L1 or S18 (spacer-18 linker) (1,1-bis(4-methoxyphenyl)-1-phenyl-2,5,8,11,14,17-hexaoxanonadecan-19-yl (2-cyanoethyl) diisopropylphosphoramidite);
b) L4 or C6 (spacer-C6 linker) (6-(bis(4-methoxyphenyl)(phenyl)methoxy)hexyl (2-cyanoethyl) diisopropylphosphoramidite);
c) L6 (1,1-bis(4-methoxyphenyl)-1-phenyl-2,5,8,11,14-pentaoxahexadecan-16-yl (2-cyanoethyl) diisopropylphosphoramidite);
d) L9 or S9 (spacer-9 linker) (2-(2-(2-(bis(4-methoxyphenyl)(phenyl)methoxy)ethoxy)ethoxy)ethyl (2-cyanoethyl) diisopropylphosphoramidite);
e) L10 or C3 (spacer-C3 linker) (3-(bis(4-methoxyphenyl)(phenyl)methoxy)propyl (2-cyanoethyl) diisopropylphosphoramidite);
f) L12(d spacer) ((2R,3S)-2-((bis(4-methoxyphenyl)(phenyl)methoxy)methyl)tetrahydrofuran-3-yl (2-cyanoethyl) diisopropylphosphoramidite);
g) L13 or C12 (spacer-C12 linker) (12-(bis(4-methoxyphenyl)(phenyl)methoxy)dodecyl (2-cyanoethyl) diisopropylphosphoramidite);
h) L14 (spacer-L14 linker) (((1r,4r)-4-((bis(4-methoxyphenyl)(phenyl)methoxy)methyl)cyclohexyl)methyl (2-cyanoethyl) diisopropylphosphoramidite);
i) L5 (spacer-L15 linker) (4-(2-(bis(4-methoxyphenyl)(phenyl)methoxy)ethyl)phenethyl (2-cyanoethyl) diisopropylphosphoramidite);
j) L16 (spacer-L16 linker) (2-(1-(2-(bis(4-methoxyphenyl)(phenyl)methoxy)ethyl)cyclohexyl)ethyl (2-cyanoethyl) diisopropylphosphoramidite);
k) C6x1 ((2S,3S,4S,5S)-2-((bis(4-methoxyphenyl)(phenyl)methoxy)methyl)-5-methoxy-4-(pent-4-yn-1-yloxy)tetrahydrofuran-3-yl (2-cyanoethyl) diisopropylphosphoramidite);
l) C6x2 ((2S,3S,4S,5S)-5-((bis(4-methoxyphenyl)(phenyl)methoxy)methyl)-2-methoxy-4-(pent-4-yn-1-yloxy)tetrahydrofuran-3-yl (2-cyanoethyl) diisopropylphosphoramidite);
m) C6x5 (2-((2-(bis(4-methoxyphenyl)(phenyl)methoxy)ethyl)(pent-4-yn-1-yl)amino)ethyl (2-cyanoethyl) diisopropylphosphoramidite), and
n) C6x7 ((9H-fluoren-9-yl)methyl (4-((2S,4R)-2-((bis(4-methoxyphenyl)(phenyl)methoxy)methyl)-4-((bis(diisopropylamino)phosphanyl)oxy)pyrrolidin-1-yl)-4-oxobutyl)carbamate).

In certain embodiments, the linking component comprises a compound structure shown in Table 1. In certain embodiments, the linking component conjugates to a nucleotide in the single-stranded oligonucleotide or the double-stranded oligonucleotide. In certain embodiments, the linking component conjugates at a nucleoside position selected from 5′-phosphate, 3′, base and 2′-H/OH of a nucleotide in the single-stranded oligonucleotide or the double-stranded oligonucleotide in certain embodiments, the linking component is Spacer phosphoramidite 18 (Phosphoramidous acid, N,N-bis(1-methylethyl)-, 19,19-bis(4-methoxyphenyl)-19-phenyl-3,6,9,12,15,18-hexaoxanonadec-1-yl 2-cyanoethyl ester).

TABLE 1
Linkers used in oligonucleotide agents

| Name | Formula | Structure |
|---|---|---|
| L1 or S18 (spacer-18 linker) | 1,1-bis(4-methoxyphenyl)-1- phenyl-2,5,8,11,14,17- hexaoxanonadecan- 19-yl (2-cyanoethyl) diisopropylphosphoramidite | 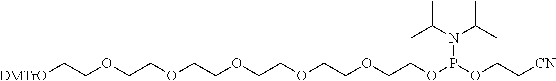 |
| L4 or C6 (spacer-C6 linker) | 6-(bis(4-methoxyphenyl) (phenyl)methoxy)hexyl (2-cyanoethyl) diisopropylphosphoramidite | 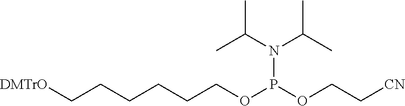 |
| L6 | 1,1-bis(4-methoxyphenyl)-1- phenyl-2,5,8,11,14- pentaoxahexadecan-16-yl (2-cyanoethyl) diisopropylphosphoramidite | 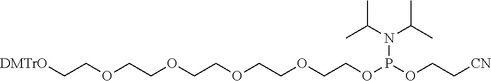 |
| L9 or S9 (spacer-9 linker) | 2-(2-(2-(bis(4- methoxyphenyl)(phenyl) methoxy) ethoxy)ethoxy)ethyl (2-cyanoethyl) diisopropylphosphoramidite | 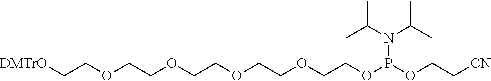 |
| L10 or C3 (spacer-C3 linker) | 3-(bis(4-methoxyphenyl) (phenyl) methoxy)propyl (2-cyanoethyl) diisopropylphosphoramidite | 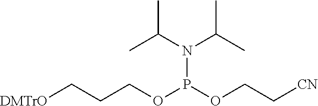 |
| L12(d spacer) | (2R, 3S)-2-((bis(4- methoxyphenyl)(phenyl) methoxy)methyl) tetrahydrofuran-3-yl (2-cyanoethyl) diisopropylphosphoramidite | 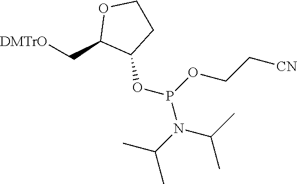 |
| L13 or C12 (spacer-C12 linker) | 12-(bis(4- methoxyphenyl)(phenyl) methoxy) dodecyl (2-cyanoethyl) diisopropylphosphoramidite | 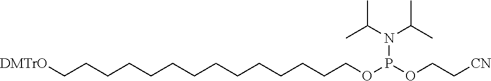 |
| L14 (spacer-L14 linker) | ((1r, 4r)-4-((bis(4- methoxyphenyl)(phenyl) methoxy) methyl) cyclohexyl)methyl (2-cyanoethyl) diisopropylphosphoramidite | 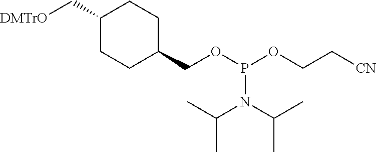 |
| L15 (spacer-L15 linker) | 4-(2-(bis(4- methoxyphenyl)(phenyl) methoxy) ethyl)phenethyl (2-cyanoethyl) diisopropylphosphoramidite | 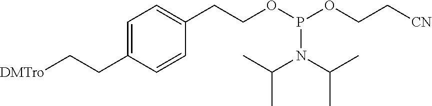 |
| L16 (spacer-L16 linker) | 2-(1-(2-(bis(4- methoxyphenyl) (phenyl)methoxy) ethyl)cyclohexyl) ethyl (2-cyanoethyl) diisopropylphosphoramidite | 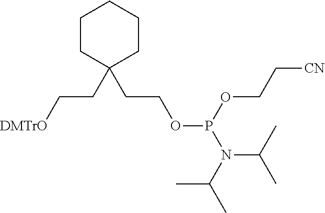 |
| C6x1 | (2S,3S,4,5S)-2-((bis(4- methoxyphenyl)(phenyl) methoxy)methyl)-5- methoxy-4-(pent-4-yn-1-yloxy) tetrahydrofuran-3- yl (2-cyanoethyl) diisopropylphosphoramidite | 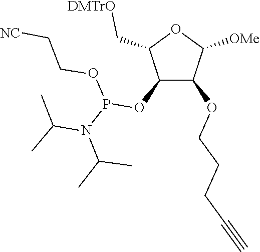 |
| C6x2 | (2S,3S,4S,5S)-5-((bis(4- methoxyphenyl )(phenyl) methoxy) methyl)-2-methoxy- 4-(pent-4-yn-1-yloxy) tetrahydrofuran-3- yl (2-cyanoethyl) diisopropylphosphoramidite | 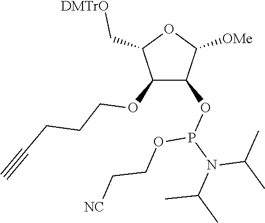 |
| C6×5 | 2-((2-(bis(4- methoxyphenyl)(phenyl) methoxy) ethyl)(pent-4- yn-1-yl)amino)ethyl (2-cyanoethyl) diisopropylphosphoramidite | 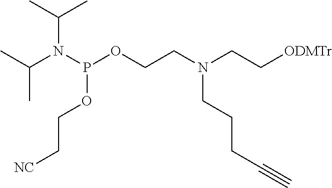 |
| C6x7 | (9H-fluoren-9-yl)methyl (4-((2S, 4R) -2-((bis(4- methoxyphenyl)(phenyl) methoxy)methyl)-4- ((bis(diisopropylamino) phosphanyl)oxy)pyrrolidin- 1-yl)-4-oxobutyl)carbamate | 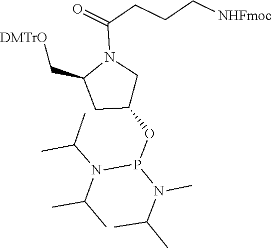 |
| Nucleoside | | 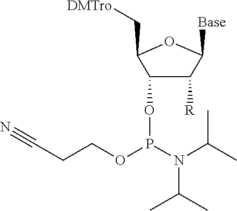 |

*n.b.: R, —Hor —OH or —OMe, or —MOE, or —F, or other 2′ chemical modifications.

In some embodiments, the double-stranded targeting oligonucleotide and the single-stranded oligonucleotide are covalently linked by a phosphodiester bond. In some embodiments, the double-stranded targeting oligonucleotide and the single-stranded oligonucleotide are covalently linked by a phosphorothioate bond.

In some embodiments, the double-stranded targeting oligonucleotide comprises a sense strand that is covalently linked to the single-stranded oligonucleotide. In some embodiments, the double-stranded targeting oligonucleotide comprises an antisense strand that is covalently linked to the single-stranded oligonucleotide.

In some embodiments, the double-stranded targeting oligonucleotide and the single-stranded oligonucleotide are covalently linked by one or more nucleotides.

Non-limiting examples of covalent linkers can be found in U.S. Patent Application Publication No. 20200332292, which is hereby incorporated by reference in its entirety. The covalent linker can join the double-stranded targeting oligonucleotide and the single-stranded oligonucleotide. In some embodiments, the covalent linker can join two sense strands, two antisense strands, one sense and one antisense strand, two sense strands and one antisense strand, two antisense strands and one sense strand, two sense and two antisense strands, an antisense strand and single-stranded oligonucleotide, a sense strand and inactivated oligonucleotide, and the like.

In certain embodiments, the covalent linker includes a nucleic acid (e.g., RNA and/or DNA) and/or a peptide. The linker can be single-stranded, double-stranded, partially single-strands, or partially double-stranded. In some embodiments the linker includes a disulfide bond. The linker can be cleavable or non-cleavable.

In certain embodiments, the covalent linker includes, e.g., dTsdTuu=(5′-2′deoxythymidyl-3′-thiophosphate-5′-2′deoxythymidyl-3′-phosphate-5′-uridyl-3‘-phosphate-S’-uridyl-3′-phosphate); rUsrU (a thiophosphate linker. 5′-uridyl-3′-thiophosphate-5′-uridyl-3′-phosphate); an rUrU linker; dTsdTaa (aadTsdT, 5′-2′deoxythymidyl-3′-thiophosphate-5′-2′deoxythymidyl-3′-phosphate-5′-adenyl-3′-phosphate-5′-adenyl-3′-phosphate); dTsdT (5′-2′deoxythyrnidyl-3′-thiophosphate-5′-2′ deoxythymidyl-3′-phosphate); or dTsdTuu=uudTsdT=5′-2′deoxythymidyl-3′-thiophosphate-5′-2′deoxythymidyl-3′-phosphate-5′-uridyl-3′-phosphate-5′-uridyl-3′-phosphate.

When the covalent linker is a RNA, the RNA linker may be composed of any combination of nucleotides. The combination of nucleotides may be adenine, uracil, guanosine, cytosine, or any combination thereof. The RNA linker may be any length. In some embodiments, the RNA linker is 2-50 nucleotides in length. When the RNA linker is 2-50 nucleotides in length, the RNA linker may be any intervening length such as 5-10, 10-15, or 15-20 nucleotides in length. In some embodiments, the covalent linker includes a polyRNA, such as poly(5′-adenyl-3′-phosphate-AAAAAAAA) or poly(5′-cytidyl-3′-phosphate-5′-uridyl-3′-phosphate-CUCUICUCU)), e.g., Xₙ single-stranded poly RNA linker wherein n is an integer from 2-50 inclusive, preferable 4-15 inclusive, most preferably 7-8 inclusive. Modified nucleotides or a mixture of nucleotides can also be present in said polyRNA linker. When the covalent linker is a DNA, the DNA linker may be composed of any combination of nucleotides. The combination of nucleotides may be adenine, thymine, guanosine, cytosine, or any combination thereof. The DNA linker may be any length. In some embodiments, the DNA linker is 1-50 nucleotides in length. When the DNA linker is 1-50 nucleotides in length, the DNA linker may be any intervening length such as 5-10, 10-15, or 15-20 nucleotides in length. The covalent linker can be a polyDNA, such as poly(5′-2′deoxythymidyl-3′-phosphate-TTTTTTTT), e.g., wherein n is an integer from 2-50 inclusive, preferable 4-15 inclusive, most preferably 7-8 inclusive Modified nucleotides or a mixture of nucleotides can also be present in said polyDNA linker, a single-stranded polyDNA linker wherein n is an integer from 2-50 inclusive, preferable 4-15 inclusive, most preferably 7-8 inclusive. Modified nucleotides or a mixture of nucleotides can also be present in said polyDNA linker.

In some embodiments, the covalent linker includes a disulfide bond, optionally a bis-hexyl-disulfide linker in one embodiment, the disulfide linker is

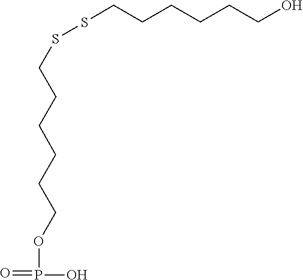

C₁₂H₂₆O₄PS₂
Exact Mass: 329.1010
Mol. Wt. 329.4362

In some embodiments, the covalent linker includes a peptide bond, e.g., include amino acids. In one embodiment, the covalent linker is a 1-10 amino acid long linker, preferably comprising 4-5 amino acids, optionally X-Gly-Phe-Gly-Y wherein X and Y represent any amino acid.

In some embodiments, the covalent linker includes HEG, a hexaethylenglycol linker.

### Orientation and Position of Covalent Linkage

Aspects of the present application include covalently linking the double-stranded targeting oligonucleotide and the non-targeting single-stranded oligonucleotide, e.g., an ACO, to form an oligonucleotide agent. In some embodiments, the orientation of the linkage and positioning of the double-stranded targeting oligonucleotide and the single-stranded oligonucleotide may enhance stability, oligonucleotide activity, or other beneficial characteristics, such as maximized target gene output, increased or decreased activity or expression (e.g., mRNA expression, protein expression, etc.) of one or more target genes.

In some embodiments, the single-stranded oligonucleotide is covalently linked to a 3′ end of the sense or antisense strand of the double-stranded target oligonucleotide; b) the single-stranded oligonucleotide is covalently linked to a 5′ end of the sense or antisense strand of the double-stranded targeting oligonucleotide, or c) the single-stranded oligonucleotide is covalently linked to an internal nucleotide between the 5′ end and the 3′ end of the sense or antisense strand of the double-stranded targeting oligonucleotide. In some embodiments, the internal nucleotide of the sense or antisense strand of the double-stranded targeting oligonucleotide is located at nucleotide position 2, 3, 4, 5, 6, 7, 8, 9, 10, 11, 12, or 13 from 5′ end of the sense or antisense strand; or located at nucleotide position 2, 3, 4, 5, 6, 7, 8, 9, 10, 11, 12, or 13 from 3′ end of the sense or antisense strand.

In some embodiments, an internal nucleotide of the sense or antisense strand of the double-stranded targeting oligonucleotide is substituted by one or more linking component or spacer which is covalently linked to the single-stranded oligonucleotide on its 5′ end or 3′ end (i.e., internal conjugated ODV). In some embodiments, the internal conjugated ODV has enhanced potency as compared to the 3′ or 5′ end conjugated ODV (i.e., ACO conjugated on 3′ or 5′ end of the sense or antisense strand of the double stranded oligonucleotide).

In certain embodiments, the 5′ end of the single-stranded oligonucleotide is conjugated to a linking component. In some embodiments, the 3′ end of the single-stranded oligonucleotide is conjugated to a linking component.

In certain embodiments, the linking component or spacer comprises a compound shown in Table 1.

Agents that Decrease the Expression of the SOD1 Gene or Protein

In some embodiments, the oligonucleotide agent decreases the expression of a SOD1 gene or protein. Administration of the oligonucleotide agent to a patient treats or delays the onset of ALS, such as familial or sporadic ALS or Leu Lou Gehrig's disease. In certain embodiments, the described oligonucleotide agent decreases the amount of a full-length SOD1 protein by, for example, deactivating/downregulating SOD transcription to decrease the amount of full-length SOD1 mRNA. In certain embodiments, full-length SOD1 protein is decreased in an amount sufficient to reduce the symptoms associated with an ALS. In certain embodiments, full-length SOD1 protein is decreased by at least 10% (e.g., at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%).

The administration may be performed in any route of administration deemed useful. In some embodiments, the administration route is locally at the site of a central nervous system location. In some embodiments, the administration route is systemic.

In certain embodiments, a double-stranded targeting oligonucleotide of the oligonucleotide agent that decreases the expression of the SOD1 gene or protein is an siRNA. The SOD1 siRNA deactivates or downregulates the expression of an SOD1 gene, its mRNA transcript or SOD1 protein in a cell in which the SOD1 gene, its mRNA transcript or SOD1 protein is normally or over-expressed.

In typical embodiments, a first strand of the SOD1 siRNA comprises a segment that has at least 75% sequence identity or sequence complementarity to a 6-60 nucleotide fragments of a select target region of the SOD1 mRNA transcript thereby effecting deactivation or downregulation of expression of the SOD1 protein.

In the present application, the SOD1 siRNA comprises a sense nucleic acid fragment and an antisense nucleic acid fragment. The sense nucleic acid fragment and the antisense nucleic acid fragment comprise complementary regions capable of forming a double-stranded nucleic acid structure that knocks down expression of the SOD1 gene in a cell by the RNA interference mechanism. Sense nucleic acid fragments and antisense nucleic acid fragments of siRNAs may be present on two different nucleic acid strands or may be present on the same nucleic acid strand. When the sense and antisense nucleic acid fragments are present on two strands, at least one strand of the siRNA has a 3′ overhang of 0-6 nucleotides in length, preferably both strands have a 3′ overhang of 2 or 3 nucleotides in length, and preferably the nucleotides of the overhang are deoxythymine (dT). When a sense nucleic acid fragment and an antisense nucleic acid fragment of an siRNA are present on the same nucleic acid strand, preferably the siRNA is a single-stranded hairpin-structured nucleic acid molecule, wherein the complementary regions of the sense nucleic acid fragment and the antisense nucleic acid fragment form a double-stranded nucleic acid structure. In such an siRNA, the sense nucleic acid fragment and antisense nucleic acid fragment are 16-60 nucleotides in length, respectively and may be 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides.

In certain embodiments of the present application, the SOD1 siRNA comprises a sense nucleic acid strand and an antisense nucleic acid strand, the sense nucleic acid strand comprising at least one region that is complementary to at least one region on the antisense nucleic acid strand to form a double-stranded nucleic acid structure capable of deactivating expression of the SOD1 protein in a cell.

In certain embodiments of the present application, the sense nucleic acid strand and the antisense nucleic acid strand are located on two different nucleic acid strands. In certain embodiments of the present application, the sense nucleic acid fragment and the antisense nucleic acid fragment are located on the same nucleic acid strand, forming a hairpin single-stranded nucleic acid molecule, wherein the complementary regions of the sense nucleic acid fragment and the antisense nucleic acid fragment form a double-stranded nucleic acid structure.

In certain embodiments of the present application, at least one of the nucleic acid strands has a 3′overhang of 0 to 6 nucleotides in length. In certain embodiments of the present application, both of the nucleic acid strands have 3′overhangs of 2-3 nucleotides in length. In certain embodiments of the present application, the sense and antisense nucleic acid strands are 16 to 35 nucleotides in length, respectively.

In some embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DS17-01M3-AC1 (me14)-L9V3 whose antisense strand has a nucleotide sequence of SEQ ID NO: 933 that has complementarity with a fragment the of the ODV structured sense strand of SEQ ID NO: 928.

In some embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DS17-02M3-AC1 (me14)-L9V3 whose antisense strand has a nucleotide sequence of SEQ ID NO: 935 that has complementarity with a fragment of the ODV structured sense strand of SEQ ID NO: 928.

In some embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DS17-03M3-AC1 (me14)-L9V3 whose antisense strand has a nucleotide sequence of SEQ ID NO: 937 that has complementarity with a fragment of the ODV structured sense strand of SEQ ID NO 936.

In some embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DS17-04M3-AC1 (me14)-L9V3 whose antisense strand has a nucleotide sequence of SEQ ID NO: 939 that has complementarity with a fragment the of the ODV structured sense strand of SEQ ID NO: 938.

In some embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DS17-04M3v-AC1 (me14)-L9V3 whose antisense strand has a nucleotide sequence of SEQ ID NO. 950 that has complementarity with a fragment the of the ODV structured sense strand of SEQ ID NO: 938.

In some embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DS17-05M3-AC1 (me14)-L9V3 whose antisense strand has a nucleotide sequence of SEQ ID NO: 941 that has complementarity with a fragment the of the ODV structured sense strand of SEQ ID NO: 940.

In some embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DS17-29M2-AC1 (me14)-L9V3 whose antisense strand has a nucleotide sequence of SEQ ID NO: 47 that has complementarity with a fragment the of the ODV structured sense strand of SEQ ID NO: 942.

In some embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DS17-01M3v-AC1 (me14)-L9V3 whose antisense strand has a nucleotide sequence of SEQ ID NO: 47 that has complementarity with a fragment the of the ODV structured sense strand of SEQ ID NO: 932.

In some embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DS17-02M3v-AC1 (me14)-L9V3 whose antisense strand has a nucleotide sequence of SEQ ID NO. 943 that has complementarity with a fragment the of the ODV structured sense strand of SEQ ID NO: 934.

In some embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DS17-03M3v-AC1 (me14)-L9V3 whose antisense strand has a nucleotide sequence of SEQ ID NO: 944 that has complementarity with a fragment the of the ODV structured sense strand of SEQ ID NO: 936.

In some embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DS17-05M3v-AC1 (me14)-L9V3 whose antisense strand has a nucleotide sequence of SEQ ID NO: 951 that has complementarity with a fragment the of the ODV structured sense strand of SEQ ID NO: 940.

In some embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DS17-04M3-asSOD1-1-L9V3 whose antisense strand has a nucleotide sequence of SEQ ID NO. 939 that has complementarity with a fragment the of the ODV structured sense strand of SEQ ID NO: 952.

In some aspects, the present application provides an isolated SOD1 gene siRNA targeting site having any contiguous 16-35 nucleotide sequence on a select target region of the SOD1 gene (full length SOD1 sequence of SEQ ID NO 895). In certain embodiments, the select target region of the SOD1 gene comprises a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequence selected from: at least a nucleotide sequence of SEQ ID NOs: 88-356.

In some embodiments, an siRNA includes a nucleotide sequence of a sense strand that is at least 60% (e.g., at least 65%, at least 70%, at least 75%, at least 80%, at least 85%, at least 90%, at least 95%, at least 97%, at least 99% or 100%) identical to the nucleotide sequence selected from: SEQ ID NOs: 357-624. In some embodiments, an siRNA includes a nucleotide sequence of an antisense strand that is at least 60% (e.g., at least 65%, at least 70%, at least 75%, at least 80%, at least 85%, at least 90%, at least 95%, at least 97%, at least 99% or 100%) identical to the nucleotide sequence selected from the group of: SEQ ID NOs 626-893.

In some embodiments, an siRNA includes a nucleotide sequence of a sense strand that is at least 600% (e.g., at least 65%, at least 70%, at least 75%, at least 80%, at least 85%, at least 90%, at least 95%, at least 97%, at least 99% or 100%) identical to the nucleotide sequence selected from: SEQ ID NOs 38, 40, 42, 44, 46, 50, 52, 54, 56, 58, 60, 62, and 64. In some embodiments, an siRNA includes a nucleotide sequence of an antisense strand that is at least 60% (e.g., at least 65%, at least 70%, at least 75%, at least 80%, at least 85%, at least 90%, at least 95%, at least 97%, at least 99% or 100%) identical to the nucleotide sequence selected from the group of: SEQ ID NOs: 39, 41, 43, 45, 47, 49, 51, 53 and 57.

In certain embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of siHTT-AC2-S1L1 (SEQ ID NO: 28) and an antisense strand having a nucleotide sequence of SEQ ID NO: 27 that has partial complementarity with the sense strand of SEQ ID NO: 28.

In certain embodiments, the oligonucleotide agent has a nucleotide sequence that is at least 60% (e.g., at least 65%, at least 70%, at least 75%, at least 80%, at least 85%, at least 90%, at least 95%, at least 97%, at least 99° % or 100%) identical to the nucleotide sequences of siApp-8-S1V1 (SEQ ID NO: 30) and an antisense strand having a nucleotide sequence of SEQ ID NO: 31 that has partial complementarity with the sense strand of SEQ ID NO: 30.

In certain embodiments, the oligonucleotide agent that is at least 60% (e.g., at least 65%, at least 70%, at least 75%, at least 80%, at least 85%, at least 90%, at least 95%, at least 97%, at least 99% or 100%) identical to the nucleotide sequences of siSOD1-231-ESC (SEQ ID NO: 38) and an antisense siRNA strand having a nucleotide sequence of SEQ ID NO: 39 that has partial complementarity with the sense strand of SEQ ID NO: 38.

In certain embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of siSOD1-231-TT (SEQ ID NO: 40) and an antisense siRNA strand having a nucleotide sequence of SEQ ID NO: 41 that has partial complementarity with the sense strand of SEQ ID NO: 40.

In certain embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of (SEQ ID NO: 42) and an antisense siRNA strand having a nucleotide sequence of SEQ ID NO: 43 that has partial complementarity with the sense strand of SEQ ID NO: 42.

In certain embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of (SEQ ID NO: 44) and an antisense siRNA strand having a nucleotide sequence of SEQ ID NO: 45 that has partial complementarity with the sense strand of SEQ ID NO: 44.

In certain embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of (SEQ ID NO: 46) and an antisense siRNA strand having a nucleotide sequence of SEQ ID NO: 47 that has partial complementarity with the sense strand of SEQ ID NO: 46.

In certain embodiments, the oligonucleotide has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of SEQ ID NO: 46 and an antisense siRNA strand having a nucleotide sequence of SEQ ID NO: 49 that has partial complementarity with the sense strand of SEQ ID NO: 46.

In certain embodiments, the oligonucleotide agent has a nucleotide sequence that is at least 60% (e.g., at least 65%, at least 70%, at least 75%, at least 80%, at least 85%, at least 90%, at least 95%, at least 97%, at least 990% or 100%) identical to the nucleotide sequences of (SEQ ID NO: 50) and an antisense strand having a nucleotide sequence of SEQ ID NO: 51 that has partial complementarity with the sense strand of SEQ ID NO: 50.

In certain embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of (SEQ ID NO: 52) and an antisense siRNA strand having a nucleotide sequence of SEQ ID NO: 53 that has partial complementarity with the sense strand of SEQ ID NO: 52.

In certain embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of SEQ ID NO: 54 and an antisense siRNA strand having a nucleotide sequence of SEQ ID NO: 47 that has partial complementarity with the sense strand of SEQ ID NO: 54.

In certain embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of SEQ ID NO: 56 and an antisense siRNA strand having a nucleotide sequence of SEQ ID NO: 57 that has partial complementarity with the sense strand of SEQ ID NO: 56.

In certain embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of siSOD1M2-AC2(N22)-S1V3v-Qu5 (SEQ ID NO: 58) and an antisense siRNA strand having a nucleotide sequence of SEQ ID NO: 57 that has partial complementarity with the sense strand of SEQ ID NO: 58.

In certain embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of siSOD1M2-AC2(N15)-S1V3v-Qu5 (SEQ ID NO: 60) and an antisense siRNA strand having a nucleotide sequence of SEQ ID NO: 57 that has partial complementarity with the sense strand of SEQ ID NO: 60.

In certain embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of siSOD1M2-AC2(N12)-S1V3v-Qu5 (SEQ ID NO: 62) and an antisense siRNA strand having a nucleotide sequence of SEQ ID NO: 57 that has partial complementarity with the sense strand of SEQ ID NO: 62.

In certain embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of siSOD1M2-AC2(N6)-S1V3v-Qu5 (SEQ ID NO 64) and an antisense siRNA strand having a nucleotide sequence of SEQ ID NO: 57 that has partial complementarity with the sense strand of SEQ ID NO: 64.

In certain embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to SEQ ID NOs: 1196-1298, and an antisense siRNA strand having a nucleotide sequence of SEQ ID NO: 57 that has partial complementarity with the sense strand of SEQ ID NOs:1196-1298.

In addition, to facilitate entry of the siRNA into a cell, chemical conjugation groups other than the ACO disclosure herein may be introduced at the ends of the sense or antisense strands of the siRNA on the basis of the above modifications to facilitate action through a cell membrane composed of lipid bilayers and mRNA regions within the nuclear membrane and nucleus.

In some embodiments, siRNAs disclosed in the present application are covalently attached to one or more conjugate groups. In some embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In some embodiments, conjugate groups impart a new property on the attached oligonucleotide, e.g., fluorophores or reporter groups that enable detection of the oligonucleotide. Certain conjugate groups and conjugate moieties have been described previously, for example cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO 1, 1991, 10, 1111-1118, Kabanov et al., FEBS Lett., 1990, 259, 327-330, Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids. 2015, 4, e220; and Nishina et al., Molecular T1herapy. 2008, 16, 734-740), or a GalNAc cluster (e.g., WO2014/179620).

In some embodiments, the siRNA of the present application relates to the sense strand or the antisense strand of the siRNA that is conjugated to one or more conjugation groups selected from: intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In some embodiments, a conjugate group comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, tingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

In some embodiments, the siRNA of the present application is conjugated to one or more conjugation groups selected from: a lipid, a fatty acid, a fluorophore, a ligand, a saccharide, a peptide, and an antibody.

In some embodiments, the siRNA of the present application relates to the sense strand or the antisense strand of the siRNA that is conjugated to one or more conjugation groups selected from a cell-penetrating peptide, polyethylene glycol, an alkaloid, a tryptamine, a benzimidazole, a quinolone, an amino acid, a cholesterol, glucose and N-acetylgalactosamine.

In some embodiments, the siRNA conjugated to one or more conjugation groups disclosed in the embodiments is directly contacted, transferred, delivered or administrated to a cell or a subject.

Oligonucleotide Agents that Increase the Expression of the SMN2 Gene or SMN12 Protein

In some embodiments, the oligonucleotide agent including the double-stranded targeting oligonucleotide and the non-targeting single-stranded oligonucleotide, e.g., an ACO, increases the expression of an SMN2 gene or protein. Administration of oligonucleotide agent to a patient treats or delays the onset of an SMN-deficiency-related condition, such as spinal muscular atrophy (SMA). In certain embodiments, the described oligonucleotide agent increases the amount of a full-length SMN protein by, for example, activating/up-regulating SMN2 transcription in conjunction with modulating splicing for exon 7 inclusion to increase the amount of full-length SMN2 mRNA. In certain embodiments, full-length SMN protein is increased in an amount sufficient to reduce the symptoms associated with an SMN-deficiency-related condition. In certain embodiments, full-length SMN protein is increased by at least 10% (e.g., at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%).

The administration may be performed in any route of administration deemed useful. In some embodiments, the administration route is locally at the site of a central nervous system location. In some embodiments, the administration route is systemic.

In certain embodiments, double-stranded targeting oligonucleotide of the oligonucleotide agent that increases the expression of the SMN2 gene or protein is an saRNA. The SMN2 saRNA activates or upregulates the expression of an SMN2 gene in a cell in which the SMN2 gene is normally, insufficiently, or incorrectly expressed.

In typical embodiments, a first strand of the SMN2 saRNA comprises a segment that has at least 75% sequence identity or sequence complementarity to a 16-35 nucleotide fragment of the promoter region of the SMN2 gene thereby effecting activation or upregulation of expression of the gene.

In certain embodiments of the present application, the SMN2 saRNA comprises a sense nucleic acid strand and an antisense nucleic acid strand, the sense nucleic acid strand comprising at least one region that is complementary to at least one region on the antisense nucleic acid strand to form a double-stranded nucleic acid structure capable of activating expression of the SMN2 gene in a cell.

In certain embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to any one of the saRNA sense strand sequences SEQ ID NOs: 66, 68, 70, 72, 74, 76, 78, 80, 82, 84, and 86, and an antisense saRNA strand having a nucleotide sequence selected from SEQ ID NO: 67, that has partial complementarity with the sense strand SEQ ID NOs: 66, 68, 70, 72, 74, 76, 78, 80, 82, 84, and 86, respectively.

In certain embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to any one of the saRNA sense strand sequence of SEQ ID NO: 66, and an antisense saRNA strand having a nucleotide sequence selected from SEQ ID NO: 67, that has partial complementarity with the sense strand selected from SEQ ID NO: 66.¶

In certain embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04M1-AC2(16)-S1L1V3v (SEQ ID NO: 70) and an antisense siRNA strand having a nucleotide sequence of SEQ ID NO: 67 that has partial complementarity with the sense strand of SEQ ID NO: 70.¶

In certain embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04M1-AC2(15)-S1L1V3v (SEQ ID NO: 72) and an antisense siRNA strand having a nucleotide sequence of SEQ ID NO: 67 that has partial complementarity with the sense strand of SEQ ID NO: 72.¶

In certain embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04M1-AC2(14)-S1L1V3v (SEQ ID NO: 74) and an antisense siRNA strand having a nucleotide sequence of SEQ ID NO: 67 that has partial complementarity with the sense strand of SEQ ID NO: 74.¶

In certain embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04M1-AC2(13)-S1L1V3v (SEQ ID NO: 76) and an antisense siRNA strand having a nucleotide sequence of SEQ ID NO: 67 that has partial complementarity with the sense strand of SEQ ID NO: 76.¶

In certain embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04M1-AC2(12)-S1L1V3v (SEQ ID NO: 78) and an antisense siRNA strand having a nucleotide sequence of SEQ ID NO: 67 that has partial complementarity with the sense strand of SEQ ID NO: 78.¶

In certain embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04M1-AC2(11)-S1L1V3v (SEQ ID NO: 80) and an antisense siRNA strand having a nucleotide sequence of SEQ ID NO: 67 that has partial complementarity with the sense strand of SEQ ID NO: 80.¶

In certain embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04M1-AC2(10)-S1L1V3v (SEQ ID NO: 82) and an antisense siRNA strand having a nucleotide sequence of SEQ ID NO: 67 that has partial complementarity with the sense strand of SEQ ID NO: 82.¶

In certain embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04M1-AC2(9)-S1L1V3v (SEQ ID NO: 84) and an antisense siRNA strand having a nucleotide sequence of SEQ ID NO: 67 that has partial complementarity with the sense strand of SEQ ID NO: 84.

In certain embodiments, the oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04M1-AC2(8)-S1L1V3v (SEQ ID NO: 86) and an antisense siRNA strand having a nucleotide sequence of SEQ ID NO: 67 that has partial complementarity with the sense strand of SEQ ID NO: 86.

### Methods of Modulating Gene Expression

In some aspects, oligonucleotide agents of the present application can be useful for therapeutic approaches to treating diseases such as spinal muscular atrophy (SMA) or ALS.

By non-limiting embodiments, the present application provides a method of decreasing or silencing the levels of mRNA transcript of a SOD1 gene or SOD1 protein in a cell or individual, comprising administering to a subject a pharmaceutical composition disclosed herein.

In some embodiments, the present application relates to a method for treating or delaying the onset or progression of Amyotrophic lateral sclerosis (ALS) in a subject, the method comprising: administering to the subject a pharmaceutical composition disclosed herein. In some embodiments, the subject has sporadic ALS (sALS). In some embodiments, the subject has familial ALS (fALS) In some embodiments, the pharmaceutical composition decreases or silences the levels of mRNA transcript of a SOD1 gene or SOD1 protein in a cell or individual.

In some embodiments, the ACO of the oligonucleotide agent improves the stability, bioavailability, biodistribution, and/or cellular uptake of the siRNA as compared to an oligonucleotide agent without the ACO. In some embodiments, the ACO of the oligonucleotide agent increases the biodistribution of siRNA within one or more target tissues as compared to an oligonucleotide agent without the ACO. In some embodiments, the ACO of the oligonucleotide agent increases the biodistribution of siRNA within two or more target tissues as compared to an oligonucleotide agent without the ACO.

In some embodiments, the one or more target tissues is selected from tissues of brain, spinal cord, muscle, spleen, lung, heart, liver, bladder, and kidney. In some embodiments, the one or more target tissues is selected from the group of: prefrontal cortex, cerebellum, and rest of brain; cervical, thoracic and lumbar in spinal cord; heart, forelimb, hindlimb, nape, and gluteus.

In some embodiments, the oligonucleotide agent of the present application achieves a decrease in full-length SOD1 protein that is less than the amount achieved by administration of the same amount of double stranded oligonucleotide such as a siRNA substance without an ODV structure used individually, with higher potency, reduced toxicity, or unwanted side effects. In some embodiments, the oligonucleotide agent of the present application achieves a decrease in full-length SOD1 protein that is less than the additive effect of treatment with the same amount of the double-stranded targeting oligonucleotide used individually.

Specifically, the oligonucleotide agent of the present application inhibits/down-regulates the SOD1 mRNA transcript by at least 10% (e.g., at least 20%, at least 30%, at least 40%, at least 50%, at least 60%, at least 70%, at least 80%, at least 90%, at least 95%, at least 96%, at least 97%, at least 98%, at least 99%, or about 100% as compared to baseline SOD1 mRNA transcript). In some embodiments, upon administering the oligonucleotide agent disclosed in the embodiments, e.g., to a cell or a subject, the SOD1 mRNA transcript is inhibited/downregulated by at least 50%, 60%, 70%, 77%, 79%, 81%, 84%, 85%, and 88% at 10 nM treatment compared to baseline SOD1 mRNA transcript in control group) in an in vitro cell line. In some embodiments, an oligonucleotide agent inhibits or downregulates the SOD1 mRNA transcript by about 90%.

In some embodiments, the expression of SOD1 gene is inhibited/downregulated by administering the oligonucleotide agent disclosed in the embodiments to a cell at a concentration of at least 0.01 nM, e.g., 0.02 nM, 0.05 nM, 0.08 nM, 0.1 nM, 0.2 nM, 0.3 nM, 0.4 nM, 0.5 nM, 0.6 nM, 0.8 nM, 1 nM, 5 nM, 10 nM, 25 nM, 50 nM, 75 nM, 100 nM, or 150 nM. In some embodiments, the SOD1 gene coded protein (SOD1 protein) is inhibited/downregulated by administering the oligonucleotide agent disclosed in the embodiments, e.g., to a cell or a subject. The knockdown of the SOD1 protein by at least at least 10% (e.g., at least 20%, at least 30%, at least 40%, at least 50%, at least 60%, at least 70%, at least 80%, at least 90%, at least 95%, at least 96%, at least 97%, at least 98%, at least 99%, or about 100%, as compared to baseline expression of the SOD1 protein). In some embodiments, an oligonucleotide agent inhibits or downregulates the expression of the SOD1 protein by about 90%. In some embodiments, the SOD1 protein is inhibited/down-regulated by administering the oligonucleotide agent disclosed in the embodiments to a cell at a concentration of at least 0.01 nM, e.g., 0.02 nM, 0.05 nM, 0.08 nM, 0.1 nM, 0.2 nM, 0.3 nM, 0.4 nM, 0.5 nM, 0.6 nM, 0.8 nM, 1 nM, 2 nM, 3 nM, 4 nM, 5 nM, 10 nM, 25 nM, 50 nM, 75 nM, 100 nM, or 150 nM.

In some embodiments, the oligonucleotide agents disclosed in the embodiments have a dose-dependent knockdown activity in cells. In some embodiments, the oligonucleotide agents knockdown the SOD1 mRNA transcript in cells with a IC₅₀ of less than 10 nM, 5 nM, 4 nM, 3 nM, 2 nM, 1 nM, 0.8 nM, 0.6 nM, 0.5 nM, 0.4 nM, 0.3 nM, 0.2 nM, 0.1 nM, 0.08 nM, 0.06 nM, 0.04 nM, 0.02 nM, 0.01 nM, 0.008 nM, or 0.005 nM.

Another aspect of the present application relates to a method for preventing or treating a disorder or condition induced by over-expression of SOD1 protein, a SOD1 gene mutation, and/or high SOD1 mRNA levels in an individual comprising administering an effective amount of the siRNA, the oligonucleotide agent, or the composition comprising the oligonucleotide agent disclosed herein to the individual. In some embodiments, the effective amount of the siRNA disclosed herein can be a concentration ranging from 0.01 nM to 50 nM, e.g., 0.01 nM, 0.02 nM, 0.05 nM, 0.08 nM, 0.1 nM, 0.2 nM, 0.3 nM, 0.4 nM, 0.5 nM, 0.6 nM, 0.8 nM, 1 nM, 5 nM, 10 nM, 25 nM, 50 nM, 75 nM, 100 nM, or 150 nM. In some embodiments, the disorder or condition is ALS. In some embodiments, the individual is a mammal. In some embodiments, the individual is a human.

In any of the embodiments provided herein, such cells may be ex vivo, such as cell lines, and the like, or may be present in mammalian bodies, such as humans. In some embodiments, the human is a subject or individual suffering from a SOD1 protein related condition or ALS.

Another aspect of the application relates to the use of an oligonucleotide agent of the present application, a nucleic acid encoding two or more oligonucleotides of the oligonucleotide agent of the present application or a composition comprising the oligonucleotide agent of the present application or a nucleic acid encoding two or more oligonucleotides of the oligonucleotide agent of the present for the preparation of a medicament for the treatment or delaying the onset of an SMN-deficiency-related condition or ALS. The subject may be a mammal, such as a human. The subject may be an infant, a child or an adult.

In certain embodiments, the oligonucleotide agent of the present application achieves a decrease in full-length SOD1 protein that is less than the amount achieved by administration of the same amount of double stranded oligonucleotide substance used individually, with reduced toxicity or unwanted side effects. In certain embodiments, the oligonucleotide agent of the present application achieves a decrease in full-length SOD1 protein that is less than the additive effect of treatment with the same amount of the double stranded targeting oligonucleotide used individually.

In certain embodiments, the effect of the oligonucleotide agent of the present application achieves a greater clinical improvement compared to the effect of the same amount of either substance used individually. In certain embodiments, the effect of the oligonucleotide agent achieves a greater than additive clinical improvement compared to the effect of the same amount of double-stranded oligonucleotide used individually.

In any of the embodiments provided herein, such oligonucleotide agent, nucleic acids encoding the oligonucleotide agent of the present application, or compositions comprising such oligonucleotide agent or nucleic acids encoding oligonucleotide agent of the present application may be introduced directly into a cell, or may be produced intracellularly upon introduction of a nucleotide sequence encoding the oligonucleotide agent into a cell, preferably a mammalian cell, more preferably a human cell. Such cells may be ex vivo, such as cell lines, and the like, or may be present in mammalian bodies, such as humans. In some embodiments, the human is a patient or individual suffering from a SMN-deficiency-related condition or ALS. In certain embodiments, a nucleic acid encoding an oligonucleotide agent or a composition comprising the aforementioned oligonucleotide agent or a nucleic acid encoding an oligonucleotide agent of the application, in respective amounts sufficient to effect treatment of ALS.

In certain embodiments, the baseline measurement is obtained from a biological sample, as defined herein, obtained from an individual prior to administering the therapy described herein. In certain embodiments, the biological sample is peripheral blood mononuclear cells, blood plasma, serum, skin tissue, cerebrospinal fluid (CSF).

### Methods of Treating ALS

Aspects of the present methods include methods of treating ALS in a subject comprising administering to the subject a pharmaceutical composition comprising an oligonucleotide agent of the present application and a pharmaceutically acceptable carrier.

Among the known genes underlining ALS, SOD1 gene still remains a major cause of fALS and has been considered to be an important ALS drug target. The human SOD1 gene is located on chromosome 21q22.11 and located from base pair 33,031,935 to base pair 33,041,241 with a genomic size of 9307 bp. SOD1 gene codes for the monomeric SOD1 protein (153 amino acids, molecular weight 16 kDa), and also encodes for the detoxifying copper/zinc binding SOD1 enzyme, which has been found to be localized mainly in the cytosol, as well as in the nucleus, peroxisomes, and mitochondria. The first description of the ALS disease dates back to at least 1824 by Charles Bell, however, SOD1 as the first risk gene of ALS was discovered in 1993. When the first SOD1 transgenic mouse model (SOD1^{G13A}) was established in 1994, indicating that the research on ALS entered a new era. All these lines of evidence indicate that SOD1 mutants cause disease most probably via a gain-of-function, and reducing its levels may be beneficial. The excessive oxidation of wild-type SOD1 induces toxic conformational changes SOD1 silencing significantly attenuated astrocyte-mediated toxicity towards motor neurons. Therefore, silencing SOD1 expression is an important strategy for the treatment of ALS.

The present application provides an oligonucleotide agent with an efficient and effective oligonucleotide delivery vehicle. The agent comprising a double-stranded siRNA targeting oligonucleotide is observed to treat ALS by inhibiting the expression of SOD1 gene through the RNAi silencing mechanism. The present application provides SOD1 siRNAs with potent inhibitory effect, when covalently linked with an ODV (ACO), which was found by the inventors to be beneficial for use in the treatment of ALS.

In some embodiments, the subject has sporadic ALS (sALS). In some embodiments, the subject has familial ALS (fALS). In some embodiments, the subject with ALS has increased or abnormal SOD1 full length protein expression. In some embodiments, the double-stranded oligonucleotide of the oligonucleotide agent decreases or silences the expression of the SOD1 gene or SOD1 protein.

In certain embodiments, the ACO of the oligonucleotide agent improves the stability, bioavailability, biodistribution, and/or cellular uptake of the double-stranded oligonucleotide as compared to an oligonucleotide agent without the ACO. In some embodiments, the ACO of the oligonucleotide agent increases the biodistribution of double stranded oligonucleotide the within one or more target tissues as compared to an oligonucleotide agent without the ACO. In some embodiments, the ACO of the oligonucleotide agent increases the biodistribution of double stranded oligonucleotide the within two or more target tissues as compared to an oligonucleotide agent without the ACO.

In some embodiments, the one or more target tissues is selected from: prefrontal conex, cerebellum, muscle, liver, and kidney.

Cells Comprising siRNA

After contacting a cell, the oligonucleotide agent disclosed herein can effectively inhibit or downregulate the expression of SOD1 gene in a cell, for example downregulate the expression by at least 10% (e.g., as compared to baseline SOD1 transcription).

In some embodiments, the present application relates to a cell comprising the oligonucleotide agent disclosed herein. In some embodiments, the cell is a mammalian cell. In some embodiments, the cell is a human cell, such as a human cell in various tissues in organs including brain, spinal cord, muscle, spleen, lung, heart, liver, bladder, and kidney in some embodiments, the cell in target tissues is selected from the group of: prefrontal cortex, cerebellum, and rest of brain, cervical, thoracic and lumbar in spinal cord; heart, forelimb, hindlimb, nape, and gluteus muscles.

The cell disclosed herein may be in vitro, or ex vivo, such as a cell line or a cell strain, or may exist in a mammalian body, such as a human body. The human body disclosed herein is a subject suffering from a disease or symptom caused by a SOD1 gene mutation, abnormal SOD1 mRNA level, and/or overexpression of SOD1 protein in CNS.

In some embodiments, the cell is from a CNS tissue of a subject suffering from ALS. In some embodiments, the cell is from a subject suffering from ALS.

### Compositions of an Oligonucleotide Agent

Another aspect of the present application provides a pharmaceutical composition comprising the double stranded targeting oligonucleotide and the non-targeting single-stranded oligonucleotide as described in the present application.

The present application provides a composition or pharmaceutical composition capable of downregulated the level of SOD1 mRNA transcript by the mechanism of action (MoA) of RNA interference, comprising the oligonucleotide agent disclosed herein, to treat or prevent onset of a SOD1 related disease (particularly ALS).

In some embodiments, the present application relates to a composition or pharmaceutical composition comprising the siRNA of the present application. In some embodiments, the present application relates to a composition or pharmaceutical composition comprising the siRNA and the ACO as described herein. In some embodiments, the present application relates to a composition or pharmaceutical composition comprising the siRNA and the ACO covalently linked by a linking component as described herein.

In some embodiments, the pharmaceutical composition further comprises at least one pharmaceutically acceptable carrier. In one embodiment, the pharmaceutically acceptable carrier includes one or more of an aqueous carrier, liposome or LNP, polymer, micelle, colloid, metal nanoparticle, non-metallic nanoparticle, bioconjugates (e.g., GalNAc), and polypeptide. In one embodiment, the aqueous carrier may be, for example, RNase-free water, or RNase-free buffer. The composition may contain 1-150 nM, for example 1-100 nM, for example 1-50 nM, for example 1-20 nM, for example 10-100 nM, 10-50 nM, 20-50 nM, 20-100 nM, for example 50 nM of the aforementioned oligonucleotides or nucleic acid encoding the oligonucleotides according to the present application.

In some embodiments, the composition comprises 1-150 nM of the oligonucleotide agent of the present application.

Another aspect of the present application relates to the use of the oligonucleotide agent as described herein, a nucleic acid encoding the oligonucleotides agent as described herein, or a composition comprising such oligonucleotide agent or a nucleic acid encoding the oligonucleotide agent as described herein, where the double-stranded targeting oligonucleotide and the single-stranded oligonucleotide are covalently linked, for the preparation of one or more compositions for modulate the expression of one or more genes or proteins expressed by a cell.

Another embodiment provides pharmaceutical compositions or medicaments comprising the agents of the present application and a therapeutically inert carrier, diluent or pharmaceutically acceptable excipient, as well as methods of using the agents of the present application to prepare such compositions and medicaments.

For the oligonucleotide agent compositions of the present application, the delivery can be optionally through parenteral infusions including intrathecal, intramuscular, intravenous, intraarterial, intraperitoneal, intravesical, intracerebroventricular, intravitreal or subcutaneous administration; or through oral administration, intranasal administration, inhaled administration, vaginal administration, or rectal administration.

A typical formulation is prepared by mixing an agent of the present application and a carrier or excipient. Suitable carriers and excipients are well known to those skilled in the art and are described in detail in, e.g., Ansel H. C. et al., Ansel's Pharmaceutical Dosage Forms and Drug Delivery Systems (2004) Lippincott, Williams & Wilkins, Philadelphia; Gennaro A. R. et al., Remington: The Science and Practice of Pharmacy (2000) Lippincott, Williams & Wilkins, Philadelphia; and Rowe R. C, Handbook of Pharmaceutical Excipients (2005) Pharmaceutical Press, Chicago. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., an agent of the present application or pharmaceutical composition thereof) or aid in the manufacturing of the pharmaceutical product (i.e., medicament).

Compositions of the present application are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

In another aspect, the application provides use of the oligonucleotide agent, according to any one of the embodiments described herein, or a composition according to any one of the embodiments described herein, in the manufacture of a medicament for the treatment of gene or protein-related condition in an individual. The use according to certain embodiments, the condition can include a SMN-deficiency-related condition that comprises ALS. The use according to certain embodiments, the condition can include a SMN-deficiency-related condition that comprises a hereditary neuromuscular disease, preferably spinal muscular atrophy. In other embodiments, the condition can include an immune-related condition, such as cancer. Also provided is the use according to certain embodiments wherein the individual is a mammal, preferably a human.

### Dose Regiments and Route of Administration

Aspects of the present application relate to a pharmaceutical composition comprising the oligonucleotide agent of the present application. In some embodiments, the pharmaceutical composition comprising the oligonucleotide agent of the present application and a pharmaceutically acceptable carrier, a therapeutically inert carrier, diluent or pharmaceutically acceptable excipient. The pharmaceutical composition disclosed herein is to be developed into a medicament preventing or treating the SOD1 protein related condition or ALS.

Aspects of the present application also relate to methods of using the oligonucleotide agents of the present application to prepare such compositions.

Another aspect of the present application relates to use of the oligonucleotide agent of the present application in manufacturing the pharmaceutical composition disclosed herein.

Another aspect of the present application relates to use of the oligonucleotide agent, according to any one of the embodiments described herein, or a composition according to any one of the embodiments described herein, in the manufacture of a medicament for the prevention or treatment of gene or protein-related symptom induced by the over-expression of SOD1 protein, a SOD1 gene mutation, and/or high SOD1 protein levels in an individual.

For the use according to certain embodiments, the condition can include a SOD1 protein-mutation-related disorder or condition that comprises ALS. For the use according to certain embodiments, the symptom induced by over-expression of abnormal SOD1 protein is ALS. Also related is the use according to certain embodiments wherein the individual is a mammal, for example a human.

The dosage at which the oligonucleotide agents or compositions of the present application can be administered can vary within wide limits and will be fitted to the individual requirements in each case. In some embodiments, a first dose of a pharmaceutical composition according to the present application is administered when the subject is less than one week old, less than one month old, less than 3 months old, less than 6 months old, less than one-year-old, less than 2 years old, less than 15 years old, or older than 15 years old.

The single dose of the oligonucleotide agent can be a single dose ranging from 0.01 mg/kg to 1000 mg/kg for example, about 0.01, 0.02, 0.05, 0.1, 0.2, 0.5, 1, 2, 2.5, 5, 7.5, 10, 12.5, 15, 17.5, 20, 25, 30, 40, 50, 75, 100, 120, 150, 200, 250, 300, 400, 500, 750, or 1000 mg/kg. The doses described herein may contain two or more of any of the oligonucleotide agent sequences described herein.

In some embodiments, the proposed dose frequency is approximate. For example, in some embodiments if the proposed dose frequency is a dose at day 1 and a second dose at day 29, an ALS patient may receive a second dose 25, 26, 27, 28, 29, 30, 31, 32, 33, or 34 days after receipt of the first dose. In some embodiments, if the proposed dose frequency is a dose at day 1 and a second dose at day 15, an ALS patient may receive a second dose 10, 11, 12, 13, 14, 15, 16, 17, 18, 19, or 20 days after receipt of the first dose. In some embodiments, if the proposed dose frequency is a dose at day 1 and a second dose at day 85, an ALS patient may receive a second dose 80, 81, 82, 83, 84, 85, 86, 87, 88, 89, or 90 days after receipt of the first dose.

In some embodiments, the dose and/or the volume of the injection will be adjusted based on the subject's age, the subject's body weight, and/or other factors that may require adjustment of the parameters of the injection.

In some embodiments, pharmaceutical compositions comprise a co-solvent system. Certain of such co-solvent systems comprise, for example, benzyl alcohol, a nonpolar surfactant, a water-miscible organic polymer, and an aqueous phase. In some embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

Examples of other compositions or components associated with the oligonucleotide agent, compositions, pharmaceutical compositions, and methods described herein include, but are not limited to: diluents, salts, buffers, chelating agents, preservatives, drying agents, antimicrobials, needles, syringes, packaging materials, tubes, bottles, flasks, beakers, and the like, for example, for using, modifying, assembling, storing, packaging, preparing, mixing, diluting, and/or preserving the components for a particular use. In embodiments where liquid forms of any of the components are used, the liquid form may be concentrated or ready to use.

In some embodiments, lipid moieties used in nucleic acid therapies can be applied in the present application for delivery of the oligonucleotide agent molecules disclosed herein. In such methods, the nucleic acid (e.g., one or more oligonucleotide agents described herein) is introduced into preformed liposomes or lipoplexes made of mixtures of cationic lipids and neutral lipids. In certain methods, oligonucleotide agent complexes with mono- or poly-cationic lipids are formed without the presence of a neutral lipid. In some embodiments, a lipid moiety is selected to increase distribution of a pharmaceutical agent to a particular cell or tissue. In some embodiments, a lipid moiety is selected to increase distribution of a pharmaceutical agent to fat tissue. In some embodiments, a lipid moiety is selected to increase distribution of a pharmaceutical agent to muscle tissue.

In some embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In some embodiments, certain organic solvents such as dimethylsulfoxide are used.

In some embodiments, pharmaceutical compositions comprise one or more tissue-specific delivery molecules designed to deliver the one or more pharmaceutical agents of the present application to specific tissues or cell types. For example, in some embodiments, pharmaceutical compositions include liposomes coated with a tissue-specific antibody.

In some embodiments, the oligonucleotide agent can be delivered or administered via a vector. Any vectors that may be used for gene delivery may be used. In some embodiments, a viral vector may be used. Non-limiting examples of viral vectors that may be used in the present application include, but are not limited to, human immunodeficiency virus; HSV, herpes simplex virus; MMSV, Moloney murine sarcoma virus; MSCV, murine stem cell virus; SFV, Semliki Forest virus, SIN, Sindbis virus, VEE, Venezuelan equine encephalitis virus; VSV, vesicular stomatitis virus; VV, vaccinia virus, AAV, adeno-associated virus; adenovirus; lentivirus; and retrovirus.

In some embodiments, the vector is a recombinant AAV vector AAV vectors are DNA viruses of relatively small size that can integrate, in a stable and site-specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsulation functions: the left-hand part of the genome, that contains the rep gene involved in viral replication and expression of the viral genes, and the right-hand part of the genome, which contains the cap gene encoding the capsid proteins of the virus.

AAV vectors may be prepared using standard methods in the art. Adeno-associated viruses of any serotype are suitable (see, e.g., Blacklow, pp 165-174 of “Parvoviruses and Human Disease” J. R. Pattison, ed. (1988); Rose, Comprehensive Virology 3:1, 1974; P. Tattersall “The Evolution of Parvovirus Taxonomy” In Parvoviruses (J R Kerr, S F Cotmore, M E Bloom, R M Linden, C R Parrish, Eds.) p 5-14, Hudder Arnold, London, U K (2006); and D E Bowles, J E Rabinowitz, R J Samulski “The Genus Dependovirus” (J R Kerr, S F Cotmore, M E Bloom, R M Linden, C R Parrish, Eds.) p 15-23, Hudder Arnold, London, UK (2006), the disclosures of which are hereby incorporated by reference herein in their entireties). Methods for purifying for vectors may be found in, for example, U.S. Pat. Nos. 6,566,118, 6,989,264, and 6,995,006 and WO/1999/011764 titled “Methods for Generating High Titer Helper-free Preparation of Recombinant AAV Vectors”, the disclosures of which are herein incorporated by reference in their entirety Preparation of hybrid vectors is described in, for example, PCT Application No. PCT/US2005/027091, the disclosure of which is herein incorporated by reference in its entirety. The use of vectors derived from the AAVs for transferring genes in vitro and in vivo has been described (See e.g., International Patent Application Publication Nos: 91/18088 and WO 93/09239; U.S. Pat. Nos. 4,797,368, 6,596,535, and 5,139,941; and European Patent No: 0488528, all of which are herein incorporated by reference in their entirety). These publications describe various AAV-derived constructs in which the rep and/or cap genes are deleted and replaced by a gene of interest, and the use of these constructs for transferring the gene of interest in vitro (into cultured cells) or in vivo (directly into an organism). The replication defective recombinant AAVs according to the application can be prepared by co-transfecting a plasmid containing the nucleic acid sequence of interest flanked by two AAV inverted terminal repeat (ITR) regions, and a plasmid carrying the AAV encapsulation genes (rep and cap genes), into a cell line that is infected with a human helper virus (for example an adenovirus). The AAV recombinants that are produced are then purified by standard techniques.

In some embodiments, the vector(s) for use in the methods of the application are encapsulated into a virus particle (e.g., AAV virus particle including, but not limited to, AAV1, AAV2, AAV3, AAV4, AAV5, AAV6, AAV7, AAV8, AAV9, AAV10, AAV11, AAV12, AAV13, AAV14, AAV15, and AAV16). Accordingly, the application may include a recombinant virus particle (recombinant because it contains a recombinant polynucleotide) comprising any of the vectors described herein. Methods of producing such particles are known in the art and are described in U.S. Pat. No. 6,596,535.

In a particular embodiment, the oligonucleotide agent shows a greater than additive effect or synergy in the treatment, prevention, delaying progression and/or amelioration of diseases caused by the SOD1 gene, and additionally for the protection of cells implicated in the pathophysiology of the disease, particularly for the treatment, prevention, delaying progression and/or amelioration ALS.

In some embodiments, the delivery of the pharmaceutical composition comprising the oligonucleotide agent can be through parenteral infusions including intrathecal, intramuscular, intravenous, intraarterial, intraperitoneal, intravesical, intracerebrovenmricular, intravitreal or subcutaneous administration, or through oral administration, intranasal administration, inhaled administration, vaginal administration, or rectal administration.

In certain embodiments, where a dose of the oligonucleotide agent is administered as an intrathecal injection by lumbar puncture, the use of a smaller gauge needle may reduce or ameliorate one or more symptoms associated with a lumbar puncture procedure. In certain embodiments, symptoms associated with a lumbar puncture include, but are not limited to, post-lumbar puncture syndrome, headache, back pain, pyrexia, constipation, nausea, vomiting, and puncture site pain. In certain embodiments, use of a 24- or 25-gauge needle for the lumbar puncture reduces or ameliorates one or more post lumbar puncture symptoms. In certain embodiments, use of a 21-, 22-, 23-, 24- or 25-gauge needle for the lumbar puncture reduces or ameliorates post-lumbar puncture syndrome, headache, back pain, pyrexia, constipation, nausea, vomiting, and/or puncture site pain.

In certain embodiments, the dose and/or the volume of the injection will be adjusted based on the patient's age, the patient's CSF volume, or the patient's age and/or estimated CSF volume. (For example, see Matsuzawa J, Matsui M, Konishi T, Noguchi K, Gur R C, Bilker W, Miyawaki T. Age-related volumetric changes of brain gray and white matter in healthy infants and children. Cereb Cortex 2001 April; 11(4):335-342, which is hereby incorporated by reference in its entirety).

### Kits

In another aspect, any of the compositions described herein can be provided in one or more kits, optionally including instructions for use of the compositions. That is, the kit can include a description of use of an oligonucleotide agent or composition in any method described herein. A “kit,” as used herein, typically defines a package, assembly, or container (such as an insulated container) including one or more of the components or embodiments of the application, and/or other components associated with the application, for example, as previously described. Any of the antes or components of the kit may be provided in liquid form (e.g., in solution), or in solid form (e.g., a dried powder, frozen, etc.).

In some cases, the kit includes one or more components, which may be within the same or in two or more receptacles, and/or in any combination thereof. The receptacle is able to contain a liquid, and non-limiting examples include bottles, vials, jars, tubes, flasks, beakers, or the like. In some cases, the receptacle is spill-proof (when closed, liquid cannot exit the receptacle, regardless of orientation of the receptacle).

Examples of other compositions or components associated with the agents, compounds and methods described herein include, but are not limited to: diluents, salts, buffers, chelating agents, preservatives, drying agents, antimicrobials, needles, syringes, packaging materials, tubes, bottles, flasks, beakers, and the like, for example, for using, modifying, assembling, storing, packaging, preparing, mixing, diluting, and/or preserving the components for a particular use. In embodiments where liquid forms of any of the components are used, the liquid form may be concentrated or ready to use.

In additional embodiments, a kit can include instructions or instructions to a website or other source in any form that are provided for using the kit in connection with the components and/or methods described herein. For instance, the instructions may include instructions for the use, modification, mixing, diluting, preserving, assembly, storage, packaging, and/or preparation of the components and/or other components associated with the kit. In some cases, the instructions may also include instructions for the delivery of the components, for example, for shipping or storage at room temperature, sub-zero temperatures, cryogenic temperatures, etc. The instructions may be provided in any form that is useful to the user of the kit, such as written or oral (e.g., telephonic), digital, optical, visual (e.g., videotape, DVD, etc.) and/or electronic communications (including Internet or web-based communications), provided in any manner.

### Materials and Methods

### General Methods

Starting materials, reagents and solvents for organic synthesis were purchased from commercial sources and used as received unless stated otherwise. Purification of reactions products was performed by column chromatography using silica gel (200-300 mesh) and eluting with hexane/ethyl acetate, DCM/MeOH. Thin layer chromatography (TLC) was carried out using precoated silica Gel GF plates and visualized using KMnO4 stains. 1H-NMR spectra were recorded at 400 or 500 MHz (Varian) using CDCl3 with TMS. Mass spectra (MS) were recorded on LC/MS (Agilent Technologies 1260 Infinity II/6120 Quadrupole) and a time-of-flight mass spectrometer by ESI or matrix assisted laser desorption/ionization (MALDI).

### Oligonucleotide Synthesis

The oligonucleotides used were synthesized on a K&A DNA synthesizer (K&A Laborgeraete GbR, Schaafheim, Germany) by using solid phase technique. Briefly, during solid phase synthesis, phosphoramidite monomers including various linkers and conjugations (0.1M in acetonitrile or dichloromethane), were added sequentially onto a solid support to generate the desired full-length oligonucleotides. Each cycle of base addition consisted of four chemical reactions including detritylation, coupling, oxidation/thiolation and capping.

Detritylations were performed using 3% dichloroacetic acid (TCA) in DCM for 45 seconds and capping was done with a 16% N-methylimidazole in THF (CAP A) and THF:acetic anhydride:2,6-lutidine, (80:10:10, v/v/v) (CAP B) for 20 seconds. Sulfurizations were carried out with 0.1 M solution of xanthane hydride in pyridine/ACN (50:50, v/v) for 3 minutes. Oxidation was performed using 0.02 M iodine in THF:pyridine:water (70:20:10, v/v/v) for 60 seconds. Phosphoramidite coupling times were 360 s for all amidites.

Deprotection I (Nucleobase Deprotection): After completion of synthesis, the solid support was then transferred to a screw-cap microcentrifuge tube. For a 1 μM synthesis scale, a mixture of 33% methylamine in ethanol and 1 ml of ammonium hydroxide was added. The tube containing the solid support was then heated in an oven at 60° C. to 65° C. for 15 min and then allowed to cool to room temperature. The cleavage solution was collected and evaporated to dryness in a speedvac.

Deprotection II (Removal of 2′-TBDMS Group): The crude RNA oligonucleotide, still carrying the 2′-TBDMS groups, was dissolved in 0.1 ml of DMSO. After adding 1 ml of Triethylamine 3HF, the tube was capped, and the mixture was shaken vigorously to ensure complete dissolution. The bottle was heated in an oven at 60° C. to 65° C. for 3 to 3.5 hours. The tube was removed from the oven and cooled to room temperature. The solution containing the completely desilylated oligonucleotide was cooled on dry ice. 2 mls of ice-cold n-butanol (−20° C.) were carefully added in 0.5 ml portions to precipitate the oligonucleotide. The precipitate was filtered, washed with 1 ml ice-cold n-butanol, and subsequently dissolved in 2M TEAA (triethylammonium acetate). The crude oligonucleotides were then purified by exchange (IEX) HPLC using a source 15Q column. Purity of the fractions were analyzed by ion exchange (IEX) HPLC using Column DNA PacrM PA100. Following the generation of desalted purified single-strand solutions, a duplex was made by annealing two complimentary single-stranded oligonucleotides, which were subsequently lyophilized to powder.

### RP-HPLC and ESI-MS

Oligonucleotides were analyzed via reverse phase chromatography (i.e., RP-HPLC) (Waters XBridge oligonucleotide BEH C18 130A) using an acetonitrile grant and detection wavelength of 260 nm to qualify oligonucleotide purity. Electrospray ionization mass spectrometry (ESI-MS) was performed on desalted oligonucleotides resuspended in water/acetonitrile (50:50) containing 1% (vol/vol) triethylamine in negative ion mode.

### Cell Culture and Treatment

SMA patient-derived fibroblasts including GM03813 (SMA type I with 3 copies of SMN2 gene) and GM09677 (SMA type I with 3 copies of SMN2 gene) cells were obtained from Coriell Institute (Camden, NJ, USA). Both cultures were maintained at 37° C. with 5% CO₂ in modified MEM medium (Gibco, Thermo Fisher Scientific, Carlsbad, CA) supplemented with 15% bovine calf serum (Sigma-Aldrich), 1% NEAA (Gibco), and 1% penicillin/streptomycin (Gibco). Mouse neural stem cell line NSC-34 (BNCC341122, Beijing, China), HEK293A (Cobioer/CBP60436, Nanjing, China), and NSC-34 (BNCC341122, Beijing, China) cells were cultured at 37° C. with 5% C2 in DIMEM (Gibco) medium supplemented with 10% bovine calf serum (Sigma-Aldrich) and 1% penicillin/streptomycin (Gibco). Primary mouse hepatocytes (PMH) were isolated from the liver of Type I SMA (Smn1^{-/-}, SMN2^{+/+}) mice.

PMH cells were cultured at 37° C. with 5% C02 in modified DMEM medium (Gibco, Thermo Fisher Scientific, Carlsbad, CA) supplemented with 10% bovine calf serum (Sigma-Aldrich) and 1% penicillin/streptomycin (Gibco). SH-SY5Y cells (SCSP-5014, Chinese Academy of Sciences, Shanghai, China) were cultured at 5% CO₂ and 37° C. in MEM medium (Gibco, Thermo Fisher Scientific, Carlsbad, CA) supplemented with 10% bovine calf serum (Sigma-Aldrich) and 1% penicillin/streptomycin (Gibco). Neuro-2a (N-2a, BNCC338529, Beijing, China) were cultured at 5% CO₂ and 37° C. in EMEM medium (Gibco, Thermo Fisher Scientific, Carlsbad, CA) supplemented with 10% bovine calf serum (Sigma-Aldrich) and 1% penicillin/streptomycin (Gibco). Human glioma cell line T98G (ATCC) cells were cultured at 37° C. with 5% C02 in modified MEM medium (Gibco, Thermo Fisher Scientific, Carlsbad, CA) supplemented with 10% bovine calf serum (Sigma-Aldrich) and 1% penicillin/streptomycin (Gibco). Human cervical carcinoma cell HeLa (ATCC) cells were cultured at 37° C. with 5% C02 in modified RPMI 1640 medium (Gibco, Thermo Fisher Scientific, Carlsbad, CA) supplemented with 10% bovine calf serum (Sigma-Aldrich) and 1% penicillin/streptomycin (Gibco). The T98G, HeLa and HEK293A cells were seeded into 24-well plates at 10-10∝cells/well. siRNAs (note: the oligonucleotide agents in the present invention are simplified as “siRNA” in the description of materials, methods and examples hereafter) were individually transfected into the cells in each well at indicated concentration, or any other concentrations with 0.3 μL of RNAiMAX (Invitrogen, Carlsbad, CA) by following the reverse transfection protocol respectively, and the transfection duration was 24 hours. Other cells were seeded into 6- and 96-well plates at a final density of 1˜2×10⁵ and 6000 cells/well, respectively. Mock (blank control) was transfected in the absence of an oligonucleotide. dsCon2 duplex was transfected as a non-specific duplex control. All oligonucleotide sequences including RNA duplexes and ODV constructs used for cell treatments are listed in Table 2, Table 3 and Table 8. ACO sequence are listed in Table 7.

TABLE 2
Oligonucleotide strand sequences and duplex compositions

| | | Target | SEQ ID | | Size |
|---|---|---|---|---|---|
| Oligonucleotide name | Category | gene(s) | NO | Sequence (5′-3′) | (nt) |
| dsCon2 | Neg. Control dsRNA | | 24 | ACUACUGAGUGACAGUAGATT | 21 |
| | | | 25 | UCUACUGUCACUCAGUAGUTT | 21 |
| siHTT-S1V1 | siRNA | HTT | 26 | mU*mA*mUmCmAmGfUmAfAfAfGmAmGmAmUmU*mA*mA | 18 |
| | | | 27 | VPmU*fU*mAmAmUfCmUmCmUmUfUmAfCmUmGmAmUmA*mU*mA | 20 |
| siHTT-AC2-S1L1 | ODV- | HTT | 28 | mU*mA*mUmCmAmGfUmAfAfAfGmAmGmAmUmU*mA*mA-S18- | 36 |
| | siRNA | | | meU*meA*meG*meA*meC*meU*meA*meG*meA*meU*meC*meA*meU* | |
| | | | | meA*meU*meG*meA*meG | |
| | | | 27 | VPmU*fU*mAmAmUfCmUmCmUmUfUmAfCmUmGmAmUmA*mU*mA | 20 |
| siApp-8-S1V1v | siRNA | App | 30 | fC*mC*fCmAfGmAfAmUfCfAfGmCfUmAfCmGfGmAfA | 19 |
| | | | 31 | VPmU*fU*mCfCmGfUmAfGmCfUmGfAmUfUmCfUmGfGmG*fC*mU | 21 |
| siApp-8-AC2(N18)-S1L1V3v | ODV- | App | 32 | fC*mC*fCmAfGmAfAmUfCfAfGmCfUmAfCmGfGmAfA-S18- | 37 |
| | siRNA | | | U*A*G*A*C*U*A*G*A*U*C*A*U*A*U*G*A*G | |
| | | | 31 | VPmU*fU*mCfCmGfUmAfGmCfUmGfAmUfUmCfUmGfGmG*fC*mU | 21 |
| siApp-8-AC2(N15)-S1L1V3v | ODV- | App | 34 | fC*mC*fCmAfGmAfAmUfCfAfGmCfUmAfCmGfGmAfA-S18- | 34 |
| | siRNA | | | U*A*G*A*C*U*A*G*A*U*C*A*U*A*U | |
| | | | 31 | VPmU*fU*mCfCmGfUmAfGmCfUmGfAmUfUmCfUmGfGmG*fC*mU | 21 |
| siApp-8-AC2(N12)-S1L1V3v | ODV- | App | 36 | fC*mC*fCmAfGmAfAmUfCfAfGmCfUmAfCmGfGmAfA-S18- | 32 |
| | siRNA | | | U*A*G*A*C*U*A*G*A*U*C*A | |
| | | | 31 | VPmU*fU*mCfCmGfUmAfGmCfUmGfAmUfUmCfUmGfGmG*fC*mU | 21 |
| siSOD1-231-E | siRNA | SOD1 | 38 | fU*mG*fAmAfGmAfGmAfGfGfCmAfUmGfUmUfG*mG*fA | 19 |
| | | | 39 | mU*fC*mCfAmAfCmAfUmGfCmCfUmCfUmCfUmUfCmA*fU*mC | 21 |
| siSOD1-231-TT | siRNA | SOD1 | 40 | fU*mG*fAmAfGmAfGmAfGfGfCmAfUmGfUmUfG*mG*fA | 19 |
| | | | 41 | mU*fC*mCfAmAfCmAfUmGfCmCfUmCfUmCfUmUfCmA*T*T | 21 |
| siSOD1-231-M1 | siRNA | SOD1 | 42 | mG*fA*mAfGmAfGmAfGfGfCfAfUmGfUmUfG*mG*fA | 18 |
| | | | 43 | mU*fC*mCfAmAfCmAfUmGfCmCfUmC*fU*mC*fU*mU*fC*mA*fU | 20 |
| siSOD1-231-S2 | siRNA | SOD1 | 44 | fG*mA*fGmAfGmGfCmAfUmGfUmUfG*mG*fA | 15 |
| | | | 45 | mU*fC*mCfAmAfCmAfUmGfCmCfUmC*fU*mC*fU*mU*fC*mA*fU | 20 |
| siSOD1-388-E | siRNA | SOD1 | 46 | fG*mG*fUmGfGmAfAmAfUfGfAmAfGmAfAmAf*GmU*fA | 19 |
| | | | 47 | mU*fA*mCfUmUfUmCfUmUfCmAfUmUfUmCfCmAfCmC*fU*mU | 21 |
| siSOD1-388-TT | siRNA | SOD1 | 48 | fG*mG*fUmGfGmAfAmAfUfGfAmAfGmAfAmAf*GmU*fA | 19 |
| | | | 49 | mU*fA*mCfUmUfUmCfUmUfCmAfUmUfUmCfCmAfCmC*T*T | 21 |
| siSOD1-388-M1 | siRNA | SOD1 | 50 | mG*fU*mGfGmAfAmAfUfGfAfAfGmAfAmAfG*mU*fA | 18 |
| | | | 51 | mU*fA*mCfUmUfUmCfUmUfCmAfUmU*fU*mC*fC*mA*fC*mC*fU | 20 |
| siSOD1-388-S2 | siRNA | SOD1 | 52 | fG*mA*fAmAfUmGfAmAfGmAfAmAfG*mU*fA | 15 |
| | | | 53 | mU*fA*mCfUmUfUmCfUmUfCmAfUmU*fU*mC*fC*mA*fC*mC*fU | 20 |
| siSOD1M2-L1 | siRNA | SOD1 | 54 | fG*mG*fUmGfGmAfAmAfUfGfAmAfGmAfAmAf*GmU*fA-S18 | 19 |
| | | | 47 | mU*fA*mCfUmUfUmCfUmUfCmAfUmUfUmCfCmAfCmC*fU*mU | |
| siSOD1M2-S1V1v-Qu5 | siRNA | SOD1 | 56 | fG*mG*fUmGfGmAfAmAfUfGfAmAfGmAfAmAf*GmU*fA-Qu5 | 19 |
| | | | 57 | VPmU*fA*mCfUmUfUmCfUmUfCmAfUmUfUmCfCmAfCmC*fU*mU | 21 |
| siSOD1M2-AC2(N22)-S1V3v-Qu5 | ODV- | SOD1 | 58 | Qu5-fG*mG*fUmGfGmAfAmAfUfGfAmAfGmAfAmAfG*mU*fA-S18- | 41 |
| | siRNA | | | U*A*G*A*C*U*A*G*A*U*C*A*U*A*U*G*A*G*U*A*G*A | |
| | | | 57 | VPmU*fA*mCfUmUfUmCfUmUfCmAfUmUfUmCfCmAfCmC*fU*mU | 21 |
| siSOD1M2-AC2(N15)-S1V3v-Qu5 | ODV- | SOD1 | 60 | Qu5-fG*mG*fUmGfGmAfAmAfUfGfAmAfGmAfAmAfG*mU*fA-S18- | 34 |
| | siRNA | | | U*A*G*A*C*U*A*G*A*U*C*A*U*A*U | |
| | | | 57 | VPmU*fA*mCfUmUfUmCfUmUfCmAfUmUfUmCfCmAfCmC*fU*mU | 21 |
| siSOD1M2-AC2(N12)-S1V3v-Qu5 | ODV- | SOD1 | 62 | Qu5-fG*mG*fUmGfGmAfAmAfUfGfAmAfGmAfAmAfG*mU*fA-S18- | 31 |
| | siRNA | | | U*A*G*A*C*U*A*G*A*U*C*A | |
| | | | 57 | VPmU*fA*mCfUmUfUmCfUmUfCmAfUmUfUmCfCmAfCmC*fU*mU | 21 |
| siSOD1M2-AC2(N6)-S1V3v-Qu5 | ODV- | SOD1 | 64 | Qu5-fG*mG*fUmGfGmAfAmAfUfGfAmAfGmAfAmAfG*mU*fA-S18- | 25 |
| | siRNA | | | U*A*G*A*C*U | |
| | | | 57 | VPmU*fA*mCfUmUfUmCfUmUfCmAfUmUfUmCfCmAfCmC*fU*mU | 21 |
| R6-04(20)-S1V1v(CM-4) | saRNA | SMN2 | 66 | mG*fA*mCfGmAfGmGfCfCfUfAfAmGfCmAfA*mC*fA | 18 |
| | | | 67 | VPmU*fG*mUfUmGfCmUfUmAfGmGfCmC*fU*mC*fG*mU*fC*mU*fC | 20 |
| R6-04M1-AC2(18)-S1L1V3v | ODV- | SMN2 | 68 | mG*fA*mCfGmAfGmGfCfCfUfAfAmGfCmAfA*mC*fA-S18- | 36 |
| | saRNA | | | meU*meA*meG*meA*meC*meU*meA*meG*mcA*meU*meC*meA*meU* | |
| | | | | meA*meU*meG*meA*meG | |
| | | | 67 | VPmU*fG*mUfUmGfCmUfUmAfGmGfCmC*fU*mC*fG*mU*fC*mU*fC | 20 |
| R6-04M1-AC2(16)-S1L1V3v | ODV- | SMN2 | 70 | mG*fA*mCfGmAfGmGfCfCfUfAfAmGfCmAfA*mC*fA-S18- | 34 |
| | saRNA | | | meU*mcA*meG*meA*meC*meU*meA*meG*meA*meU*meC*meA*meU* | |
| | | | | meA*meU*meG | |
| | | | 67 | VPmU*fG*mUfUmGfCmUfUmAfGmGfCmC*fU*mC*fG*mU*fC*mU*fC | 20 |
| R6-04M1-AC2(15)-S1L1V3v | ODV- | SMN2 | 72 | mG*fA*mCfGmAfGmGfCfCfUfAfAmGfCmAfA*mC*fA-S18- | 33 |
| | saRNA | | | meU*meA*meG*meA*meC*mcU*meA*meG*meA*meU*meC*meA*meU* | |
| | | | | meA*meU | |
| | | | 67 | VPmU*fG*mUfUmGfCmUfUmAfGmGfCmC*fU*mC*fG*mU*fC*mU*fC | 20 |
| R6-04M1-AC2(14)-S1L1V3v | ODV- | SMN2 | 74 | mG*fA*mCfGmAfGmGfCfCfUfAfAmGfCmAfA*mC*fA-S18- | 32 |
| | saRNA | | | meU*meA*meG*meA*meC*meU*meA*meG*meA*meU*meC*meA*meU* | |
| | | | | meA | |
| | | | 67 | VPmU*fG*mUfUmGfCmUfUmAfGmGfCmC*FU*mC*FG*mU*fC*mU*fC | 20 |
| R6-04M1-AC2(13)-S1L1V3v | ODV- | SMN2 | 76 | mG*fA*mCfGmAfGmGfCfCfUfAfAmGfCmAfA*mC*fA-S18- | 31 |
| | saRNA | | | meU*meA*meG*meA*meC*meU*meA*meG*meA*meU*meC*meA*meU | |
| | | | 67 | VPmU*fG*mUfUmGfCmUfUmAfGmGfCmC*fU*mC*fG*mU*fC*mU*fC | 20 |
| R6-04M1-AC2(12)-S1L1V3v | ODV- | SMN2 | 78 | mG*fA*mCfGmAfGmGfCfCfUfAfAmGfCmAfA*mC*fA-S18- | 30 |
| | saRNA | | | meU*meA*meG*meA*meC*meU*meA*meG*meA*meU*meC*meA | |
| | | | 67 | VPmU*fG*mUfUmGfCmUfUmAfGmGfCmC*fU*mC*fG*mU*fC*mU*fC | 20 |
| R6-04M1-AC2(11)-S1L1V3v | ODV- | SMN2 | 80 | mG*fA*mCfGmAfGmGfCfCfUfAfAmGfCmAfA*mC*fA-S18- | 29 |
| | saRNA | | | meU*meA*meG*meA*meC*meU*meA*meG*meA*meU*meC | |
| | | | 67 | VPmU*fG*mUfUmGfCmUfUmAfGmGfCmC*fU*mC*fG*mU*fC*mU*fC | 20 |
| R6-04M1-AC2(10)-S1L1V3v | ODV- | SMN2 | 82 | mG*fA*mCfGmAfGmGfCfCfUfAfAmGfCmAfA*mC*fA-S18- | 28 |
| | saRNA | | | meU*meA*meG*meA*meC*meU*meA*meG*meA*meU | |
| | | | 67 | VPmU*fG*mUfUmGfCmUfUmAfGmGfCmC*fU*mC*fG*mU*fC*mU*fC | 20 |
| R6-04M1-AC2(9)-S1L1V3v | ODV- | SMN2 | 84 | mG*fA*mCfGmAfGmGfCfCfUfAfAmGfCmAfA*mC*fA-S18- | 27 |
| | saRNA | | | meU*meA*meG*meA*meC*meU*meA*meG*meA | |
| | | | 67 | VPmU*fG*mUfUmGfCmUfUmAfGmGfCmC*fU*mC*fG*mU*fC*mU*fC | 20 |
| R6-04M1-AC2(8)-S1L1V3v | ODV- | SMN2 | 86 | mG*fA*mCfGmAfGmGfCfCfUfAfAmGfCmAfA*mC*fA-S18- | 27 |
| | saRNA | | | meU*meA*meG*meA*meC*meU*meA*meG | |
| | | | 67 | VPmU*fG*mUfUmGfCmUfUmAfGmGfCmC*fU*mC*fG*mU*fC*mU*fC | 20 |

n.b.: upper case, RNA; *, phosphorothioate (PS) backbone modification; f, 2′-fluoro; m, 2′-O-methyl (2′-OMe); me, 2′-O-methoxyetbyl (2′MOE); VP, 5′-(E)-vinylphosphonate; C, 5′-methyl cytosine; U, 5′-methyl uracil, S18, spacer-18 linker; Qu5, Quasar570

TABLE 3
Oligonucleotide strand sequences and duplex compositions

| | SEQ | | SEQ | | SEQ | |
|---|---|---|---|---|---|---|
| | ID | Target sequence | ID | | ID | |
| siRNA | NO | (5′-3′) | NO | Sense (5′-3′) | NO | Antisense (5′-3′) |
| siSOD1-5 | 88 | cgacgaaggccgtgtgcgt | 357 | CGACGAAGGCCGUGUGCGUTT | 626 | ACGCACACGGCCUUCGUCGTT |
| siSOD1-8 | 89 | cgaaggccgtgtgcgtgct | 358 | CGAAGGCCGUGUGCGUGCUTT | 627 | AGCACGCACACGGCCUUCGTT |
| siSOD1-10 | 90 | aaggccgtgtgcgtgctga | 359 | AAGGCCGUGUGCGUGCUGATT | 628 | UCAGCACGCACACGGCCUUTT |
| siSOD1-11 | 91 | aggccgtgtgcgtgctgaa | 360 | AGGCCGUGUGCGUGCUGAATT | 629 | UUCAGCACGCACACGGCCUTT |
| siSOD1-17 | 92 | tgtgcgtgctgaagggcga | 361 | UGUGCGUGCUGAAGGGCGATT | 630 | UCGCCCUUCAGCACGCACATT |
| siSOD1-35 | 93 | acggcccagtgcagggcat | 362 | ACGGCCCAGUGCAGGGCAUTT | 631 | AUGCCCUGCACUGGGCCGUTT |
| siSOD1-37 | 94 | ggcccagtgcagggcatca | 363 | GGCCCAGUGCAGGGCAUCATT | 632 | UGAUGCCCUGCACUGGGCCTT |
| siSOD1-38 | 95 | gcccagtgcagggcatcat | 364 | GCCCAGUGCAGGGCAUCAUTT | 633 | AUGAUGCCCUGCACUGGGCTT |
| siSOD1-40 | 96 | ccagtgcagggcatcatca | 365 | CCAGUGCAGGGCAUCAUCATT | 634 | UGAUGAUGCCCUGCACUGGTT |
| siSOD1-41 | 97 | cagtgcagggcatcatcaa | 366 | CAGUGCAGGGCAUCAUCAATT | 635 | UUGAUGAUGCCCUGCACUGTT |
| siSOD1-42 | 98 | agtgcagggcatcatcaat | 367 | AGUGCAGGGCAUCAUCAAUTT | 636 | AUUGAUGAUGCCCUGCACUTT |
| siSOD1-43 | 99 | gtgcagggcatcatcaatt | 368 | GUGCAGGGCAUCAUCAAUUTT | 637 | AAUUGAUGAUGCCCUGCACTT |
| siSOD1-44 | 100 | tgcagggcatcatcaattt | 369 | UGCAGGGCAUCAUCAAUUUTT | 638 | AAAUUGAUGAUGCCCUGCATT |
| siSOD1-45 | 101 | gcagggcatcatcaatttc | 370 | GCAGGGCAUCAUCAAUUUCTT | 639 | GAAAUUGAUGAUGCCCUGCTT |
| siSOD1-46 | 102 | cagggcatcatcaatttcg | 371 | CAGGGCAUCAUCAAUUUCGTT | 640 | CGAAAUUGAUGAUGCCCUGTT |
| siSOD1-47 | 103 | agggcatcatcaatttcga | 372 | AGGGCAUCAUCAAUUUCGATT | 641 | UCGAAAUUGAUGAUGCCCUTT |
| siSOD1-50 | 104 | gcatcatcaatttcgagca | 373 | GCAUCAUCAAUUUCGAGCATT | 642 | UGCUCGAAAUUGAUGAUGCTT |
| siSOD1-51 | 105 | catcatcaatttcgagcag | 374 | CAUCAUCAAUUUCGAGCAGTT | 643 | CUGCUCGAAAUUGAUGAUGTT |
| siSOD1-52 | 106 | atcatcaatttcgagcaga | 375 | AUCAUCAAUUUCGAGCAGATT | 644 | UCUGCUCGAAAUUGAUGAUTT |
| siSOD1-53 | 107 | tcatcaatttcgagcagaa | 376 | UCAUCAAUUUCGAGCAGAATT | 645 | UUCUGCUCGAAAUUGAUGATT |
| siSOD1-56 | 108 | tcaatttcgagcagaagga | 377 | UCAAUUUCGAGCAGAAGGATT | 646 | UCCUUCUGCUCGAAAUUGATT |
| SISOD1-57 | 109 | caatttcgagcagaaggaa | 378 | CAAUUUCGAGCAGAAGGAATT | 647 | UUCCUUCUGCUCGAAAUUGTT |
| siSOD1-58 | 110 | aatttcgagcagaaggaaa | 379 | AAUUUCGAGCAGAAGGAAATT | 648 | UUUCCUUCUGCUCGAAAUUTT |
| siSOD1-59 | 111 | atttcgagcagaaggaaag | 380 | AUUUCGAGCAGAAGGAAAGTT | 649 | CUUUCCUUCUGCUCGAAAUTT |
| siSOD1-60 | 112 | tttcgagcagaaggaaagt | 381 | UUUCGAGCAGAAGGAAAGUTT | 650 | ACUUUCCUUCUGCUCGAAATT |
| siSOD1-61 | 113 | ttcgagcagaaggaaagta | 382 | UUCGAGCAGAAGGAAAGUATT | 651 | UACUUUCCUUCUGCUCGAATT |
| siSOD1-62 | 114 | tcgagcagaaggaaagtaa | 383 | UCGAGCAGAAGGAAAGUAATT | 652 | UUACUUUCCUUCUGCUCGATT |
| siSOD1-63 | 115 | cgagcagaaggaaagtaat | 384 | CGAGCAGAAGGAAAGUAAUTT | 653 | AUUACUUUCCUUCUGCUCGTT |
| siSOD1-64 | 116 | gagcagaaggaaagtaatg | 385 | GAGCAGAAGGAAAGUAAUGTT | 654 | CAUUACUUUCCUUCUGCUCTT |
| siSOD1-66 | 117 | gcagaaggaaagtaatgga | 386 | GCAGAAGGAAAGUAAUGGATT | 655 | UCCAUUACUUUCCUUCUGCTT |
| siSOD1-69 | 118 | gaaggaaagtaatggacca | 387 | GAAGGAAAGUAAUGGACCATT | 656 | UGGUCCAUUACUUUCCUUCTT |
| siSOD1-72 | 119 | ggaaagtaatggaccagtg | 388 | GGAAAGUAAUGGACCAGUGTT | 657 | CACUGGUCCAUUACUUUCCTT |
| siSOD1-73 | 120 | gaaagtaatggaccagtga | 389 | GAAAGUAAUGGACCAGUGATT | 658 | UCACUGGUCCAUUACUUUCTT |
| siSOD1-74 | 121 | aaagtaatggaccagtgaa | 390 | AAAGUAAUGGACCAGUGAATT | 659 | UUCACUGGUCCAUUACUUUTT |
| siSOD1-75 | 122 | aagtaatggaccagtgaag | 391 | AAGUAAUGGACCAGUGAAGTT | 660 | CUUCACUGGUCCAUUACUUTT |
| siSOD1-77 | 123 | gtaatggaccagtgaaggt | 392 | GUAAUGGACCAGUGAAGGUTT | 661 | ACCUUCACUGGUCCAUUACTT |
| siSOD1-79 | 124 | aatggaccagtgaaggtgt | 393 | AAUGGACCAGUGAAGGUGUTT | 662 | ACACCUUCACUGGUCCAUUTT |
| siSOD1-80 | 125 | atggaccagtgaaggtgtg | 394 | AUGGACCAGUGAAGGUGUGTT | 663 | CACACCUUCACUGGUCCAUTT |
| siSOD1-85 | 126 | ccagtgaaggtgtggggaa | 395 | CCAGUGAAGGUGUGGGGAATT | 664 | UUCCCCACACCUUCACUGGTT |
| siSOD1-88 | 127 | gtgaaggtgtggggaagca | 396 | GUGAAGGUGUGGGGAAGCATT | 665 | UGCUUCCCCACACCUUCACTT |
| siSOD1-89 | 128 | tgaaggtgtggggaagcat | 397 | UGAAGGUGUGGGGAAGCAUTT | 666 | AUGCUUCCCCACACCUUCATT |
| siSOD1-90 | 129 | gaaggtgtggggaagcatt | 398 | GAAGGUGUGGGGAAGCAUUTT | 667 | AAUGCUUCCCCACACCUUCTT |
| siSOD1-91 | 130 | aaggtgtggggaagcatta | 399 | AAGGUGUGGGGAAGCAUUATT | 668 | UAAUGCUUCCCCACACCUUTT |
| siSOD1-92 | 131 | aggtgtggggaagcattaa | 400 | AGGUGUGGGGAAGCAUUAATT | 669 | UUAAUGCUUCCCCACACCUTT |
| siSOD1-93 | 132 | ggtgtggggaagcattaaa | 401 | GGUGUGGGGAAGCAUUAAATT | 670 | UUUAAUGCUUCCCCACACCTT |
| siSOD1-94 | 133 | gtgtggggaagcattaaag | 402 | GUGUGGGGAAGCAUUAAAGTT | 671 | CUUUAAUGCUUCCCCACACTT |
| siSOD1-95 | 134 | tgtggggaagcattaaagg | 403 | UGUGGGGAAGCAUUAAAGGTT | 672 | CCUUUAAUGCUUCCCCACATT |
| siSOD1-96 | 135 | gtggggaagcattaaagga | 404 | GUGGGGAAGCAUUAAAGGATT | 673 | UCCUUUAAUGCUUCCCCACTT |
| siSOD1-98 | 136 | ggggaagcattaaaggact | 405 | GGGGAAGCAUUAAAGGACUTT | 674 | AGUCCUUUAAUGCUUCCCCTT |
| siSOD1-99 | 137 | gggaagcattaaaggactg | 406 | GGGAAGCAUUAAAGGACUGTT | 675 | CAGUCCUUUAAUGCUUCCCTT |
| siSOD1-100 | 138 | ggaagcattaaaggactga | 407 | GGAAGCAUUAAAGGACUGATT | 676 | UCAGUCCUUUAAUGCUUCCTT |
| siSOD1-102 | 139 | aagcattaaaggactgact | 408 | AAGCAUUAAAGGACUGACUTT | 677 | AGUCAGUCCUUUAAUGCUUTT |
| siSOD1-104 | 140 | gcattaaaggactgactga | 409 | GCAUUAAAGGACUGACUGATT | 678 | UCAGUCAGUCCUUUAAUGCTT |
| siSOD1-105 | 141 | cattaaaggactgactgaa | 410 | CAUUAAAGGACUGACUGAATT | 679 | UUCAGUCAGUCCUUUAAUGTT |
| siSOD1-106 | 142 | attaaaggactgactgaag | 411 | AUUAAAGGACUGACUGAAGTT | 680 | CUUCAGUCAGUCCUUUAAUTT |
| siSOD1-107 | 143 | ttaaaggactgactgaagg | 412 | UUAAAGGACUGACUGAAGGTT | 681 | CCUUCAGUCAGUCCUUUAATT |
| siSOD1-108 | 144 | taaaggactgactgaaggc | 413 | UAAAGGACUGACUGAAGGCTT | 682 | GCCUUCAGUCAGUCCUUUATT |
| siSOD1-114 | 145 | actgactgaaggcctgcat | 414 | ACUGACUGAAGGCCUGCAUTT | 683 | AUGCAGGCCUUCAGUCAGUTT |
| siSOD1-117 | 146 | gactgaaggcctgcatgga | 415 | GACUGAAGGCCUGCAUGGATT | 684 | UCCAUGCAGGCCUUCAGUCTT |
| siSOD1-118 | 147 | actgaaggcctgcatggat | 416 | ACUGAAGGCCUGCAUGGAUTT | 685 | AUCCAUGCAGGCCUUCAGUTT |
| siSOD1-119 | 148 | ctgaaggcctgcatggatt | 417 | CUGAAGGCCUGCAUGGAUUTT | 686 | AAUCCAUGCAGGCCUUCAGTT |
| siSOD1-120 | 149 | tgaaggcctgcatggattc | 418 | UGAAGGCCUGCAUGGAUUCTT | 687 | GAAUCCAUGCAGGCCUUCATT |
| siSOD1-122 | 150 | aaggcctgcatggattcca | 419 | AAGGCCUGCAUGGAUUCCATT | 688 | UGGAAUCCAUGCAGGCCUUTT |
| siSOD1-123 | 151 | aggcctgcatggattccat | 420 | AGGCCUGCAUGGAUUCCAUTT | 689 | AUGGAAUCCAUGCAGGCCUTT |
| siSOD1-125 | 152 | gcctgcatggattccatgt | 421 | GCCUGCAUGGAUUCCAUGUTT | 690 | ACAUGGAAUCCAUGCAGGCTT |
| siSOD1-126 | 153 | cctgcatggattccatgtt | 422 | CCUGCAUGGAUUCCAUGUUTT | 691 | AACAUGGAAUCCAUGCAGGTT |
| siSOD1-127 | 154 | ctgcatggattccatgttc | 423 | CUGCAUGGAUUCCAUGUUCTT | 692 | GAACAUGGAAUCCAUGCAGTT |
| siSOD1-128 | 155 | tgcatggattccatgttca | 424 | UGCAUGGAUUCCAUGUUCATT | 693 | UGAACAUGGAAUCCAUGCATT |
| siSOD1-129 | 156 | gcatggattccatgttcat | 425 | GCAUGGAUUCCAUGUUCAUTT | 694 | AUGAACAUGGAAUCCAUGCTT |
| siSOD1-131 | 157 | atggattccatgttcatga | 426 | AUGGAUUCCAUGUUCAUGATT | 695 | UCAUGAACAUGGAAUCCAUTT |
| siSOD1-132 | 158 | tggattccatgttcatgag | 427 | UGGAUUCCAUGUUCAUGAGTT | 696 | CUCAUGAACAUGGAAUCCATT |
| siSOD1-133 | 159 | ggattccatgttcatgagt | 428 | GGAUUCCAUGUUCAUGAGUTT | 697 | ACUCAUGAACAUGGAAUCCTT |
| siSOD1-134 | 160 | gattccatgttcatgagtt | 429 | GAUUCCAUGUUCAUGAGUUTT | 698 | AACUCAUGAACAUGGAAUCTT |
| siSOD1-137 | 161 | tccatgttcatgagtttgg | 430 | UCCAUGUUCAUGAGUUUGGTT | 699 | CCAAACUCAUGAACAUGGATT |
| siSOD1-138 | 162 | ccatgttcatgagtttgga | 431 | CCAUGUUCAUGAGUUUGGATT | 700 | UCCAAACUCAUGAACAUGGTT |
| siSOD1-140 | 163 | atgttcatgagtttggaga | 432 | AUGUUCAUGAGUUUGGAGATT | 701 | UCUCCAAACUCAUGAACAUTT |
| siSOD1-141 | 164 | tgttcatgagtttggagat | 433 | UGUUCAUGAGUUUGGAGAUTT | 702 | AUCUCCAAACUCAUGAACATT |
| siSOD1-142 | 165 | gttcatgagtttggagata | 434 | GUUCAUGAGUUUGGAGAUATT | 703 | UAUCUCCAAACUCAUGAACTT |
| siSOD1-148 | 166 | gagtttggagataatacag | 435 | GAGUUUGGAGAUAAUACAGTT | 704 | CUGUAUUAUCUCCAAACUCTT |
| siSOD1-150 | 167 | gtttggagataatacagca | 436 | GUUUGGAGAUAAUACAGCATT | 705 | UGCUGUAUUAUCUCCAAACTT |
| siSOD1-154 | 168 | ggagataatacagcaggct | 437 | GGAGAUAAUACAGCAGGCUTT | 706 | AGCCUGCUGUAUUAUCUCCTT |
| siSOD1-156 | 169 | agataatacagcaggctgt | 438 | AGAUAAUACAGCAGGCUGUTT | 707 | ACAGCCUGCUGUAUUAUCUTT |
| siSOD1-157 | 170 | gataatacagcaggctgta | 439 | GAUAAUACAGCAGGCUGUATT | 708 | UACAGCCUGCUGUAUUAUCTT |
| siSOD1-158 | 171 | ataatacagcaggctgtac | 440 | AUAAUACAGCAGGCUGUACTT | 709 | GUACAGCCUGCUGUAUUAUTT |
| siSOD1-159 | 172 | taatacagcaggctgtacc | 441 | UAAUACAGCAGGCUGUACCTT | 710 | GGUACAGCCUGCUGUAUUATT |
| siSOD1-160 | 173 | aatacagcaggctgtacca | 442 | AAUACAGCAGGCUGUACCATT | 711 | UGGUACAGCCUGCUGUAUUTT |
| siSOD1-162 | 174 | tacagcaggctgtaccagt | 443 | UACAGCAGGCUGUACCAGUTT | 712 | ACUGGUACAGCCUGCUGUATT |
| siSOD1-163 | 175 | acagcaggctgtaccagtg | 444 | ACAGCAGGCUGUACCAGUGTT | 713 | CACUGGUACAGCCUGCUGUTT |
| siSOD1-165 | 176 | agcaggctgtaccagtgca | 445 | AGCAGGCUGUACCAGUGCATT | 714 | UGCACUGGUACAGCCUGCUTT |
| siSOD1-171 | 177 | ctgtaccagtgcaggtcct | 446 | CUGUACCAGUGCAGGUCCUTT | 715 | AGGACCUGCACUGGUACAGTT |
| siSOD1-173 | 178 | gtaccagtgcaggtcctca | 447 | GUACCAGUGCAGGUCCUCATT | 716 | UGAGGACCUGCACUGGUACTT |
| siSOD1-175 | 179 | accagtgcaggtcctcact | 448 | ACCAGUGCAGGUCCUCACUTT | 717 | AGUGAGGACCUGCACUGGUTT |
| siSOD1-176 | 180 | ccagtgcaggtcctcactt | 449 | CCAGUGCAGGUCCUCACUUTT | 718 | AAGUGAGGACCUGCACUGGTT |
| siSOD1-177 | 181 | cagtgcaggtcctcacttt | 450 | CAGUGCAGGUCCUCACUUUTT | 719 | AAAGUGAGGACCUGCACUGTT |
| siSOD1-178 | 182 | agtgcaggtcctcacttta | 451 | AGUGCAGGUCCUCACUUUATT | 720 | UAAAGUGAGGACCUGCACUTT |
| siSOD1-179 | 183 | gtgcaggtcctcactttaa | 452 | GUGCAGGUCCUCACUUUAATT | 721 | UUAAAGUGAGGACCUGCACTT |
| siSOD1-180 | 184 | tgcaggtcctcactttaat | 453 | UGCAGGUCCUCACUUUAAUTT | 722 | AUUAAAGUGAGGACCUGCATT |
| siSOD1-181 | 185 | gcaggtcctcactttaatc | 454 | GCAGGUCCUCACUUUAAUCTT | 723 | GAUUAAAGUGAGGACCUGCTT |
| siSOD1-182 | 186 | caggtcctcactttaatcc | 455 | CAGGUCCUCACUUUAAUCCTT | 724 | GGAUUAAAGUGAGGACCUGTT |
| siSOD1-183 | 187 | aggtcctcactttaatcct | 456 | AGGUCCUCACUUUAAUCCUTT | 725 | AGGAUUAAAGUGAGGACCUTT |
| siSOD1-184 | 188 | ggtcctcactttaatcctc | 457 | GGUCCUCACUUUAAUCCUCTT | 726 | GAGGAUUAAAGUGAGGACCTT |
| siSOD1-185 | 189 | gtcctcactttaatcctct | 458 | GUCCUCACUUUAAUCCUCUTT | 727 | AGAGGAUUAAAGUGAGGACTT |
| siSOD1-186 | 190 | tcctcactttaatcctcta | 459 | UCCUCACUUUAAUCCUCUATT | 728 | UAGAGGAUUAAAGUGAGGATT |
| siSOD1-187 | 191 | cctcactttaatcctctat | 460 | CCUCACUUUAAUCCUCUAUTT | 729 | AUAGAGGAUUAAAGUGAGGTT |
| siSOD1-188 | 192 | ctcactttaatcctctatc | 461 | CUCACUUUAAUCCUCUAUCTT | 730 | GAUAGAGGAUUAAAGUGAGTT |
| siSOD1-189 | 193 | tcactttaatcctctatcc | 462 | UCACUUUAAUCCUCUAUCCTT | 731 | GGAUAGAGGAUUAAAGUGATT |
| siSOD1-190 | 194 | cactttaatcctctatcca | 463 | CACUUUAAUCCUCUAUCCATT | 732 | UGGAUAGAGGAUUAAAGUGTT |
| siSOD1-192 | 195 | ctttaatcctctatccaga | 464 | CUUUAAUCCUCUAUCCAGATT | 733 | UCUGGAUAGAGGAUUAAAGTT |
| siSOD1-196 | 196 | aatcctctatccagaaaac | 465 | AAUCCUCUAUCCAGAAAACTT | 734 | GUUUUCUGGAUAGAGGAUUTT |
| siSOD1-197 | 197 | atcctctatccagaaaaca | 466 | AUCCUCUAUCCAGAAAACATT | 735 | UGUUUUCUGGAUAGAGGAUTT |
| siSOD1-198 | 198 | tcctctatccagaaaacac | 467 | UCCUCUAUCCAGAAAACACTT | 736 | GUGUUUUCUGGAUAGAGGATT |
| siSOD1-201 | 199 | tctatccagaaaacacggt | 468 | UCUAUCCAGAAAACACGGUTT | 737 | ACCGUGUUUUCUGGAUAGATT |
| siSOD1-208 | 200 | agaaaacacggtgggccaa | 469 | AGAAAACACGGUGGGCCAATT | 738 | UUGGCCCACCGUGUUUUCUTT |
| siSOD1-209 | 201 | gaaaacacggtgggccaaa | 470 | GAAAACACGGUGGGCCAAATT | 739 | UUUGGCCCACCGUGUUUUCTT |
| siSOD1-210 | 202 | aaaacacggtgggccaaag | 471 | AAAACACGGUGGGCCAAAGTT | 740 | CUUUGGCCCACCGUGUUUUTT |
| siSOD1-211 | 203 | aaacacggtgggccaaagg | 472 | AAACACGGUGGGCCAAAGGTT | 741 | CCUUUGGCCCACCGUGUUUTT |
| siSOD1-212 | 204 | aacacggtgggccaaagga | 473 | AACACGGUGGGCCAAAGGATT | 742 | UCCUUUGGCCCACCGUGUUTT |
| siSOD1-213 | 205 | acacggtgggccaaaggat | 474 | ACACGGUGGGCCAAAGGAUTT | 743 | AUCCUUUGGCCCACCGUGUTT |
| siSOD1-215 | 206 | acggtgggccaaaggatga | 475 | ACGGUGGGCCAAAGGAUGATT | 744 | UCAUCCUUUGGCCCACCGUTT |
| siSOD1-216 | 207 | cggtgggccaaaggatgaa | 476 | CGGUGGGCCAAAGGAUGAATT | 745 | UUCAUCCUUUGGCCCACCGTT |
| siSOD1-218 | 208 | gtgggccaaaggatgaaga | 477 | GUGGGCCAAAGGAUGAAGATT | 746 | UCUUCAUCCUUUGGCCCACTT |
| siSOD1-219 | 209 | tgggccaaaggatgaagag | 478 | UGGGCCAAAGGAUGAAGAGTT | 747 | CUCUUCAUCCUUUGGCCCATT |
| siSOD1-220 | 210 | gggccaaaggatgaagaga | 479 | GGGCCAAAGGAUGAAGAGATT | 748 | UCUCUUCAUCCUUUGGCCCTT |
| siSOD1-222 | 211 | gccaaaggatgaagagagg | 480 | GCCAAAGGAUGAAGAGAGGTT | 749 | CCUCUCUUCAUCCUUUGGCTT |
| siSOD1-224 | 212 | caaaggatgaagagaggca | 481 | CAAAGGAUGAAGAGAGGCATT | 750 | UGCCUCUCUUCAUCCUUUGTT |
| siSOD1-225 | 213 | aaaggatgaagagaggcat | 482 | AAAGGAUGAAGAGAGGCAUTT | 751 | AUGCCUCUCUUCAUCCUUUTT |
| SISOD1-227 | 214 | aggatgaagagaggcatgt | 483 | AGGAUGAAGAGAGGCAUGUTT | 752 | ACAUGCCUCUCUUCAUCCUTT |
| siSOD1-228 | 215 | ggatgaagagaggcatgtt | 484 | GGAUGAAGAGAGGCAUGUUTT | 753 | AACAUGCCUCUCUUCAUCCTT |
| siSOD1-229 | 216 | gatgaagagaggcatgttg | 485 | GAUGAAGAGAGGCAUGUUGTT | 754 | CAACAUGCCUCUCUUCAUCTT |
| siSOD1-231 | 217 | tgaagagaggcatgttgga | 486 | UGAAGAGAGGCAUGUUGGATT | 755 | UCCAACAUGCCUCUCUUCATT |
| siSOD1-233 | 218 | aagagaggcatgttggaga | 487 | AAGAGAGGCAUGUUGGAGATT | 756 | UCUCCAACAUGCCUCUCUUTT |
| siSOD1-234 | 219 | agagaggcatgttggagac | 488 | AGAGAGGCAUGUUGGAGACTT | 757 | GUCUCCAACAUGCCUCUCUTT |
| siSOD1-235 | 220 | gagaggcatgttggagact | 489 | GAGAGGCAUGUUGGAGACUTT | 758 | AGUCUCCAACAUGCCUCUCTT |
| siSOD1-236 | 221 | agaggcatgttggagactt | 490 | AGAGGCAUGUUGGAGACUUTT | 759 | AAGUCUCCAACAUGCCUCUTT |
| siSOD1-237 | 222 | gaggcatgttggagacttg | 491 | GAGGCAUGUUGGAGACUUGTT | 760 | CAAGUCUCCAACAUGCCUCTT |
| siSOD1-241 | 223 | catgttggagacttgggca | 492 | CAUGUUGGAGACUUGGGCATT | 761 | UGCCCAAGUCUCCAACAUGTT |
| siSOD1-242 | 224 | atgttggagacttgggcaa | 493 | AUGUUGGAGACUUGGGCAATT | 762 | UUGCCCAAGUCUCCAACAUTT |
| siSOD1-243 | 225 | tgttggagacttgggcaat | 494 | UGUUGGAGACUUGGGCAAUTT | 763 | AUUGCCCAAGUCUCCAACATT |
| siSOD1-244 | 226 | gttggagacttgggcaatg | 495 | GUUGGAGACUUGGGCAAUGTT | 764 | CAUUGCCCAAGUCUCCAACTT |
| siSOD1-245 | 227 | ttggagacttgggcaatgt | 496 | UUGGAGACUUGGGCAAUGUTT | 765 | ACAUUGCCCAAGUCUCCAATT |
| siSOD1-246 | 228 | tggagacttgggcaatgtg | 497 | UGGAGACUUGGGCAAUGUGTT | 766 | CACAUUGCCCAAGUCUCCATT |
| siSOD1-247 | 229 | ggagacttgggcaatgtga | 498 | GGAGACUUGGGCAAUGUGATT | 767 | UCACAUUGCCCAAGUCUCCTT |
| siSOD1-249 | 230 | agacttgggcaatgtgact | 499 | AGACUUGGGCAAUGUGACUTT | 768 | AGUCACAUUGCCCAAGUCUTT |
| siSOD1-252 | 231 | cttgggcaatgtgactgct | 500 | CUUGGGCAAUGUGACUGCUTT | 769 | AGCAGUCACAUUGCCCAAGTT |
| siSOD1-254 | 232 | tgggcaatgtgactgctga | 501 | UGGGCAAUGUGACUGCUGATT | 770 | UCAGCAGUCACAUUGCCCATT |
| siSOD1-256 | 233 | ggcaatgtgactgctgaca | 502 | GGCAAUGUGACUGCUGACATT | 771 | UGUCAGCAGUCACAUUGCCTT |
| siSOD1-257 | 234 | gcaatgtgactgctgacaa | 503 | GCAAUGUGACUGCUGACAATT | 772 | UUGUCAGCAGUCACAUUGCTT |
| siSOD1-258 | 235 | caatgtgactgctgacaaa | 504 | CAAUGUGACUGCUGACAAATT | 773 | UUUGUCAGCAGUCACAUUGTT |
| siSOD1-260 | 236 | atgtgactgctgacaaaga | 505 | AUGUGACUGCUGACAAAGATT | 774 | UCUUUGUCAGCAGUCACAUTT |
| siSOD1-261 | 237 | tgtgactgctgacaaagat | 506 | UGUGACUGCUGACAAAGAUTT | 775 | AUCUUUGUCAGCAGUCACATT |
| siSOD1-262 | 238 | gtgactgctgacaaagatg | 507 | GUGACUGCUGACAAAGAUGTT | 776 | CAUCUUUGUCAGCAGUCACTT |
| siSOD1-263 | 239 | tgactgctgacaaagatgc | 508 | UGACUGCUGACAAAGAUGCTT | 777 | GCAUCUUUGUCAGCAGUCATT |
| siSOD1-264 | 240 | gactgctgacaaagatgct | 509 | GACUGCUGACAAAGAUGCUTT | 778 | AGCAUCUUUGUCAGCAGUCTT |
| siSOD1-266 | 241 | ctgctgacaaagatgctgt | 510 | CUGCUGACAAAGAUGCUGUTT | 779 | ACAGCAUCUUUGUCAGCAGTT |
| siSOD1-270 | 242 | tgacaaagatgctgtggcc | 511 | UGACAAAGAUGCUGUGGCCTT | 780 | GGCCACAGCAUCUUUGUCATT |
| siSOD1-272 | 243 | acaaagatgctgtggccga | 512 | ACAAAGAUGCUGUGGCCGATT | 781 | UCGGCCACAGCAUCUUUGUTT |
| siSOD1-273 | 244 | caaagatgctgtggccgat | 513 | CAAAGAUGCUGUGGCCGAUTT | 782 | AUCGGCCACAGCAUCUUUGTT |
| siSOD1-275 | 245 | aagatgctgtggccgatgt | 514 | AAGAUGCUGUGGCCGAUGUTT | 783 | ACAUCGGCCACAGCAUCUUTT |
| siSOD1-276 | 246 | agatgctgtggccgatgtg | 515 | AGAUGCUGUGGCCGAUGUGTT | 784 | CACAUCGGCCACAGCAUCUTT |
| siSOD1-279 | 247 | tgctgtggccgatgtgtct | 516 | UGCUGUGGCCGAUGUGUCUTT | 785 | AGACACAUCGGCCACAGCATT |
| siSOD1-280 | 248 | gctgtggccgatgtgtcta | 517 | GCUGUGGCCGAUGUGUCUATT | 786 | UAGACACAUCGGCCACAGCTT |
| siSOD1-281 | 249 | ctgtggccgatgtgtctat | 518 | CUGUGGCCGAUGUGUCUAUTT | 787 | AUAGACACAUCGGCCACAGTT |
| siSOD1-282 | 250 | tgtggccgatgtgtctatt | 519 | UGUGGCCGAUGUGUCUAUUTT | 788 | AAUAGACACAUCGGCCACATT |
| siSOD1-283 | 251 | gtggccgatgtgtctattg | 520 | GUGGCCGAUGUGUCUAUUGTT | 789 | CAAUAGACACAUCGGCCACTT |
| siSOD1-284 | 252 | tggccgatgtgtctattga | 521 | UGGCCGAUGUGUCUAUUGATT | 790 | UCAAUAGACACAUCGGCCATT |
| siSOD1-285 | 253 | ggccgatgtgtctattgaa | 522 | GGCCGAUGUGUCUAUUGAATT | 791 | UUCAAUAGACACAUCGGCCTT |
| siSOD1-286 | 254 | gccgatgtgtctattgaag | 523 | GCCGAUGUGUCUAUUGAAGTT | 792 | CUUCAAUAGACACAUCGGCTT |
| siSOD1-287 | 255 | ccgatgtgtctattgaaga | 524 | CCGAUGUGUCUAUUGAAGATT | 793 | UCUUCAAUAGACACAUCGGTT |
| siSOD1-288 | 256 | cgatgtgtctattgaagat | 525 | CGAUGUGUCUAUUGAAGAUTT | 794 | AUCUUCAAUAGACACAUCGTT |
| siSOD1-299 | 257 | ttgaagattctgtgatctc | 526 | UUGAAGAUUCUGUGAUCUCTT | 795 | GAGAUCACAGAAUCUUCAATT |
| siSOD1-300 | 258 | tgaagattctgtgatctca | 527 | UGAAGAUUCUGUGAUCUCATT | 796 | UGAGAUCACAGAAUCUUCATT |
| siSOD1-301 | 259 | gaagattctgtgatctcac | 528 | GAAGAUUCUGUGAUCUCACTT | 797 | GUGAGAUCACAGAAUCUUCTT |
| siSOD1-302 | 260 | aagattctgtgatctcact | 529 | AAGAUUCUGUGAUCUCACUTT | 798 | AGUGAGAUCACAGAAUCUUTT |
| siSOD1-303 | 261 | agattctgtgatctcactc | 530 | AGAUUCUGUGAUCUCACUCTT | 799 | GAGUGAGAUCACAGAAUCUTT |
| siSOD1-304 | 262 | gattctgtgatctcactct | 531 | GAUUCUGUGAUCUCACUCUTT | 800 | AGAGUGAGAUCACAGAAUCTT |
| siSOD1-305 | 263 | attctgtgatctcactctc | 532 | AUUCUGUGAUCUCACUCUCTT | 801 | GAGAGUGAGAUCACAGAAUTT |
| siSOD1-306 | 264 | ttctgtgatctcactctca | 533 | UUCUGUGAUCUCACUCUCATT | 802 | UGAGAGUGAGAUCACAGAATT |
| siSOD1-307 | 265 | tctgtgatctcactctcag | 534 | UCUGUGAUCUCACUCUCAGTT | 803 | CUGAGAGUGAGAUCACAGATT |
| siSOD1-309 | 266 | tgtgatctcactctcagga | 535 | UGUGAUCUCACUCUCAGGATT | 804 | UCCUGAGAGUGAGAUCACATT |
| siSOD1-311 | 267 | tgatctcactctcaggaga | 536 | UGAUCUCACUCUCAGGAGATT | 805 | UCUCCUGAGAGUGAGAUCATT |
| siSOD1-314 | 268 | tctcactctcaggagacca | 537 | UCUCACUCUCAGGAGACCATT | 806 | UGGUCUCCUGAGAGUGAGATT |
| siSOD1-315 | 269 | ctcactctcaggagaccat | 538 | CUCACUCUCAGGAGACCAUTT | 807 | AUGGUCUCCUGAGAGUGAGTT |
| siSOD1-316 | 270 | tcactctcaggagaccatt | 539 | UCACUCUCAGGAGACCAUUTT | 808 | AAUGGUCUCCUGAGAGUGATT |
| siSOD1-317 | 271 | cactctcaggagaccattg | 540 | CACUCUCAGGAGACCAUUGTT | 809 | CAAUGGUCUCCUGAGAGUGTT |
| siSOD1-318 | 272 | actctcaggagaccattgc | 541 | ACUCUCAGGAGACCAUUGCTT | 810 | GCAAUGGUCUCCUGAGAGUTT |
| siSOD1-319 | 273 | ctctcaggagaccattgca | 542 | CUCUCAGGAGACCAUUGCATT | 811 | UGCAAUGGUCUCCUGAGAGTT |
| siSOD1-320 | 274 | tctcaggagaccattgcat | 543 | UCUCAGGAGACCAUUGCAUTT | 812 | AUGCAAUGGUCUCCUGAGATT |
| siSOD1-322 | 275 | tcaggagaccattgcatca | 544 | UCAGGAGACCAUUGCAUCATT | 813 | UGAUGCAAUGGUCUCCUGATT |
| siSOD1-323 | 276 | caggagaccattgcatcat | 545 | CAGGAGACCAUUGCAUCAUTT | 814 | AUGAUGCAAUGGUCUCCUGTT |
| siSOD1-324 | 277 | aggagaccattgcatcatt | 546 | AGGAGACCAUUGCAUCAUUTT | 815 | AAUGAUGCAAUGGUCUCCUTT |
| siSOD1-325 | 278 | ggagaccattgcatcattg | 547 | GGAGACCAUUGCAUCAUUGTT | 816 | CAAUGAUGCAAUGGUCUCCTT |
| siSOD1-326 | 279 | gagaccattgcatcattgg | 548 | GAGACCAUUGCAUCAUUGGTT | 817 | CCAAUGAUGCAAUGGUCUCTT |
| siSOD1-327 | 280 | agaccattgcatcattggc | 549 | AGACCAUUGCAUCAUUGGCTT | 818 | GCCAAUGAUGCAAUGGUCUTT |
| siSOD1-333 | 281 | ttgcatcattggccgcaca | 550 | UUGCAUCAUUGGCCGCACATT | 819 | UGUGCGGCCAAUGAUGCAATT |
| siSOD1-335 | 282 | gcatcattggccgcacact | 551 | GCAUCAUUGGCCGCACACUTT | 820 | AGUGUGCGGCCAAUGAUGCTT |
| siSOD1-338 | 283 | tcattggccgcacactggt | 552 | UCAUUGGCCGCACACUGGUTT | 821 | ACCAGUGUGCGGCCAAUGATT |
| siSOD1-344 | 284 | gccgcacactggtggtcca | 553 | GCCGCACACUGGUGGUCCATT | 822 | UGGACCACCAGUGUGCGGCTT |
| siSOD1-347 | 285 | gcacactggtggtccatga | 554 | GCACACUGGUGGUCCAUGATT | 823 | UCAUGGACCACCAGUGUGCTT |
| siSOD1-348 | 286 | cacactggtggtccatgaa | 555 | CACACUGGUGGUCCAUGAATT | 824 | UUCAUGGACCACCAGUGUGTT |
| siSOD1-349 | 287 | acactggtggtccatgaaa | 556 | ACACUGGUGGUCCAUGAAATT | 825 | UUUCAUGGACCACCAGUGUTT |
| siSOD1-350 | 288 | cactggtggtccatgaaaa | 557 | CACUGGUGGUCCAUGAAAATT | 826 | UUUUCAUGGACCACCAGUGTT |
| siSOD1-351 | 289 | actggtggtccatgaaaaa | 558 | ACUGGUGGUCCAUGAAAAATT | 827 | UUUUUCAUGGACCACCAGUTT |
| siSOD1-352 | 290 | ctggtggtccatgaaaaag | 559 | CUGGUGGUCCAUGAAAAAGTT | 828 | CUUUUUCAUGGACCACCAGTT |
| siSOD1-353 | 291 | tggtggtccatgaaaaagc | 560 | UGGUGGUCCAUGAAAAAGCTT | 829 | GCUUUUUCAUGGACCACCATT |
| siSOD1-354 | 292 | ggtggtccatgaaaaagca | 561 | GGUGGUCCAUGAAAAAGCATT | 830 | UGCUUUUUCAUGGACCACCTT |
| siSOD1-356 | 293 | tggtccatgaaaaagcaga | 562 | UGGUCCAUGAAAAAGCAGATT | 831 | UCUGCUUUUUCAUGGACCATT |
| siSOD1-357 | 294 | ggtccatgaaaaagcagat | 563 | GGUCCAUGAAAAAGCAGAUTT | 832 | AUCUGCUUUUUCAUGGACCTT |
| siSOD1-359 | 295 | tccatgaaaaagcagatga | 564 | UCCAUGAAAAAGCAGAUGATT | 833 | UCAUCUGCUUUUUCAUGGATT |
| siSOD1-360 | 296 | ccatgaaaaagcagatgac | 565 | CCAUGAAAAAGCAGAUGACTT | 834 | GUCAUCUGCUUUUUCAUGGTT |
| siSOD1-361 | 297 | catgaaaaagcagatgact | 566 | CAUGAAAAAGCAGAUGACUTT | 835 | AGUCAUCUGCUUUUUCAUGTT |
| siSOD1-363 | 298 | tgaaaaagcagatgacttg | 567 | UGAAAAAGCAGAUGACUUGTT | 836 | CAAGUCAUCUGCUUUUUCATT |
| siSOD1-365 | 299 | aaaaagcagatgacttggg | 568 | AAAAAGCAGAUGACUUGGGTT | 837 | CCCAAGUCAUCUGCUUUUUTT |
| siSOD1-366 | 300 | aaaagcagatgacttgggc | 569 | AAAAGCAGAUGACUUGGGCTT | 838 | GCCCAAGUCAUCUGCUUUUTT |
| siSOD1-367 | 301 | aaagcagatgacttgggca | 570 | AAAGCAGAUGACUUGGGCATT | 839 | UGCCCAAGUCAUCUGCUUUTT |
| siSOD1-368 | 302 | aagcagatgacttgggcaa | 571 | AAGCAGAUGACUUGGGCAATT | 840 | UUGCCCAAGUCAUCUGCUUTT |
| siSOD1-369 | 303 | agcagatgacttgggcaaa | 572 | AGCAGAUGACUUGGGCAAATT | 841 | UUUGCCCAAGUCAUCUGCUTT |
| siSOD1-370 | 304 | gcagatgacttgggcaaag | 573 | GCAGAUGACUUGGGCAAAGTT | 842 | CUUUGCCCAAGUCAUCUGCTT |
| siSOD1-371 | 305 | cagatgacttgggcaaagg | 574 | CAGAUGACUUGGGCAAAGGTT | 843 | CCUUUGCCCAAGUCAUCUGTT |
| siSOD1-372 | 306 | agatgacttgggcaaaggt | 575 | AGAUGACUUGGGCAAAGGUTT | 844 | ACCUUUGCCCAAGUCAUCUTT |
| siSOD1-375 | 307 | tgacttgggcaaaggtgga | 576 | UGACUUGGGCAAAGGUGGATT | 845 | UCCACCUUUGCCCAAGUCATT |
| siSOD1-376 | 308 | gacttgggcaaaggtggaa | 577 | GACUUGGGCAAAGGUGGAATT | 846 | UUCCACCUUUGCCCAAGUCTT |
| siSOD1-377 | 309 | acttgggcaaaggtggaaa | 578 | ACUUGGGCAAAGGUGGAAATT | 847 | UUUCCACCUUUGCCCAAGUTT |
| SISOD1-378 | 310 | cttgggcaaaggtggaaat | 579 | CUUGGGCAAAGGUGGAAAUTT | 848 | AUUUCCACCUUUGCCCAAGTT |
| siSOD1-379 | 311 | ttgggcaaaggtggaaatg | 580 | UUGGGCAAAGGUGGAAAUGTT | 849 | CAUUUCCACCUUUGCCCAATT |
| siSOD1-380 | 312 | tgggcaaaggtggaaatga | 581 | UGGGCAAAGGUGGAAAUGATT | 850 | UCAUUUCCACCUUUGCCCATT |
| siSOD1-381 | 313 | gggcaaaggtggaaatgaa | 582 | GGGCAAAGGUGGAAAUGAATT | 851 | UUCAUUUCCACCUUUGCCCTT |
| siSOD1-382 | 314 | ggcaaaggtggaaatgaag | 583 | GGCAAAGGUGGAAAUGAAGTT | 852 | CUUCAUUUCCACCUUUGCCTT |
| siSOD1-383 | 315 | gcaaaggtggaaatgaaga | 584 | GCAAAGGUGGAAAUGAAGATT | 853 | UCUUCAUUUCCACCUUUGCTT |
| siSOD1-384 | 316 | caaaggtggaaatgaagaa | 585 | CAAAGGUGGAAAUGAAGAATT | 854 | UUCUUCAUUUCCACCUUUGTT |
| siSOD1-386 | 317 | aaggtggaaatgaagaaag | 586 | AAGGUGGAAAUGAAGAAAGTT | 855 | CUUUCUUCAUUUCCACCUUTT |
| siSOD1-387 | 318 | aggtggaaatgaagaaagt | 587 | AGGUGGAAAUGAAGAAAGUTT | 856 | ACUUUCUUCAUUUCCACCUTT |
| siSOD1-388 | 319 | ggtggaaatgaagaaagta | 588 | GGUGGAAAUGAAGAAAGUATT | 857 | UACUUUCUUCAUUUCCACCTT |
| siSOD1-389 | 320 | gtggaaatgaagaaagtac | 589 | GUGGAAAUGAAGAAAGUACTT | 858 | GUACUUUCUUCAUUUCCACTT |
| siSOD1-397 | 321 | gaagaaagtacaaagacag | 590 | GAAGAAAGUACAAAGACAGTT | 859 | CUGUCUUUGUACUUUCUUCTT |
| siSOD1-399 | 322 | agaaagtacaaagacagga | 591 | AGAAAGUACAAAGACAGGATT | 860 | UCCUGUCUUUGUACUUUCUTT |
| siSOD1-400 | 323 | gaaagtacaaagacaggaa | 592 | GAAAGUACAAAGACAGGAATT | 861 | UUCCUGUCUUUGUACUUUCTT |
| siSOD1-402 | 324 | aagtacaaagacaggaaac | 593 | AAGUACAAAGACAGGAAACTT | 862 | GUUUCCUGUCUUUGUACUUTT |
| siSOD1-405 | 325 | tacaaagacaggaaacgct | 594 | UACAAAGACAGGAAACGCUTT | 863 | AGCGUUUCCUGUCUUUGUATT |
| siSOD1-408 | 326 | aaagacaggaaacgctgga | 595 | AAAGACAGGAAACGCUGGATT | 864 | UCCAGCGUUUCCUGUCUUUTT |
| siSOD1-409 | 327 | aagacaggaaacgctggaa | 596 | AAGACAGGAAACGCUGGAATT | 865 | UUCCAGCGUUUCCUGUCUUTT |
| siSOD1-410 | 328 | agacaggaaacgctggaag | 597 | AGACAGGAAACGCUGGAAGTT | 866 | CUUCCAGCGUUUCCUGUCUTT |
| siSOD1-411 | 329 | gacaggaaacgctggaagt | 598 | GACAGGAAACGCUGGAAGUTT | 867 | ACUUCCAGCGUUUCCUGUCTT |
| siSOD1-412 | 330 | acaggaaacgctggaagtc | 599 | ACAGGAAACGCUGGAAGUCTT | 868 | GACUUCCAGCGUUUCCUGUTT |
| siSOD1-414 | 331 | aggaaacgctggaagtcgt | 600 | AGGAAACGCUGGAAGUCGUTT | 869 | ACGACUUCCAGCGUUUCCUTT |
| siSOD1-415 | 332 | ggaaacgctggaagtcgtt | 601 | GGAAACGCUGGAAGUCGUUTT | 870 | AACGACUUCCAGCGUUUCCTT |
| siSOD1-416 | 333 | gaaacgctggaagtcgttt | 602 | GAAACGCUGGAAGUCGUUUTT | 871 | AAACGACUUCCAGCGUUUCTT |
| siSOD1-417 | 334 | aaacgctggaagtcgtttg | 603 | AAACGCUGGAAGUCGUUUGTT | 872 | CAAACGACUUCCAGCGUUUTT |
| siSOD1-418 | 335 | aacgctggaagtcgtttgg | 604 | AACGCUGGAAGUCGUUUGGTT | 873 | CCAAACGACUUCCAGCGUUTT |
| siSOD1-420 | 336 | cgctggaagtcgtttggct | 605 | CGCUGGAAGUCGUUUGGCUTT | 874 | AGCCAAACGACUUCCAGCGTT |
| siSOD1-421 | 337 | gctggaagtcgtttggctt | 606 | GCUGGAAGUCGUUUGGCUUTT | 875 | AAGCCAAACGACUUCCAGCTT |
| siSOD1-423 | 338 | tggaagtcgtttggcttgt | 607 | UGGAAGUCGUUUGGCUUGUTT | 876 | ACAAGCCAAACGACUUCCATT |
| siSOD1-424 | 339 | ggaagtcgtttggcttgtg | 608 | GGAAGUCGUUUGGCUUGUGTT | 877 | CACAAGCCAAACGACUUCCTT |
| siSOD1-425 | 340 | gaagtcgtttggcttgtgg | 609 | GAAGUCGUUUGGCUUGUGGTT | 878 | CCACAAGCCAAACGACUUCTT |
| siSOD1-426 | 341 | aagtcgtttggcttgtggt | 610 | AAGUCGUUUGGCUUGUGGUTT | 879 | ACCACAAGCCAAACGACUUTT |
| siSOD1-429 | 342 | tcgtttggcttgtggtgta | 611 | UCGUUUGGCUUGUGGUGUATT | 880 | UACACCACAAGCCAAACGATT |
| siSOD1-430 | 343 | cgtttggcttgtggtgtaa | 612 | CGUUUGGCUUGUGGUGUAATT | 881 | UUACACCACAAGCCAAACGTT |
| siSOD1-431 | 344 | gtttggcttgtggtgtaat | 613 | GUUUGGCUUGUGGUGUAAUTT | 882 | AUUACACCACAAGCCAAACTT |
| siSOD1-432 | 345 | tttggcttgtggtgtaatt | 614 | UUUGGCUUGUGGUGUAAUUTT | 883 | AAUUACACCACAAGCCAAATT |
| siSOD1-433 | 346 | ttggcttgtggtgtaattg | 615 | UUGGCUUGUGGUGUAAUUGTT | 884 | CAAUUACACCACAAGCCAATT |
| siSOD1-434 | 347 | tggcttgtggtgtaattgg | 616 | UGGCUUGUGGUGUAAUUGGTT | 885 | CCAAUUACACCACAAGCCATT |
| siSOD1-435 | 348 | ggcttgtggtgtaattggg | 617 | GGCUUGUGGUGUAAUUGGGTT | 886 | CCCAAUUACACCACAAGCCTT |
| siSOD1-436 | 349 | gcttgtggtgtaattggga | 618 | GCUUGUGGUGUAAUUGGGATT | 887 | UCCCAAUUACACCACAAGCTT |
| siSOD1-437 | 350 | cttgtggtgtaattgggat | 619 | CUUGUGGUGUAAUUGGGAUTT | 888 | AUCCCAAUUACACCACAAGTT |
| siSOD1-443 | 351 | gtgtaattgggatcgccca | 620 | GUGUAAUUGGGAUCGCCCATT | 889 | UGGGCGAUCCCAAUUACACTT |
| siSOD1-444 | 352 | tgtaattgggatcgcccaa | 621 | UGUAAUUGGGAUCGCCCAATT | 890 | UUGGGCGAUCCCAAUUACATT |
| siSOD1-445 | 353 | gtaattgggategcccaat | 622 | GUAAUUGGGAUCGCCCAAUTT | 891 | AUUGGGCGAUCCCAAUUACTT |
| siSOD1-446 | 354 | taattgggategcccaata | 623 | UAAUUGGGAUCGCCCAAUATT | 892 | UAUUGGGCGAUCCCAAUUATT |
| siSOD1-447 | 355 | aattgggategcccaataa | 624 | AAUUGGGAUCGCCCAAUAATT | 893 | UUAUUGGGCGAUCCCAAUUTT |

n.b.: Target sequence is identical to the identified sense sequence but the nucleotide ″U″ is converted to ″T″ and excluding the overhang 2 nucleotides ″TT″.

TABLE 7
ACO Sequences

| | | Size | SEQ ID |
|---|---|---|---|
| ACO Name | ACO Sequence (5′-3′) | (nt) | NO |
| N22 | L1-U*A*G*A*C*U*A*G*A*U*C*A*U*A*U*G*A*G*U*A*G*A | 22 | 1 |
| N20 | L1-U*A*G*A*C*U*A*G*A*U*C*A*U*A*U*G*A*G*U*A | 20 | 2 |
| N18 | L1-U*A*G*A*C*U*A*G*A*U*C*A*U*A*U*G*A*G | 18 | 3 |
| N16 | L1-U*A*G*A*C*U*A*G*A*U*C*A*U*A*U*G | 16 | 4 |
| N15 | L1-U*A*G*A*C*U*A*G*A*U*C*A*U*A*U | 15 | 5 |
| N14 | L1-U*A*G*A*C*U*A*G*A*U*C*A*U*A | 14 | 6 |
| N12 | L1-U*A*G*A*C*U*A*G*A*U*C*A | 12 | 7 |
| N10 | L1-U*A*G*A*C*U*A*G*A*U | 10 | 8 |
| N8 | L1-U*A*G*A*C*U*A*G | 8 | 9 |
| N6 | L1-U*A*G*A*C*U | 6 | 10 |
| AC2(22) | L1- | 22 | 11 |
| | meU*meA*meG*meA*meC*meU*meA*meG*meA*meU*meC*meA*meU*meA*meU*meG* | | |
| | meA*meG*meU*meA*meG*meA | | |
| AC2(18) | L1-meU*meA*meG*meA*meC*meU*meA*meG*meA*meU*meC*meA*meU*meA*meU*meG* | 18 | 12 |
| | meA*meG | | |
| AC2(16) | L1-meU*meA*meG*meA*meC*meU*meA*meG*meA*meU*meC*meA*meU*meA*meU*meG | 16 | 13 |
| AC2(15) | L1-meU*meA*meG*meA*meC*meU*meA*meG*meA*meU*meC*meA*mcU*meA*meU | 15 | 14 |
| AC2(14) | L1-meU*meA*meG*meA*meC*meU*meA*meG*meA*meU*meC*meA*meU*meA | 14 | 15 |
| AC2(13) | L1- | 13 | 16 |
| | meU*meA*meG*meA*meC*meU*meA*meG*meA*meU*meC*meA*meU | | |
| AC2(12) | L1-meU*meA*meG*meA*meC*meU*meA*meG*meA*meU*meC*meA | 12 | 17 |
| AC2(11) | L1-meU*meA*meG*meA*meC*meU*meA*meG*meA*meU*meC | 11 | 18 |
| AC2(10) | L1-meU*meA*meG*meA*meC*meU*meA*meG*meA*meU | 10 | 19 |
| AC2(9) | L1-meU*meA*meG*meA*meC*meU*meA*meG*meA | 9 | 20 |
| AC2(8) | L1-meU*meA*meG*meA*meC*meU*meA*meG | 8 | 21 |
| AC2(6) | L1-meU*meA*meG*meA*meC*meU | 6 | 22 |

¶n.b .: upper case, RNA; *, phosphorothioate (PS) backbone modification; me, 2′-O-methoxyethyl (2′MOE); C, 5′ methyl cytosine; U, 5′-methyl uracil; L1, spacer-18 linker.

TABLE 8
Oligonucleotide strand sequences and duplex compositions

| | | | SEQ | | |
|---|---|---|---|---|---|
| | | Target | ID | | Size |
| Oligonucleotide name | Type | gene | NO | Sequence (5′-3′) | (nt) |
| asSOD1(Tofersen) | ASO | SOD1 | 975 | meC*meAmeG*meGmeA*t*a*c*a*t*t*t*c*t*a*meCmeA*meGmeC*meT | 20 |
| DS17-0001 | siRNA | SOD1 | 384 | CGAGCAGAAGGAAAGUAAUTT | 21 |
| | | | 653 | AUUACUUUCCUUCUGCUCGTT | 21 |
| DS17-0002 | siRNA | SOD1 | 372 | AGGGCAUCAUCAAUUUCGATT | 21 |
| | | | 641 | UCGAAAUUGAUGAUGCCCUTT | 21 |
| DS17-0003 | siRNA | SOD1 | 409 | GCAUUAAAGGACUGACUGATT | 21 |
| | | | 678 | UCAGUCAGUCCUUUAAUGCTT | 21 |
| DS17-0004 | siRNA | SOD1 | 357 | CGACGAAGGCCGUGUGCGUTT | 21 |
| | | | 626 | ACGCACACGGCCUUCGUCGTT | 21 |
| DS17-0005 | siRNA | SOD1 | 486 | UGAAGAGAGGCAUGUUGGATT | 21 |
| | | | 755 | UCCAACAUGCCUCUCUUCATT | 21 |
| DS17-0029 | siRNA | SOD1 | 588 | GGUGGAAAUGAAGAAAGUATT | 21 |
| | | | 857 | UACUUUCUUCAUUUCCACCTT | 21 |
| DS17-01N3 | siRNA | SOD1 | 912 | UCGAGCAGAAGGAAAGUAAU | 20 |
| | | | 913 | AUUACUUUCCUUCUGCUCGAAA | 22 |
| DS17-02N3 | siRNA | SOD1 | 914 | CAGGGCAUCAUCAAUUUCGA | 20 |
| | | | 915 | UCGAAAUUGAUGAUGCCCUGCA | 22 |
| DS17-03N3 | siRNA | SOD1 | 916 | AGCAUUAAAGGACUGACUGA | 20 |
| | | | 917 | UCAGUCAGUCCUUUAAUGCUUC | 22 |
| DS17-04N3 | siRNA | SOD1 | 918 | GCGACGAAGGCCGUGUGCGU | 20 |
| | | | 919 | ACGCACACGGCCUUCGUCGCCA | 22 |
| DS17-05N3 | siRNA | SOD1 | 920 | GCAAUGUGACUGCUGACAAA | 20 |
| | | | 921 | UUUGUCAGCAGUCACAUUGCCC | 22 |
| DS17-01M3 | siRNA | SOD1 | 922 | mU*fC*mGfAmGfCmAfGfAfAfGfGmAfAmAfGmUfA*mA*fU | 20 |
| | | | 923 | pmA*fU*mUfAmCfUmUfUmCfCmUfUmCfUmGfCmUfCmGfA*mA*fA | 22 |
| DS17-02M3 | siRNA | SOD1 | 924 | mC*fA*mGfGmGfCmAfUfCfAfUfCmAfAmUfUmUfC*mG*fA | 20 |
| | | | 925 | pmU*fC*mGfAmAfAmUfUmGfUmUfGmAfUmGfCmCfCmUfG*mC*fA | 22 |
| DS17-03M3 | siRNA | SOD1 | 926 | mA*fG*mCfAmUfUmAfAfAfGfGfAmCfUmGfAmCfU*mG*fA | 20 |
| | | | 927 | pmU*fC*mAfGmUfCmAfGmUfCmCfUmUfUmAfAmUfGmCfU*mU*fC | 22 |
| DS17-04M3 | siRNA | SOD1 | 928 | mG*fC*mGfAmCfGmAfAfGfGfCfCmGfUmGfUmGfC*mG*fU | 20 |
| | | | 929 | pmA*fC*mGfCmAfCmAfCmGfGmCfCmUfUmCfGmUfCmGfC*mC*fA | 22 |
| DS17-05M3 | siRNA | SOD1 | 930 | mG*fC*mAfAmUfGmUfGfAfCfUfGmCfUmGfAmCfA*mA*fA | 20 |
| | | | 931 | pmU*fU*mUfGmUfCmAfGmCfAmGfUmCfAmCfAmUfUmGfC*mC*fC | 22 |
| DS17-01M3-AC1(me14)-L9V3 | ODV-siRNA | SOD1 | 932 | mU*fC*mGfAmGfCmAfGfAfAfGfGmAfAmAfGmUfA*mA*fU-S9- | 34 |
| | | | 933 | mA*fU*mUfAmCfUmUfUmCfCmUfUmCfUmGfCmUfCmGfA*mA*fA | 22 |
| DS17-02M3-AC1(me14)-L9V3 | ODV-siRNA | SOD1 | 934 | mC*fA*mGfGmGfCmAfUfCfAfUfCmAfAmUfUmUfC*mG*fA-S9- | 34 |
| | | | | meU*meU*meG*meU*meA*meU*meU*meC*meU*meA*meU*meG*meU*meU | |
| | | | 935 | mU*fC*mGfAmAfAmUfUmGfUmUfGmAfUmGfCmCfCmUfG*mC*fA | 22 |
| DS17-03M3-AC1(me14)-L9V3 | ODV-siRNA | SOD1 | 936 | mA*fG*mCfAmUfUmAfAfAfGfGfAmCfUmGfAmCfU*mG*fA-S9- | 34 |
| | | | | meU*meU*meG*meU*meA*meU*meU*meC*meU*meA*meU*meG*meU*meU | |
| | | | 937 | mU*fC*mAfGmUfCmAfGmUfCmCfUmUfUmAfAmUfGmCfU*mU*fC | 22 |
| DS17-04M3-AC1(me14)-L9V3 | ODV-siRNA | SOD1 | 938 | mG*fC*mGfAmCfGmAfAfGfGfCfCmGfUmGfUmGfC*mG*fU-S9- | 34 |
| | | | | meU*meU*meG*meU*meA*meU*meU*meC*meU*meA*meU*meG*meU*meU | |
| | | | 939 | mA*fC*mGfCmAfCmAfCmGfGmCfCmUfUmCfGmUfCmGfC*mC*LA | 22 |
| DS17-05M3-AC1(me14)-L9V3 | ODV-siRNA | SOD1 | 940 | mG*fC*mAfAmUfGmUfGfAfCfUfGmCfUmGfAmCfA*mA*fA-S9- | 34 |
| | | | | meU*meU*meG*meU*meA*meU*meU*meC*meU*meA*meU*mcG*meU*meU | |
| | | | 941 | mU*fU*mUfGmUfCmAfGmCfAmGfUmCfAmCfAmUfUmGfC*mC*fC | 22 |
| DS17-29M2-AC1(me14)-L9V3 | ODV-siRNA | SOD1 | 942 | fG*mG*fUmGfGmAfAmAfUfGfAmAfGmAfAmAf*GmU*fA-S9- | 34 |
| | | | | meU*meU*meG*meU*meA*meU*meU*meC*meU*meA*meU*meG*meU*meU | |
| | | | 47 | mU*fA*mCfUmUfUmCfUmUfCmAfUmUfUmCfCmAfCmC*fU*mU | 21 |
| DS17-01M3v-AC1(me14)- | ODV-siRNA | SOD1 | 932 | mU*fC*mGfAmGfCmAfGfAfAfGfGmAfAmAfGmUfA*mA*fU-S9- | 34 |
| | | | | meU*meU*meG*meU*meA*meU*meU*meC*meU*meA*meU*meG*meU*meU | |
| L9V3 | | | 47 | VPmA*fU*mUfAmCfUmUfUmCfCmUfUmCfUmGfCmUfCmGfA*mA*fA | 22 |
| DS17-02M3v-AC1(me14)- | ODV-siRNA | SOD1 | 934 | mC*fA*mGfGmGfCmAfUfCfAfUfCmAfAmUfUmUfC*mG*fA-S9- | 34 |
| | | | | meU*meU*meG*meU*meA*meU*meU*meC*meU*meA*meU*meG*meU*meU | |
| L9V3 | | | 943 | VPmU*fC*mGfAmAfAmUfUmGfUmUfGmAfUmGfCmCfCmUfG*mC*fA | 22 |
| DS17-03M3v-AC1(me14)- | ODV-siRNA | SOD1 | 936 | mA*fG*mCfAmUfUmAfAfAfGfGfAmCfUmGfAmCfU*mG*fA-S9- | 34 |
| | | | | meU*meU*meG*meU*meA*meU*meU*meC*meU*meA*meU*meG*meU*meU | |
| L9V3 | | | 944 | VPmU*fC*mAfGmUfCmAfGmUfCmCfUmUfUmAfAmUfGmCfU*mU*fC | 22 |
| DS17-04M3v-AC1(me14)- | ODV-siRNA | SOD1 | 938 | mG*fC*mGfAmCfGmAfAfGfGfCfCmGfUmGfUmGfC*mG*fU-S9- | 34 |
| | | | | meU*meU*meG*meU*meA*meU*meU*meC*meU*meA*meU*meG*meU*meU | |
| L9V3 | | | 950 | VPmA*fC*mGfCmAfCmAfCmGfGmCfCmUfUmCfGmUfCmGfC*mC*LA | 22 |
| DS17-05M3v-AC1(me14)- | ODV-siRNA | SOD1 | 940 | mG*fC*mAfAmUfGmUfGfAfCfUfGmCfUmGfAmCfA*mA*fA-S9- | 34 |
| L9V3 | | | 951 | VPmU*fU*mUfGmUfCmAfGmCfAmGfUmCfAmCfAmUfUmGfC*mC*fC | 22 |
| DS17-04M3-asSOD1-1-L9V3 | siRNA + ASO | SOD1 | 952 | mG*fC*mGfAmCfGmAfAfGfGfCfCmGfUmGfUmGfC*mG*fU-S9- | 40 |
| | | | | meC*meAmeG*meGmeA*t*a*c*a*t*t*t*c*t*a*meCmeA*meGmeC*meT | |
| | | | 939 | mA*fC*mGfCmAfCmAfCmGfGmCfCmUfUmCfGmUfCmGfC*mC*LA | 22 |
| AC1-L9V3 | ACO | n/a | 953 | UUGUAUUCUAUGUU | 14 |
| ACI(me14)-L9V3 | ACO | n/a | 954 | S9- | 14 |
| | | | | meU*meU*meG*meU*meA*meU*meU*meC*meU*meA*meU*meG*meU*meU | |
| DS17-04M3(Scr)-AC1(me14)- | ODV- | n/a | 955 | fC*mG*fGmUfGmGfAmGfGfCfCfUfGmGfCmGfCmA*fA*mU-S9- | 34 |
| | scrambled | | | meU*meU*meG*meU*meA*meU*meU*meC*meU*meA*meU*meG*meU*meU | |
| L9V3 | duplex | | 956 | mG*fC*mCfAmCfCmUfCmCfGmGfAmCfCmGfCmGfUmUfA*mC*fA | 22 |
| | control | | | | |
| dsCon2M3-AC1(me14)-L9V3 | ODV-control | n/a | 957 | mG*fA*mCfUmAfCmUfGmAfGfUfGmAfCmAfGmUfA*mG*fA-S9- | 34 |
| | duplex | | | meU*meU*meG*meU*meA*meU*meU*meC*meU*meA*meU*meG*meU*meU | |
| | | | 958 | mU*fC*mUfAmCfUmGfUmCfAmCfUmCfAmGfUmAfGmUfC*mG*fU | 22 |
| DS17-Vo149 | siRNA | SOD1 | 959 | AUGGCGACGAAGGCCGUGU | 19 |
| | | | 960 | ACACGGCCUUCGUCGCCAU | 19 |
| DS17-Vo149(c) | siRNA | SOD1 | 961 | AUGGCGACGAAGGCCGUGCdTdT | 21 |
| | | | 962 | UCACGGCCUUCGUCGCCAUdTdT | 21 |
| DS17-Vo153 | siRNA | SOD1 | 963 | CGACGAAGGCCGUGUGCGU | 19 |
| | | | 964 | ACGCACACGGCCUUCGUCG | 19 |
| DS17-Vo153(c) | siRNA | SOD1 | 965 | CGACGAAGGCCGUGUGCGCdTdT | 23 |
| | | | 966 | UCGCACACGGCCUUCGUCGdTdT | 23 |
| DS17-A1289 | siRNA | SOD1 | 967 | GUGGCCGAUGUGUCUAUUGAAGA | 23 |
| | | | 968 | UCUUCAAUAGACACAUCGGCCAC | 23 |
| DS17-A1102 | siRNA | SOD1 | 969 | GUGGGGAAGCAUUAAAGGACUGA | 23 |
| | | | 970 | UCAGUCCUUUAAUGCUUCCCCAC | 23 |
| DS17-Vo195&60 | siRNA | SOD1 | 971 | AGGGCAUCAUCAAUUUCGA | 20 |
| | | | 972 | UCGAAAUUGAUGAUGCCCU | 20 |
| DS17-Vo195(D-2763) | siRNA | SOD1 | 973 | AGGGCAUCAUCAAUUUCGCATdT | 21 |
| | | | 974 | UCGAAAUUGAUGAUGCCCUdTdT | 21 |
| DS17-A1148 | siRNA | SOD1 | 945 | UGCAGGGCAUCAUCAAUUU | 20 |
| | | | 946 | AAAUUGAUGAUGCCCUGCA | 19 |
| DS17-A1194 | siRNA | SOD1 | 69 | AGGGCAUCAUCAAUUUCGA | 19 |
| | | | 71 | UCGAAAUUGAUGAUGCCCU | 19 |
| DS17-A1290 | siRNA | SOD1 | 73 | GCAGGGCAUCAUCAAUUUC | 20 |
| | | | 75 | GAAAUUGAUGAUGCCCUGC | 19 |
| DS17-A1405 | siRNA | SOD1 | 77 | CCAGUGCAGGGCAUCAUCA | 20 |
| | | | 79 | UGAUGAUGCCCUGCACUGG | 19 |
| DS17-A1447 | siRNA | SOD1 | 81 | AUCAUCAAUUUCGAGCAGA | 19 |
| | | | 83 | UCUGCUCGAAAUUGAUGAU | 19 |
| DS17-A1600 | siRNA | SOD1 | 85 | CAGGGCAUCAUCAAUUUCG | 19 |
| | | | 87 | CGAAAUUGAUGAUGCCCUG | 19 |

n.b.: Uppercase, RNA; Lowercase, DNA; *, phosphorothioate (PS) backbone modification; f, 2′-fluoro; m, 2′-O-methyl (2′-OMe); me, 2′-O-methoxyethyl (2′MOE); p, 5′-phosphate; C, 5′ methyl cytosine; U, 5′-methyl uracil; c, 5′ methyl cytosine; VP, 5′-(E)-vinylphosphonate; S9, Spacer-9 linker.

### RT-qPCR

### One-Step Reverse Transcription-Quantitative Polymerase Chain Reaction (One-Step RT-qPCR)

At the end of transfection, medium was discarded, and cells were washed with 150 μL of PBS once per well. After discarding the PBS, 100 μL of cell lysis was added into each well and incubated at room temperature for 5 min. 0.5 μL of the cell lysis was taken from each well and analyzed by RT-qPCR using One Step TB Green™ PrimeScrip™ RT-PCR kit II (Takara, RR086A) in a Roche Lightcycler 480 real-time PCR machine PCR reactions was prepared using Echo 525 Acoustic Liquid Handler (Beckman Coulter). Each transfection sample was amplified in 3 repeat wells. PCR reaction conditions are shown in Table 9.

TABLE 9
PCR reaction preparation.

| Reagent | Volume (μL) |
|---|---|
| 2 × One Step TB Green RT-PCR buffer 4 | 2.5 |
| PrimeScript ™ 1 step enzyme Mix 2 | 0.2 |
| Forward and Reverse Primers Mix (5 μM) | 0.4 |
| dH₂O without RNase | 1.6 |
| Crude lysate (RNA) | 0.4 |
| Total volume | 5.1 |

The reaction conditions were as follows: reverse transcription reaction (stage 1). 42° C. for 5 min, 95° C. for 10 sec; PCR reaction (stage 2): 95° C. for 5 sec, 59° C. for 20 sec, 72° C. for 10 sec; 40 cycles of amplification; and melting curve (stage 3). Human or mouse SOD1 gene was amplified as target genes. TBP and HPRT1 were served as reference genes were also amplified as internal controls for RNA loading. All primer sequences are listed in Table 4 and Table 10.

TABLE 4
Primer sequences for RT-PCR.

| Primer | | SEQ | | Product size |
|---|---|---|---|---|
| Title | Gene | ID NO | Sequence (5′-3′) | (bp) |
| Htt F | Mouse Htt | 896 | GCACGGCATCCTCTATGTGT | 121 |
| Htt R | | 897 | AATGTTCACGCAGTGGGCTA | |
| App F | Mouse App | 898 | CGAGAGAGAATGTCCCAGGTC | 142 |
| App R | | 899 | TCTCATTGGCTGCTTCCTGT | |
| SOD1 F | Human | 900 | AAGCATTAAAGGACTGACTGAAGG | 154 |
| SOD1 R | SOD1 | 901 | CAAGTCTCCAACATGOCTCTC | |
| Sod1 F | Mouse Sod1 | 902 | GGACAATACACAAGGCTGTACC | 125 |
| Sodl R | | 903 | CGTCCTTTCCAGCAGTCACA | |
| SMN2FL F | Human | 904 | TCATACTGGCTATTATATGGGTTTT | 95 |
| SMN2FL R | SMN2 | 905 | TGCTCTATGCCAGCATTTCTC | |
| SMNΔ7 F | Human | 906 | GCTATTATATGGAAATGCTGGCATAG | 73 |
| SMNΔ7 R | SMN2 | 907 | TTCCAGATCTGTCTGATCGTTTCT | |
| TBP F | Human TBP | 908 | TGCTCACCCACCAACAATTTAG | 139 |
| TBP R | | 909 | TCTGCTCTGACTTTAGCACCTG | |
| Tbp F | Mouse Tbp | 910 | GCTCTGGAATTGTACCGCAG | 126 |
| Tbp R | | 911 | CTGCAGCAAATCGCTTGGGA | |

TABLE 10
Primer sequences for RT-qPCR.

| | | SEQ | | Product |
|---|---|---|---|---|
| Primer | Gene | ID NO | Sequence (5′-3′) | size (bp) |
| SOD1 F | Human SOD1 | 900 | AAGCATTAAAGGACTGACTGAAGG | 154 |
| SOD1 R | | 901 | CAAGTCTCCAACATGCCTCTC | |
| Sod1 F | Mouse Sod1 | 902 | GGACAATACACAAGGCTGTACC | 125 |
| Sod1 R | | 903 | CGTCCTTTCCAGCAGTCACA | |
| TBP F | Human TBP | 908 | TGCTCACCCACCAACAATTTAG | 139 |
| TBP R | | 909 | TCTGCTCTGACTTTAGCACCTG | |
| HPRT1 F | Human | 23 | AAAGATGGTCAAGGTCGCAAG | 120 |
| HPRT1 R | HPRT1 | 29 | TAGTCAAGGGCATATCCTACAAC | |
| Tbp F | Mouse Thp | 910 | GCTCTGGAATTGTACCGCAG | 126 |
| Tbp R | | 911 | CTGCAGCAAATCGCTTGGGA | |
| R17-04-AS- | / | 33 | GTCGTATCCAGTGCAGGGTCCGAGG | 1 |
| SL-RT | | | TATTCGCACTGGATACGACTGGCGA | |
| R17-04-AS- | | 35 | atttaACGCACACGGCCTTC | 60 |
| SL-F2 | | | | |
| SL-RT- | | 37 | GTGCAGGGTCCGAGGT | |
| qPCR-Uni- | | | | |
| R1 | | | | |
| olMR0113 | / | 55 | CATCAGCCCTAATCCATCTGA | 236 |
| olMR0114 | | 356 | CGCGACTAACAATCAAAGTGA | |
| olMR7338 | / | 625 | CTAGGCCACAGAATTGAAAGATCT | 324 |
| olMR7339 | | 894 | GTAGGTGGAAATTCTAGCATCATCC | |

### Two Step RT-qPCR

For the sake of quantifying mRNA expression in cells, total cellular RNA was isolated from treated cells using a RNeasy Plus Mini kit (Qiagen, Hilden, Germany) according to its manual. The resultant RNA (1 g) was reverse transcribed into cDNA by using a PrimeScript RT kit containing gDNA Eraser (Takara, Shlga, Japan). The resultant cDNA was amplified in a Roche LightCycler 480 Multiwell Plate 384 (Roche, ref: 4729749001, US) using SYBR Premix Ex Taq 11 (Takara, Shlga, Japan) reagents and primers which specifically amplified target genes of interest. Reaction conditions were as follows: reverse transcription reaction (stage 1): 42° C. for 5 min, 95° C. for 10 sec; PCR reaction (stage 2): 95° C. for 5 sec, 60° C. for 30 sec, 72° C. for 10 sec; 40 cycles of amplification; Melting curve (stage 3). PCR reaction conditions are shown in Table 11 and Table 12.

TABLE 11
RT reaction

| | Volume (μL) |
|---|---|
| Reaction-1 (Takara, RR047A) | |
| 5 × gDNA Eraser Buffer | 2 |
| gDNA Eraser | 1 |
| Total RNA (1 μg) + RNase Free dH₂O | 7 |
| Total Volume | 10 |
| 42° C. 5 min, store at 4° C. | |
| Reagent-2 (Takara, RR047A) | |
| 5 × PrimeScript Buffer2 | 4 |
| PrimeScript RT Enzyme Mix I | 1 |
| RT Prime Mix | 1 |
| RNase free dH₂O | 4 |
| Reaction-1 | 10 |
| Total Volume | 20 |

37° C. 15 min, 85° C. 5 sec, store at 4° C.

TABLE 12
RT-qPCR reaction

| Reagent (Takara, RR820A) | Volume (μL) |
|---|---|
| SYBR Premix Ex Taq II (2×) | 5 |
| PCR Primer (forward + reverse) 5 μM | 1 |
| cDNA (RT product) | 4 |
| Total | 10 |

To calculate the expression level (Erel) of SOD1 mRNA in an siRNA-transfected sample relative to control treatment (Mock), the Ct values of the target gene and the two internal reference genes were substituted into formula III,

$$( formula III )$$

$${E}_{rel} = 2 ( CtTm - CtTs ) / ( ( 2 ( CtR 1 m - CtR 1 ) s * 2 ( CtR 2 m - CtR 2 ) ( 1 / 2 ) s ) )$$

wherein CtTₘ was the Ct value of the target gene from the mock-treated sample; CtTₛ was the Ct value of the target gene from the siRNA-treated sample, (CtR1ₘ was the Ct value of the internal reference gene 1 from the mock-treated sample; CtR1ₛ was the Ct value of the internal reference gene 1 from the siRNA-treated sample; CtR2ₘ was the Ct value of the internal reference gene 2 from the mock-treated sample, and CtR2ₛ was the Ct value of the internal reference gene 2 from the siRNA-treated sample.

### Primary Mouse Hepatocyte (PMH) Isolation and Free Uptake Assay

C57BL/6J mice (Beijing Vital River Laboratory Animal Technology Co., Ltd.) were anesthetized with isoflurane and perfused by initial flushing reagent and digestion reagent successively, followed by placing liver into 10 cm dish containing culture medium. Teared apart the lobes of the liver using two pairs of forceps and obtained the suspension to filter through a 70-75-micron membrane for collecting the cell suspension in 50 mL conical tube. Then, spined at 4° C. for 2 minutes at 100×g in a swinging-arm centrifuge and removed the supernatant and pipetted 20 mL cold PBS to wash cells (Repeat wash for a total of two times). Tested the cell viability using 0.4% trypan blue and seeded cell to the cell culture plates which coated collagen 14˜12 hours before cell seeding to make sure at least 60% cell confluence in culture medium. Make sure all cells grow to their full size and yield a final confluence of 90-95% after seeding cell to carry out the experiment.

### ELISA Assay

To assess immunostimulatory activity of oligonucleotides, ICR mice (Code ID: 201, Beijing Vital River Laboratory Animal Technology Co., Ltd.) were treated by DS17-04M3, AC1-L9V3 and DS17-04M3-AC1(me14)-L9V3 and mouse IL-1β, TNF-α IFN-γ protein expression levels was detected in the serum of treated mice OD value using IL-1β (70-EK201B/3-96, MULTI SCIENCES, China), TNF-α (1217202, Shanghai, China) and IFN-γ (70-EK280/3-96, 70-EK280/3-96, China) ELISA kits by following the instructions provided by the manufacturer of the kits. To test the toxicity after stimulation of oligonucleotides, the serum of ICR mice was harvested and detected the enzyme activity of Alanine transaminase (ALT), aspartate transaminase (AST), and Creatinine (CREA) using AL T kit (OSR6107, Beckman Coulter, US), AST kit (OSR6209, Beckman Coulter, US), and CREA kit (OSR6212, Beckman Coulter, US) by AU480 Chemistry Analyzer (Beckman Coulter, US), respectively, according to the instructions of the kits.

### Immunoblot Analysis

Total cellular protein samples were harvested using RIPA Buffer supplemented with protease inhibitors. Sample concentrations were accessed by the BCA protein assay kit (Beyotime, P0010, Shanghai, China). Protein aliquots (10 ug/well) were resolved via sodium dodecyl sulfate (SDS) polyacrylamide gel electrophoresis (PAGE) and transferred to 0.45 μm polyvinylidene difluoride (PVDF) membranes for immunodetection. Diluted primary antibodies specific to SMN (CST, 19276, USA) or α/β-Tubulin (CST, 2148s, USA) were incubated at 4° C. overnight. Corresponding species-specific anti-IgG horseradish peroxidase secondary antibodies (CST, 7074s and 7076s, USA) were used to visualize protein bands via chemiluminescence on an Image Lab docking station (BIO-RAD, Chemistry Doc MP Imaging System). Protein levels were quantified by optical densitometry of detected bands using ImageJ software.

### Propidium Iodide (P) Staining

HEK293A cells were cultured in 96-well plates following siRNA treatment for 24 hours. Cells were washed with cold PBS and lysed using 40 μL/well of Cell Lysis Buffer (0.25% Igeal CA-630, 140 mM NaCl, 2 mM DTT, 10 mM Tris, pH 7.4) containing 1.5 μM PI. Plates were incubated on ice for 5 minutes prior to measuring optical density (OD) on a microplate reader system (Infinite M2000 Pro) at 535 nm excitation and 615 nm emission wavelengths.

### Stem-Loop RT-qPCR

To quantify oligonucleotides in biological samples, animal tissues were harvested using Heat-Lysis method and stored lysate at −80° C. Duplex, antisense strand and formulated siRNAs (20 μM) were used for the preparation of serial 10-fold dilutions into 95° C. boiled tissue (100 mg/mL) or plasma (1:10 diluted) in 1× lysis buffer. Concentrations in nM were converted to ng/mL using the corresponding siRNAs molecular weights. Two non-template controls were included in all experiments. The first control contains the water used to prepare the transcription master mix and the second contains the lysis buffer used as diluent for samples and standards.

Reverse transcription reactions were performed using a Takara Reverse Transcription kit (Takara, RR037A). A total of 4 μl of cDNA (1:40 dilution) from the previous step was added into the PCR amplification reaction mix (0.5 μM forward primer, 0.5 μM reverse primer, 2×TG Green premix Ex Taq II). The qPCR reaction was run with the option ‘Standard Curve’ in a Light cycler 480. Stem-loop RT-qPCR reaction conditions are shown in Table 13 and Table 14.

TABLE 13
Stem-loop RT reaction

| Reagent (RR037A, Takara) | Volume(μL) | Program |
|---|---|---|
| 5x PrimeScript Buffer | 2 | 16° C. 30 min |
| PrimeScript RT Enzyme Mix I | 0.5 | 42° C. 30 min |
| SL RT primer (1 μM) | 0.5 | 85° C. 5 min |
| RNA/lysate | 1 | Hold at 4° C. as needed |
| RNase free H₂O | 6 | |
| Total volume | 10 | |

TABLE 14
Stem-loop RT-qPCR reaction

| Reagent (RR820A, Takara) | Volume(μL) | Program | |
|---|---|---|---|
| 2 x TB Green premix Ex TaqII | 5 | 95° C. 30 s | 1 cycle |
| Forward primer (10 μM) | 0.5 | 95° C. 5 s | 40 cycles |
| Uni-R primer (10 μM) | 0.5 | 60° C. 20 s | |
| RT product (1:40) | 4 | 72° C. 10 s | |

| | | |
|---|---|---|
| Total volume | 10 | Melting curve |

### Animal Procedures

All animal procedures were conducted by certified laboratory personnel using protocols consistent with local and state regulations and approved by the Institutional Animal Care and Use Committee. Initial breeding pairs of SMA mice were obtained from Jackson Laboratories (Bar Harbor, ME, USA). This model was created by integration of human SMN2 transgene to compensate for homozygous knock-out of mouse Smn gene (Hsieh-L1 et al. 2000). Tail snips were gathered at postnatal day 0 (PND 0) for genotyping by PCR and grouped as Type I SMA mice (Smn^{-/-}, SMN2^{+/-}), Type 111 SMA mice (Snm^{-/-}, SMN2^{+/+}), and heterozygous (Het) controls (Smn, SMN2^{+/-}). C57BL/6 mice were purchased from JOINN Biologics (Suzhou, Jiangsu, China). Bilateral intracerebral ventricle (ICV) injection was performed under anesthesia via 2% isoflurane at a depth of 1.5 mm or 3.6 mm with 29-gauge syringe for pup (2 μL for each side) and adult mice (5 μL for each side), respectively Subcutaneous (SC) injections were placed under the skin in the intrascapular area of pup and adult mice.

### Intracerebral Ventricle (ICV) Injection

Animals were anesthetized by isoflurane inhalation. The eyes of the animal were treated with ocular lubricant. The fur of the scalps and anterior backs were clipped and placed in a stereotaxic apparatus. Buprenorphine was administered subcutaneously prior to incision (0.1 mg/kg). An 1.5-cm, slightly off-center incision was made in the scalp A 25-gauge needle attached to a Hamilton syringe was placed at bregma level and the needle was then moved to the appropriate anterior/posterior and medial/lateral coordinates (0.2 mm anterior/posterior and 1 mm to the right medial/lateral). The proper amount of injection solution was injected at rates of approximately 1 μl/second for a total of 10 μL. This flow rate has been shown to deliver sufficient compound with consistency and no side effect to the animal. The incision was sutured closed using 1 horizontal mattress stitch with 5-0 absorbable suture.

### Intratracheal Instillation (ITI) Injection

Animals were anesthetized by avertin and individually secured on an inclined wood platform. The trachea was exposed by a 1-cm incision on the ventral neck skin. The test articles were dissolved in 50 μL saline and then directly instilled the solution into lung via endotracheal intubation with an 18-gauge plastic catheter. To ensure that the articles was delivered into the mice lung and provided an effective distribution, 150 μL pre-filled air was rapidly pushed into lung via a 1-mL syringe with the blunted needle after instilling the article solution. The catheter without syringe was placed in the trachea of mice to assist breathing. The incision of neck was sutured and swabbed with povidone iodine. The mice were placed on the inclined wood platform for 4 hours by anesthetizing with avertin until the article solution fully absorbed in lung. The saline group were performed same surgical procedure served as a vehicle control.

### Rotarod Behavior Test

To assess the motor coordination, strength and balance of SOD1^{G93A} mice after dosing Tofersen and DS17-04M3-AC1(me14)-L9V3 on PND 46, SOD1^{G93A} mice were tested on Rotarod device (XR-6C, Shanghai Xin Run Information Technology Co., LTD, China) 1-2 times per week. SOD1^{G93A} mice were trained on Rotarod device as follows. 1) The first speed was set 5 revolutions per minute (rpm) and a duration time at 5 seconds. 2) The second speed was set at 20 rpm and a duration time at 100 seconds 3) The third speed was set at 25 rpm and a duration time at 100 seconds. 4) The fourth speed was set at 30 rpm and a duration time at 100 seconds. 5) The fifth speed was set at 20 rpm and a duration time at 300 seconds. SOD1^{G93A} mice were trained until they could remain on the apparatus for 300 seconds without falling. After training, SOD1^{G93A} mice were placed on the rotarod with an accelerating mode (5 to 30 rpm in 5 min) for a maximum time of 300 seconds. SOD1^{G93A} mice were given three trails with an intertrial interval of 30 min and recorded the average time spent on the rotarod.

It has been discovered in this disclosure that after being introduced into a cell, the siRNA can effectively decrease the expression level of SOD1 mRNA and SOD1 protein. The present application will be further illustrated with reference to specific examples and drawings below. It should be understood that these examples are merely intended to illustrate the present application rather than limit the scope of the present application. In the following examples, study methods without specific conditions were generally in accordance with conventional conditions, such as conditions described in Sambrook, et al., Molecular Cloning: Laboratory Manual (New York: Cold Spring Harbor Laboratory Press, 1989), or conditions recommended by the manufacturer.

### In Vivo Imaging of Fluorescein Labeled Oligonucleotide in Animal Organs

Mice were terminally anesthetized on select days following treatment and perfused with cold PBS to wash out residual Quasar 570 (Qu5) dye in circulation. Major organs/tissues (i.e., muscle, liver, lung, heart, kidney, spinal cord, and specified areas of the brain) were harvested and imaged on an IVIS (in vivo imaging system) system at excitation/emission wavelengths of 520/570 nm. Quasar 570 signal was quantified by encircling each tissue or organ within a standardized region of interest (ROI) and measuring fluorescence intensity (p/s/cm2/sr) via Living Image® software (Caliper Life Science, USA).

### EXAMPLES

The following examples are set forth so as to provide those of ordinary skill in the art with a complete disclosure and description of how to make and use the present invention and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); i.m., intramuscular(ly); i.p., intraperitoneal(ly); s.c., subcutaneous(ly), i.e.v, or icv or ICV, intracerebroventricular and the like.

### Example 1: Design and Synthesis of ODV Duplexes

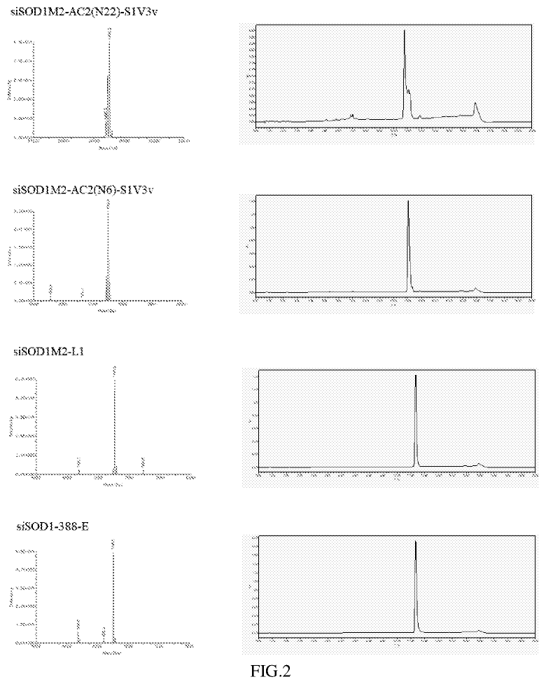

All single-stranded oligonucleotide sequences with chemical modifications including nucleic acid analogs, backbone substitutions, linkers, and ACO conjugates were synthesized on solid-support as single molecular entities. ODV duplexes were subsequently created by annealing complimentary single-stranded oligonucleotides. FIG. 1 illustrates example molecular structures of ODV duplexes after annealing. Quality control of synthesis was performed on purified duplexes using mass spectroscopy and RP-HPLC. FIG. 2 shows the mass spectrograms and elusion profiles of example ODV duplexes with ACOs at 6 (siSOD1M2-AC2(N6)-S1V3v) or 22 nucleotides (siSOD1M2-AC2(N22)-S1V3v) tethered by a Spacer 18 linker (hexaethylene glycol). The spectrum data confirms on-support synthesis of both ODV-siRNAs was efficient and complete with observed MW of 9411.5 Da (calculated MW 9410.86 Da) and 14884.8 Da (calculated MW 14883.05 Da), respectively. In comparison, the same siRNA duplexes without ACOs had an observed MW of 7065.1 Da (calculated MW 7065.08 Da) without the S18 linker (siSOD1-388-E) and an observed MW of 7409.3 Da (calculated MW 7409.38 Da) with S18 (siSOD1M2-L1). RP-HPLC analytics also confirm ODV processing after synthesis yielded relatively pure duplexes as elusion profiles contain single peaks at ˜11 mins (FIG. 2).

### Example 2: ODV-siRNA Inhibits Htt mRNA Expression in Mouse Brain and Spinal Cord

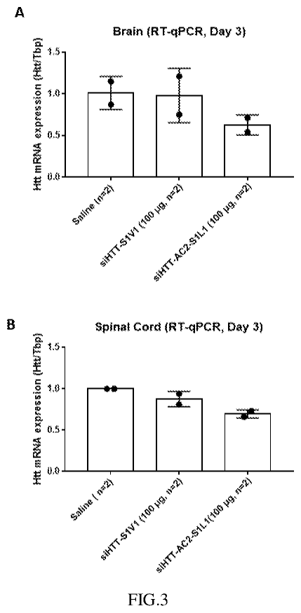

Using medicinal chemistry, a fully-modified siRNA duplex was synthesized with knockdown activity targeting the mouse Htt transcript (siHTT-S1V1). The chemistry provided benefits needed for in vivo function (e.g., prolonged stability and elimination of immune stimulation), but still failed to demonstrate any significant response in vivo following local injection into the CNS. In an attempt to improve local diffusion and in vivo activity, an ODV variant (siHTT-AC2-S1L1) linked to an 18-mer ACO possessing 2′MOE and PS chemistry at every position was synthesized. Both siHTT-S1V1 and siHTT-AC2-S1L1 were injected into PND4 C57BL/6 pup mice. Brain and spinal cord were harvested 72 h later from the injected mice and Htt expression was detected from the harvested tissue after RNA isolation and RT reaction. As shown in FIG. 3A-B, ICV injection of siHTT-S1V1 did not significantly impact Htt expression in the brain or spinal cord compared to saline controls 3 days following treatment. However, siHTT-AC2-S1L1 reduced target gene expression by approximate 37% and 30% in the brain and spinal cord, respectively. This data provides evidence that the ODV strategy can endow siRNA with in vivo activity in the CNS via local injection

### Example 3: ODV does not Interfere with siRNA Knockdown Activity In Vitro

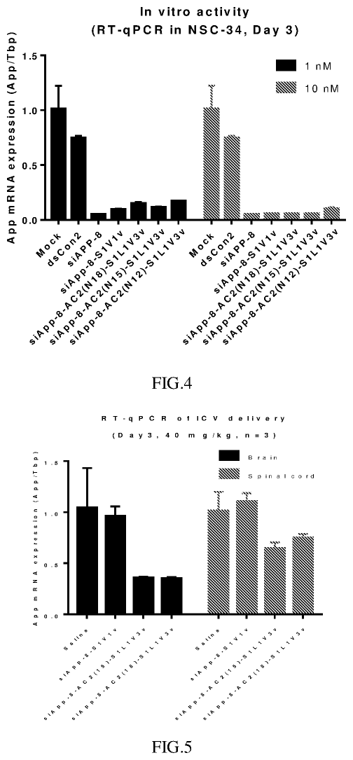

To test the impact ODV design has on siRNA knockdown activity, a series of ODV-siRNA variants with ACOs of different sizes (i.e., 12, 15, or 18 nucleotides in length) denoted as AC2(N12), AC2(N15) and AC2(N18), respectively were synthesized (Table 2). All ACOs were linked to the 3′-terminus of the passenger strand within a parental duplex siApp-8-S1V1v; a medicinally modified version of siAPP-8 with chemistry throughout. As shown in FIG. 4, siRNA knockdown activity was not impaired by ACO addition regardless of length.

Reduction in App mRNA levels ranged between 95-83% at 1 nM and 90-95% at 10 nM treatments in NSC-34 cells for all variants.

### Example 4: ODV-siRNA Possesses In Vivo Activity in the CNS

In vivo knockdown of App mRNA in CNS tissue was evaluated in C57BL/6 pup mice (PND4) via ICV injection. As shown in FIG. 5, siRNA without ACO (App-8-S1V1v) had no detectable impact on App levels in either brain or spinal cord tissue 3 days following injection. In contrast, both ODV variants siApp-8-AC2(N15)-S1L1V3v and siApp-8-AC2(N18)-S1L1V3v caused greater than 60% and 25% reductions in App mRNA within the brain and spinal cord, respectively. This data provides additional evidence that ODV-siRNA possesses in vivo activity in the CNS via local injection.

### Example 5: ODV Composition does not Interfere with In Vitro Activity of saRNA in Inducing Gene Activation

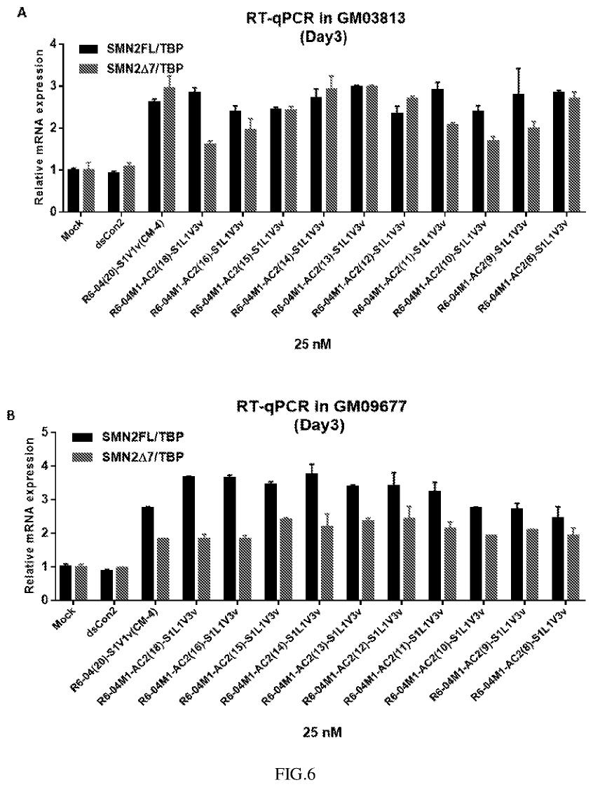

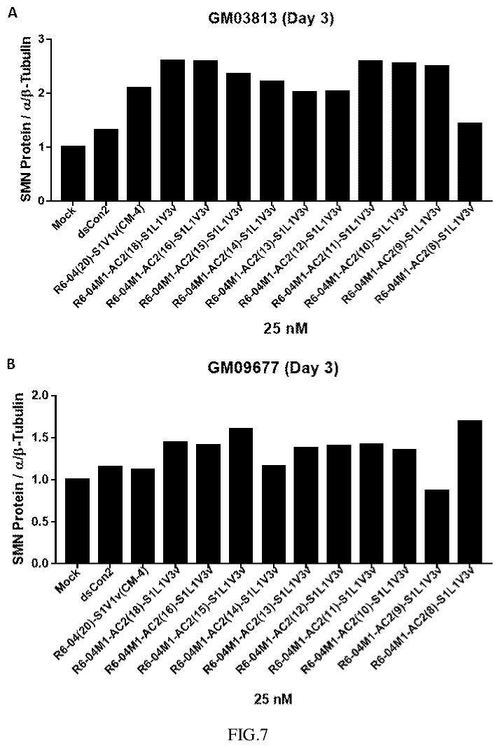

To test the impact ODV design has on saRNA activity, a series of ODV-saRNA variants with ACOs ranging in size from 8 to 18 nucleotides in length [denoted as AC2(8)-AC2(18)] were synthesized (Table 2). All ACOs were linked to the 3′-terminus of the passenger strand of R6-04(20)-S1V1v(CM-4); a medicinally modified saRNA that activates human SMN2 gene expression. Primary human fibroblasts derived from SMA Type-2 (GM03813 cells) and SMA Type-1 (GM09677 cells) patients were transfected with each ODV-saRNA and expression levels of fill length (SMN2FL) and Δ7 (SMV2Δ7) splicing variants were evaluated by RT-qPCR. Only SMN2FL transcript is responsible for producing functional SMN protein, while Δ7 is translated into a non-functional protein susceptible to rapid turnover. As shown in FIG. 6A-B, all ODV-saRNAs increased gene expression similar to R6-04(20)-S1V1v(CM-4) in both GM03813 and GM09677 cells. Immunoblot analysis also showed a measurable increase in full-length SMN protein in most all treatments, although, protein increases were only subtle at day 3 in GM09677 cells (FIG. 7A-B). Much like siRNA, ACOs of various length do not have a significant deleterious effect on saRNA activity.

To determine if ODV-saRNA activity is also conserved in context to human SMN2 transgene upregulation, primary mouse hepatocytes (PMH) derived from the Type III SMA (Smn^{-/-}, SMWN2^{+/+}) mouse model were treated in Nitro and expression levels of full length (SMN2FL) and Δ7 (SAMN2Δ7) splicing variants were evaluated by RT-qPCR. As shown in FIG. 8, all ODV-saRNAs increased SMN2 transgene output similar to or better than R6-04(20)-S1V1v(CM-4) in PMH cells.

### Example 6: Design of ODV-siRNAs to Target SOD1 in the CNS

Sequence for human SOD1 transcript (NM_000454.5) was retrieved from the NCBI nucleotide database. It included a 465 bp open reading frame (ORF) located between nucleotides 78 and 542, which served as the template for siRNA design (Table 5).

TABLE 5
SOD1 cDNA sequence for siRNA design

| | Size | SEQ ID |
|---|---|---|
| SOD1 sequence (5′-3′) | (nt) | NO |
| atggcgacgaaggccgtgtgcgtgctgaagggcgacggcccagtgcagggcatcatcaatttcgagcag | 465 | 895 |
| aaggaaagtaatggaccagtgaaggtgtggggaagcattaaaggactgactgaaggcctgcatggattcc | | |
| atgttcatgagtttggagataatacagcaggctgtaccagtgcaggtcctcactttaatcctctatccagaaaa | | |
| cacggtgggccaaaggatgaagagaggcatgttggagacttgggcaatgtgactgctgacaaagatgctg | | |
| tggccgatgtgtctattgaagattctgtgatctcactctcaggagaccattgcatcattggccgcacactggtg | | |
| gtccatgaaaaagcagatgacttgggcaaaggtggaaatgaagaaagtacaaagacaggaaacgctgga | | |
| agtcgtttggcttgtggtgtaattgggatcgcccaataa | | |

A total of 268 siRNA duplexes were synthesized, each with a length of 19 base-pairs and a GC content of 35%-65% and with no more than 4 repetitive nucleotides (Table 3). HEK293A cells were treated with 0.1 nM and 10 nM of the siRNAs, 24 hours later, the knockdown activity was evaluated using RT-qPCR high throughput screening (HTS). SOD1 mRNA knockdown activity of each siRNA was plotted in FIG. 9. Out of the 268 siRNAs, 121 demonstrated almost complete knockdown activity in reducing SOD1 mRNA levels by more than 90%. As an indicator of potency, 69 (25.7%) and 15 (5.6%) siRNAs reduced SOD1 levels at the concentration of 0.1 nM by over 50% and 75%, respectively. Both criteria allowed for the identification of the top 30 performing siRNAs in which complete dose response curves for SOD1 knockdown confirmed potencies (IC₅₀ values) in the low picomolar range (Table 6).

TABLE 6
Potency of the top 30 best performing SOD1 siRNAs
in knocking down SOD1 mRNA expression

| | SOD1 mRNA (% of Mock) | | |
|---|---|---|---|
| siRNA | 0.1 nM | 10 nM | IC₅₀ (pM) |
| siSOD1-63 | 20.67% | 2.76% | 1.99 |
| siSOD1-47 | 8.74% | 1.86% | 2.51 |
| siSOD1-104 | 13.49% | 1.63% | 3.32 |
| siSOD1-5 | 7.56% | 1.64% | 3.73 |
| siSOD1-258 | 17.26% | 1.62% | 3.74 |
| siSOD1-90 | 19.24% | 3.12% | 4.84 |
| siSOD1-10 | 24.35% | 3.22% | 4.96 |
| siSOD1-134 | 18.51% | 1.74% | 5.57 |
| siSOD1-8 | 23.88% | 2.58% | 5.75 |
| siSOD1-190 | 24.89% | 2.65% | 6.07 |
| siSOD1-254 | 14.03% | 2.31% | 7.00 |
| siSOD1-284 | 29.61% | 2.49% | 7.37 |
| siSOD1-184 | 19.34% | 2.14% | 7.75 |
| siSOD1-288 | 19.03% | 1.92% | 7.82 |
| siSOD1-98 | 21.99% | 2.56% | 9.40 |
| siSOD1-260 | 32.95% | 3.42% | 9.57 |
| siSOD1-129 | 27.72% | 1.79% | 9.71 |
| siSOD1-231 | 21.75% | 3.51% | 11.64 |
| siSOD1-287 | 33.25% | 5.41% | 12.82 |
| siSOD1-285 | 35.97% | 2.48% | 12.90 |
| siSOD1-156 | 24.60% | 2.42% | 13.72 |
| siSOD1-262 | 35.91% | 4.35% | 15.29 |
| siSOD1-388 | 28.77% | 4.20% | 16.27 |
| siSOD1-228 | 36.31% | 2.59% | 16.60 |
| siSOD1-241 | 19.30% | 1.85% | 20.30 |
| siSOD1-96 | 28.40% | 3.20% | 21.26 |
| siSOD1-229 | 27.59% | 4.57% | 22.74 |
| siSOD1-179 | 26.89% | 2.77% | 22.81 |
| siSOD1-242 | 28.51% | 3.92% | 24.47 |
| siSOD1-387 | 26.39% | 4.69% | 26.64 |

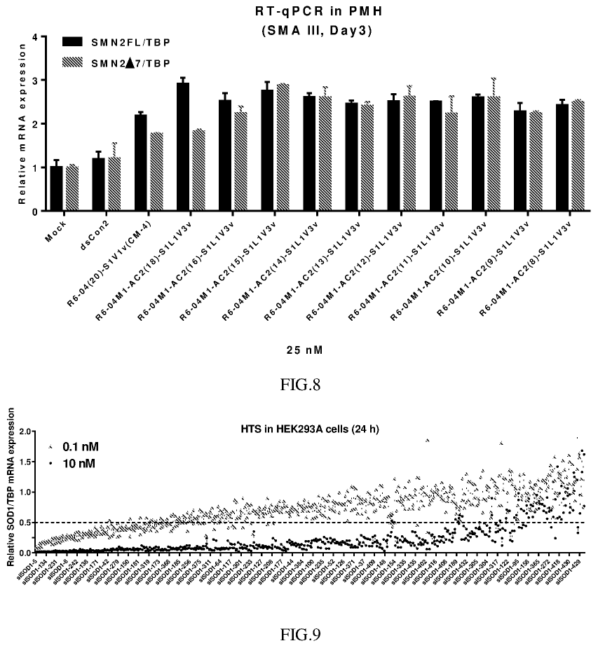

Subsequent analysis of cytotoxicity via PI staining was performed to further identify duplexes with potential safety concerns. As shown in FIG. 10, HEK293A cells tolerated doses at multiples well beyond 100×IC₅₀ values in context to SOD1 knockdown for the top 30 siRNAs. Overall, target sequence selection, siRNA design, and screening criteria identified a panel of strong candidates for further drug development.

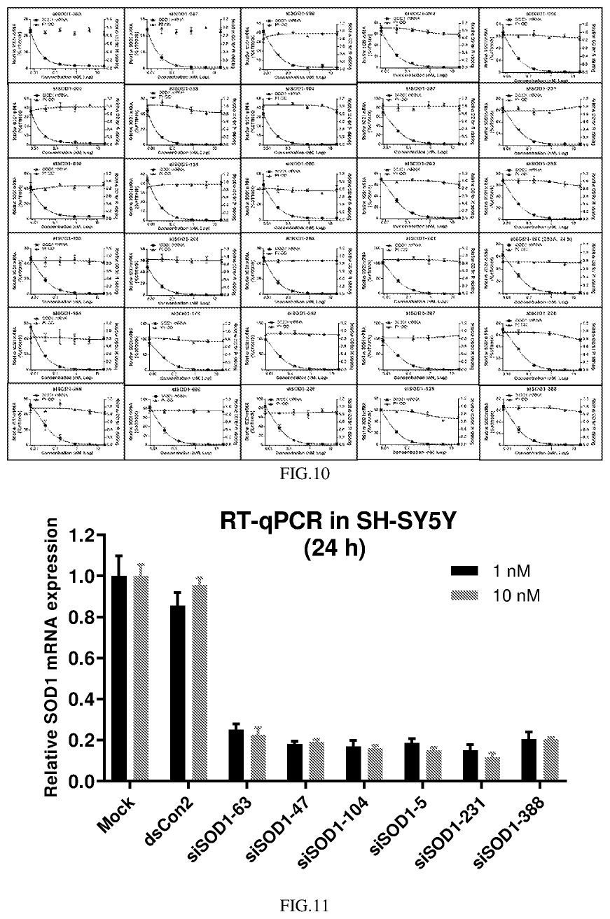

In order to validate activity in model cell lines for neurodegenerative disorders (e.g., ALS), six siRNAs (i.e., siSOD1-63, siSOD1-47, siSOD1-104, siSOD1-5, siSOD1-231 and siSOD1-388) were selected from the top 30 and analyzed in the human neuroblastoma cell line, SH-SY5Y. FIG. 11 shows that knockdown activity was conserved for all six siRNAs reducing SOD1 levels by more than 75% at either 1 nM or 10 nM treatment concentrations. Knockdown was also validated in two motor neuron-like cell lines of mouse origin (i.e., NSC-32 and N-2a) for siRNAs with conserved target sequence in mouse Sod1 transcript (i.e., siSOD1-231, siSOD1-229, siSOD1-388, and siSOD1-387). As shown in FIG. 12A-B, reductions in mouse Sod1 levels were robust approaching near complete knockdown at 10 nM for all tested siRNAs. Based on this data, two exemplary siRNAs (i.e., siSOD1-231 and siSOD1-388) were selected for further development by applying medicinal chemistry.

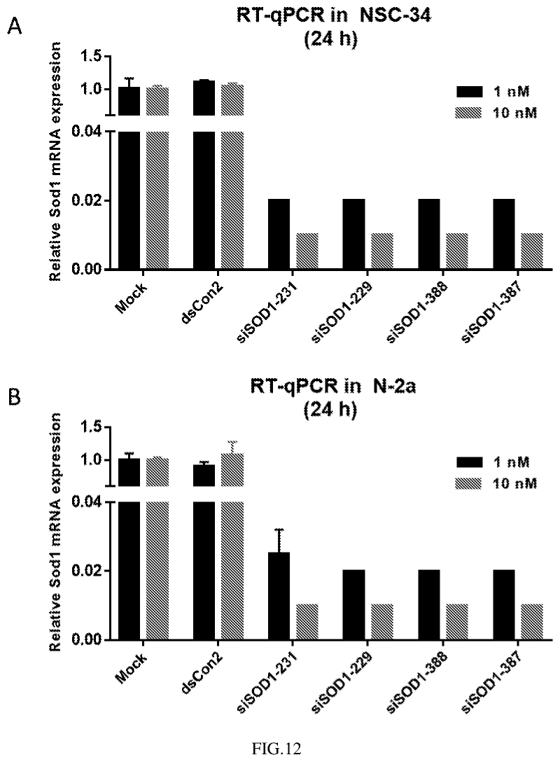

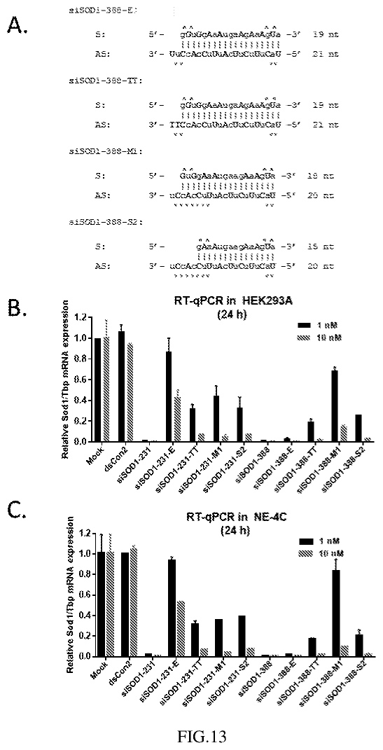

Variants of siSOD1-231 and siSOD1-388 duplexes were synthesized using several different modification patterns comprised of phosphorothioate (PS) backbone substitutions and nucleic acid analogs (i.e., 2′fluoro, 2′-O-methyl, and DNA) in combination with duplex structure asymmetries (Table 2). FIG. 13A illustrates the different chemistries and structural modifications applied to both siRNAs using siSOD1-388 as model sequence. Knockdown activity in HEK293A cells revealed siSOD1-388 appeared to tolerate most of the modification patterns in comparison to unmodified duplex with only siSOD1-388-M1 having the most notable loss in activity; whereas, all chemically modified variants of siSOD1-231 were compromised at 1 nM treatments with siSOD1-231-E having the greatest impairment overall (FIG. 13B). This knockdown profile was also conserved in the mouse neuroepithelial cell line, NE-4C (FIG. 13C). Taken together, siSOD1-388-E was the best overall performer and selected for downstream ODV optimization. Interestingly, the same modification pattern used in siSOD1-231-E nearly abolished its knockdown activity (FIG. 13B-C) implying that the deleterious effects of chemistry are also contingent on duplex sequence.

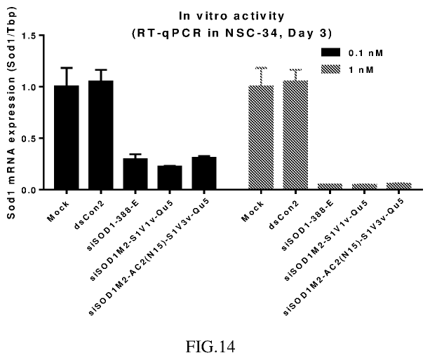

An ODV example of siSOD1-388-E was subsequently created by adding a 5′-vinylphosphonate (5′-VP) to its guide strand and a 15 nucleotide ACO on the 3′-terminus of its passenger strand (Table 2). Qu5 fluorophore was conjugated to the ODV-siRNA (siSOD1M2-AC2(N15)-S1V3v-Qu5) and an ACO-absent variant (siSOD1M2-S1V1v-Qu5) to allow biodistribution analytics following injection into animals. Knockdown performance was confirmed by transfection into NSC-34 cells in which ACO addition had no impact on ODV-siRNA activity at 0.1 or 1 nM treatments in vitro (FIG. 14). Furthermore, neither the 5′VP modification nor Qu5-labelling impaired siSOD1M2-AC2(N15)-S1V3v-Qu5 or siSOD1M2-S1V1v-Qu5 in comparison to unmodified duplex (siSOD1-388-E) (FIG. 14). In vivo analysis in C57BL/6 pup mice (PND4) following treatment via ICV injection at day 3 revealed ODV-siRNA biodistribution signal was enriched across CNS tissue (i.e., brain and spinal cord) compared to siSOD1M2-S1V1v-Qu5 (FIG. 15A-B). Pup mice have an underdeveloped blood-brain barrier allowing for possible systemic exposure and diffusion to peripheral tissues. While both siSOD1M2-AC2(N15)-S1V3v-Qu5 and siSOD1M2-S1V1v-Qu5 were detected in liver and kidney (i.e., consistent with organ perfusion and clearance), the ODV-siRNA was selectively enriched in muscle and other peripheral tissues (albeit at a lower detectable signal) in pup mice (FIG. 15A-B). Analysis of in vivo knockdown activity revealed similar results in that both siSOD1M2-AC2(N15)-S1V3v-Qu5 and siSOD1M2-S1V1v-Qu5 reduced Sod1 levels across CNS tissue with ODV-siRNA providing better performance; however, only siSOD1M2-AC2(N15)-S1V3v-Qu5 activity was preferably detected in peripheral tissue (FIG. 15C). Collectively, this data supports ODV implementation enhances tissue diffusion and distribution, whereas such effects are typically not observed with standard siRNA design.

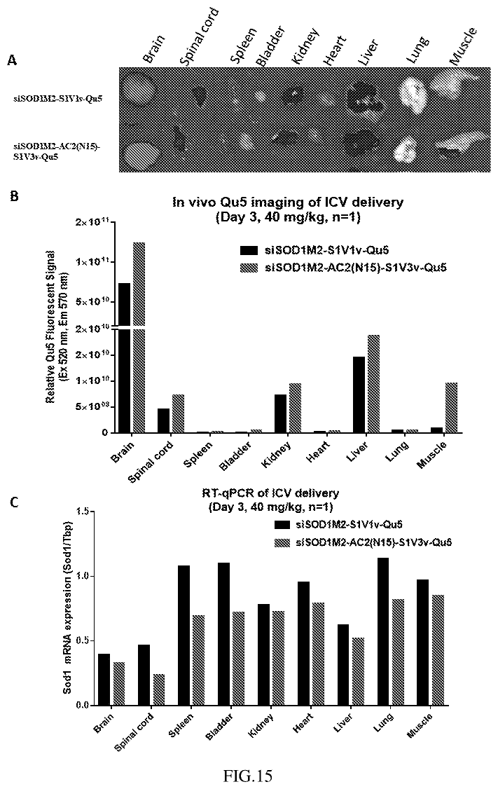

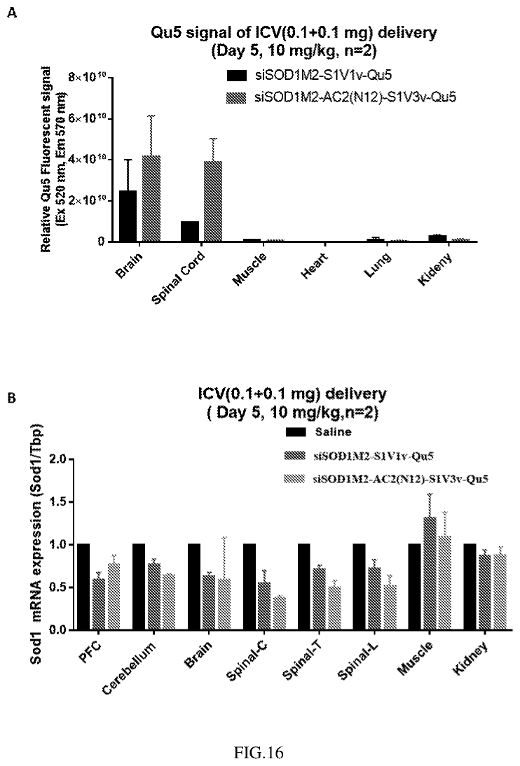

In adult animals, Qu5 signal was largely confined to the CNS following ICV injection at day 5 (FIG. 16A). Overall, siSOD1M2-AC2(N12)-S1V3v-Qu5 biodistribution was enriched over siSOD1M2-S1V1v-Qu5 in both brain and spinal cord tissue. Knockdown activity was also evaluated in several CNS tissues (i.e., prefrontal cortex [PFC], cerebellum, rest of brain [Brain], cervical [Spinal-C], thoracic [Spinal-T], and lumbar [Spinal-L]vertebrae) and peripheral tissues including muscle and kidney. As shown in FIG. 1613, knockdown was detected in all CNS tissues in which ODV-siRNA generally provided better performance. In summary, ODV design provided broader performance improvement across different CNS tissues following local delivery.

### Example 7: ODV Provides a Durable Response in the Cerebellum Tunable by ACO Length

ODV variants comprising ACOs with a length of 6 (siSOD1M2-AC2(N6)-S1V3v-Qu5) or 22 nucleotides (siSOD1M2-AC2(N22)-S1V3v-Qu5) were synthesized to evaluate knockdown kinetics in CNS (Table 2). Adult C57BL/6 mice were treated via ICV injection with each ODV-siRNA and Sod1 knockdown was quantified by RT-qPCR in tissues of the CNS (i.e., frontal cortex, cerebellum, rest of brain tissue, cervical, thoracic, and lumbar vertebrae) and liver at day 10 and 25 post-treatment. As shown in FIG. 17, knockdown was robust and sustained in the cerebellum maintaining an approximate 70% reduction in Sod1 mRNA levels at day 25 for both ODV-siRNA variants. However, knockdown activity at day 10 was broadly distributed throughout the CNS for siSOD1M2-AC2(N6)-S1V3v-Qu5, while predominantly focused in the cerebellum for siSOD1M2-AC2(N22)-S1V3v-Qu5. Distribution also appeared contained within the CNS as activity was nominal in the liver. This data indicates ODV, in general, provides a durable response primarily within the cerebellum; however, distribution to other tissues in the CNS may be affected by ACO length.

### Example 8. SiRNA Knockdown of Human SOD1 mRNA in HEK293A Cells

To verify knockdown of SOD1 mRNA, 6 siRNAs targeting the SOD1 transcript were transfected into HEK293A cells at 0.1 and 1 nM for 24 hours. As shown in FIG. 18, DS17-0001, DS17-0002, DS17-0003, DS17-0004, DS17-0005 and DS17-0029 reduced SOD) mRNA levels by 83%, 84%, 77%, 80%, 79% and 62% at 0.1 nM and 96%, 96%, 96%, 96%, 96% and 93% at 1 nM treatment, respectively. For each of the tested siRNAs, knockdown activity was dose-dependent resulting in partial knockdown (i.e., ˜62-83%) at 0.1 nM and near maximal activity (i.e., greater than 90%) at 1 nM treatments in HEK293A cells.

### Example 9. DS17-04N3 has Superior Knockdown Potency Compared to Exemplary siRNA Designs Disclosed in Prior Art

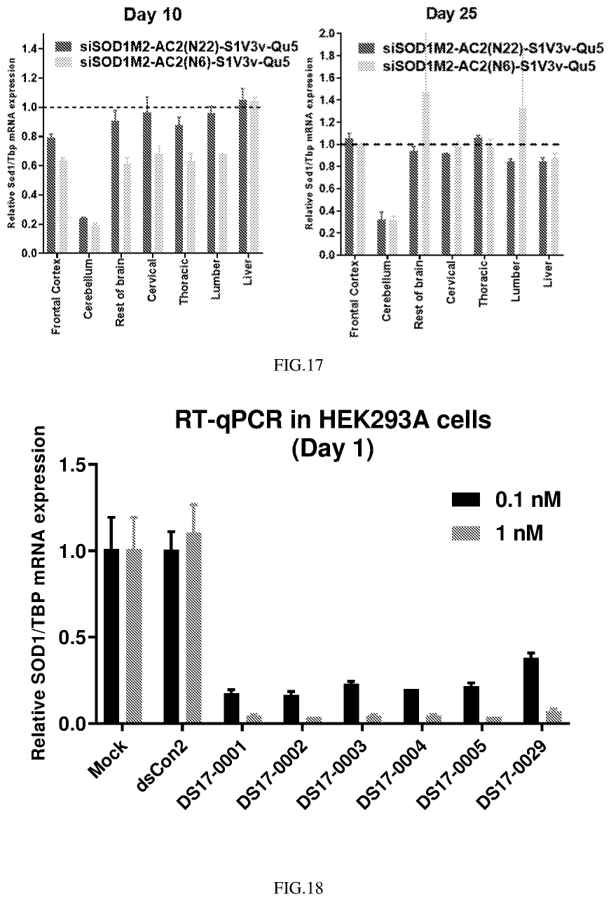

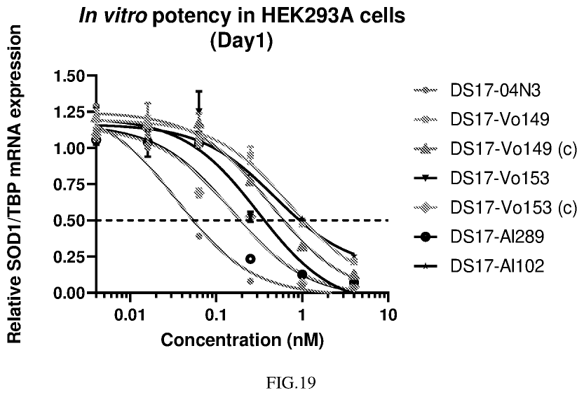

Knockdown activity of DS17-04N3 was compared to 6 other exemplary siRNA designs disclosed in prior art (i.e., DS17-Vo149, DS17-Vo149(c), DS17-Vo153, DS17-Vol 53(c), DS17-Al289, and DS17-Al102) (Table 8). All siRNAs targeted the same overlapping cognate sequence in the SOD1 transcript. SOD1 levels were assessed via RT-qPCR to quantify knockdown activity at each dose concentration (i.e., 0.004, 0.016, 0.063, 0.250, 1.000 and 4.000 nM) in HEK293A cells 24 hours after treatment. Plotting knockdown activity generated dose response curves in which IC₅₀ values (dotted line) were extrapolated to define potency for each of the tested siRNAs (FIG. 19). As shown in Table 15, DS17-04N3 was the most potent siRNA having the lowest IC₅₀ value at 0.05 nM. The other 6 siRNAs were ˜3- to 15-fold less potent than DS17-04N3 by comparison.

TABLE 15
IC₅₀ value of DS17-04N3 compared to 6
other exemplary siRNAs in HEK293A cells

| Duplex | IC₅₀ (nM) |
|---|---|
| DS17-04N3 | 0.050 |
| DS17-Vo149 | 0.745 |
| DS17-Vo149(c) | 0.445 |
| DS17-Vo153 | 0.236 |
| DS17-Vo153(c) | 0.153 |
| DS17-Al289 | 0.180 |
| DS17-Al102 | 0.640 |

### Example 10. DS17-02N3 Design has Superior Knockdown Activity in HeLa and HEK293A Cells

DS17-02N3 also shares a common target site in human SOD1 mRNA with 8 other siRNA duplexes disclosed in prior art (i.e., DS17-Vo195&60, DS17-Vo195 (D-2763), DS17-Al148, DS17-Al194, DS17-Al290, DS17-Al405, DS17-Al447, and DS17-Al600) (Table 8).

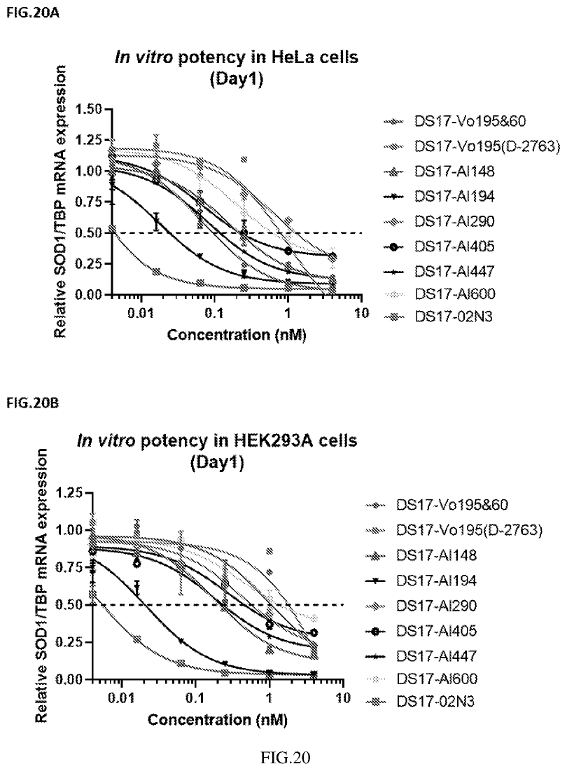

Knockdown activity was compared via RT-qPCR at escalating concentrations (i.e., 0.004, 0.016, 0.063, 0.250, 1.000 and 4.000 nM) to generate dose response curves in HeLa (FIG. 20A) and HEK293A (FIG. 20B) cells 24 hours after transfection. All siRNAs demonstrated dose-dependent knockdown and IC₅₀ values (dotted lines) were extrapolated to compare siRNA potency (FIG. 20A-B). As summarized in Table 16, DS17-02N3 had superior potency in comparison to the other siRNA designs with the lowest IC₅₀ values at 0.001 nM and 0.005 nM in HeLa and HEK293A cells, respectively.

TABLE 16
IC₅₀ comparison in HeLa and HEK293A cells of DS17-02N3
and 8 other siRNA duplexes disclosed in prior art.

| | IC₅₀ (nM) | IC₅₀ (nM) |
|---|---|---|
| Duplex | in HeLa | in HEK293A |
| DS17-Vo195&60 | 0.072 | 1.022 |
| DS17-Vo195(D-2763) | 0.971 | 5.736 |
| DS17-Al148 | 0.159 | 0.177 |
| DS17-Al194 | 0.018 | 0.021 |
| DS17-Al290 | 0.630 | 0.428 |
| DS17-Al405 | 0.070 | 0.272 |
| DS17-Al447 | 0.082 | 0.187 |
| DS17-Al600 | 0.199 | 0.455 |
| DS17-02N3 | 0.001 | 0.005 |

### Example 11. Chemical Modification of siRNA Provides Consistent and Potent Knockdown Activity in T98G and HEK293A Cells

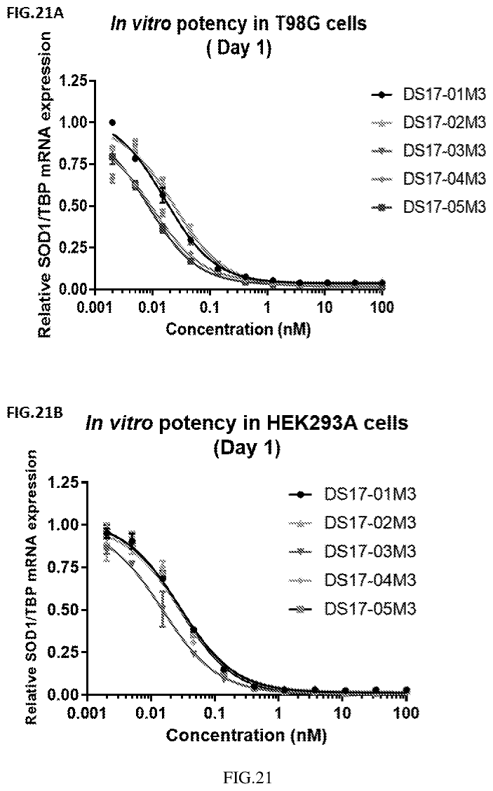

Chemically modified variants of DS17-0001, DS17-0002, DS17-0003, DS17-0004, and DS17-0005 were synthesized by implementing a consensus modification pattern and applying the same structural designs used in both DS17-04N3 and DS17-02N3. The sequence of these variants are listed in Table 8. Knockdown activity of each siRNA variant (i.e., DS17-01M3, DS17-02M3, DS17-03M3, DS17-04M3, and DS17-05M3) (Table 8) was assessed via RT-qPCR at escalating concentrations (i.e., 0.002, 0.005, 0.015, 0.046, 0.137, 0.412, 1.235, 3.704, 11.111, 33.333 and 100 nM) to generate dose response curves in T98G (FIG. 21A) and HEK293A (FIG. 21B) cells 24 hours after transfection. In both cell lines, SOD1 knockdown was concentration dependent with IC₅₀ values in the lower picomolar range (i.e., less than ˜30 pM) for each of the tested siRNA duplexes. As summarized in Table 17, both DS17-04M3 and DS17-05M3 had similar potencies with IC₅₀ extrapolations equaling 0.008 nM in T98G cells, while DS17-03M3 was marginally the more potent siRNA in HEK293A cells with an IC₅₀ of 0.015 nM

TABLE 17
IC₅₀ values for SOD1 knockdown by chemically
modified siRNAs in T98G and HEK293A cells.

| | IC₅₀ (nM) | IC₅₀ (nM) |
|---|---|---|
| Chemically modified siRNA | in T98G | in HEK293A |
| DS17-01M3 | 0.018 | 0.029 |
| DS17-02M3 | 0.022 | 0.028 |
| DS17-03M3 | 0.022 | 0.015 |
| DS17-04M3 | 0.008 | 0.027 |
| DS17-05M3 | 0.008 | 0.028 |

### Example 12. Assessment of SOD1 Knockdown Activity and Safety of ODV-siRNAs in HEK293A Cells

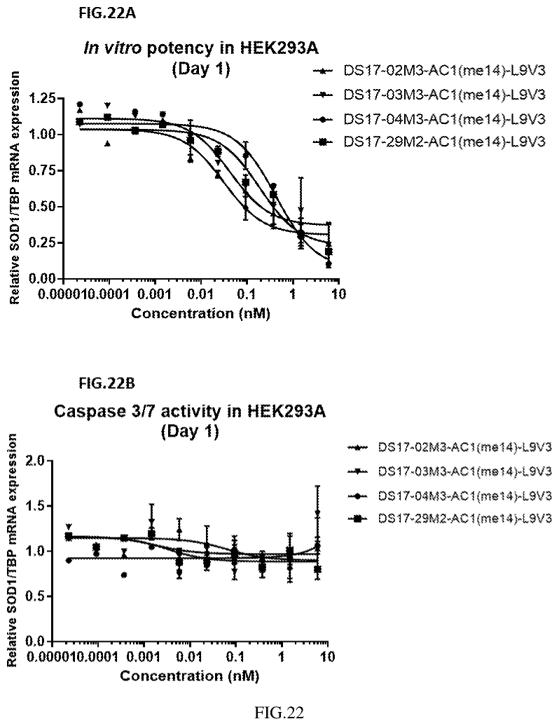

ODV variants of DS17-02M3, DS17-03M3, DS17-04M3, and DS17-29M3 were synthesized by conjugating a 14-nucleotide ACO onto the 3′-terminus of the passenger (sense) strand within each duplex (Table 8). SOD1 mRNA levels were quantified via RT-qPCR at escalating concentrations (i.e., 0.00002, 0.00009, 0.00037, 0.0015, 0.0059, 0.023, 0.094, 0.375, 1.5 and 6 nM) to generate dose response curves for each ODV-siRNA (i.e., DS17-02M3-AC1(me14)-L9V3, DS17-03M3-AC1(me14)-L9V3, DS17-04M3-AC1(me14)-L9V3, and DS17-29M2-AC1(me14)-L9V3) in HEK293A cells 24 hours after transfection. FIG. 22A indicates that SOD1 knockdown was dose-dependent for each ODV-siRNA with ICs, values in the higher picomolar range (i.e., 83-313 μM). Table 18 summarizes the potency of each ODV-siRNA with DS17-02M3-AC1(me14)-L9V3 having the lowest IC₅₀ value at 0.083 nM in HEK293A cells. Caspase 3/7 activity was also quantified to determine the induction of unwanted cytotoxicity as a marker of safety. As shown in FIG. 22B, caspase 3/7 activity was not detected at any of the tested dose concentrations up to 6 nM indicating that each ODV-siRNA was generally free of cytotoxic effects.

TABLE 18
IC₅₀ values of ODV-siRNAs in HEK293A cells.

| ODV-siRNA | IC₅₀ (nM) |
|---|---|
| DS17-02M3-AC1(me14)-L9V3 | 0.083 |
| DS17-03M3-AC1(me14)-L9V3 | 0.204 |
| DS17-04M3-AC1(me14)-L9V3 | 0.283 |
| DS17-29M2-AC1(me14)-L9V3 | 0.313 |

### Example 13. In Vivo Knockdown Activity of ODV-siRNAs in CNS Tissues of SOD1^{G93A} Mice

To test the knockdown activity in vivo, adult SOD1^{G93A} mice at PND 46 were treated via ICV injection with ODV-siRNA (i.e., DS17-01M3-AC1(me14)-L9V3, DS17-04M3-AC1(me14)-L9V3, or DS17-05M3-AC1(me14)-L9V3) at a total dose of 0.4 mg. A non-specific ODV-siRNA (i.e., dsCon2M3-AC1(me14)-L9V3) was used as a negative control, while treatment with an ASO identical in composition to the drug Toferson (BIIB067) served as a reference for SOD1 knockdown activity in vivo. All test articles were formulated in artificial CSF (aCSF) in which treatment with aCSF alone functioned as a procedural control to establish baseline expression in CNS tissues Mice were sacrificed on day 7 after treatment and CNS tissues from the brain (i.e., frontal cortex, cerebellum, and rest of brain tissue) and spinal cord (i.e., cervical, thoracic, and lumbar) were harvested for mRNA expression analysis via RT-qPCR.

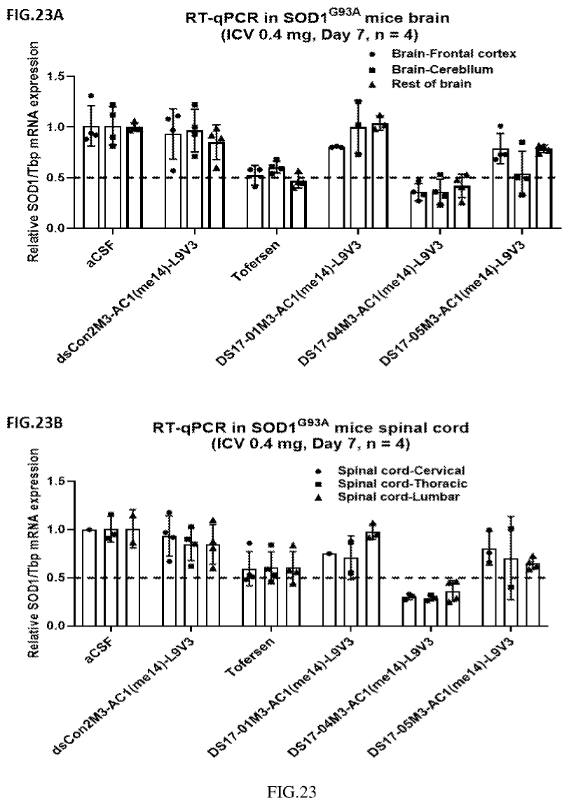

As shown in FIG. 23A, Toferson decreased SOD1 levels in the frontal cortex, cerebellum, and the rest of brain tissue by 48%, 39%, and 53%, respectively, whereas DS17-04M3-AC1(me14)-L9V3 resulted in knockdown of 64%, 64%, and 58% in the same tissues. Knockdown by DS17-05M3-AC1(me14)-L9V3 was more modest measuring a respective decline by 21%, 46%, and 21% in SOD1 levels, while DS17-01M3-AC1(me14)-L9V3 measured a 20% knockdown only in the frontal cortex. Knockdown activity in tissues from the spinal cord followed a similar trend in which DS17-04M3-AC1(me14)-L9V3 had better overall activity provided a 70%, 70%, and 64% reduction in SOD levels in cervical, thoracic, and lumbar tissues, respectively (FIG. 23B). Collectively, data indicates that ODV-siRNA (e.g., DS17-04M3-AC1(me14)-L9V3) can knock down SOD1 across broad CNS tissues via ICV injection with activities similar to or better than Toferson.

### Example 14. Therapeutic Efficacy of ODV-siRNA in Adult SOD1^{G93A} Mice

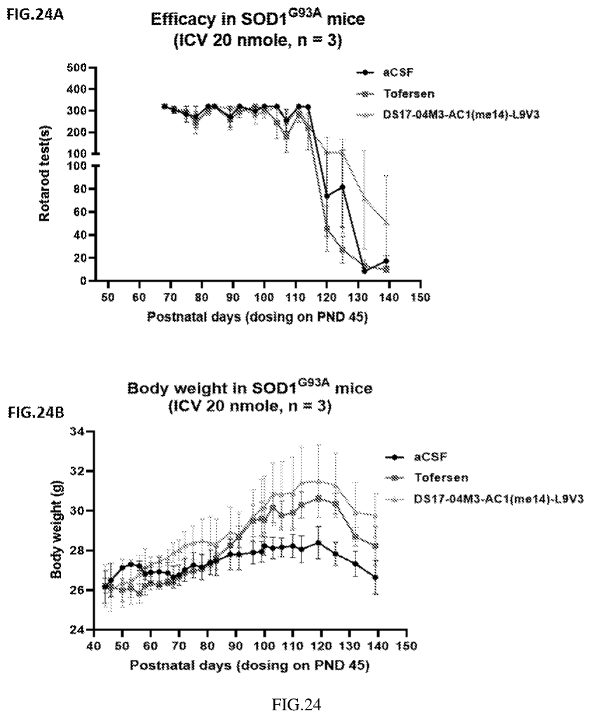

Equal molar quantities (20 nmole) of DS17-04M3-AC1(me14)-L9V3 or Tofersen were administered in a single dose via ICV injection into adult SOD1^{G93A} mice on PND 46. Two parameters for the clinical phenotype of ALS including rotarod performance and body weight were measured ˜1-2 times per week (or as indicated) until PND 140 in order to evaluate therapeutic efficacy. As shown in FIG. 24A, disease onset was not detected until PND 120 via rotarod test in which only the DS17-04M3-AC1(me14)-L9V3 treatment group showed latency in disease progression up to PND 140 in comparison to aCSF control treatments. However, gains in body weight were substantially greater in animals treated with DS17-04M3-AC1(me14)-L9V3 or Tofersen compared to control animals (aCSF) up until PND 120 (FIG. 24B). Upon disease onset, weight loss was also offset by the higher peak body weights in both DS17-04M3-AC1(me14)-L9V3 and Toferson treatment groups with DS17-04M3-AC1(me14)-L9V3 providing a greater therapeutic effect (FIG. 24B). Only one animal death occurred at PND 139 in the control group, while all remaining animals were terminated at the end of the study on PND 140. Overall, the data shows that a single dose of ODV-siRNA (i.e., DS17-04M3-AC1(me14)-L9V3) has comparatively greater therapeutic efficacy than Toferson in SOD^{G93A} mice.

### Example 15. Pharmacokinetics in Brain Tissue from Adult SOD1^{G93A} Mice Following ODV-siRNA Treatment Via ICV Injection

Adult SOD1^{G93A} mice were treated with 0.4 mg DS17-04M3-AC1(me14)-L9V3 via ICV injection and brain tissues were harvested on day 1, 7, 28, or 56 after treatment. DS17-04M3-AC1(me14)-L9V3 was detected in the tissue preps by selectively amplifying its guide strand via primer-specific RT-qPCR. Concentrations in the cerebellum and cumulative brain tissue were plotted to evaluate pharmacokinetics (PK) FIG. 25 depicts DS17-04M3-AC1(me14)-L9V3 levels peaked at day 1 and maintained a steady state from day 7 to 56 Concentrations were estimated to be at 334, 66, 82 and 56 μg/g in the cerebellum and 418, 234, 135 and 164 μg/g in rest of brain tissue at day 1, 7, 28, and 56, respectively.

### Example 16. Dose-Dependent Knockdown of SOD1 by ODV-siRNA in Adult SOD1^{G93A} Mice

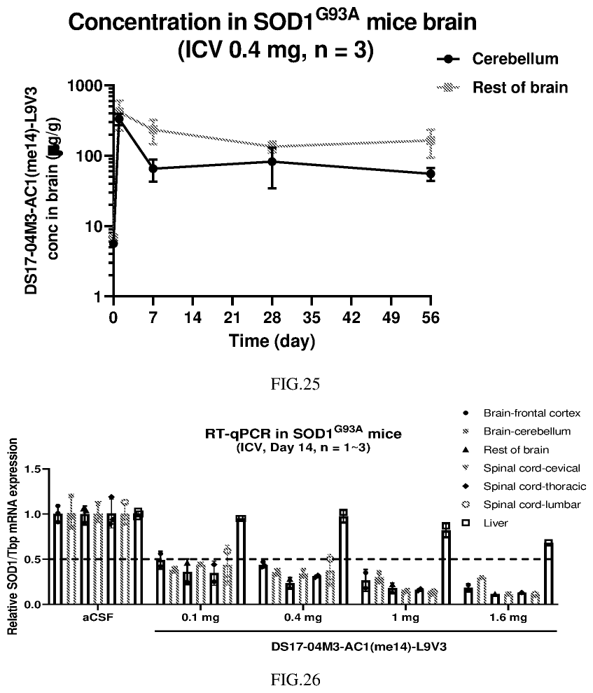

Dose response activity in the CNS was evaluated in adult SOD1^{G93A} mice following ICV injection of 0.1, 0.4, 1.0 or 1.6 mg DS17-04M3-AC1(me14)-L9V3. Mice were sacrificed 14 days after treatment in which brain (i.e., frontal cortex, cerebellum, and rest of brain tissues), spinal cord (i.e., cervical, thoracic, and lumbar), and peripheral (i.e., liver) tissues were harvested for expression analysis of SOD1 via RT-qPCR Treatment with aCSF alone served as a vehicle control to establish baseline expression levels. As shown in FIG. 26, SOD1 knockdown was dose-dependent, with its level decreased by ≥70% after 1.0 mg and 1.6 mg treatment in all CNS tissues, whereas the knockdown activity in the liver was substantially lower, resulting in a maximal SOD1 reduction of only ˜32% at the highest dose. Table 19 summarizes the mean knockdown activity of DS17-04M3-AC1(me14)-L9V3 in all evaluated tissues at the indicated doses. Overall, knockdown was well localized within the CNS in which drainage to peripheral tissue (i.e., liver) did not produce ample activity.

TABLE 19
Percent knockdown of SOD1 mRNA in mouse
CNS and liver tissues by ODV-siRNA.

Mean Knockdown (%)
DS17-04M3-AC1(me14)-L9V3

| Tissue | 0.1 mg | 0.4 mg | 1 mg | 1.6 mg |
|---|---|---|---|---|
| Brain-frontal cortex | 51 | 56 | 73 | 82 |
| Brain-cerebellum | 62 | 65 | 70 | 70 |
| Rest of brain | 64 | 77 | 82 | 88 |
| Spinal cord-cervical | 56 | 66 | 86 | 89 |
| Spinal cord-thoracic | 66 | 69 | 84 | 87 |
| Spinal cord-lumbar | 57 | 63 | 87 | 89 |
| Liver | 5 | 2 | 17 | 32 |

### Example 17. Enhancement of ODV-siRNA Potency with Chemical Modification at the 5′-Position in the Guide Strand

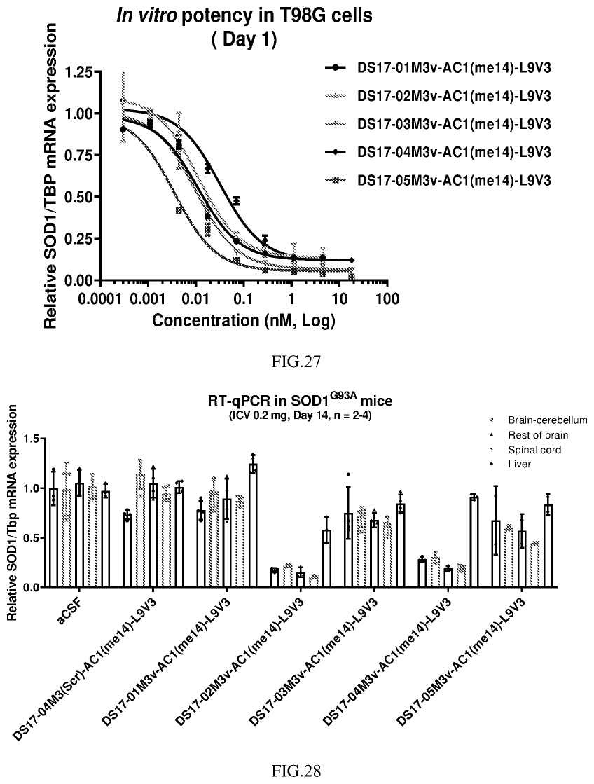

Vinylphosphonate (VP) modification at the 5′-position (5′VP) in the guide strand of ODV-siRNA was tested in vitro for impact on knockdown activity. SOD1 levels were quantified via RT-qPCR at escalating concentrations (i.e., 0.0003, 0.0011, 0.0044, 0.0176, 0.0703, 0.2813, 1.1250, 4.5 and 18 nM) to generate dose response curves for each 5′VP modified variant of ODV-siRNA (i.e., DS17-01M3v-AC1(me14)-L9V3, DS17-02M3v-AC1(me14)-L9V3, DS17-03M3v-AC1(me14)-L9V3, DS17-04M3v-AC1(me14)-L9V3 and DS17-05M3v-AC1(me14)-L9V3) in T98G cells 24 hours after transfection. FIG. 27 indicates that the 5′VP modification was well-tolerated in which SOD1 knockdown was concentration dependent for each ODV-siRNA with IC₅₀ values in the low picomolar range (i.e., 4-42 pM). Potency results are summarized in Table 20.

TABLE 20
IC₅₀ values of 5′VP-modified variants
of ODV-siRNAs in T98G cells.

| Duplex | IC₅₀ (nM) |
|---|---|
| DS17-01M3v-AC1(me14)-L9V3 | 0.019 |
| DS17-02M3v-AC1(me14)-L9V3 | 0.019 |
| DS17-03M3v-AC1(me14)-L9V3 | 0.013 |
| DS17-04M3v-AC1(me14)-L9V3 | 0.042 |
| DS17-05M3v-AC1(me14)-L9V3 | 0.004 |

### Example 18. In Vivo Knockdown Activity of 5′VP-Modified ODV-siRNAs in the CNS Tissues of SOD1^{G93A} Mice

To test the knockdown activity in vivo, adult SOD1^{G93A} mice at PND 46 were treated via ICV injection with 5′VP-modified ODV-siRNA (i.e., DS17-01M3v-AC1(me14)-L9V3, DS17-02M3v-AC1(me14)-L9V3, DS17-03M3v-AC1(me14)-L9V3, DS17-04M3v-AC1(me14)-L9V3 and DS17-05M3v-AC1(me14)-L9V3) at a total dose of 0.2 mg. A scrambled ODV-siRNA (i.e., DS17-04M3(Scr)-AC1(me14)-L9V3) was used as a non-specific control, while treatment with aCSF alone functioned as a vehicle control to establish baseline expression Mice were sacrificed on day 14 after treatment and tissues from the brain (i.e., cerebellum and rest of brain), spinal cord, and liver were harvested for mRNA expression analysis via RT-qPCR. As shown in FIG. 28, DS17-02M3v-AC1(me14)-L9V3 provided potent knockdown of SOD1 reducing mRNA levels by 85%. 78%, 90%, and 42% in the cerebellum, rest of brain, spinal cord, and liver, respectively. DS17-04M3v-AC1(me14)-L9V3 treatment also had substantial knockdown activity measuring reductions of 70%, 81%, 81%, and 9% in the same respective tissues. Knockdown by DS17-03M3v-AC1(me14)-L9V3 and DS17-05M3v-AC1(me14)-L9V3 was more modest causing SOD1 levels to decrease between 29-56% within CNS tissues, whereas DS17-01M3v-AC1(me14)-L9V3 only provided up to 13% knockdown at the 0.2 mg dose.

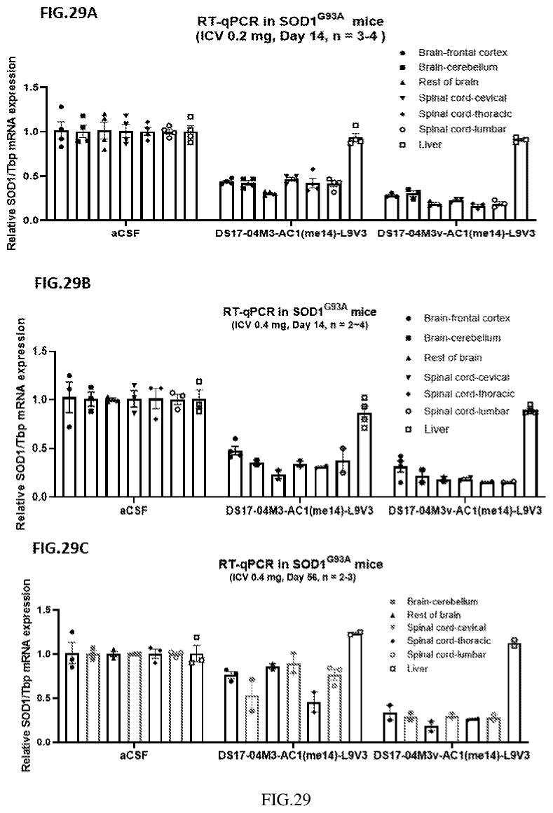

Direct comparison of knockdown activity by ODV-siRNA with (i.e., DS17-04M3v-AC1(me14)-L9V3) or without (i.e., DS17-04M3-AC1(me14)-L9V3) the 5′VP modification was also evaluated in tissues of the brain (i.e., frontal cortex, cerebellum, and rest of brain), spinal cord (i.e., cervical, thoracic, and lumbar), and periphery (i.e., liver). As shown in FIG. 29A-B, DS17-04M3v-AC1(me14)-L9V3 at both 0.2 and 0.4 mg doses had greater knockdown activity across all CNS tissues compared to its non-5′VP variant (i.e., DS17-04M3-AC1(me14)-L9V3) day 14 following treatment. This observation was further accentuated when evaluating knockdown activity at day 56 in which DS17-04M3v-AC1(me14)-L9V3 provided a much more durable response (FIG. 29C). In contrast, 5′VP modification did not appear to further exaggerate knockdown in the liver via CNS drainage suggesting 5′VP also improved the activity differential between local and peripheral tissue (FIG. 29A-C).

### Example 19. Potent and Durable Knockdown of SOD1 by ODV-siRNA in CNS Tissues of SOD1^{G93A} Mice

To further assess knockdown durability of ODV-siRNA in comparison to Toferson, adult SOD1^{G93A} mice at PND 46 were administered in a single dose via ICV injection equal molar quantities (20 nmole) of DS17-04M3-AC1(me14)-L9V3, DS17-04M3v-AC1(me14)-L9V3, or Toferson. All test articles were formulated in aCSF in which treatment of aCSF alone functioned as a vehicle control to establish baseline expression in isolated tissues. Mice were sacrificed on day 56 after treatment and tissues from the brain (i.e., frontal cortex, cerebellum, and rest of brain tissue), spinal cord (i.e., cervical, thoracic, and lumbar), and liver were harvested for SOD1 mRNA expression analysis via RT-qPCR.

As shown in FIG. 30A-F, 5′VP-modified ODV-siRNA (i.e., DS17-04M3v-AC1(me14)-L9V3) provided both the greatest maximal and most durable knockdown response in all CNS tissues reducing SOD1 levels by ˜68-81% and ˜53-69% at 2 and 8 weeks, respectively. Conversely, Toferson had the weakest activity at the 20 nmole dose in which maximal activity in its most responsive tissue (i.e., rest of brain) only measured 50% and 14% knockdown at 2 and 8 weeks, respectively (FIG. 30C). Expression analysis in the liver also revealed that knockdown was well localized within the CNS for all oligonucleotide treatments in which the 20 nmole dose did not produce ample activity via drainage to peripheral tissue at any time point (FIG. 30G). Overall, data indicates that both ODV-siRNA duplexes showed enhanced activity and durability for SOD1 knockdown compared to Toferson at an equivalent molar dose, while the 5′VP-modified variant had better potency and knockdown durability in vivo.

### Example 20. Acute Toxicity and Immunostimulatory Activity Assessment of SOD1 ODV-siRNA in ICR Mice

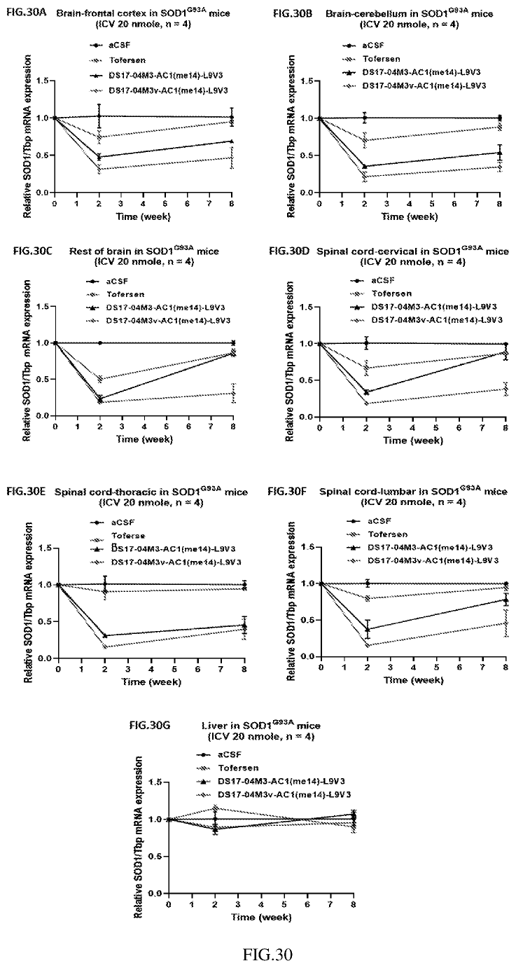

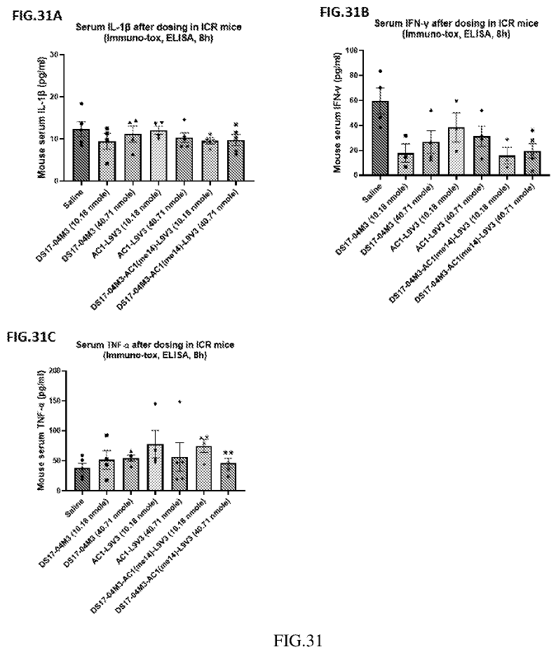

To assess in vivo safety, ICR mice approximately 6 weeks of age were treated via SC injection with equal molar quantities of ODV-siRNA (DS17-04M3-AC1(me14)-L9V3), siRNA without ACO conjugate (DS17-04M3), or ACO component alone (AC1-L9V3) at either 10.18 or 40.71 nmole to measure innate immunostimulation following systemic exposure to oligonucleotide treatment. Saline served as a vehicle control to establish baseline levels of cytokines in serum. Sequences for each oligonucleotide are listed in Table 8. Mice were sacrificed 8 hours after treatment and serum was harvested to detect IL-1β, IFN-γ, and TNF-α protein levels via ELISA. As shown in FIG. 31A-C, no significant change in any of the tested cytokines was detected compared to saline control. This data implies neither DS17-04M3-AC1(me14)-L9V3 nor any of its components (i.e., DS17-04M3 and AC1-L9V3) have immunostimulatory activity.

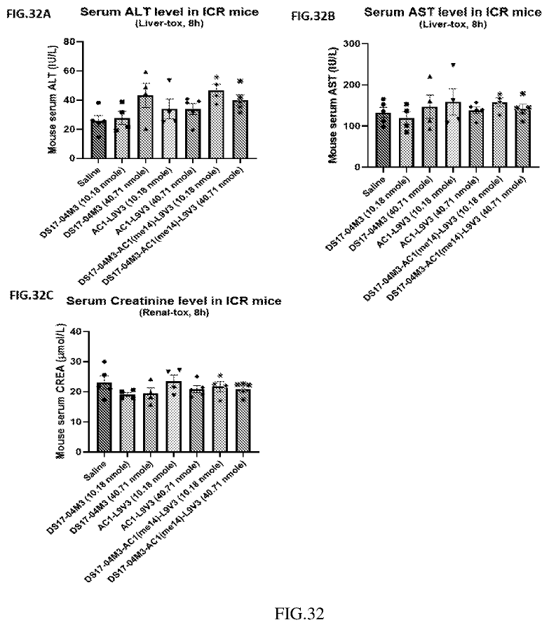

Serum markers for hepatic (i.e., ALT and AST) and renal (i.e., creatine) toxicity were also assessed in serum samples at 8 hours following SC injection. Levels of ALT (FIG. 32A) and AST (FIG. 32B) for all treatment groups were within the biochemical normal reference range and deviating from saline controls by less than 2-fold indicative of natural variability. Similarly, creatine (CREA) levels showed no significant change in comparison to saline treatment controls (FIG. 32C). This data indicates that no acute damage to the liver or kidney was detected following systemic exposure to DS17-04M3-AC1(me14)-L9V3 or any of its components (i.e., DS17-04M3 and AC1-L9V3).

### Example 21. Targeting the 3′UTR of Human SOD1 mRNA with siRNAs and Gapmer ASOs

To further expand the library of siRNAs, as well as identify novel ASOs beyond Toferson, sequence for human SOD1 mRNA (NM_000454.5) was retrieved from the NCBI database that included its 3′-untranslated region (3′UTR). An upstream portion of the 3′UTR located between nucleotide numbers 543-712 (from the first nucleotide of SOD1 3′ UTR to 170^{th} nucleotide in the 3′ UTR) (underlined) was used as a template for both siRNA and ASO design (Table 21).

TABLE 21
Human SOD1 3′UTR sequence used for siRNA and ASO design

| | Size | SEQ |
|---|---|---|
| Human SOD1 3′UTR sequence (5′-3′) | (nt) | ID NO |
| acattcccttggatgtagtctgaggccccttaactcatctgttatcctgctagctgtagaaatgtatcctg | 353 | 59 |
| ataaacattaaacactgtaatcttaaaagtgtaattgtgtgactttttcagagttgctttaaagtacctgtag | | |
| tgagaaactgatttatgatcacttggaagatttgtatagttttataaaactcagttaaaatgtctgtttcaat | | |
| gacctgtattttgccagacttaaatcacagatgggtattaaacttgtcagaatttctttgtcattcaagcct | | |
| gtgaataaaaaccctgtatggcacttattatgaggctattaaaagaatccaaattcaaactaaa | | |

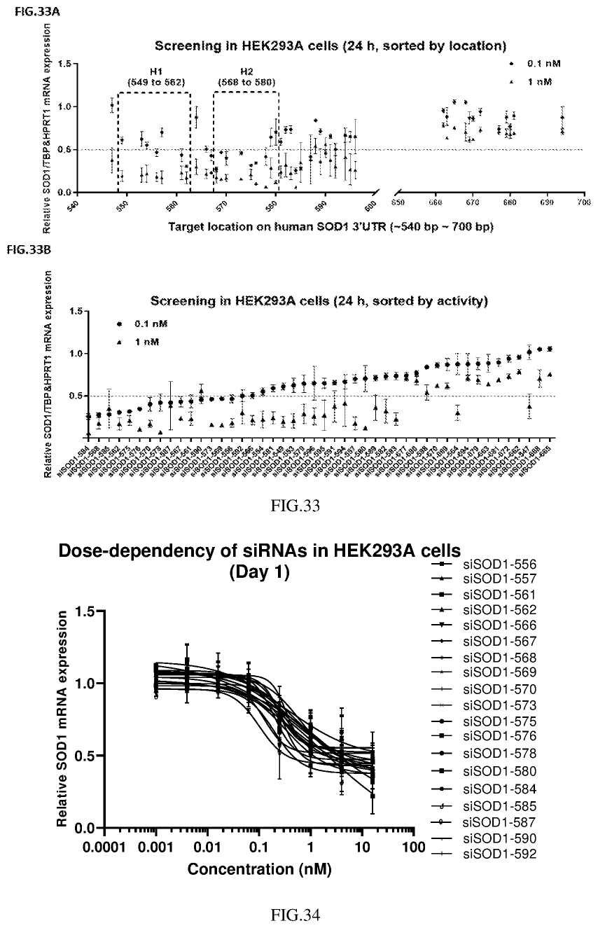

A total of 46 siRNA duplexes were designed and synthesized with asymmetric overhangs having a GC content between 35-65% and no more than 4 repetitive nucleotides (Table 22). Knockdown activity was evaluated via RT-qPCR at both 0.1 and 1 nM concentrations in HEK293A cells 24 hours after transfection. Sorting knockdown data by target site location within the 3′UTR revealed two “hotspots” (referred to as H1 and H2) that contained a majority of the best performing siRNAs (FIG. 33A). H1 is a 14 bp nucleic acid region on the target sequence of the siRNAs, where the 5′-ends of the functional siRNAs are located at the region of 549 to 562, while H2 is a 13 bp nucleic acid region on the target sequence of the siRNAs, where the 5′-ends of the functional siRNAs are located in the region of nucleotide number 568 to 580 relative to the TSS (Table 23). Data ranked by knockdown activity is summarized in FIG. 33B in which the top 19 siRNAs reduced SOD1 levels by ≥45% and ≥70% at 0.1 and 1 nM treatments, respectively. Dose response curves were subsequently generated via RT-qPCR using 8 escalating concentrations (i.e., 0.001, 0.004, 0.016, 0.063, 0.250, 1, 4 and 16 nM) to characterize potency (FIG. 34). As summarized in Table 24, IC₅₀ values for the top 19 performers were all below 7 nM in HEK293A cells.

TABLE 22
siRNA duplex sequence targeting the 3′UTR of human SOD1 mRNA

| Oligo- | SEQ | | SEQ | | SEQ | |
|---|---|---|---|---|---|---|
| nucleotide | ID | Target sequence | ID | | ID | |
| name | NO | (5′-3′) | NO | Sense (5′-3′) | NO | Antisense (5′-3′) |
| siSOD1-547 | 1068 | tcagactacatccaagggaat | 976 | UCCCUUGGAUGUAGUCUGA | 1022 | UCAGACUACAUCCAAGGGAau |
| siSOD1-549 | 1069 | cctcagactacatccaaggga | 977 | CCUUGGAUGUAGUCUGAGG | 1023 | CCUCAGACUACAUCCAAGGga |
| siSOD1-553 | 1070 | ggggcctcagactacatccaa | 978 | GGAUGUAGUCUGAGGCCCC | 1024 | GGGGCCUCAGACUACAUCCaa |
| siSOD1-554 | 1071 | aggggcctcagactacatcca | 979 | GAUGUAGUCUGAGGCCCCU | 1025 | AGGGGCCUCAGACUACAUCca |
| siSOD1-556 | 1072 | taaggggcctcagactacatc | 980 | UGUAGUCUGAGGCCCCUUA | 1026 | UAAGGGGCCUCAGACUACAuc |
| siSOD1-557 | 1073 | ttaaggggcctcagactacat | 981 | GUAGUCUGAGGCCCCUUAA | 1027 | UUAAGGGGCCUCAGACUACau |
| siSOD1-561 | 1074 | tgagttaaggggcctcagact | 982 | UCUGAGGCCCCUUAACUCA | 1028 | UGAGUUAAGGGGCCUCAGAcu |
| siSOD1-562 | 1075 | atgagttaaggggcctcagac | 983 | CUGAGGCCCCUUAACUCAU | 1029 | AUGAGUUAAGGGGCCUCAGac |
| siSOD1-564 | 1076 | agatgagttaaggggcctcag | 984 | GAGGCCCCUUAACUCAUCU | 1030 | AGAUGAGUUAAGGGGCCUCag |
| siSOD1-566 | 1077 | acagatgagttaaggggcctc | 985 | GGCCCCUUAACUCAUCUGU | 1031 | ACAGAUGAGUUAAGGGGCCuc |
| siSOD1-567 | 1078 | aacagatgagttaaggggcct | 986 | GCCCCUUAACUCAUCUGUU | 1032 | AACAGAUGAGUUAAGGGGCcu |
| siSOD1-568 | 1079 | taacagatgagttaaggggcc | 987 | CCCCUUAACUCAUCUGUUA | 1033 | UAACAGAUGAGUUAAGGGGcc |
| siSOD1-569 | 1080 | ataacagatgagttaaggggc | 988 | CCCUUAACUCAUCUGUUAU | 1034 | AUAACAGAUGAGUUAAGGGgc |
| siSOD1-570 | 1081 | gataacagatgagttaagcgc | 989 | CCUUAACUCAUCUGUUAUC | 1035 | GAUAACAGAUGAGUUAAGGgg |
| siSOD1-573 | 1082 | caggataacagatgagttaag | 990 | UAACUCAUCUGUUAUCCUG | 1036 | CAGGAUAACAGAUGAGUUAag |
| siSOD1-575 | 1083 | agcaggataacagatgagtta | 991 | ACUCAUCUGUUAUCCUGCU | 1037 | AGCAGGAUAACAGAUGAGUua |
| siSOD1-576 | 1084 | tagcaggataacagatgagtt | 992 | CUCAUCUGUUAUCCUGCUA | 1038 | UAGCAGGAUAACAGAUGAGuu |
| siSOD1-578 | 1085 | gctagcaggataacagatgag | 993 | CAUCUGUUAUCCUGCUAGC | 1039 | GCUAGCAGGAUAACAGAUGag |
| siSOD1-579 | 1086 | agctagcaggataacagatga | 994 | AUCUGUUAUCCUGCUAGCU | 1040 | AGCUAGCAGGAUAACAGAUga |
| siSOD1-580 | 1087 | cagctagcaggataacagatc | 995 | UCUGUUAUCCUGCUAGCUG | 1041 | CAGCUAGCAGGAUAACAGAug |
| siSOD1-581 | 1088 | acagctagcaggataacagat | 996 | CUGUUAUCCUGCUAGCUGU | 1042 | ACAGCUAGCAGGAUAACAGau |
| siSOD1-582 | 1089 | tacagctagcaggataacaga | 997 | UGUUAUCCUGCUAGCUGUA | 1043 | UACAGCUAGCAGGAUAACAga |
| siSOD1-583 | 1090 | ctacagctagcaggataacag | 998 | GUUAUCCUGCUAGCUGUAG | 1044 | CUACAGCUAGCAGGAUAACag |
| siSOD1-584 | 1091 | tctacagctagcaggataaca | 999 | UUAUCCUGCUAGCUGUAGA | 1045 | UCUACAGCUAGCAGGAUAAca |
| siSOD1-585 | 1092 | ttctacagctagcaggataac | 1000 | UAUCCUGCUAGCUGUAGAA | 1046 | UUCUACAGCUAGCAGGAUAac |
| siSOD1-587 | 1093 | atttctacagctagcaggata | 1001 | UCCUGCUAGCUGUAGAAAU | 1047 | AUUUCUACAGCUAGCAGGAua |
| siSOD1-588 | 1094 | catttctacagctagcaggat | 1002 | CCUGCUAGCUGUAGAAAUG | 1048 | CAUUUCUACAGCUAGCAGGau |
| siSOD1-589 | 1095 | acatttctacagctagcagga | 1003 | CUGCUAGCUGUAGAAAUGU | 1049 | ACAUUUCUACAGCUAGCAGga |
| siSOD1-590 | 1096 | tacatttctacagctagcagg | 1004 | UGCUAGCUGUAGAAAUGUA | 1050 | UACAUUUCUACAGCUAGCAgg |
| siSOD1-591 | 1097 | atacatttctacagctagcag | 1005 | GCUAGCUGUAGAAAUGUAU | 1051 | AUACAUUUCUACAGCUAGCag |
| siSOD1-592 | 1098 | gatacatttctacagctagca | 1006 | CUAGCUGUAGAAAUGUAUC | 1052 | GAUACAUUUCUACAGCUAGca |
| siSOD1-594 | 1099 | aggatacatttctacagctag | 1007 | AGCUGUAGAAAUGUAUCCU | 1053 | AGGAUACAUUUCUACAGCUag |
| siSOD1-595 | 1100 | caggatacatttctacagcta | 1008 | GCUGUAGAAAUGUAUCCUG | 1054 | CAGGAUACAUUUCUACAGCua |
| siSOD1-596 | 1101 | tcaggatacatttctacagct | 1009 | CUGUAGAAAUGUAUCCUGA | 1055 | UCAGGAUACAUUUCUACAGcu |
| siSOD1-662 | 1102 | gtactttaaagcaactctgaa | 1010 | CAGAGUUGCUUUAAAGUAC | 1056 | GUACUUUAAAGCAACUCUGaa |
| siSOD1-663 | 1103 | ggtactttaaagcaactctga | 1011 | AGAGUUGCUUUAAAGUACC | 1057 | GGUACUUUAAAGCAACUCUga |
| siSOD1-665 | 1104 | caggtactttaaagcaactct | 1012 | AGUUGCUUUAAAGUACCUG | 1058 | CAGGUACUUUAAAGCAACUcu |
| siSOD1-668 | 1105 | ctacaggtactttaaagcaac | 1013 | UGCUUUAAAGUACCUGUAG | 1059 | CUACAGGUACUUUAAAGCAac |
| siSOD1-669 | 1106 | actacaggtactttaaagcaa | 1014 | GCUUUAAAGUACCUGUAGU | 1060 | ACUACAGGUACUUUAAAGCaa |
| siSOD1-670 | 1107 | cactacaggtactttaaagca | 1015 | CUUUAAAGUACCUGUAGUG | 1061 | CACUACAGGUACUUUAAAGca |
| siSOD1-672 | 1108 | ctcactacaggtactttaaag | 1016 | UUAAAGUACCUGUAGUGAG | 1062 | CUCACUACAGGUACUUUAAag |
| siSOD1-677 | 1109 | agtttctcactacaggtactt | 1017 | GUACCUGUAGUGAGAAACU | 1063 | AGUUUCUCACUACAGGUACuu |
| siSOD1-679 | 1110 | tcagtttctcactacaggtac | 1018 | ACCUGUAGUGAGAAACUGA | 1064 | UCAGUUUCUCACUACAGGUac |
| siSOD1-680 | 1111 | atcagtttctcactacaggta | 1019 | CCUGUAGUGAGAAACUGAU | 1065 | AUCAGUUUCUCACUACAGGua |
| siSOD1-681 | 1112 | aatcagtttctcactacaggt | 1020 | CUGUAGUGAGAAACUGAUU | 1066 | AAUCAGUUUCUCACUACAGgu |
| siSOD1-694 | 1113 | ccaagtgatcataaatcagtt | 1021 | CUGAUUUAUGAUCACUUGG | 1067 | CCAAGUGAUCAUAAAUCAGuu |

n.b.: Target sequence is identical to the identified antisense sequence but the nucleotide ″U″ is converted to ″T″. Antisense sequence includes 2-nt nucleotides natural overhang from the corresponding nucleotides on the DNA target.

TABLE 23
“Hotspot” region sequence in buman SOD1 3′UTR.

| | | SEQ ID |
|---|---|---|
| Hotspot | Hotspot sequences of siRNA on human SOD1 3′UTR (5′-3′) | NO |
| H1 (549 to 562) | ccttggatgtagtctgaggccccttaactcat | 61 |
| H2 (568 to 580) | ccccttaactcatctgttatcctgctagctg | 63 |
| Hotspot | Hotspot sequences of ASO on human SOD1 3′UTR (5′-3′) | |
| H3 (552 to 566) | tggatgtagtctgaggccccttaactcatctg | 65 |

TABLE 24
IC₅₀ values of top 19 siRNAs targeting SOD1 3′UTR

| Duplex | IC₅₀ (nM) |
|---|---|
| siSOD1-556 | 3.369 |
| siSOD1-557 | 2.78 |
| siSOD1-561 | 1.431 |
| siSOD1-562 | 3.984 |
| siSOD1-566 | 1.975 |
| siSOD1-567 | 2.915 |
| siSOD1-568 | 2.482 |
| siSOD1-569 | 1.376 |
| siSOD1-570 | 3.578 |
| siSOD1-573 | 1.439 |
| siSOD1-575 | 1.734 |
| siSOD1-576 | 1.989 |
| siSOD1-578 | 3.42 |
| siSOD1-580 | 2.169 |
| siSOD1-584 | 6.505 |
| siSOD1-585 | 1.1 |
| siSOD1-587 | 2.921 |
| siSOD1-590 | 4.49 |
| siSOD1-592 | 2.406 |

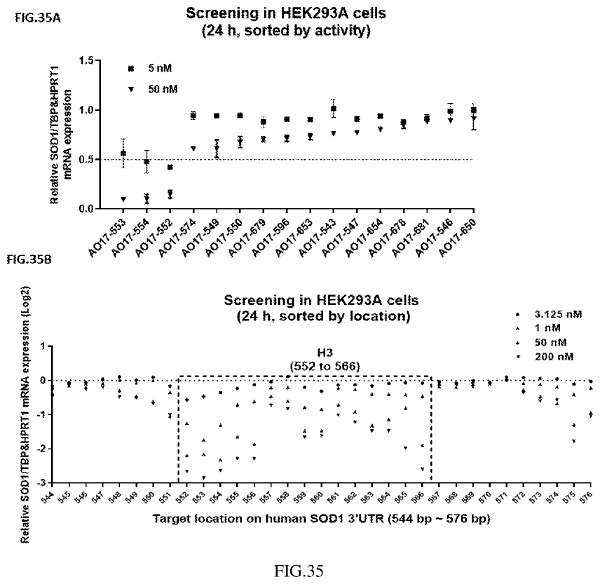

To test knockdown of SOD1 mRNA using ASOs, 16 representative ASOs (i.e., AO17-543, -546, -547, -549, -550, -552, -553, -554, -574, -596, -653, -650, -654, -678, -679, and -681) were synthesized out of 46 designs (Table 25) spanning the 3′UTR region inclusive to the underlined sequence in Table 21. All ASOs were designed using a 4-104 modification pattern in which 4 tandem 2′Ome-modified nucleotides flanked an internal stretch of 10 deoxyribose nucleic acids (DNA) on either side to form ‘gapmer’ ASOs 18 nucleotides in length. HEK293A cells were transfected at 5 and 50 nM concentrations with each ASO and knockdown activity was assessed 24 hours later via RT-qPCR. Sorting data by maximal knockdown activity revealed AO17-552, -554 and -553 were the best overall performers reducing SOD1 mRNA levels by ˜50% and ˜90% at 5 and 50 nM treatments, respectively (FIG. 35A). The next 3 best performing ASOs (i.e., AO17-549, -550, and -574) had a drop in activity measuring ˜40% reduction in SOD1 levels at only the higher 50 nM treatment concentration. All other ASOs had only a modest or no detectable impact on SOD1 expression levels.

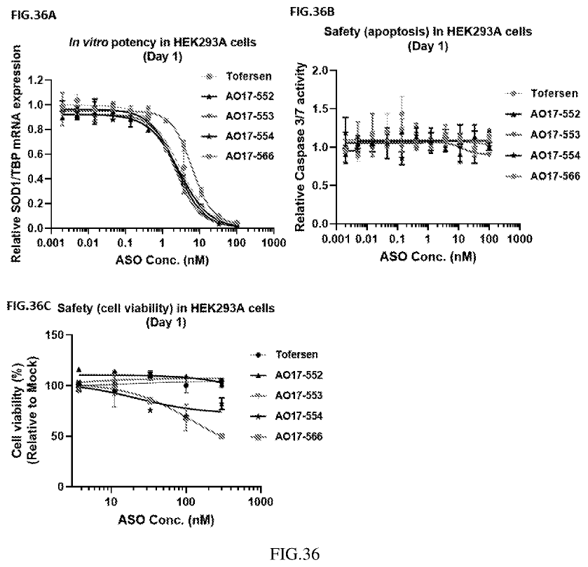

Based on target location of the 6 best performing ASOs, an additional 27 ‘gapmers’ were selected for synthesis out of the 41 designs listed in Table 25 targeting the region between nucleotide numbers 544-593 in the 3′UTR. Knockdown activity was evaluated via RT-qPCR at 4 concentrations (i.e., 3.125, 1, 50, and 200 nM) in HEK293A cells 24 hours after transfection. Sorting knockdown data by target site location revealed an ASO-specific “hotspot” (referred to as H3) within the 3′UTR that contained a majority of the best performing ASOs (FIG. 35B). H3 is a 15 bp nucleic acid region on the target sequence of the ASOs, where the 5′-ends of the functional ASOs are located in the region of nucleotide number 552 to 566 relative to the TSS (Table 23). Dose response curves were subsequently generated via RT-qPCR for each of the top 4 performers (i.e., AO17-552, 553, 554, and 556) using 11 escalating concentrations (i.e., 0.002, 0.005, 0.015, 0.046, 0.137, 0.412, 1.235, 3.704, 11.111, 33.333 and 100 nM) to characterize ASO potency compared to Toferson (FIG. 36A). As summarized in Table 26, IC₅₀ values were all in the low nanomolar range (i.e., 2.23-6.22 nM) in which AO17-553 and AO17-554 were the most potent ASOs overall. Subsequent analyses for cytotoxicity were performed to determine impact on apoptosis and cell viability as markers for safety. As shown in FIG. 36B, there was no marked increase in apoptosis up to 100 nM for any of the tested ASOs as no significant changes in caspase 3/7 activity were detected after treatment in HEK293A cells. Cell viability was quantified by CCK8 assay at 5 escalating doses (i.e., 3.704, 11.111, 33.333, 100, and 300 nM) in HEK293A cells 1 day after transfection. As shown in FIG. 36C, cell viability was unaffected at any dose for Toferson, AO17-552, and AO17-553, whereas AO17-554 and AO-556 had moderate cytotoxicity measuring a maximal loss of ˜30% and ˜50% in cell viability, respectively.

TABLE 25
SOD1 ASOs sequence targeting human SOD1 3′UTR

| Oligo- | | | SEQ | | SEQ | | |
|---|---|---|---|---|---|---|---|
| nucleotide | | Target | ID | Target sequence | ID | | |
| name | Type | gene | NO | (5′-3′) | NO | Sequence (5′-3′) | Size (nt) |
| AO17-543 | ASO | SOD1 | 1114 | ctacatccaagggaatgt | 1155 | meC*meU*meA*meC*a*t*c*c*a*a*g*g*g*a*meA*meU*meG*meU | 18 |
| AO17-544 | ASO | SOD1 | 1115 | actacatccaagggaatg | 1156 | meA*meC*meU*meA*c*a*t*c*c*a*a*g*g*g*meA*meA*meU*meG | 18 |
| AO17-545 | ASO | SOD1 | 1116 | gactacatccaagggaat | 1157 | meG*meA*meC*meU*a*c*a*t*c*c*a*a*g*g*meG*meA*meA*meU | 18 |
| A017-546 | ASO | SOD1 | 1117 | agactacatccaagggaa | 1158 | meA*meG*meA*meC*t*a*c*a*t*c*c*a*a*g*meG*meG*meA*meA | 18 |
| AO17-547 | ASO | SOD1 | 1118 | cagactacatccaaggga | 1159 | meC*meA*meG*meA*c*t*a*c*a*t*c*c*a*a*meG*meG*meG*meA | 18 |
| AO17-548 | ASO | SOD1 | 1119 | tcagactacatccaaggg | 1160 | meU*meC*meA*meG*a*c*t*a*c*a*t*c*c*a*meA*meG*meG*meG | 18 |
| AO17-549 | ASO | SOD1 | 1120 | ctcagactacatccaagc | 1161 | meC*meU*meC*meA*g*a*c*t*a*c*a*t*c*c*meA*meA*meG*meG | 18 |
| AO17-550 | ASO | SOD1 | 1121 | cctcagactacatccaag | 1162 | meC*meC*meU*meC*a*g*a*c*t*a*c*a*t*c*meC*meA*meA*meG | 18 |
| A017-551 | ASO | SOD1 | 1122 | gcctcagactacatccaa | 1163 | meG*meC*meC*meU*c*a*g*a*c*t*a*c*a*t*meC*meC*meA*meA | 18 |
| AO17-552 | ASO | SOD1 | 1123 | ggcctcagactacatcca | 1164 | meG*meG*meC*meC*t*c*a*g*a*c*t*a*c*a*meU*meC*meC*meA | 18 |
| AO17-553 | ASO | SOD1 | 1124 | gggcctcagactacatec | 1165 | meG*meG*meG*meC*c*t*c*a*g*a*c*t*a*c*meA*meU*meC*meC | 18 |
| AO17-554 | ASO | SOD1 | 1125 | ggggcctcagactacatc | 1166 | meG*meG*meG*meG*c*c*t*c*a*g*a*c*t*a*meC*meA*meU*meC | 18 |
| AO17-555 | ASO | SOD1 | 1126 | aggggcctcagactacat | 1167 | meA*meG*meG*meG*g*c*c*t*c*a*g*a*c*t*meA*meC*meA*meU | 18 |
| A017-556 | ASO | SOD1 | 1127 | aaggggcctcagactaca | 1168 | meA*meA*meG*meG*g*g*c*c*t*c*a*g*a*c*meU*meA*meC*meA | 18 |
| A017-557 | ASO | SOD1 | 1128 | taaggggcctcagactac | 1169 | meU*meA*meA*meG*g*g*g*c*c*t*c*a*g*a*meC*meU*meA*meC | 18 |
| AO17-558 | ASO | SOD1 | 1129 | ttaaggggcctcagacta | 1170 | meU*meU*meA*meA*g*g*g*g*c*c*t*c*a*g*meA*meC*meU*meA | 18 |
| AO17-559 | ASO | SOD1 | 1130 | gttaaggggcctcagact | 1171 | meG*meU*meU*meA*a*g*g*g*g*c*c*t*c*a*meG*meA*meC*meU | 18 |
| AO17-560 | ASO | SOD1 | 1131 | agttaaggggcctcagac | 1172 | meA*meG*meU*meU*a*a*g*g*g*g*c*c*t*c*meA*meG*meA*meC | 18 |
| A017-561 | ASO | SOD1 | 1132 | gagttaaggggcctcaga | 1173 | meG*meA*meG*meU*t*a*a*g*g*g*g*c*c*t*meC*meA*meG*meA | 18 |
| AO17-562 | ASO | SOD1 | 1133 | tgagttaaggggcctcag | 1174 | meU*meG*meA*meG*t*t*a*a*g*g*g*g*c*c*meU*meC*meA*meG | 18 |
| AO17-563 | ASO | SOD1 | 1134 | atgagttaaggggcctca | 1175 | meA*meU*meG*meA*g*t*t*a*a*g*g*g*g*c*meC*meU*meC*meA | 18 |
| AO17-564 | ASO | SOD1 | 1135 | gatgagttaaggggcctc | 1176 | meG*meA*meU*meG*a*g*t*t*a*a*g*g*g*g*meC*meC*meU*meC | 18 |
| AO17-565 | ASO | SOD1 | 1136 | agatgagttaaggggcct | 1177 | meA*meG*meA*meU*g*a*g*t*t*a*a*g*g*g*meG*meC*meC*meU | 18 |
| A017-566 | ASC | SOD1 | 1137 | cagatgagttaaggggcc | 1178 | meC*meA*meG*meA*t*g*a*g*t*t*a*a*g*g*meG*meG*meC*meC | 18 |
| AO17-567 | ASO | SOD1 | 1138 | acagatgagttaaggggc | 1179 | meA*meC*meA*meG*a*t*g*a*g*t*t*a*a*g*meG*meG*meG*meC | 18 |
| AO17-568 | ASO | SOD1 | 1139 | aacagatgagttaagggg | 1180 | meA*meA*meC*meA*g*a*t*g*a*g*t*t*a*a*meG*meG*meG*meG | 18 |
| AO17-569 | ASO | SOD1 | 1140 | taacagatgagttaaggg | 1181 | meU*meA*meA*meC*a*g*a*t*g*a*g*t*t*a*meA*meG*meG*meG | 18 |
| AO17-570 | ASO | SOD1 | 1141 | ataacagatgagttaagg | 1182 | meA*meU*meA*meA*c*a*g*a*t*g*a*g*t*t*meA*meA*meG*meG | 18 |
| AO17-571 | ASO | SOD1 | 1142 | gataacagatgagttaag | 1183 | meG*meA*meU*meA*a*c*a*g*a*t*g*a*g*t*meU*meA*meA*meG | 18 |
| AO17-572 | ASO | SOD1 | 1143 | ggataacagatgagttaa | 1184 | meG*meG*meA*meU*a*a*c*a*g*a*t*g*a*g*meU*meU*meA*meA | 18 |
| A017-573 | ASO | SOD1 | 1144 | aggataacagatgagtta | 1185 | meA*meG*meG*meA*t*a*a*c*a*g*a*t*g*a*meG*meU*meU*meA | 18 |
| AO17-574 | ASO | SOD1 | 1145 | caggataacagatgagtt | 1186 | meC*meA*meG*meG*a*t*a*a*c*a*g*a*t*g*meA*meG*meU*meU | 18 |
| AO17-575 | ASO | SOD1 | 1146 | gcaggataacagatgagt | 1187 | meG*meC*meA*meG*g*a*t*a*a*c*a*g*a*t*meG*meA*meG*meU | 18 |
| AO17-576 | ASO | SOD1 | 1147 | agcaggataacagatgag | 1188 | meA*meG*meC*meA*g*g*a*t*a*a*c*a*g*a*meU*meG*meA*meG | 18 |
| AO17-596 | ASO | SOD1 | 1148 | caggatacatttctacag | 1189 | meC*meA*meG*meG*a*t*a*c*a*t*t*t*c*t*meA*meC*meA*meG | 18 |
| A017-650 | ASO | SOD1 | 1149 | actctgaaaaagtcacac | 1190 | meA*meC*meU*meC*t*g*a*a*a*a*a*g*t*c*meA*meC*meA*meC | 18 |
| A017-653 | ASO | SOD1 | 1150 | gcaactctgaaaaagtca | 1191 | meG*meC*meA*meA*c*t*c*t*g*a*a*a*a*a*meG*meU*meC*meA | 18 |
| AO17-654 | ASO | SOD1 | 1151 | agcaactctgaaaaagtc | 1192 | meA*meG*meC*meA*a*c*t*c*t*g*a*a*a*a*meA*meG*meU*meC | 18 |
| AO17-678 | ASO | SOD1 | 1152 | agtttctcactacaggta | 1193 | meA*meG*meU*meU*t*c*t*c*a*c*t*a*c*a*meG*meG*meU*meA | 18 |
| AO17-679 | ASO | SOD1 | 1153 | cagtttctcactacaggt | 1194 | meC*meA*meG*meU*t*t*c*t*c*a*c*t*a*c*meA*meG*meG*meU | 18 |
| A017-681 | ASO | SOD1 | 1154 | atcagtttctcactacag | 1195 | meA*meU*meC*meA*g*t*t*t*c*t*c*a*c*t*meA*meC*meA*meG | 18 |

n.b.: Uppercase, RNA; Lowercase, DNA; *, phosphorothioate (PS) backbone modification; me, 2′-O-methoxyethyl (2′MOE); ;C, 5′ methyl cytosine; U, 5-methyl uracil, c, 5′ methyl cytosine.

TABLE 26
IC₅₀ values of top 4 ASOs targeting SOD1 3'UTR

| ASOs | IC₅₀ (nM) |
|---|---|
| Tofersen | 2.80 |
| AO17-552 | 2.81 |
| AO17-553 | 2.23 |
| AO17-554 | 2.41 |
| AO17-556 | 6.22 |

### Example 22. Synthesis of Linking Component Compounds for Linking ACO with Duplex Oligonucleotide at an Internal Position of its Sense or Antisense Strand

Compound list:

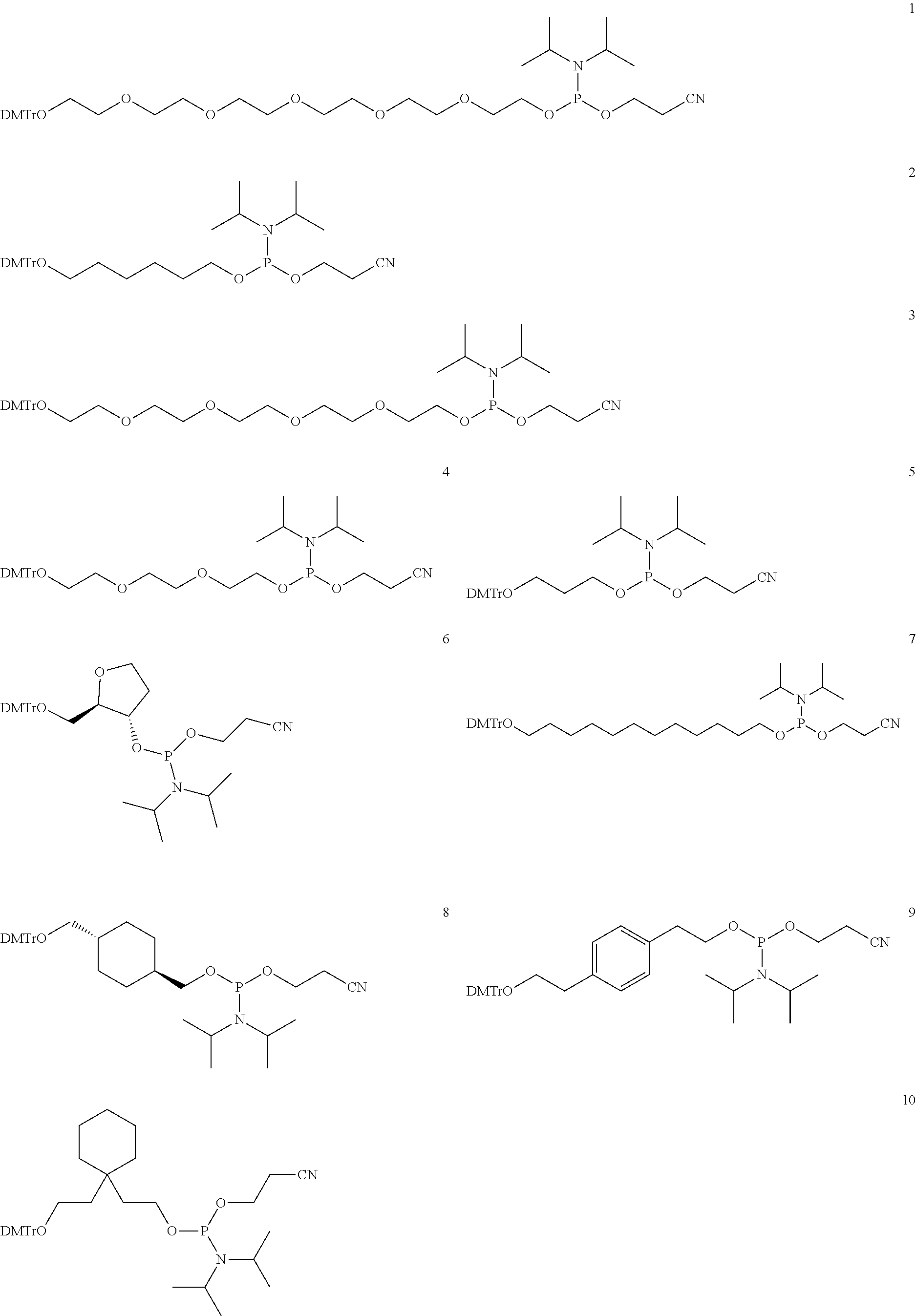

Compounds 1, 2, 3, 4, 5, 6, 7 are commercially available. All these compounds were applied as monomer/spacer in oligo synthesis.

Compound 8 Synthesis Compound 8 was prepared in this Example by using the following procedures.

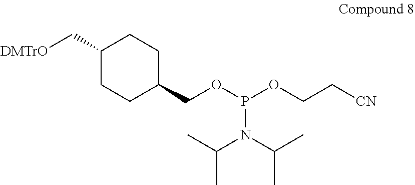

### (1) Preparation of compound 16 from the starting compound ((1r,4r)-cyclohexane-1,4-diyl)dimethanol 15

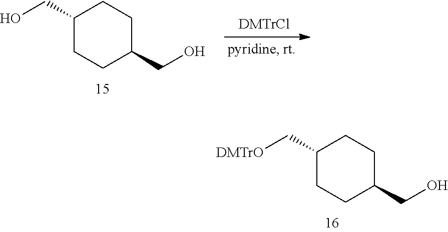

To a solution of ((1r,4r)-cyclohexane-1,4-diyl)dimethanol 15 (10 g, 69.3 mmol, 1.0 eq) in anhydrous pyridine (200 mL) under nitrogen atmosphere was added DMTrCl (23.48 g, 69.3 mmol, 1.0 eq) slowly. The reaction mixture was stirred at room temperature for 6 h then concentrated under reduced pressure, the resultant residue was purified by flash chromatography (silica gel, gradient eluent: 1-3% of MeOH/DCM) to provide compound 16 (12.1 g, 39% yield) as yellow oil. The product was characterized with mass spectrometry and ¹H NMR. MW calc.: 446.25; MW Found: 303.09 [DMT]⁻, 144.15 [DMT off+H]⁺. ¹H NMR (400 MHz, CDCl₃) δ 7.49 (d, J=7.4 Hz, 2H), 7.37 (t, J=6.0 Hz, 4H), 7.34-7.31 (m, 2H), 7.23 (dd, J=4.7, 2.4 Hz, 1H), 6.88-6.85 (m, 4H), 3.83 (s, 6H), 3.53-3.49 (m, 2H), 2.93 (dd, J=6.2, 3.8 Hz, 2H), 1.91 (dd, J=13.0, 5.0 Hz, 4H), 1.63 (d, J=3.3 Hz, 1H), 1.43 (s, 1H), 1.03 (dd, J=12.7, 7.9 Hz, 41H).

### (2) Preparation of Compound 8 from Compound 16

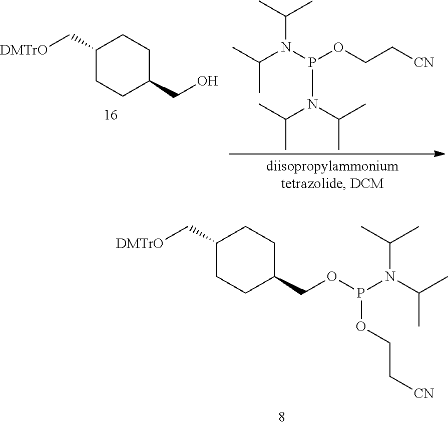

To a solution of compound 16 (3.4 g, 7.65 mmol, 1.0 eq) and diisopropylammonium tetrazolide (2.6 g, 15.3 mmol, 2.0 eq) in anhydrous Dichloromethane (DCM, 30 mL) under nitrogen atmosphere was added 3-((bis(diisopropylamino)phosphaneyl)oxy)propanenitrile (4.6 g, 15.3 mmol, 2.0 eq.) at room temperature. The reaction mixture was stirred for 3 h. The mixture was extracted two times with DCM, then washed with brine and dried with anhydrous Na₂SO₄. The organic layer was concentrated under reduced pressure and the resultant residue was purified with flash chromatography (silica gel, gradient eluent. 1-20% of EtOAc/Hexane, 1% Et N) to provide compound 8 (3.65 g, 75% yield) as yellow oil. The product was characterized with mass spectrometry and ¹H NMR. MW calc.: 646.35; MW Found 303.09 [DMT]⁻, 302.36 [DMT and one isopropyl off]⁺. ¹H NMR (400 MHz, CDCl₃) δ 7.47 (d, J=7.3 Hz, 2H), 7.35 (t, J=6.0 Hz, 4H), 7.30 (d, J=8.4 Hz, 2H), 7.24-7.20 (m, 1H), 6.87-6.83 (m, 4H), 3.94-3.83 (m, 2H), 3.82 (s, 6H), 3.64 (ddt, J=13.6, 10.1, 6.8 Hz, 2H), 3.52 (dt, J=9.7, 7.4 Hz, 1H), 3.43 (dt, J=9.9, 7.1 Hz, 11H), 2.91 (d, J=6.3 Hz, 2H), 2.67 (t, J=6.5 Hz, 2H), 1.88 (dd, J=24.8, 6.2 Hz, 4H), 1.64-1.55 (m, 2H), 1.22 (dd, J=6.8, 3.2 Hz, 12H), 1.02 (t, J=10.4 Hz, 4H).

### Compound 9 Synthesis

Compound 9 was prepared in this Example by using the following procedures.

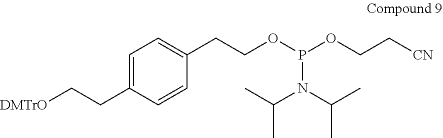

### (1) Preparation of compound 18 from the starting compound 2,2′-(1,4-phenylene)bis(ethan-1-ol) 17

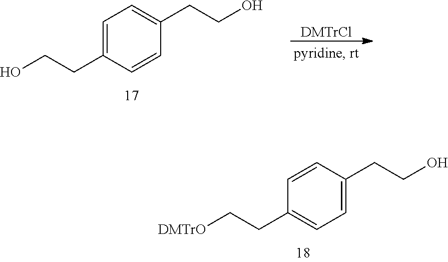

To a solution of 2,2′-(1,4-phenylene)bis(ethan-1-ol) 17 (3 g, 18.0 mmol, 1.0 eq) in anhydrous pyridine (50 mL) under nitrogen atmosphere was added DMTrCl (6.1 g, 18.0 mmol, 1.0 eq) slowly. The reaction mixture was stirred at room temperature for 6 h then concentrated under reduced pressure, the resultant residue was purified by flash chromatography (silica gel, gradient eluent: 1-3% of MeOH/DCM) to provide compound 6 (3.74 g, 44% yield) as yellow oil. The product was characterized with mass spectrometry and ¹H NMR MW calc: 468.23; MW Found: 303.16 [DMT]⁻, 491.31 [M+Na]⁺. ¹H NMR (400 MHz, CDCl₃) δ 7.42 (dd, J=5.3, 3.3 Hz, 2H), 7.33-7.29 (m, 4H), 7.28 (d, J=7.8 Hz, 2H), 7.22 (s, 11H), 7.18 (s, 4H), 6.85-6.81 (m, 4H), 3.88 (dd, J=8.3, 4.8 Hz, 2H), 3.81 (s, 6H), 3.31 (t, J=7.0 Hz, 2H), 2.93-2.87 (m, 4H).

### (2) Preparation of Compound 9 from Compound 18

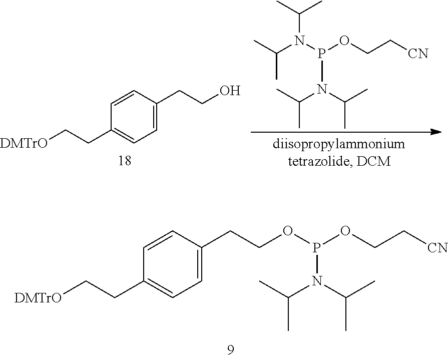

To a solution of compound 18 (884 mg, 1.89 mmol, 1.0 eq) and diisopropylammonium tetrazolide (647 mg, 3.78 mmol, 2.0 eq) in anhydrous DCM (10 mL) under nitrogen atmosphere was added 3-((bis(diisopropylamino)phosphaneyl)oxy)propanenitrile (1.14 g, 3.78 mmol, 2.0 eq.) at room temperature. The reaction mixture was stirred for 3 h. The mixture was extracted two times with DCM, then washed with brine and dried with anhydrous Na₂SO₄. The organic layer was concentrated under reduced pressure and the resultant residue was purified with flash chromatography (silica gel, gradient eluent. 1-30% of EtOAc/Hexane, 1% Et₃N) to provide compound 9 (292 mg, 23% yield) as yellow oil. The product was characterized with mass spectrometry and ¹H NMR. MW calc.: 668.34; MW Found 303.11 [DMT]⁻. ¹H NMR (400 MHz, CDCl₃) δ7.41-7.38 (m, 2H), 7.29 (d, J=7.9 Hz, 6H), 7.23 (d, J=7.1 Hz, 1H), 7.18-7.14 (m, 4H), 6.85-6.80 (m, 4H), 3.96-3.82 (m, 2H), 3.81 (d, J=6.8 Hz, 6H), 3.78-3.72 (m, 2H), 3.65-3.55 (m, 2H), 3.29 (t, J=7.0 Hz, 2H), 2.92 (dt, J=16.8, 7.1 Hz, 4H), 2.52 (td, J=6.5, 2.9 Hz, 2H), 1.18 (dd, J=14.4, 6.8 Hz, 12H).

### Compound 10 Synthesis

Compound 10 was prepared in this Example by using the following procedures.

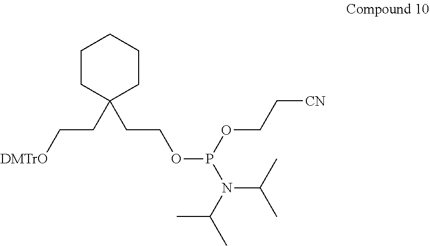

### (1) Preparation of compound 20 from the reduction of starting compound 2,2′-(cyclohexane-1,1-diyl)diacetic acid 19

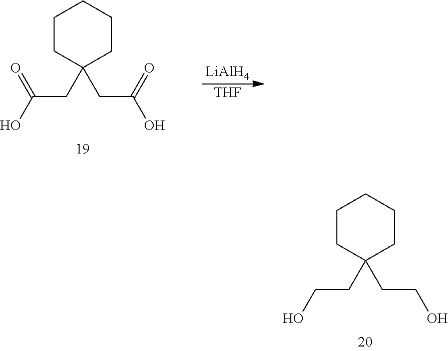 [SMILES: O=C(O)CC1(CC(=O)O)CCCCC1 ; "LiAlH4" ; OCCC1(CCO)CCCCC1 ; "THF"]

To a solution of compound 19 (10 g, 50 mmol, 1.0 eq) in anhydrous THF (200 mL) under nitrogen atmosphere and ice bath was added LiAlH4 (5.7 g, 150 mmol, 3.0 eq). The mixture was then transferred to room temperature after 10 minutes and stirred for about 1 h. Then the reaction was transferred to ice bath, saturated potassium sodium tartrate solution (100 mL) was added slowly into the mixture. After 30 minutes, the reaction was extracted 3 times by Et2O, then the organic phase was combined and washed by brine, dried over Na2SO4, and concentrated. The product was characterized with mass spectrometry. MW calc.: 172.15; MW Found: 173.22 [M+H]+.

### (2) Preparation of Compound 21 from Compound 20

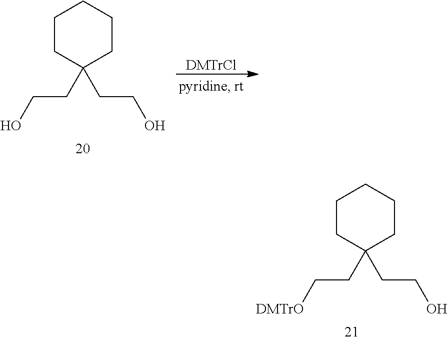

To a solution of compound 20 (3 g, 17.4 mmol, 1.0 eq) in anhydrous pyridine (50 mL) under nitrogen atmosphere was added DMTrCl (4.7 g, 13.92 mmol, 0.8 eq) slowly. The reaction mixture was stirred at room temperature for 6 hi then concentrated under reduced pressure. The resultant residue was purified by flash chromatography (silica gel, gradient eluent: 1-3% of MeOH/DCM) to provide compound 21 (3.0 g, 36% yield) as yellow oil. The product was characterized with mass spectrometry and ¹H NMR. MW calc.: 474.28; MW Found 303.12 [DMT]⁻. ¹H NMR (400 MHz, CDCl₃) δ 7.46-7.41 (m, 2H), 7.35-7.30 (m, 4H), 7.28 (d, J=2.3 Hz, 2H), 7.19-7.15 (m, 1H), 6.84-6.80 (m, 4H), 3.78 (s, 6H), 3.59 (dd, J=9.5, 5.8 Hz, 2H), 3.11 (t, J=7.2 Hz, 2H), 1.45-1.34 (m, 10H), 1.20 (dd, J=6.8, 3.4 Hz, 4H).

### (3) Preparation of Compound 10 from Compound 21

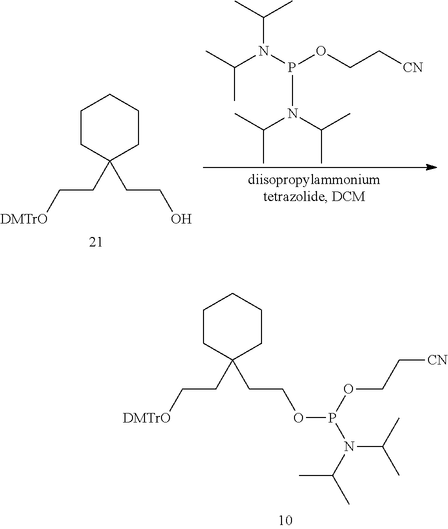

To a solution of compound 21 (1.3 g, 2.74 mmol, 1.0 eq) and diisopropylammonium tetrazolide (938 mg, 5.48 mmol, 2.0 eq) in anhydrous DCM (15 mL) under nitrogen atmosphere was added 3-((bis(diisopropylamino)phosphaneyl)oxy)propanenitrile (1.65 g, 5.48 mmol, 2.0 eq.) at room temperature. The reaction mixture was stirred for 3 h. The mixture was extracted two times with DCM, then washed with brine and dried with anhydrous Na₂SO₄. The organic layer was concentrated under reduced pressure and the resultant residue was purified with flash chromatography (silica gel, gradient eluent: 1-20% of EtOAc/Hexane, 1% Et₃N) to provide compound 10 (425 mg, 23% yield) as yellow oil. The product was characterized with mass spectrometry and ¹H NMR MW calc.: 674.38; MW Found: 303.23 [DMT]⁻, 396.30 [DMT and one isopropyl off+Na]⁺. ¹H NMR (400 MHz, CDCl₃) δ 7.45-7.40 (m, 2H), 7.34-7.29 (m, 4H), 7.29-7.25 (m, 2H), 7.22-7.16 (m, 1H), 6.86-6.79 (m, 4H), 3.79 (s, 6H), 3.78-3.71 (m, 2H), 3.65-3.50 (m, 41H), 3.10 (t, J=7.4 Hz, 2H), 2.58 (t, J=6.6 Hz, 2H), 1.66 (t, J=7.4 Hz, 2H), 1.51 (t, J=7.6 Hz, 2H), 1.37 (dd, J=18.4, 4.5 Hz, 6H), 1.22 (s, 4H), 1.17 (d, J=6.8 Hz, 6H), 1.13 (d, J=6.8 Hz, 6H).

All compounds in this example are listed in Table 1.

### Example 23. Design of Various ODV-siSOD1 Structures

A series of ACO sequences with varying lengths, nucleotide compositions, and chemical modifications were conjugated to Sod1 siRNA duplex (siSOD1, RD-12559) on the 3′ terminus of its passenger strand using the L9 linker or alternative spacer as indicated resulting in a total of 90 ODV-siRNA variants categorized into 12 groups. All ACO) variants are listed by group in Table 27, while each ODV-siSOD1 duplex can be found in Table 28.

Group A (Linker group) includes 8 compounds in which the siRNA and ACO components were fixed, but conjugated together using different linkers including S18, C3, C6, C12, L14, L15, L16, UUACA and UUCUU.

Group B (Palindromic AC1 sequence group) includes 15 compounds in which the siRNA and linker (L9) were fixed, but different palindromic AC1 sequence and length were used based on AC1 sequence. All nucleic acid chemistries remained unchanged compared to AC1 including 2′MOE modification and PS backbone at every position within each ACO.

Group C (PS modification group) includes 6 compounds in which the siRNA, linker (L9), and ACO sequence remained fixed, but varied in the number of PS backbone modifications (from 2 to 12) in the ACO.

Group D (2′-Ome modification group) includes 7 compounds in which the siRNA, linker (L9), and ACO sequence remained fixed, but varied in the number of 2′Ome substitutions (from 2 to 14) for 2′MOE.

Group E (ACO size group) includes 8 compounds in which the siRNA and linker (L9) remained fixed, but ACO composition changed only in size ranging from 13 to 6 nucleotide in length.

Group F (Adenine rich group) includes 5 compounds in which the siRNA, linker (L9), and ACO size and chemistry pattern remained fixed, but the total number of adenine nucleotides range from 5 to 9 in the ACO sequence.

Group G (Cytosine rich group) includes 6 compounds in which the siRNA, linker (L9), and ACO size and chemistry pattern remained fixed, but the total number of cytosine nucleotides range from 5 to 10 in the ACO sequence.

Group H (Guanine rich group) includes 5 compounds in which the siRNA, the linker (L9), and ACO size and chemistry pattern remained fixed, but the total number of guanine nucleotides range from 5 to 9 in the ACO sequence.

Group I (Uracil rich group) includes 6 compounds in which the siRNA, linker (L9), and ACO size and chemistry pattern remained fixed, but the total number of uracil nucleotides range from 5 to 10 in the ACO sequence.

Group J (Purine rich group) includes 8 compounds in which the siRNA, linker (L9), and ACO size and chemistry pattern remained fixed, but the total number of purines range from 9 to 11 in the ACO sequence.

Group K (Pyrimidine rich group) includes 8 compounds in which the siRNA, linker (L9), and ACO size and chemistry pattern remained fixed, but the total number of pyrimidines range from 9 to 12 in the ACO sequence.

Group L (Balanced pur:pyr group) includes 6 compounds in which the siRNA, linker (L9), and ACO size and chemistry pattern remained fixed, but the ACO contains an equal ratio of purines to pyrimidines comprised of different sequences.

### Example 24. In Vitro Uptake and Safety Assessment of ODV-siSOD1 Compounds in PMH Cells

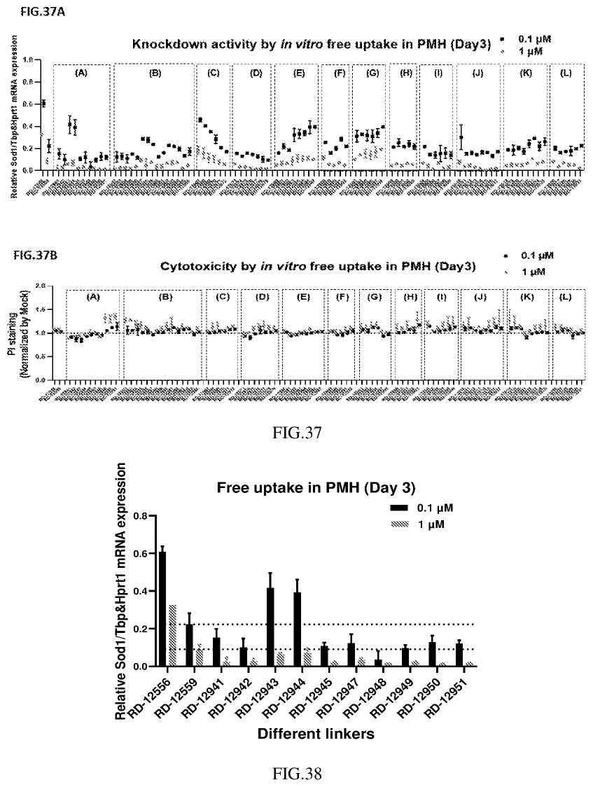

Free uptake of each ODV-siSOD1 variant listed in Table 28 was tested by directly adding each duplex to freshly isolated PMH (primary mouse hepatocyte) cells at 0.1 μM or 1 μM concentrations without the use of any additional transfection reagent. siSOD1 without ACO conjugate (RD-12556) served as a non-conjugate control, whereas DS17-04M3-AC1(me14)-L9V3 (RD-12559) served as a positive control for ODV-siRNA knockdown activity. RNA extracts were prepared 3 days after treatment and Sod1 levels were quantified via RT-qPCR. In general, knockdown of Sod1 was dose-dependent for all ODV-siSOD1 variants in which each ACO conjugate outperformed the siSOD1 only control; however, changes in ACO composition or its linker in some instances did impact free uptake activity (FIG. 37A). Cell viability was also assessed in parallel for all 90 ODV compounds via PI staining to determine if modification of ODV-siSOD1 composition impacted cytotoxicity in viro. As shown in FIG. 37B, any measurable changes in cytotoxicity were nominal all falling below a 1.5-fold increase in staining implying that all the ODV-siRNA variants, in general, are equally safe.

### Example 25. Effects of ACO Composition and Linker Choice on ODV-siRNA in PMH Cells

Group A (Linker group) included 10 compounds (i.e., RD-12941, RD-12942, RD-12943, RD-12944, RD-12945, RD-12947, RD-12948, RD-12949, RD-12950 and RD-12951). All compounds in this group except RD-12943 and RD-12944 had better knockdown activity over the exemplary ODV-siRNA control (RD-12559) suggesting ODV-siRNA can tolerate several different linker types in which certain varieties can provide greater potency (FIG. 37A, FIG. 38). For instance, RD-12948 containing the C12 linker had near maximal knockdown activity at both 0.1 μM and 1 μM treatment concentrations, whereas natural RNA linkers (e.g., RD-12943 and RD-12944) significantly compromised delivery (FIG. 38).

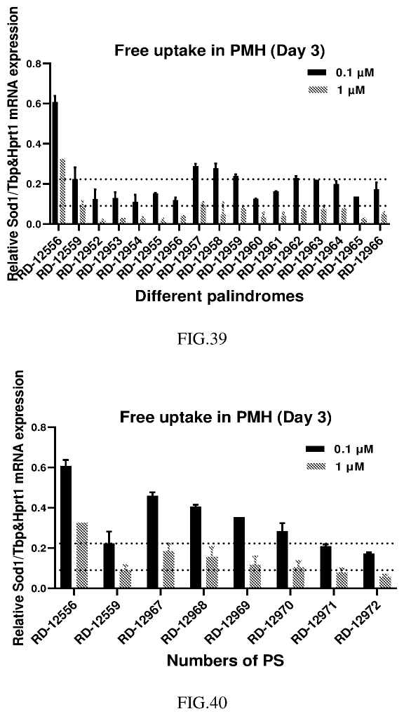

Group B (Palindromic AC1 sequence group) included 15 compounds containing sequence derivatives based on the exemplary ACO called AC1 (i.e., RD-12952, RD-12953, RD-12954, RD-12955, RD-12956, RD-12957, RD-12958, RD-12959, RD-12960, RD-12961, RD-12962, RD-12963, RD-12964, RD-12965, and RD-12966). As shown in FIG. 57, the AC1 sequence contains a perfect palindromic sequence with a 2-nt central spacer between the two palindromic wings and based on AC1, a series of palindromic ACO sequences were designed. All compounds in this group had knockdown activity equal to or better than the exemplary ODV-siRNA control (RD-12559) suggesting the palindromic feature in the ACO may provide benefits for cellular uptake and in vivo activity and can be independently optimized (FIG. 37A, FIG. 39). For instance, RD-12952, RD-12953, RD-12954, RD-12955, and RD-12956 have a varying palindromic ACO (FIG. 57) and superior activity at both contractions compared to the exemplary ODV-siRNA control (RD-12559). RD-12957 and RD-12958 with AC1 variants shorter in size at a respective 6 and 8 nucleotides in length had reduced activity compared to larger variants in this groups supporting ACO size impacts free uptake (FIG. 39).

Group C (PS modification group) included 6 compounds (i.e., RD-12967, RD-12968, RD-12969, RD-12970, RD-12971 and RD-12972) each with the same 14-nt ACO containing either 2, 4, 6, 8, 10 or 12 PS modifications, respectively. As shown in FIG. 40, increasing the number of PS modification in the ACO resulted in improved knockdown activity of the corresponding ODV-siSOD1. These results demonstrate a clear correlation in free uptake with the number of PS modifications in which delivery efficacy of ODV-siRNAs may be optimized by adjustment in PS chemistry.

Group D (2′Ome modification group) included 7 compounds (i.e., RD-12973, RD-12974, RD-12975, RD-12976, RD-12977, RD-12978 and RD-12979) each with the same 14-nt ACO containing either 2, 4, 6, 8, 10 or 12 substitutions of 2′MOE chemistry for 2′Ome, respectively. All compounds in this group had knockdown activity equal to or better than the exemplary ODV-siRNA control (RD-12559) suggesting 2′Ome modification is interchangeable for 2′MOE and may offer benefits in cell uptake when included into the ACO sequence (FIG. 37A, FIG. 41).

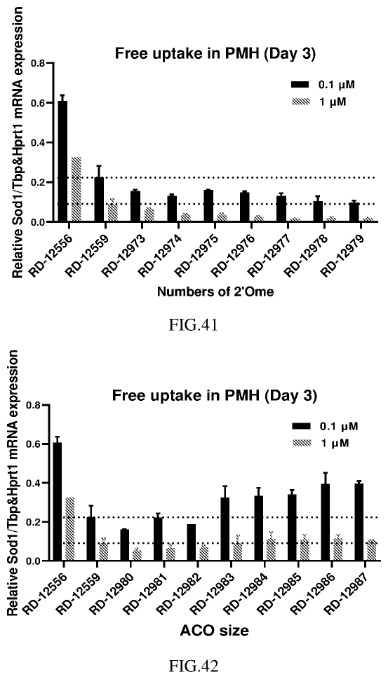

Group E (ACO size group) included 8 compounds (i.e., RD-12980, RD-12981, RD-12982, RD-12983, RD-12984, RD-12985, RD-12986 and RD-12987) derived by truncating the 14-nt ACO in RD-12559 to 13, 12, 11, 10, 9, 8, 7, or 6 nucleotides in length, respectively. As shown in FIG. 42, knockdown activity of the ODV-siRNAs was approximately equal to the exemplary ODV-siRNA control (RD-12559) at ACO lengths greater than 10 nucleotides.

Shorter ACO lengths at 10 to 6 nucleotides correlated with reduced ODV-siSOD1 knockdown activity suggesting a reduction in cellular uptake and reenforcing the importance of ACO size on delivery.

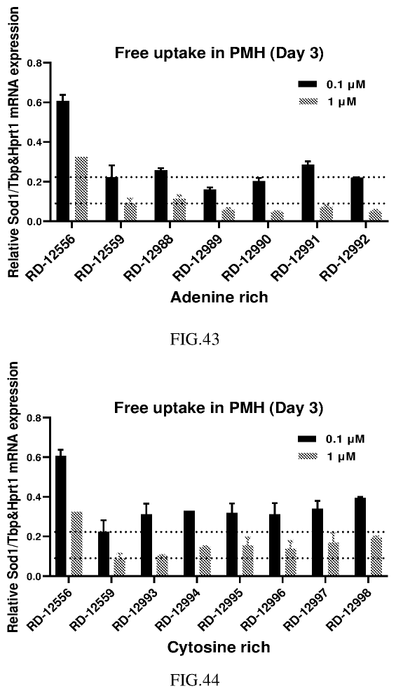

The remaining groups were designed to further investigate nucleotide composition on uptake and knockdown activity of ODV-siRNA. Group F (Adenine rich group) included 5 compounds (i.e., RD-12988, RD-12989, RD-12990, RD-12991, and RD-12992) each with a different 14-nt ACO sequence containing either 5, 6, 7, 8, or 9 total adenine nucleotides, respectively. All compounds in this group had knockdown activity similar to the exemplary ODV-siRNA control (RD-12559) suggesting ACO sequence is amendable to variable adenosine content for cellular uptake (FIG. 37A, FIG. 43).

Group G (Cytosine rich group) included 6 compounds (i.e., RD-12993, RD-12994, RD-12995, RD-12996, RD-12997, and RD-12997) each with a different 14-nt ACO sequence containing either 5, 6, 7, 8, 9, or 10 total cytosine nucleotides, respectively. As shown in FIG. 44, compounds in this group had a marginal loss in knockdown activity compared to the exemplary ODV-siRNA control (RD-12559) as cytosine content increased in the ACO sequence. This data suggests limiting cytosine content in ACO sequence may be beneficial for free uptake and knockdown activity for ODV-siRNA.

Group H (Guanine rich group) included 5 compounds (i.e., RD-12999, RD-13000, RD-13001, RD-13002, and RD-13003) each with a different 14-nt ACO sequence containing either 5, 6, 7, 8, or 9 total guanine nucleotides, respectively. All compounds in this group had knockdown activity equal to or slight better than the exemplary ODV-siRNA control (RD-12559) suggesting ACO sequence is amendable to variable guanine content for cellular uptake (FIG. 37A, FIG. 45).

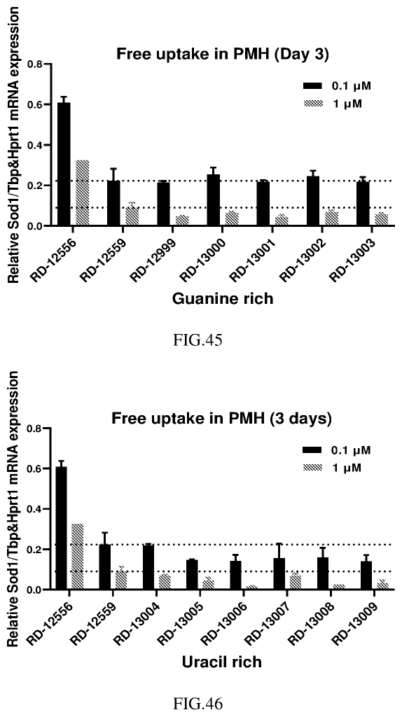

Group I (Uracil rich group) included 6 compounds (i.e., RD-13004, RD-13005, RD-13006, RD-13007, RD-13008, and RD-13009) each with a different 14-nt ACO sequence containing either 5, 6, 7, 8, 9, or 10 total uracil nucleotides, respectively. As shown in FIG. 46, knockdown activity generally improved with uracil suggesting ACO sequence is amendable to variable uracil content for cellular uptake.

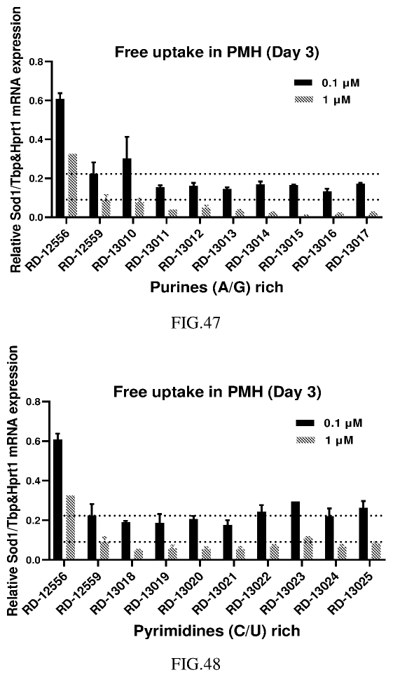

Group J (Purine rich group) included 8 compounds each with a different 14-nt ACO sequence containing 9 purines (i.e., RD-13010, RD-13011, and RD-13012), 10 purines (i.e., RD-13013, RD-13014, and RD-13015), or 11 purines (i.e., RD-13016 and RD-13017) comprised of different amounts of adenosine and guanine. In general, knockdown activity improved as purine content increased ACO sequence (FIG. 37A, FIG. 47).

Group K (Pyrimidine rich group) included 8 compounds each with a different 14-nt ACO sequence containing 9 pyrimidines (i.e., RD-13018, RD-13019, and RD-13020), 10 pyrimidines (i.e., RD-13021 and RD-13022), 11 pyrimidines (i.e., RD-13023 and RD-13024), or 12 pyrimidines (i.e., RD-13025) comprised of different amounts of cytosine and uracil. As shown in FIG. 48, compounds in this group had knockdown activity similar to the exemplary ODV-siRNA control (RD-12559) with a slight trend toward reduced activity as pyrimidine content reached 11 and 12 nucleotides in manner similar to Group G (Cytosine rich group).

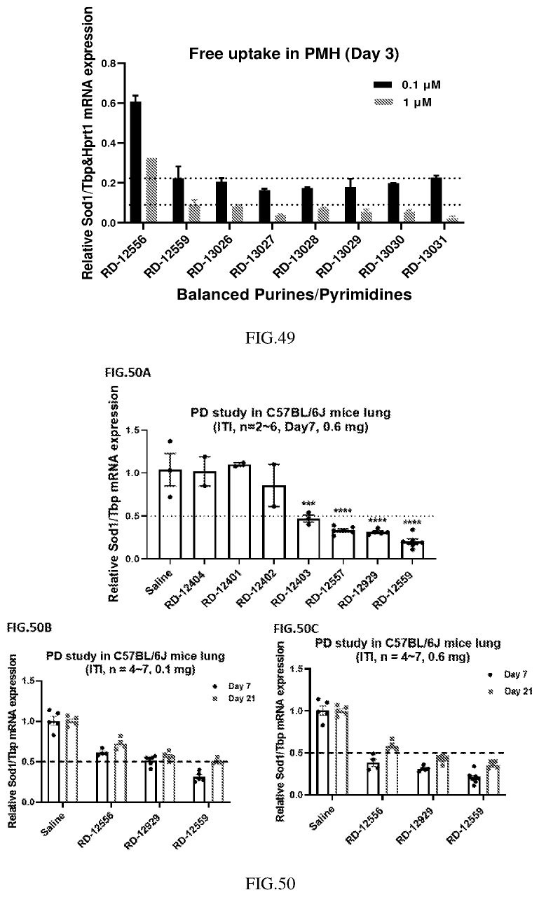

Group L (Balanced purine:pyrimidine group) included 6 compounds (i.e., RD-13026. RD-13027, RD-13028, RD-13029, RD-13030, and RD-13031) each with a different 14-nt ACO sequence containing a fixed 1:1 ratio of purines to pyrimidines, respectively. All compounds in this group had comparatively similar knockdown activity further supporting that a specific sequence is not necessary for cellular uptake when controlling for purine and pyrimidine content within ACO (FIG. 49).

Overall, these results indicate that a specific ACO sequence is not the driver of cellular uptake, but its nucleotide content can impact delivery and ODV-siRNA knockdown activity. For instance, purine rich content appears to provide better free uptake in comparison to cytosine or pyrimidine rich ACO sequence. The major influence on free uptake appears to be ACO length, chemistry (i.e., PS quantity and 2′moeity content), and linker selection within ODV-siRNA.

### Example 26. In Vivo Knockdown Activity of ODV-siSOD1 in Mouse Lung

To validate ODV-siRNA activity in the lung, adult C57BL/6J mice were treated with several ODV-siSOD1s with (i.e., RD-12557 and RD-12559) or without (i.e., RD-12403 and RD-12929) the 5′VP modification via ITI administration at a total dose of 0.1 or 0.6 mg. A non-specific ODV-siRNA without the 5′VP modification (i.e., RD-12404) served as negative control for knockdown activity. ACO-absent siRNAs with (i.e., RD-12556) or without (i.e., RD-12402) the 5′VP modification served as comparison controls for ODV chemistry. RD-12401 devoid of any medicinal chemistry and ACO conjugation served as an unmodified siSOD1 control. All test articles were formulated in saline in which treatment with saline alone served as procedural control to establish baseline expression levels of mouse Sod1. All sequences are listed in Table 29.

As shown in FIG. 50A, neither RD-12401 nor RD-12402 significantly reduced Sod1 levels compared to baseline on day 7 following treatment at 0.6 mg total dose in lung tissue. However, ACO-conjugation in absence of 5′VP modification (i.e., RD-12403 and RD-12929) measured a respective 53% and 69% reduction in Sod1 levels. Addition of 5′VP further reduced knockdown activity to 67% and 80% for cognate ODV-siRNAs RD-12557 and RD-12559, respectively. The combination of both ACO conjugate and the 5′VP appear to provide enhanced knockdown activity in lung tissue for ODV-siSOD1. In support, treatment with of RD-12559 at 0.1 or 0.6 mg had greater overall knockdown activity and durability at day 7 and 21 after treatment compared to non-ACO cognate RD-12556 or non-5′VP variant RD-12929 (FIG. 50B-C).

### Example 27. In Vivo Knockdown Activity of ODV-siSOD1 in Mouse Muscle

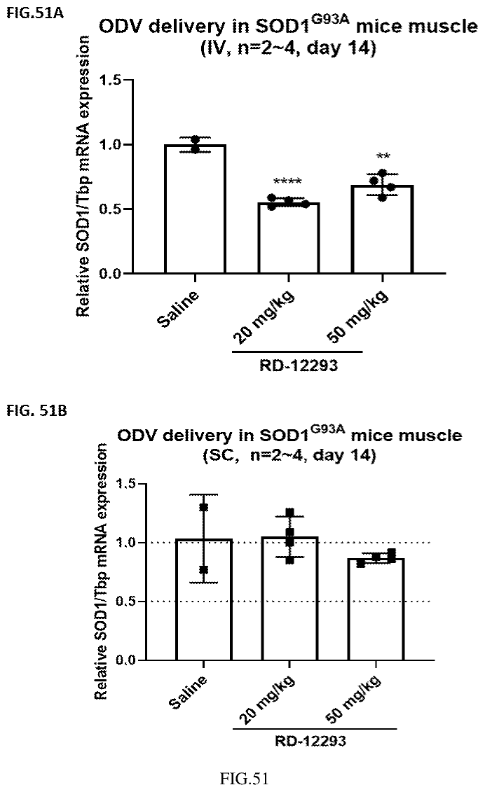

To test ODV-siRNA activity in muscle, adult SOD1^{G93A} mice at PND 46 were treated with ODV-siSOD1 (i.e., RD-12293) at 20 or 50 mg/kg via IV or SC injection. Saline served as a procedural control to establish baseline expression levels of SOD1 mRNA. Mice were sacrificed on day 14 after treatment and knockdown activity was quantified in muscle tissue via RT-qPCR. As shown in FIG. 51A, IV injection of RD-12293 provided modest knockdown of SOD1 in mouse muscle reducing mRNA levels by ˜44% and ˜31% at 20 mg/kg and 50 mg/kg, respectively. Conversely, SC injection had far less measurable activity in muscle reducing SOD1 levels by only 13% at the highest dose (FIG. 51B). These results indicate that route of administration can impact ODV-siRNA potency in which IV injection of RD-12293 had better knockdown activity in mouse muscle compared SC injection for systemic delivery.

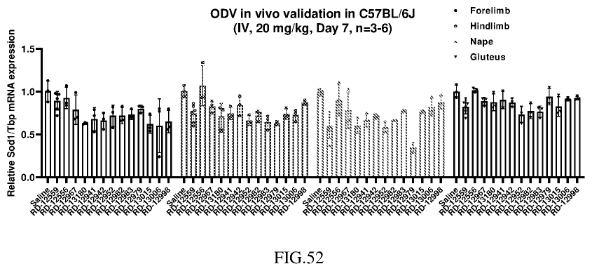

ODV-siRNA knockdown activity in muscle was further screened in adult C57BL/6J mice via IV injection using several representatives from each of the groups of ACO design variants listed in Table 27. RD-12559 served as an exemplary ODV-siRNA with the L9 linker and 5′VP modification, while RD-12556 functioned as its non-ACO cognate control. RD-13180 was updated version of RD-12559 in which 2 additional PS backbone modifications were placed directly flanking the L9 linker on either end in order to further enhance nuclease resilience (Table 30). Mice were sacrificed on day 7 following treatment and Sod) mRNA knockdown was quantified in several different muscle tissues (i.e., Forelimb, Hindlimb, Nape, and Gluteus) via RT-qPCR. As shown in FIG. 52, all compounds except the non-ACO control siRNA (i.e., RD-12556) provided variable levels of Sod1 knockdown compared to saline treatment in the different muscle tissues ranging 21-40%, 16-37%, 12-65%, and 7-27% in the forelimb, hindlimb, nape, and gluteus, respectively. Of note, RD-12979 from Group D (2′Ome modification group) with all ACO nucleotides containing 2′Ome substitutions had enrichment for knockdown activity selectively in nape muscle tissue reducing Sod) levels by 65%. In addition, the extra PS content flanking the L9 linker in RD13180 appeared to further improve knockdown in forelimb and hindlimb tissues compared to RD-12559. Overall, these results indicate that ACO conjugation can provide knockdown activity for siRNA that is otherwise not feasible. Adopting different ODV designs may also serve as a foundation for targeting specific muscle tissues (i.e., nape) or as a general method for optimizing for potency in muscle.

### Example 28. In Vivo Knockdown Activity of ODV-siSOD1 in CNS Tissue in C57BL/6J Mice

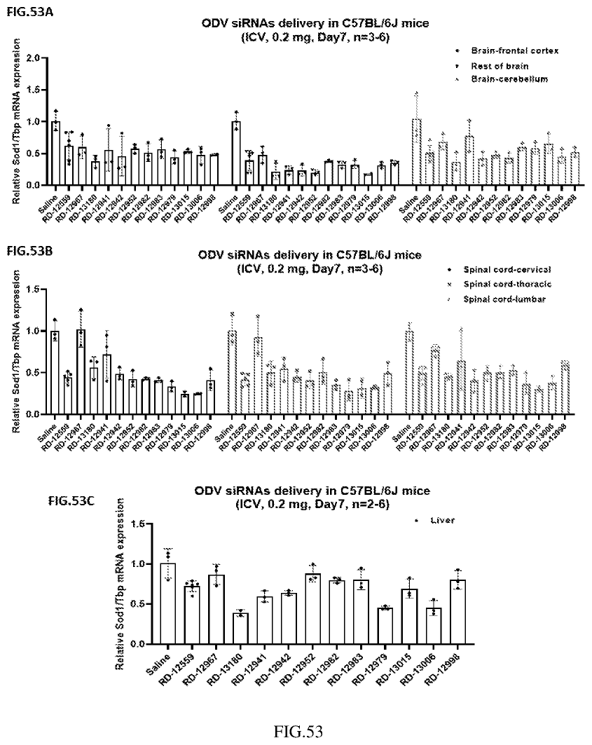

ODV-siRNA knockdown activity was also screened in CNS tissue of adult C57BL/6J mice via unilateral ICV injection with several representatives from each of the groups of ACO design variants listed in Table 27 at a 20 mg/kg total dose. RD-12559 served as an exemplary ODV-siRNA with 5′VP modification and L9 linker with a fully-modified ACO containing 2′MOE nucleotides and PS backbone. Mice were sacrificed on day 7 following treatment and Sod1 mRNA knockdown was quantified in tissues of the brain (i.e., frontal cortex, cerebellum, and rest of brain), spinal cord (i.e., cervical, thoracic, and lumbar), and periphery (i.e., liver) via RT-qPCR. As shown in FIG. 53A, all compounds provided variable levels of Sod) knockdown compared to saline treatment in the different brain tissues ranging 30-62%, 22-63%, and 52-80% in the frontal cortex, cerebellum, and rest of brain, respectively. In the spinal cord, only RD-12967 from Group C (PS modification group) containing 2 PS modification within its ACO had little-to-no activity in all spinal cord tissues suggesting PS quantity impacts tissue biodistribution in the CNS (FIG. 53B). Excluding RD-12967, knockdown activity in the spinal cord ranged between 28-75%, 45-72%, and 40-69% in cervical, thoracic, and lumbar tissue, respectively. Knockdown in the liver measured drainage to peripheral tissue and served as a marker for CNS retention. Overall, most compounds were generally well retained in the CNS reducing Sod) levels by less than 50% except RD-13180, RD12979 and RD-13006, which measured a respective 61%, 55%, and 55% reduction in the liver (FIG. 53C). Of note, RD-13180, RD12979 and RD-13006 were also generally better performers in the CNS tissues suggesting these ODV-siRNA compounds may be better suited for broad tissue biodistribution via systemic routes of administration. For local administration, retention in the treated tissue is typically more ideal for mitigating toxicities in distal organs in context to ODV-siRNA, RD-13006 of Group I (Uracil rich group) with 7 total uracil nucleotides and RD-12979 from Group D (2′Ome modification group) containing all 2′Ome substitutions within its ACO provided an apparent balance of potency in both brain and spinal tissues with substantially less activity in liver tissue (FIG. 53A-C).

### Example 29. In Vivo Knockdown Activity of ODV-siSOD1 in C57BL/6J Mice Following Systemic Administration

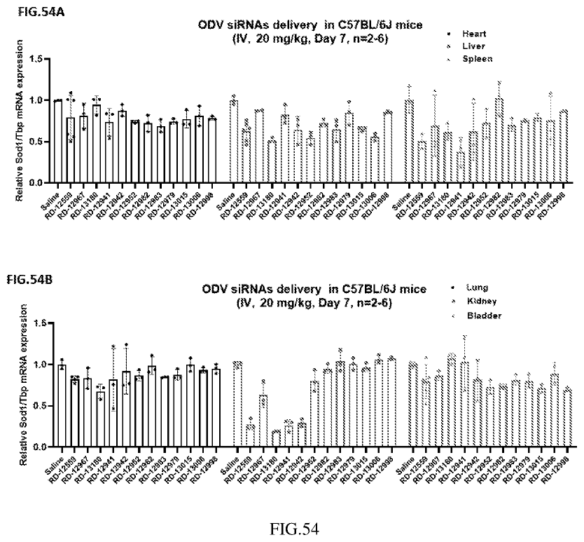

ODV-siSOD1 activity was further screened in tissue following systemic administration of several representatives from the groups of ACO design variants listed in Table 27 at 20 mg/kg via IV injection RD-12559 served as an exemplary ODV-siRNA with 5′VP modification and L9 linker with fully-modified ACO containing 2′MOE nucleotides and PS backbone. Mice were sacrificed on day 7 following treatment and Sod) mRNA knockdown was quantified in tissues form heart, liver, spleen, lung, kidney, and bladder. As shown in FIG. 54A-B, ACO composition impacted distribution of knockdown activity in the analyzed tissues. In heart, knockdown was modest in which activity did not exceed 31% reductions in Sod1 levels, which was achieved by RD-12983 from Group E (ACO size group) containing an ACO at 10-nt in length (FIG. 54A). RD-13180, RD-12952, and RD-13006 measured a respective 48%, 45%, and 44% reductions in Sod1 levels compared saline control (FIG. 54A). Both RD-13180 and RD-13006 were predicted as being ideal ODV-siRNA candidates for systemic routes of administration based on CNS drainage data following ICV injection (FIG. 53C). Knockdown in spleen ranged between 0% by RD-12982 from Group E (ACO size group) and 62% by RD-12941 from Group A (Linker group) indicating ODV-siRNA design can effectively direct targeting to the spleen by linker choice and/or ACO composition (FIG. 54A). In lung, knockdown was again modest in which activity did not exceed 32% reductions in Sod1 levels, which was achieved by RD-13180 (FIG. 54B). The kidney had the greatest overall range in knockdown activity in which RD-12559, RD-13180, RD-12941 and RD-12942 reduced Sod) levels by 72%, 81%, 74%, and 70%, respectively, whereas the other tested ODV-siSOD1 compounds did not exceed 37% knockdown (FIG. 54B). Like spleen, this data suggests ODV-siRNA design can effectively direct targeting to the kidney. In bladder, detection of activity was generally similar for all ODV-siSOD1 compounds and did not exceed was 29% reductions in Sod1 levels.

### Example 30. The Preparation of Compounds of the Present Disclosure

### Compound List

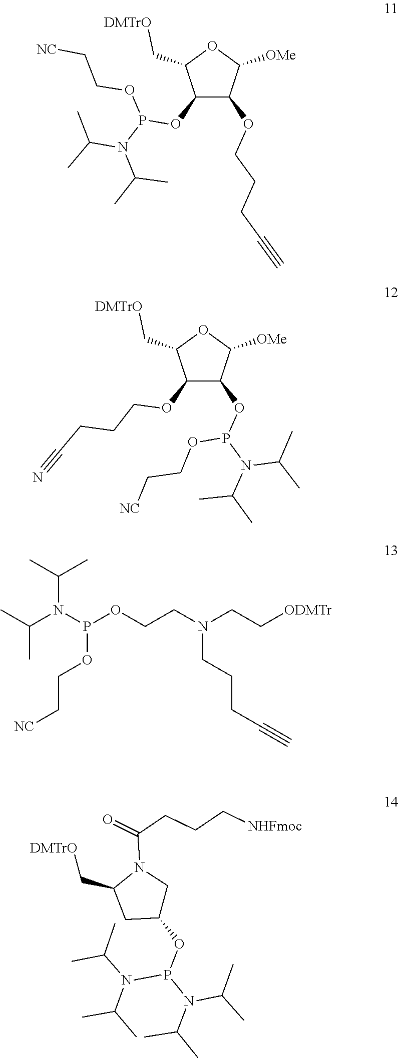

### Compounds 11 and 12 Synthesis

Compounds 11 and 12 were prepared in this Example by using the following procedures.

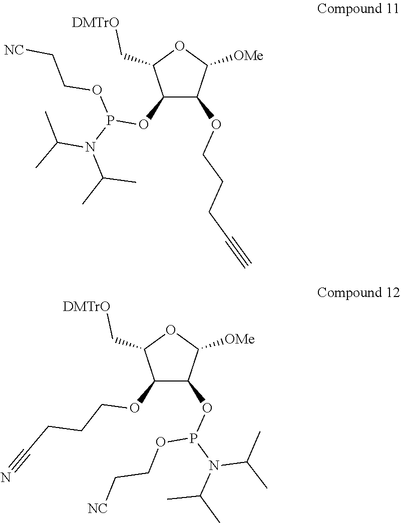

### (1) Preparation of compound 23 and 24 from the starting compound (2S,3R,4S,5S)-2-(hydroxymethyl)-5-methoxytetrahydrofuran-3,4-diol 22

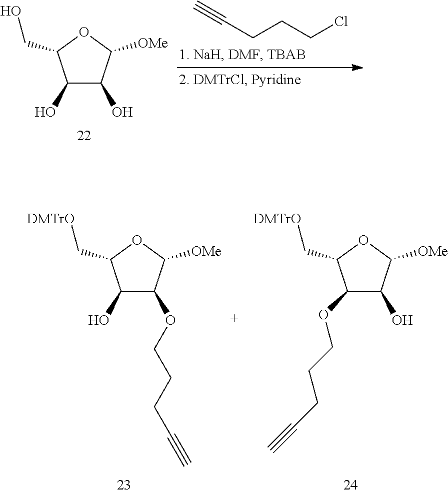

Compound 22 (4.8 g, 29 mmol, 1.0 eq) was dissolved in anhydrous DMF (200 mL) under nitrogen atmosphere. The solution was cooled to 5° C., and NaH (1.54 g, 38.6 mmol, 60% dispersion in mineral oil, 1.3 eq) was subsequently added, followed by the addition of Tetrabutylammonium bromide (TBAB) (1.87 g, 5.8 mmol, 0.2 eq) and 5-chloro-1-pentyne (3.89 mL, 36.83 mmol, 1.27 eq). The reaction mixture was allowed to stir overnight at 55° C. Then the reaction liquid was filtered and concentrated under reduced pressure. After that, water (100 mL) was added into the mixture, then extracted 3 times by ethyl acetate, the organic phase was washed 3 times by saturated lithium chloride solution. After dried by anhydrous NaSO₄ and concentrated under reduced pressure, the yellow oil formed then was directly dissolved in anhydrous pyridine (100 mL) under nitrogen atmosphere. DMTrCl (11.8 g, 34.8 mmol, 1.2 eq) was added slowly. The reaction mixture was stirred at room temperature for 6 h then concentrated under reduced pressure. The resultant residue was purified by flash chromatography (silica gel, gradient eluent: 1-30% of EtOAc/Hexane) to provide compound 23 (2.43 g, 15.7% yield) and 24 (1.49 g, 10% yield). The products were characterized with mass spectrometry and ¹H NMR.

Compound 23 MW calc: 532.25; MW Found: 555.8 [M+Na]. ¹H NMR (400 MHz, CDCl₃) δ 7.50 (dd, J=7.9, 3.8 Hz, 2H), 7.38 (dd, J=8.2, 3.4 Hz, 4H), 7.27 (d, J=6.7 Hz, 2H), 7.19-7.15 (m, 1H), 6.83-6.81 (m, 4H), 4.96-4.90 (m, 1H), 4.23 (s, 1H), 4.13-4.03 (m, 2H), 3.78 (s, 6H), 3.75 (d, J=3.3 Hz, 2H), 3.71-3.62 (m, 2H), 3.38 (s, 3H), 3.17 (t, J=5.0 Hz, 1H), 2.34-2.30 (m, 2H), 1.87-1.82 (m, 2H).

Compound 24 MW calc: 532.25; MW Found: 303.4 [DMT]⁻, 253.3 [DMT off+Na]⁺. ¹H NMR (400 MHz, CDCl₃) δ 7.52-7.46 (m, 2H), 7.40-7.34 (m, 41H), 7.29-7.26 (m, 2H), 7.21 (dd, J=8.3, 3.2 Hz, 1H), 6.83 (t, J=5.6 Hz, 4H), 4.95-4.83 (m, 1H), 4.15-4.04 (m, 3H), 3.79 (s, 6H), 3.78-3.70 (m, 2H), 3.60 (dt, J=8.5, 5.7 Hz, 1H), 3.54-3.48 (m, 1H), 3.37 (s, 3H), 3.20-3.15 (m, 1H), 2.80-2.65 (m, 1H), 2.31-2.15 (m, 2H), 1.73 (tdd, J=9.5, 6.5, 2.7 Hz, 2H).

### (2) Preparation of Compound 11 from Compound 23

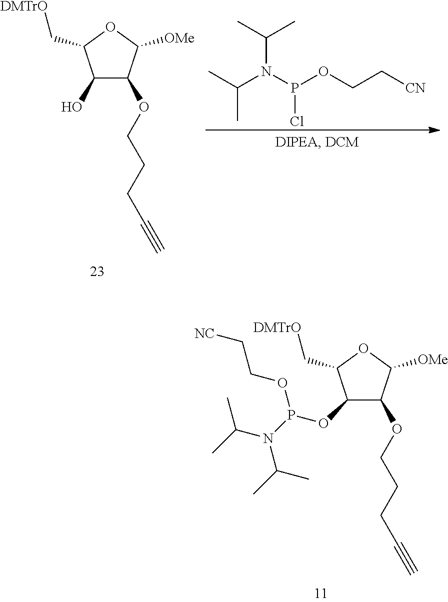

To a solution of compound 23 (300 mg, 0.56 mmol, 1.0 eq) and N,N-Diisopropylethylamine (DIPEA) (139 μL, 0.84 mmol, 1.5 eq) in anhydrous DCM (5 mL) under nitrogen atmosphere was added 3-((chloro(diisopropylamino)phosphaneyl)oxy)propanenitrile (199 mg, 0.84 mmol, 1.5 eq) at room temperature. The reaction mixture was stirred for 1 h. The mixture was extracted two times with DCM, then washed with brine and dried with anhydrous Na₂SO₄. The organic layer was concentrated under reduced pressure and the resultant residue was purified with flash chromatography (silica gel, gradient eluent: 1-20% of EtOAc/Hexane, 1% Et₃N) to provide compound 11 (196 mg, 48% yield) as colorless oil.

The product was characterized with mass spectrometry and ¹H NMR. MW calc.: 732.35; MW Found: 733.2 [M+H]⁺. ¹H NMR (400 MHz, CDCl₃) δ 7.53-7.48 (m, 2H), 7.38 (dd, J=8.6, 6.4 Hz, 4H), 7.31-7.27 (m, 2H), 7.22-7.18 (m, 1H), 6.83-6.79 (m, 4H), 4.97-4.91 (m, 1H), 4.25-4.21 (m, 1H), 3.79 (s, 6H), 3.74 (dd, J=6.0, 2.8 Hz, 2H), 3.70-3.61 (m, 2H), 3.58-3.45 (m, 4H), 3.41 (s, 3H), 3.10 (dd, J=10.1, 5.2 Hz, 1H), 2.62 (dd, J=6.5, 3.5 Hz, 1H), 2.39-2.26 (m, 4H), 1.95 (dd, J=5.4, 2.6 Hz, 1H), 1.83-1.78 (m, 2H), 1.17-0.95 (m, 12H).

### (3) Preparation of Compound 12 from Compound 24

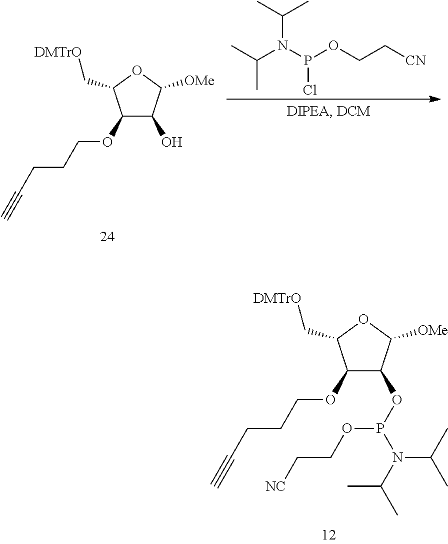

To a solution of compound 24 (300 mg, 0.56 mmol, 1.0 eq) and DIPEA (139 μL, 0.84 mmol, 1.5 eq) in anhydrous DCM (5 mL) under nitrogen atmosphere, was added 3-((chloro(diisopropylamino)phosphaneyl)oxy)propanenitrile (199 mg, 0.84 mmol, 1.5 eq) at room temperature. The reaction mixture was stirred for 1 h. The mixture was extracted two times with DCM, then washed with brine and dried with anhydrous Na₂SO₄. The organic layer was concentrated under reduced pressure and the resultant residue was purified with flash chromatography (silica gel, gradient eluent: 1-20% of EtOAc/Hexane, 1% Et₃N) to provide compound 12 (127 mg, 31% yield) as colorless oil. The product was characterized with mass spectrometry and ¹H NMR. MW calc.: 732.35; MW Found: 733.2 [M+H]⁺. ¹H NMR (400 MHz, CDCl₃) δ 7.53-7.49 (m, 2H), 7.38 (dd, J=6.3, 2.6 Hz, 4H), 7.29 (d, J=7.2 Hz, 2H), 7.22-7.19 (m, 1H), 6.85-6.81 (m, 41H), 5.02 (s, 1H), 4.20 (d, J=8.2 Hz, 1H), 4.17-3.99 (m, 2H), 3.91-3.85 (m, 1H), 3.78 (s, 6H), 3.76-3.69 (m, 2H), 3.60 (ddd, J=11.7, 8.0, 5.0 Hz, 3H), 3.41 (s, 3H), 3.12-3.07 (m, 1H), 2.64 (t, J 6.4 Hz, 2H), 2.38-2.29 (m, 1H), 2.23-2.01 (m, 2H), 1.87 (t, J=2.7 Hz, 1H), 1.68 (td, J=13.6, 6.5 Hz, 2H), 1.25-1.14 (m, 12H).

### Compound 13 Synthesis

Compound 13 was prepared in this Example by using the following procedures.

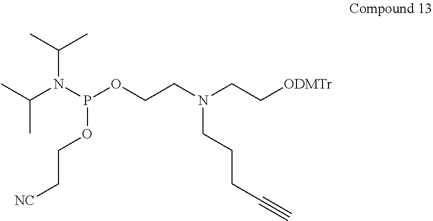

(1) Preparation of Compound 26 from the Starting Compound Diethanolamine 25.

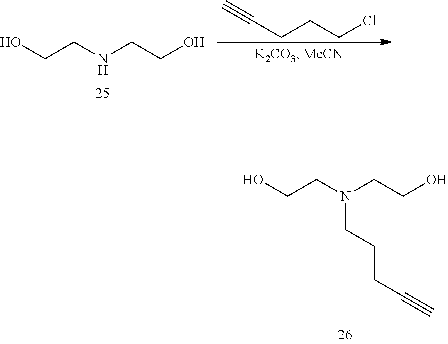

To a solution of diethanolamine 25 (7.16 g, 68 mmol, 1.0 eq) in 110 mL of MeCN under ice bath was added anhydrous potassium carbonate (47.1 g, 341 mmol, 5.0 eq) with vigorous stirring. After 30 minutes of stirring, dropwise addition of 5-chloro-1-pentyne (7.2 mL, 68 mmol, 1.0 eq) was carried out over 5 minutes. The reaction was then left to stir for 3 days at 60° C. Then the reaction liquid was filtered and concentrated under reduced pressure, the resultant residue was purified by flash chromatography (silica gel, gradient eluent. 1-8% of MeOH/DCM) to provide compound 26 (2.85 g, 24% yield). The product was characterized with mass spectrometry and ¹H NMR. MW calc.: 171.13; MW Found: 172.3 [M+H]⁺. ¹H NMR (400 MHz, CDCl₃) δ 3.64-3.55 (m, 4H), 3.13 (s, 11H), 2.67-2.52 (m, 61H), 2.24 (td, J=6.9, 2.6 Hz, 2H), 1.73-1.61 (m, 2H).

(2) Preparation of Compound 27 from the Compound 26.

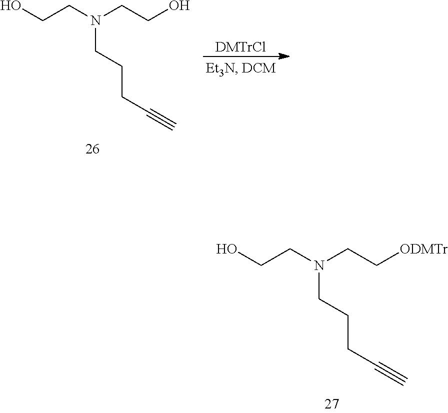

To a solution of compound 26 (2.8 g, 16.35 mmol, 1.0 eq) in 30 ml of DCM was added Et₃N (2.27 mL, 16.35 mmol, 1.0 eq) with stirring. Then the DMTrCl (4.43 g, 13.08 mmol, 0.8 eq) was added slowly. The reaction mixture was stirred at room temperature for 6 h then concentrated under reduced pressure. The residue was purified by flash chromatography (silica gel, gradient eluent: 1-3% of MeOH/DCM) to provide compound 27 (2.98 g, 48% yield). The product was characterized with mass spectrometry and ¹H NMR. MW calc.: 473.26; MW Found: 474.2 [M+H]⁺. ¹H NMR (400 MHz, CDCl₃) δ 7.44 (d, J=7.5 Hz, 2H), 7.32 (t, J=5.9 Hz, 4H), 7.29-7.24 (m, 2H), 7.20 (t, J=7.3 Hz, 1H), 6.83 (t, J=5.8 Hz, 4H), 3.78 (s, 6H), 3.53 (t, J=5.2 Hz, 2H), 3.18 (t, J=5.8 Hz, 2H), 2.69 (t, J=5.8 Hz, 2H), 2.63-2.53 (m, 4H), 2.17 (td, J=7.0, 2.6 Hz, 2H), 1.90 (t, J=2.6 Hz, 1H), 1.65 (p, J=7.0 Hz, 2H).

(3) Preparation of Compound 13 from the Compound 27.

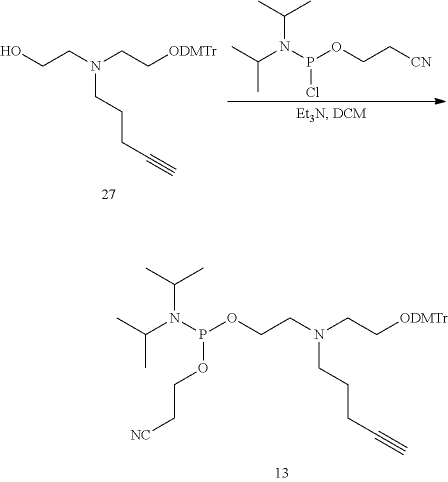

To a solution of compound 27 (1.23 g, 2.6 mmol, 1.0 eq) and Et₃N (1.81 mL, 13 mmol, 5.0 eq) in anhydrous DCM (20 mL) under nitrogen atmosphere, was added 3-((chloro(diisopropylamino)phosphaneyl)oxy)propanenitrile (1.85 g, 7.8 mmol, 3.0 eq) at room temperature. The reaction mixture was stirred for 30 min. The mixture was extracted two times with DCM, then washed with brine and dried with anhydrous Na₂SO₄. The organic layer was concentrated under reduced pressure and the resultant residue was purified with flash chromatography (silica gel, gradient eluent 1-30% of EtOAc/Hexane, 1% Et₃N) to provide compound 13 (1.12 g, 64% yield) as yellow oil. The product was characterized with mass spectrometry and ¹H NMR. MW calc.: 673.36; MW Found: 633.2 [one isopropyl off+H]⁻. ¹H NMR (400 MHz, CDCl₃) δ7.46-7.42 (m, 2H), 7.35-7.30 (m, 4H), 7.29-7.23 (m, 2H), 7.22-7.16 (m, 1H), 6.84-6.77 (m, 4H), 3.79 (d, J=5.1 Hz, 1H), 3.78 (s, 6H), 3.62 (qdd, J=17.0, 9.1, 4.9 Hz, 4H), 3.13 (t, J=6.4 Hz, 2H), 2.75-2.70 (m, 3H), 2.57 (td, J=6.7, 1.6 Hz, 4H), 2.18 (td, J=7.1, 2.6 Hz, 2H), 2.04 (s, 1H), 1.87 (t, J=2.6 Hz, 1H), 1.62 (p, J=7.1 Hz, 2H), 1.25 (t, J=7.1 Hz, 1H), 1.16 (dd, J=11.4, 6.8 Hz, 12H).

### Compounds 14 Synthesis

Compound 14 was prepared in this Example by using the following procedures.

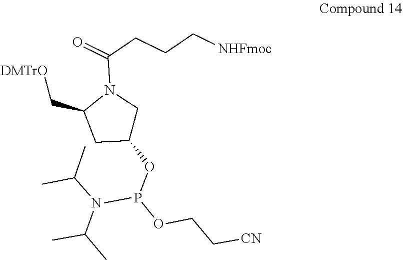

(1) Preparation of Compound 29 from the Starting Compound Fmoc-L-Hydroxyproline 28.

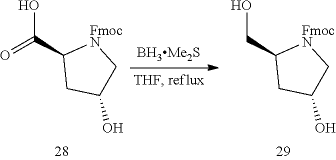

To a solution of Fmoc-L-hydroxyproline 28 (13.3 g, 37.6 mmol, 1.0 eq) in anhydrous THF (250 mL), was added borane-methyl sulfide complex (8.0 mL of 10 M in THF, 80 mmol, 2.1 eq) slowly at room temperature. The reaction mixture was stirred for 5 min at room temperature and then heated to reflux for about 1 h. Methanol (15 mL) was carefully added to the reaction mixture, which was refluxed for 15 min. After that, the reaction mixture was concentrated under reduced pressure. Then the crude products were evaporated three times with methanol (100 mL each). The crude product 29 was directly used in the next step without further purification.

(2) Preparation of Compound 30 from the Compound 29.

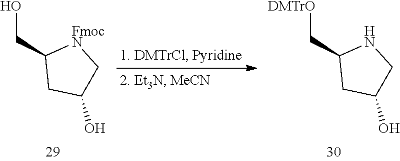

To a solution of compound 29 (37.6 mmol, 1.0 eq) in anhydrous pyridine (200 mL), was added DMTrCl (14 g, 41.4 mmol, 1.1 eq) slowly at ice bath. The reaction was stirred under nitrogen atmosphere overnight and then concentrated under reduced pressure. The crude product was dissolved in dry MeCN (300 mL) then the mixture was added Et₃N (15.6 mL, 113 mmol, 3.0 eq) and heated to 60° C. for 4 h. After concentrated under reduced pressure, the resulting residue was purified by flash chromatography (silica gel, gradient eluent: 1-8% of MeOH/DCM) to provide the desired product 30 (7.57 g, 48% yield) as yellow solid. The product was characterized with mass spectrometry and ¹H NMR. MW calc.: 419.21; MW Found: 303.2 [DMT]⁻. ¹H NMR (400 MHz, CDCl₃) δ7.41 (d, J=7.4 Hz, 2H), 7.30 (d, J=8.8 Hz, 4H), 7.28-7.22 (m, 2H), 7.18 (t, J=7.2 Hz, 11H), 6.80 (d, J=8.8 Hz, 4H), 4.34 (s, 1H), 3.75 (d, J=11.1 Hz, 6H), 3.60 (dd, J=12.7, 6.7 Hz, 1H), 3.10-2.92 (m, 5H), 2.86 (d, J=11.5 Hz, 1H), 1.85 (dd, J=13.5, 7.1 Hz, 1H), 1.63 (ddd, J=13.7, 7.9, 5.9 Hz, 1H).

(3) Preparation of Compound 31 from the Compound 30.

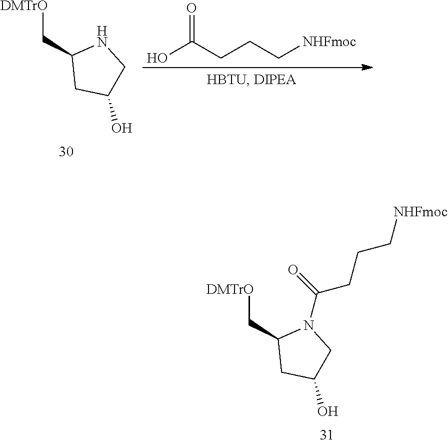

Compound 30 (500 mg, 1.19 mmol, 1.0 eq) was dissolved in 5 mL DCM, then 4-((((9H-fluoren-9-yl)methoxy)carbonyl)amino)butanoic acid (465 mg, 1.43 mmol, 1.2 eq), HBTU (903 mg, 2.38 mmol, 2.0 eq) and DIPEA (671 uL, 4.05 mmol, 3.4 eq) were added into the reaction under nitrogen atmosphere. The reaction mixture was stirred overnight at room temperature. Then 10 mL H₂O was added into the reaction, the mixture was extracted by DCM (3*10 mL) and the organic phase was combined, dried over Na₂SO₄, and concentrated. The resulting residue was purified by flash chromatography (silica gel, gradient eluent: 1-5% of MeOH/DCM) to provide the desired product 31 (740 mg, 85% yield) as yellow solid. The product was characterized with mass spectrometry and ¹H NMR. MW calc.: 726.33; MW Found: 425.2 [DMT off+H]⁺. ¹H NMR (400 MHz, CDCl₃) δ 7.74 (d, J=7.5 Hz, 2H), 7.58 (d, J=7.4 Hz, 2H), 7.38 (t, J=7.4 Hz, 2H), 7.32-7.23 (m, 7H), 7.24-7.10 (m, 4H), 6.88-6.72 (m, 4H), 4.52-4.25 (m, 4H), 4.18 (t, J=6.8 Hz, 1H), 3.78 (s, 6H), 3.52-3.38 (m, 3H), 3.27-3.10 (m, 3H), 2.42-2.19 (m, 2H), 2.04 (dd, J=13.6, 7.5 Hz, 1H), 1.84 (d, J=6.5 Hz, 1H), 1.69 (ddd, J=13.6, 9.1, 4.5 Hz, 1H), 1.48-1.27 (m, 2H).

(4) Preparation of Compound 14 from the Compound 31.

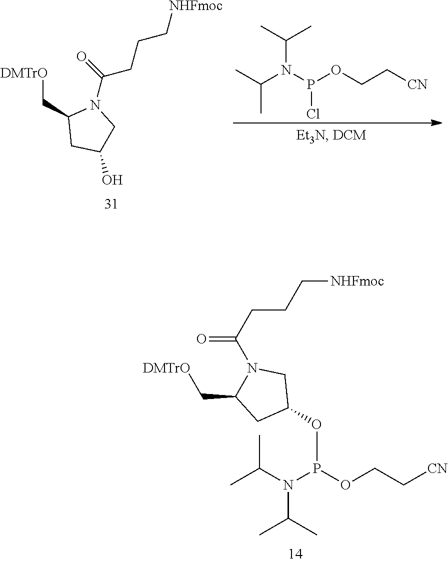

To a solution of compound 31 (400 mg, 0.55 mmol, 1.0 eq) and Et₃N (382 μL, 2.75 mmol, 5.0 eq) in anhydrous DCM (5 mL) under nitrogen atmosphere, was added 3-((chloro(diisopropylamino)phosphaneyl)oxy)propanenitrile (390 mg, 1.65 mmol, 3.0 eq) at room temperature. The reaction mixture was stirred for 1 h. The mixture was extracted two times with DCM, then washed with brine and dried with anhydrous Na₂SO₄. The organic layer was concentrated under reduced pressure and the resultant residue was purified with flash chromatography (silica gel, gradient eluent: 1-3% of MeOH/DCM, 1% Et₃N) to provide compound 14 (420 mg, 82% yield) as yellow oil. The product was characterized with mass spectrometry and ¹H NMR MW calc.: 926.44, MW Found 403.3 [DMT and Fmoc off+H]⁺. ¹H NMR (400 MHz, CDCl₃) δ 7.75 (d, J=7.5 Hz, 2H), 7.56 (d, J=7.4 Hz, 2H), 7.36 (t, J=7.4 Hz, 2H), 7.31-7.22 (m, 7H), 7.25-7.12 (m, 4H), 6.88-6.70 (m, 4H), 4.53-4.27 (m, 4H), 4.17 (t, J=6.8 Hz, 1H), 3.78 (s, 6H), 3.51-3.39 (m, 3H), 3.26-3.11 (m, 3H), 3.05 (t, J=6.4 Hz, 2H), 2.57 (td, J=6.7, 1.6 Hz, 4H), 2.41-2.18 (m, 2H), 2.05 (dd, J=13.6, 7.5 Hz, 1H), 1.85 (d, J=6.5 Hz, 1H), 1.67 (ddd, J=13.6, 9.1, 4.5 Hz, 1H), 1.47-1.28 (m, 2H), 1.16 (dd, J=11.4, 6.8 Hz, 12H).

All compounds in this example are listed in Table 1.

### General Synthesis Method of Oligonucleotide.

### Single Strand Synthesis Method

The single strand oligonucleotide was synthesized on a K&A DNA synthesizer (K&A Laborgeraete GbR, chaafheim, Germany) by a solid phase synthesis technique.

The starting material was universal solid support or special solid support commercially available or synthesis as disclosure in previous context. In general, phosphoramidite monomers including various linkers and conjugates (0.1M in acetonitrile or dichloromethane), were added sequentially onto a solid support in the DNA synthesizer to generate the desired full-length oligonucleotides. Each cycle of amidite addition consisted of four chemical reactions including detritylation, coupling, oxidation/thiolation and capping. In first step, the detritylation was performed by using 3% dichloroacetic acid (TCA) in DCM for 45 seconds. In the second step, Phosphoramidite coupling was conducted for 6 minutes for all amidites by 12 eq.; In the third step, oxidation was performed by using 0.02 M iodine in THF:pyridine:water (70:20:10, v/v/v) for 1 minute; if phosphorothioate modification needed then replace oxidation by thiolation which was carried out with 0.1 M solution of xanthane hydride in pyridine:ACN (50:50, v/v) for 3 minutes; In the fourth step, the capping was performed by using a THF acetic anhydride Pyridine (80:10:10, v/v/v) (CAP A) and N-methylimidazole:THF (10:90, v/v), (CAP B) for 20 seconds. The Cycles of four chemical reaction will be depended by the length of single of oligonucleotide.

Deprotection 1 (Nucleobase Deprotection): After completion of the synthesis, the solid support was transferred to a screw-cap microcentrifuge tube. For a 1 μmol synthesis scale, 1 ml of a mixture of methylamine and ammonium hydroxide was added. The tube containing the solid support was then heated in an oven at 60° C. to 65° C. for 15 min and then allowed to cool to room temperature. The cleavage solution was collected and evaporated to dryness in a speedvac to provide crude single strand of oligonucleotide.

Deprotection II (Removal of 2′-TBDMS Group): If The crude RNA oligonucleotide, still carrying the 2′-TBDMS groups, then dissolved in 0.1 ml of DMSO. After adding 1 ml of Triethylamine trihydrofluoride, the tube was capped, and the mixture was shaken vigorously to ensure complete dissolution and then heated in an oven at 65° C. for 15 minutes. The tube was removed from the oven and cooled down to room temperature. The solution containing the completely desilylated oligonucleotide was cooled on dry ice. 2 ml of ice-cold n-butanol (−20° C.) were carefully added in 0.5 ml portions to precipitate the oligonucleotides. The precipitate was filtered, washed with 1 mL ice-cold n-butanol, and subsequently dissolved in 0.01 M Tris(hydroxymethyl)aminomethanol hydrochloride buffer.

### Single Strand Purification

The purification of oligonucleotides was performed on an AKTA explorer 10 equipped with a Source 15Q 4.6/100 PE column using the following conditions: buffer A: (10 mM Tris-HCl, 1 mM EDTA, pH 7.5), B: (10 mM Tris-HCl, 1 mM EDTA, 2M NaCl, pH 7.5), gradient: 10% B to 60% B in 25 min, flow rate: 1 mL/min. The pure oligonucleotides were collected and desalting by a HiPrep 26/10 Desalting column.

### Annealing to Form Duplex

For duplex, after the generation of desalted purified single strand solutions, Passenger strand and Guide strand were mixed by equal volumes at equimolar concentration in the tube Place the tube in a heatblock at 95° C. for 5 min and then cool to room temperature then were subsequently lyophilized to powder.

All oligo synthesis were manufactured following General synthesis method of oligonucleotide.

### General Conjugate Reaction and Click Chemistry

Single strand of SS1, SS3, SS6, SS7, SS8 were synthesized by following single strand synthesis method. After purification, they were conjugated with terminal alkyne or azide to from click chemistry. All single strands in this example are listed in Table 31.

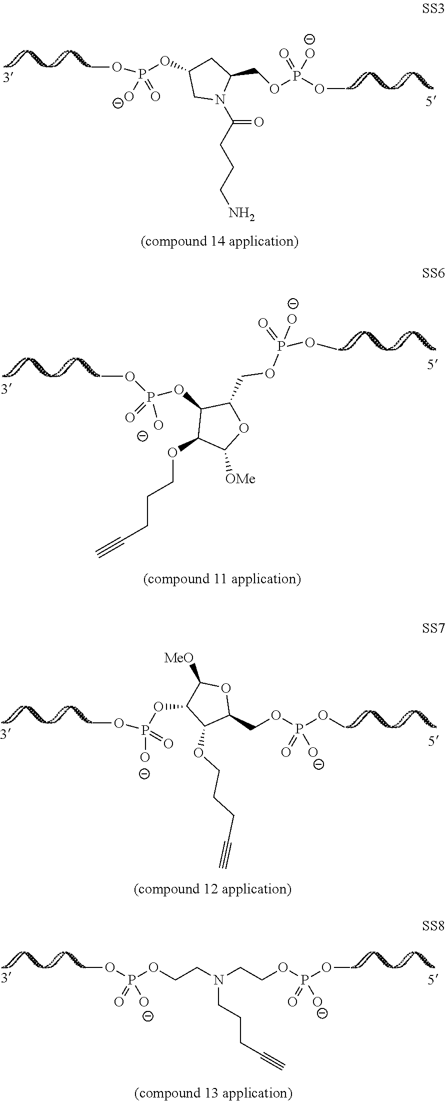

### Example 31. Preparation of RD-13351 by Click Chemistry

### (1) Preparation of Azido Conjugate Single Strand SS2

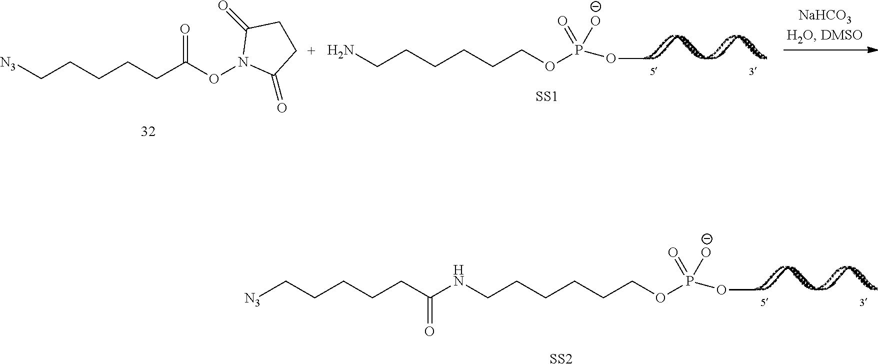

To a solution of 6-azidohexanoic acid (100 mg, 0.64 mmol, 1.0 eq) and EDCI (245 mg, 1.28 mmol, 2.0 eq) in anhydrous DCM (5 mL) under nitrogen atmosphere, was added 1-hydroxypyrrolidine-2,5-dione (147 mg, 1.28 mmol, 2.0 eq) at room temperature. The reaction mixture was stirred overnight and extracted two times with DCM, then washed with brine and dried with anhydrous Na₂SO₄. The organic layer was concentrated under reduced pressure to provide the crude compound 32 used directly in next step without purification.

The single strand of SS1 (3.68 mg, 0.65 umol, 1.0 eq) was dispersed in 800 μL dd water. Then 89 μL 1M NaHCO₃ solution and a stock solution of 32 (123 μL of 28 mg/mL DMSO stock solution, 21 eq) were added. Two hours later, 5 M NaCl (10% by volume, 101 μL) and cold ethanol (2.2 mL) were added to this reaction, then this mixture was stored in −20° C. refrigerator for 1 h to precipitate the RNA. Then the RNA precipitate was diluted to 1 mL dd water and subjected to Millipore Amicon Ultra-15 Centrifugal Filter Units (3000-MW cutoff). The ultrafiltrate was concentrated under reduced pressure and then purified by HPLC to afford azido conjugate single strand of SS2 (0.27 umol, 41% yield). The product was characterized with mass spectrometry MW calc. 5827; MW Found: 5828.7

### (2) Preparation of DBCO Conjugate Single Strand SS4

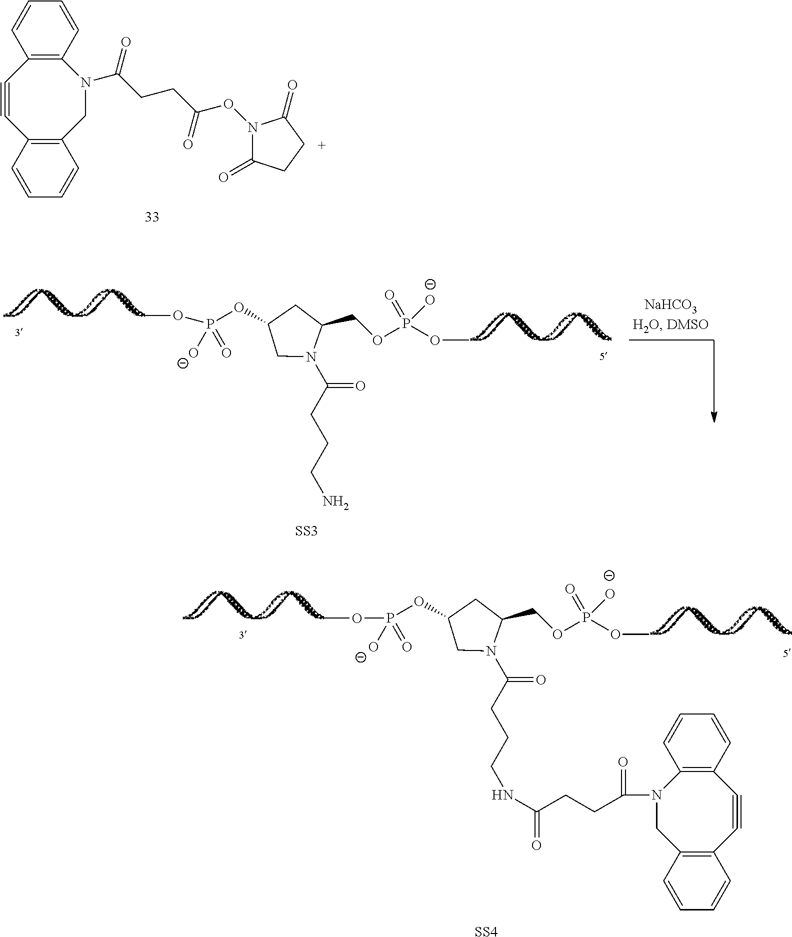

To a solution of 11,12-Didehydro-gamma-oxodibenzo[b,f]azocine-5(6H)-butanoic (DBCO) Acid (150 mg, 0.49 mmol, 1.0 eq) and EDCI (188 mg, 0.98 mmol, 2.0 eq) in anhydrous DCM (5 mL) under nitrogen atmosphere, was added 1-hydroxypyrrolidine-2,5-dione (113 mg, 0.98 mmol, 2.0 eq) at room temperature. The reaction mixture was stirred overnight and extracted two times with DCM, then washed with brine and dried with anhydrous Na₂SO₄. The organic layer was concentrated under reduced pressure to provide the DBCO derivative 33.

The single strand SS3 (18 mg, 2.7 μmol, 1.0 eq) was dispersed in 2700 μL dd water. Then 300 μL 1M NaHCO₃ solution and a stock solution of compound 33 (530 μL of 43 mg/nL DMSO stock solution, 21 eq) were added. Two hours later, 5 M NaCl (10% by volume, 353 μL) and cold ethanol (7.8 mL) were added to this reaction, then this mixture was stored in −20° C. refrigerator for 1 h to precipitate the RNA. Then the RNA precipitate was diluted to 2 mL dd water and subjected to Millipore Amicon Ultra-15 Centrifugal Filter Units (3000-MW cutoff). The ultrafiltrate was concentrated under reduced pressure and then purified by HPLC to afford DBCO conjugate single stand SS4 (0.28 μmol, 10% yield). The product was characterized with mass spectrometry. MW calc.: 6968, MW Found: 6970.1.

### (1) Preparation of Passenger Strand SS5 by Click Chemistry

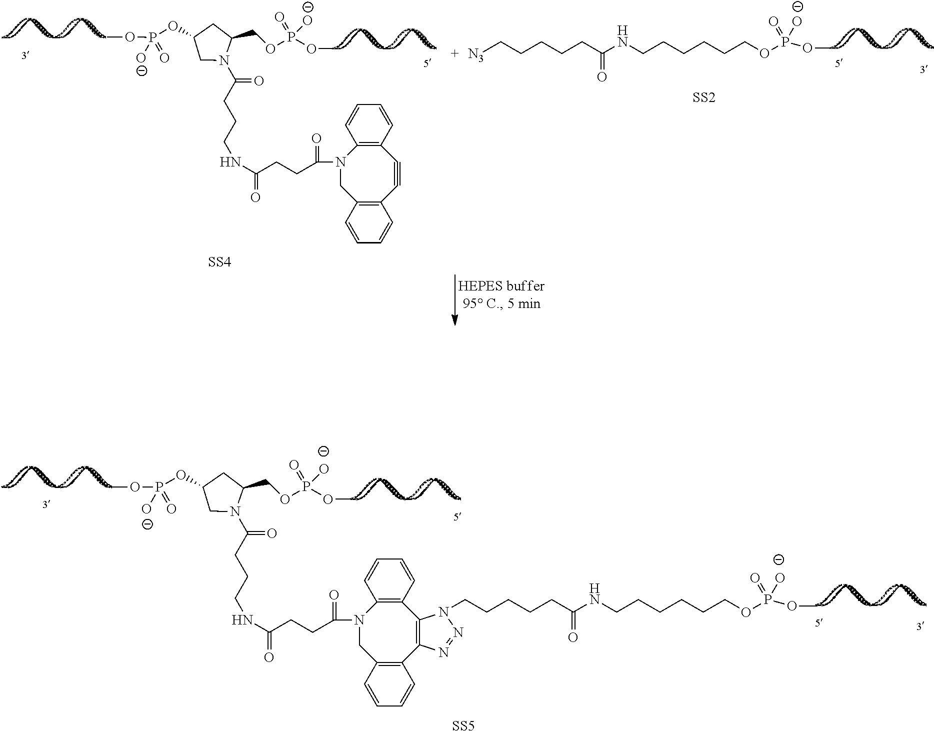

DBCO conjugate SS4 (35 nmol) and azido conjugate SS2 (14 nmol) were mixed in a NaCl solution (0.2 M, 221 μL) containing EDTA (0.5 M, 2.8 μL) and HEPES (0.1 M, pH 7.5, 56 μL) and were heated for 5 min to 95° C. then cooled to room temperature by 1° C./min and left for 2 hi at room temperature. The mixture was subjected to Millipore Amicon Ultra-15 Centrifugal Filter Units (3000-MW cutoff). The ultrafiltrate was concentrated under reduced pressure and then purified by HPLC to form SS5 (8.4 μmol, 60% yield). The product was characterized with mass spectrometry. MW calc.: 12795; MW Found: 12797.3 Passenger strand SS5 was annealing with guide (antisense) strand to form a duplex RD-13351. The single strands in this example are listed in Table 31. The linkers in Example are listed in Table 1.

TABLE 31
Sequences used in Example 31

| Name of | | |
|---|---|---|
| single | | |
| strand and | | |
| duplex | SEQ ID NO | Sequences (5′-3′) |
| SS1 | 1299 | meU*meU*meG*meU*meA*meU*meU*meC*meU*meA*meU*meG*meU* |
| | | meU |
| SS3 | 1380 | mA*fG*mGfUmG-C6x7-mAfAfAfUfGfAmAfGmAfAmAfG*mU*fA |
| SS6 | 1291 | mA*fG*mGfUmG-C6x1-mAfAfAfUfGfAmAfGmAfAmAfG*mU*fA |
| SS7 | 1292 | mA*fG*mGfUmG-C6x2-mAfAfAfUfGfAmAfGmAfAmAfG*mU*fA |
| SS8 | 1293 | mA*fG*mGfUmG-C6x5-mAfAfAfUfGfAmAfGmAfAmAfG*mU*fA |
| RD-13351 | 1294 | mA*fG*mGfUmG-(C6x7- |
| | | meU*meU*meG*meU*meA*meU*meU*meC*meU*meA*meU*meG*meU* |
| | | meU)-mAfAfAfUfGfAmAfGmAfAmAfG*mU*fA |
| | 57 | VPmU*fA*mCfUmUfUmCfUmUfCmAfUmUfUmCfCmAfCmCfU*mU*fU |

n.b.: upper case, RNA; *, phosphorothioate (PS) backbone modification; me, 2′-O-methoxyethyl (2′MOE); m, 2′-O-methyl (2′-OMe); me, 2′-O-methoxyethyl (2′MOE); f, 2′-fluoro; VP, 5′-(E)-vinylphosphonate, C, 5′-methyl cytosine; U, 5′-methyl uracil; C6x1, C6x1 linker; C6x2, C6x2 linker; C6x5, C6x5 linker; C6x7, C6x7 linker.

### Example 32. Assessment of Knockdown Activity and Cytotoxicity of ODV-siSOD1 with Internally Conjugated ACOs in PMH Cells

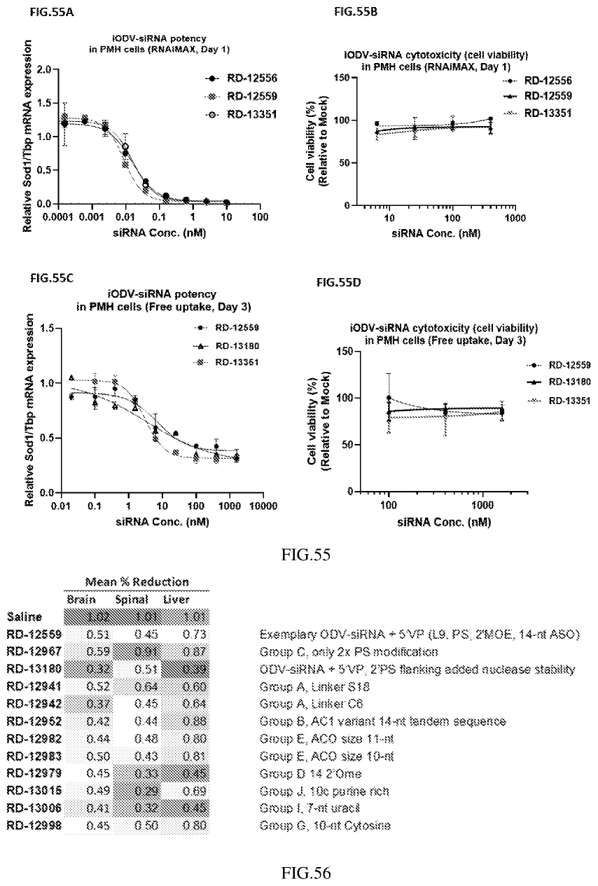

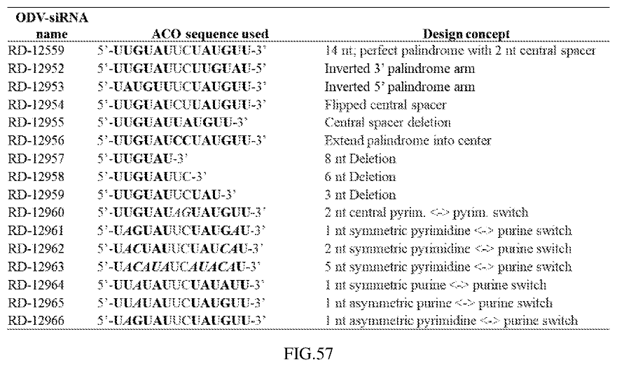

ODV-siSOD1 compounds were synthesized with ACO sequence conjugated via C6x7 linker at nucleotide position 6 internal to the passenger strand (see Table 31). To test the impact internal ASO conjugation had on siRNA activity, internal conjugate ODV-siRNA (iODV-siRNA) RD-13351 was compared to parental siRNA without ACO conjugate (i.e., RD-12556) and conventional ODV-siRNA variant with its 3′-terminal ACO (i.e., RD-12559). ODV-siRNA sequence composition is also listed in Table 30. Sod1 levels were assessed via RT-qPCR to quantify knockdown activity at escalating concentrations (i.e., 0.000005, 0.00002, 0.00006, 0.0024, 0.0098, 0.039, 0.156, 0.625, 2.5 and 10 nM) in PMH cells 24 hours after transfection using Lipofectamine RNAiMax. Plotting knockdown activity generated dose response curves in which IC₅₀ values were extrapolated to define potency for each of the tested siRNAs (FIG. 55A). As summarized in Table 32, neither potency nor activity were impaired by internal conjugation of the ACO. Cell viability via CCK8 assay was also assessed at escalating doses (i.e., 6.25, 25, 100, and 400 nM) as a marker for cytotoxicity. As shown in FIG. 55B, no significant changes in cell viability were detected up to 400 nM indicating internal conjugation of ACO did not alter in vitro cytotoxicity.

TABLE 32
IC₅₀ and Emax values of ODV-siRNA in PMH cells

| siRNAs | IC₅₀ (nM) | Emax knockdown activity (%) |
|---|---|---|
| RD-12556 | 0.01572 | 97 |
| RD-12559 | 0.00891 | 98 |
| RD-13351 | 0.01616 | 98 |

Free uptake of iODV-siRNA (i.e., RD-13351) was subsequent tested in comparison to conventional ODV-siRNAs (i.e., RD-12559 and RD-13180) at escalating concentrations (i.e., 0.02, 0.1, 0.39, 1.56, 6.25, 25, 100, 400, and 1600 nM) in PMH cells. Sod1 levels were assessed via RT-qPCR to quantify knockdown activity 3 days after treatment in which data was plotted to generated dose response curves to extrapolate IC₅₀ values (FIG. 55C). As shown in Table 33, both potency and activity were similar for iODV-siRNA compared to both conventional ODV-siRNA compounds. Cell viability was also assessed at three doses (i.e., 100, 400, and 1600 nM) to determine cytotoxicity as consequence of free uptake. As shown in FIG. 55D, no differences in cell viability were detected up to 1600 nM supporting that the iODV-siRNA structure does not alter cytotoxicity in comparison to 3′-terminal ACO conjugation. Taken together, these results indicate that internal conjugation of ACO sequence is well-tolerated for ODV-siRNA.

TABLE 33
IC₅₀ and Emax values of following free-uptake
of ODV-siRNAs in PMH cells

| siRNAs | IC₅₀ (nM) | Emax knockdown activity (%) |
|---|---|---|
| RD-12559 | 6.977 | 68 |
| RD-13180 | 3.382 | 65 |
| RD-13351 | 3.086 | 69 |

TABLE 27
List of novel ODV designs

| | | Name of | | | | | | Knockdown | |
|---|---|---|---|---|---|---|---|---|---|
| | | ODV | | | | | | activity | |
| | | compound | | | SEQ | | | (%) of ODV | |
| Design | | that uses | | | ID | | Size | compound | |
| group | Variable | the design | Design | Linker | NO | ACO sequence (5′-3′) used in ODV compound | (bp) | 0.1 μM | 1 μM |
| Control | n/a | RD-12556 | Duplex control | n/a | / | n/a | n/a | 39.25 | 67.50 |
| | n/a | RD-12559 | ODV positive | S9 | 1299 | meU*meU*meG*meU*meA*meU*meU*meC*meU*meA*meU*meG*meU | 14 | 77.70 | 91.00 |
| | | | control | | | *meU | | | |
| A | Linker | RD-12941 | Linker (S18) | S18 | 1299 | meU*meU*meG*meU*meA*meU*meU*meC*meU*meA*meU*meG*meU | 14 | 84.70 | 97.75 |
| | | | | | | *meU | | | |
| | | RD-12942 | Linker (C6) | C6 | 1299 | meU*meU*meG*meU*meA*meU*meU*meC*meU*meA*meU*meG*meU | 14 | 89.95 | 97.45 |
| | | | | | | *meU | | | |
| | | RD-12943 | Linker (UUACA) | UUACA | 1299 | meU*meU*meG*meU*meA*meU*meU*meC*meU*meA*meU*meG*meU | 14 | 58.35 | 93.75 |
| | | | | | | *meU | | | |
| | | RD-12944 | Linker (UUCUU) | UUCUU | 1299 | meU*meU*meG*meU*meA*meU*meU*meC*meU*meA*meU*meG*meU | 14 | 60.80 | 92.95 |
| | | | | | | *meU | | | |
| | | RD-12945 | Linker (C3) | C3 | 1299 | meU*meU*meG*meU*meA*meU*meU*meC*meU*meA*meU*meG*meU | 14 | 89.15 | 97.75 |
| | | | | | | *meU | | | |
| | | RD-12947 | Linker (d Spacer) | d Spacer | 1299 | meU*meU*meG*meU*meA*meU*meU*meC*meU*meA*meU*meG*meU | 14 | 87.75 | 96.65 |
| | | | | | | *meU | | | |
| | | RD-12948 | Linker (C12) | C12 | 1299 | meU*meU*meG*meU*meA*meU*meU*meC*meU*meA*meU*meG*meU | 14 | 96.60 | 98.55 |
| | | | | | | *meU | | | |
| | | RD-12949 | Linker (L14) | L14 | 1299 | meU*meU*meG*meU*meA*meU*meU*meC*meU*meA*meU*meG*meU | 14 | 90.30 | 97.60 |
| | | | | | | *meU | | | |
| | | RD-12950 | Linker (L15) | L15 | 1299 | meU*meU*meG*meU*meA*meU*meU*meC*meU*meA*meU*meG*meU | 14 | 87.15 | 98.50 |
| | | | | | | *meU | | | |
| | | RD-12951 | Linker (L16) | L16 | 1299 | meU*meU*meG*meU*meA*meU*meU*meC*meU*meA*meU*meG*meU | 14 | 87.95 | 98.50 |
| | | | | | | *meU | | | |
| B | Palindrome | RD-12952 | 5′-UUGUAUUCUUGUAU-3′ | L9 | 1300 | meU*meU*meG*meU*meA*meU*meU*meC*meU*meU*meG*meU*meA | 14 | 87.55 | 98.55 |
| | | | | | | *meU | | | |
| | | RD-12953 | 5′-UAUGUUUCUAUGUU-3′ | L9 | 1301 | meU*meA*meU*meG*meU*meU*meU*meC*meU*meA*meU*meG*meU | 14 | 87.05 | 97.10 |
| | | | | | | *meU | | | |
| | | RD-12954 | 5′-UUGUAUCUUAUGUU-3′ | L9 | 1302 | meU*meU*meG*meU*meA*meU*meC*meU*meU*meA*meU*meG*meU | 14 | 89.05 | 97.45 |
| | | | | | | *meU | | | |
| | | RD-12955 | 5′-UUGUAUUAUGUU-3′ | L9 | 1303 | meU*meU*meG*meU*meA*meU*meU*meA*meU*meG*meU*meU | 12 | 84.85 | 98.65 |
| | | RD-12956 | 5′-UUGUAUCCUAUGUU-3′ | L9 | 1304 | meU*meU*meG*meU*meA*meU*meC*meC*meC*meU*meA*meG*meU | 14 | 88.15 | 96.25 |
| | | | | | | *meU | | | |
| | | RD-12957 | 5′-UUGUAU-3′ | L9 | 1305 | meU*meU*meG*meU*meA*meU | 6 | 71.15 | 90.45 |
| | | RD-12958 | 5′-UUGUAUUC-3′ | L9 | 1306 | meU*meU*meG*meU*meA*meU*meU*meC | 8 | 72.25 | 95.35 |
| | | RD-12959 | 5′-UUGUAUUCUAU-3′ | L9 | 1307 | meU*meU*meG*meU*meA*meU*meU*meC*meU*meA*meU | 11 | 76.10 | 92.30 |
| | | RD-12960 | 5′-UUGUAUAGUAUGUU-3′ | L9 | 1308 | meU*meU*meG*meU*meA*meU*meA*meG*meU*meA*meU*meG*meU | 14 | 87.35 | 96.40 |
| | | | | | | *meU | | | |
| | | RD-12961 | 5′-UAGUAUUCUAUGAU-3′ | L9 | 1309 | meU*meA*meG*meU*meA*meU*meU*meC*meU*meA*meU*meG*meA | 14 | 83.70 | 96.05 |
| | | | | | | *meU | | | |
| | | RD-12962 | 5′-UACUAUUCUAUCAU-3′ | L9 | 1310 | meU*meA*meC*meU*meA*meU*meU*meC*meU*meA*meU*meC*meA | 14 | 77.10 | 92.95 |
| | | | | | | *meU | | | |
| | | RD-12963 | 5′-UACAUAUCAUACAU-3′ | L9 | 1311 | meU*meA*meC*meA*meU*meA*meU*meC*meA*meU*meA*meC*meA | 14 | 78.00 | 92.60 |
| | | | | | | *meU | | | |
| | | RD-12964 | 5′-UUAUAUUCUAUAUU-3′ | L9 | 1312 | meU*meU*meA*meU*meA*meU*meU*meC*meU*meA*meU*meA*meU | 14 | 80.20 | 92.45 |
| | | | | | | *meU | | | |
| | | RD-12965 | 5′-UUAUAUUCUAUGUU-3′ | L9 | 1313 | meU*meU*meA*meU*meA*meU*meU*meC*meU*meA*meU*meG*meU | 14 | 86.40 | 97.60 |
| | | | | | | *meU | | | |
| | | RD-12966 | 5′-UAGUAUUCUAUGUU-3′ | L9 | 1314 | meU*meA*meG*meU*meA*meU*meU*meC*meU*meA*meU*meG*meU | 14 | 82.70 | 95.25 |
| | | | | | | *meU | | | |
| C | PS | RD-12967 | 2 PS backbone | L9 | 1315 | meU*meUmeGmeUmeAmeUmeUmeCmeUmeAmeUmeGmeU*meU | 14 | 54.05 | 81.50 |
| | modifi- | RD-12968 | 4 PS backbone | L9 | 1316 | meU*meU*meGmeUmeAmeUmeUmeCmeUmeAmeUmeG*meU*meU | 14 | 59.45 | 84.30 |
| | cation | RD-12969 | 6 PS backbone | L9 | 1317 | meU*meU*meG*meUmeAmeUmeUmeCmeUmeAmeU*meG*meU*meU | 14 | 64.70 | 88.35 |
| | | RD-12970 | 8 PS backbone | L9 | 1318 | meU*meU*meG*meU*meAmeUmeUmeCmeUmeA*meU*meG*meU*meU | 14 | 71.55 | 89.55 |
| | | RD-12971 | 10 PS backbone | L9 | 1319 | meU*meU*meG*meU*meA*meUmeUmeCmeU*meA*meU*meG*meU*me | 14 | 79.10 | 92.25 |
| | | | | | | U | | | |
| | | RD-12972 | 12 PS backbone | L9 | 1320 | meU*meU*meG*meU*meA*meU*meU*meC*meU*meA*meU*meG*meU* | 14 | 82.80 | 94.15 |
| | | | | | | meU | | | |
| D | 2′-Ome | RD-12973 | 2-2′Ome | L9 | 1321 | meU*meU*meG*meU*meA*meU*mU*mC*meU*meA*meU*meG*meU* | 14 | 84.45 | 93.70 |
| | modifi- | | | | | meU | | | |
| | cation | RD-12974 | 4-2′Ome | L9 | 1322 | meU*meU*meG*meU*meA*mU*mU*mC*mU*meA*meU*meG*meU*me | 14 | 86.95 | 96.50 |
| | | | | | | U | | | |
| | | RD-12975 | 6-2′Ome | L9 | 1323 | meU*meU*meG*meU*mA*mU*mU*mC*mU*mA*meU*meG*meU*meU | 14 | 84.05 | 96.60 |
| | | RD-12976 | 8-2′Ome | L9 | 1324 | meU*meU*meG*mU*mA*mU*mU*mC*mU*mA*mU*meG*meU*meU | 14 | 85.20 | 97.50 |
| | | RD-12977 | 10-2′Ome | L9 | 1325 | meU*meU*mG*mU*mA*mU*mU*mC*mU*mA*mU*mG*meU*meU | 14 | 87.05 | 98.50 |
| | | RD-12978 | 12-2′Ome | L9 | 1326 | meU*mU*mG*mU*mA*mU*mU*mC*mU*mA*mU*mG*mU*meU | 14 | 89.70 | 98.05 |
| | | RD-12979 | 14-2′Ome | L9 | 1327 | mU*mU*mG*mU*mA*mU*mU*mC*mU*mA*mU*mG*mU*mU | 14 | 90.35 | 98.30 |
| E | ACO size | RD-12980 | ACO size: 13-nt | L9 | 1328 | meU*meU*meG*meU*meA*meU*meU*meC*meU*meA*meU*meG*meU | 13 | 83.95 | 94.70 |
| | | RD-12981 | ACO size: 12-nt | L9 | 1329 | meU*meU*meG*meU*meA*meU*meU*meC*meU*meA*meU*meG | 12 | 77.90 | 93.50 |
| | | RD-12982 | ACO size: 11-nt | L9 | 1330 | meU*meU*meG*meU*meA*meU*meU*meC*meU*meA*meU | 11 | 81.30 | 93.20 |
| | | RD-12983 | ACO size: 10-nt | L9 | 1331 | meU*meU*meG*meU*meA*meU*meU*meC*meU*meA | 10 | 67.65 | 90.95 |
| | | RD-12984 | ACO size: 9-nt | L9 | 1332 | meU*meU*meG*meU*meA*meU*meU*meC*meU | | 66.70 | 88.90 |
| | | RD-12985 | ACO size: 8-nt | L9 | 1333 | meU*meU*meG*meU*meA*meU*meU*meC | 00 | 65.95 | 89.10 |
| | | RD-12986 | ACO size: 7-nt | L9 | 1334 | meU*meU*meG*meU*meA*meU*meU | | 60.65 | 88.60 |
| | | RD-12987 | ACO size: 6-nt | L9 | 1335 | meU*meU*meG*meU*meA*meU | 0 | 60.30 | 89.75 |
| F | Adenine | RD-12988 | Adenine rich: 5-nt | L9 | 1336 | meC*meG*meG*meA*meA*meG*meU*meA*meC*meC*meC*meA*meA | 14 | 74.25 | 88.50 |
| | rich | | | | | *meC | | | |
| | | RD-12989 | Adenine rich: 6-nt | L9 | 1337 | meA*meA*meU*meA*meA*meC*meU*meA*meC*meG*meU*meG*meU | 14 | 83.90 | 94.25 |
| | | | | | | *meA | | | |
| | | RD-12990 | Adenine rich: 7-nt | L9 | 1338 | meA*meA*meU*meA*meC*meG*meA*meA*meA*meC*meC*meU*meG | 14 | 79.75 | 95.20 |
| | | | | | | *meA | | | |
| | | RD-12991 | Adenine rich: 8-nt | L9 | 1339 | meA*meC*meG*meA*meC*meA*meU*meC*meA*meA*meA*meU*meA | 14 | 71.40 | 92.80 |
| | | | | | | *meA | | | |
| | | RD-12992 | Adenine rich: 9-nt | L9 | 1340 | meA*meA*meA*meU*meA*meA*meU*meA*meA*meG*meA*meA*meC | 14 | 77.95 | 94.95 |
| | | | | | | *meG | | | |
| G | Cytosine | RD-12993 | Cytosine rich: 5-nt | L9 | 1341 | meC*meC*meA*meA*meC*meC*meA*meU*meC*meG*meG*meA*meU | 14 | 68.75 | 89.85 |
| | rich | | | | | *meU | | | |
| | | RD-12994 | Cytosine rich: 6-nt | L9 | 1342 | meC*meG*meU*meC*meC*meC*meU*meC*meG*meG*meG*meG*meA | 14 | 67.10 | 85.45 |
| | | | | | | *meC | | | |
| | | RD-12995 | Cytosine rich: 7-nt | L9 | 1343 | meC*meC*meA*meA*meA*meC*meC*meG*meC*meA*meG*meC*meU | 14 | 68.15 | 84.45 |
| | | | | | | *meC | | | |
| | | RD-12996 | Cytosine rich: 8-nt | L9 | 1344 | meC*meG*meC*meC*meA*meC*meC*meC*meU*meC*meU*meG*meG* | 14 | 68.80 | 86.10 |
| | | | | | | meC | | | |
| | | RD-12997 | Cytosine rich: 9-nt | L9 | 1345 | meC*meA*meC*meC*meC*meC*meC*meC*meA*meC*meA*meU*meC* | 14 | 65.90 | 83.05 |
| | | | | | | meG | | | |
| | | RD-12998 | Cytosine rich: 10-nt | L9 | 1346 | meC*meC*meG*meC*meU*meC*meC*meC*meC*meC*meC*meU*meA* | 14 | 60.45 | 80.65 |
| | | | | | | meC | | | |
| H | Guanine | RD-12999 | Guanine rich: 5-nt | L9 | 1347 | meG*meG*meC*meA*meC*meA*meA*meG*meG*meA*meU*meU*meC | 14 | 78.50 | 95.40 |
| | rich | | | | | *mG | | | |
| | | RD-13000 | Guanine rich: 6-nt | L9 | 1348 | meG*meG*meU*meG*meU*meG*meG*meA*meA*meG*meC*meC*meU | 14 | 74.65 | 93.65 |
| | | | | | | *meA | | | |
| | | RD-13001 | Guanine rich: 7-nt | L9 | 1349 | meG*meA*meG*meU*meU*meC*meG*meG*meA*meU*meG*meG*meG | 14 | 78.15 | 95.55 |
| | | | | | | *meC | | | |
| | | RD-13002 | Guanine rich: 8-nt | L9 | 1350 | meG*meA*meG*meC*meG*meG*meG*meG*meU*meG*meC*meG*meC | 14 | 75.45 | 93.20 |
| | | | | | | *meC | | | |
| | | RD-13003 | Guanine rich: 9-nt | L9 | 1351 | meG*meG*meU*meG*meG*meG*meC*meG*meU*meC*meG*meG*meU | 14 | 78.30 | 94.25 |
| | | | | | | *meG | | | |
| I | Uracil | RD-13004 | Uracil rich: 5-nt | L9 | 1352 | meU*meG*meG*meG*meU*meA*meG*meG*meU*meC*meU*meC*meG | 14 | 78.20 | 93.15 |
| | rich | | | | | *meU | | | |
| | | RD-13005 | Uracil rich: 6-nt | L9 | 1353 | meU*meU*meG*meU*meC*meC*meG*meU*meU*meC*meG*meC*meU | 14 | 85.25 | 95.55 |
| | | | | | | *meA | | | |
| | | RD-13006 | Uracil rich: 7-nt | L9 | 1354 | meU*meA*meU*meG*meU*meC*meG*meU*meA*meU*meU*meG*meU | 14 | 85.75 | 98.50 |
| | | | | | | *meA | | | |
| | | RD-13007 | Uracil rich: 8-nt | L9 | 1355 | meU*meA*meU*meU*meG*meC*meU*meC*meG*meC*meU*meU*meU | 14 | 84.50 | 93.05 |
| | | | | | | *meU | | | |
| | | RD-13008 | Uracil rich: 9-nt | L9 | 1356 | meU*meU*meU*meC*meG*meU*meU*meU*meG*meC*meU*meG*meU | 14 | 84.10 | 97.40 |
| | | | | | | *meU | | | |
| | | RD-13009 | Uracil rich: 10-nt | L9 | 1357 | meU*meG*meU*meU*meA*meC*meG*meU*meU*meU*meU*meU*meU | 14 | 85.95 | 96.75 |
| | | | | | | *meU | | | |
| J | Purine | RD-13010 | Purines rich: 9a | L9 | 1358 | meG*meG*meG*meU*meU*meA*meA*meA*meA*meC*meC*meC*meG | 14 | 69.75 | 92.30 |
| | rich | | | | | *mG | | | |
| | | RD-13011 | Purines rich: 9b | L9 | 1359 | meA*meA*meC*meG*meA*meA*meC*meC*meG*meA*meG*meU*meG | 14 | 84.65 | 96.10 |
| | | | | | | *meU | | | |
| | | RD-13012 | Purines rich: 9c | L9 | 1360 | meU*meG*meC*meA*meU*meC*meG*meG*meA*meU*meG*meA*meA | 14 | 83.90 | 95.20 |
| | | | | | | *meA | | | |
| | | RD-13013 | Purines rich: 10a | L9 | 1361 | meA*meC*meG*meG*meA*meA*meG*meA*meG*meG*meC*meG*meC | 14 | 85.50 | 96.90 |
| | | | | | | *meC | | | |
| | | RD-13014 | Purines rich: 10b | L9 | 1362 | meG*meU*meA*meA*meG*meU*meA*meC*meG*meC*meA*meG*meG | 14 | 83.25 | 97.80 |
| | | | | | | *meG | | | |
| | | RD-13015 | Purines rich: 10c | L9 | 1363 | meG*meU*meA*meA*meA*meG*meU*meG*meA*meG*meC*meA*meU | 14 | 83.55 | 99.55 |
| | | | | | | *meA | | | |
| | | RD-13016 | Purines rich: Ila | L9 | 1364 | meA*meA*meU*meA*meG*meA*meG*meC*meG*meA*meG*meG*meA | 14 | 86.70 | 98.25 |
| | | | | | | *meU | | | |
| | | RD-13017 | Purines rich: 11b | L9 | 1365 | meC*meG*meC*meA*meG*meA*meG*meA*meA*meU*meG*meA*meA | 14 | 82.80 | 97.80 |
| | | | | | | *meA | | | |
| K | Pyrimidine | RD-13018 | Pyrimidines rich: 9a | L9 | 1366 | meU*meG*meC*meG*meU*meC*meG*meA*meA*meU*meU*meC*meU | 14 | 81.05 | 95.40 |
| | rich | | | | | *mC | | | |
| | | RD-13019 | Pyrimidines tich: 9b | L9 | 1367 | meU*meC*meA*meC*meU*meU*meA*meU*meG*meC*meA*meC*meG | 14 | 81.35 | 94.35 |
| | | | | | | *meC | | | |
| | | RD-13020 | Pyrimidines rich: 9c | L9 | 1368 | meC*meC*meU*meU*meA*meU*meU*meA*meC*meA*meG*meA*meU | 14 | 79.50 | 94.70 |
| | | | | | | *meC | | | |
| | | RD-13021 | Pyrimidines rich: 10a | L9 | 1369 | meU*meU*meC*meC*meC*meC*meU*meU*meG*meA*meU*meA*meA | 14 | 82.45 | 94.70 |
| | | | | | | *mC | | | |
| | | RD-13022 | Pyrimidines rich: 10b | L9 | 1370 | meC*meC*meU*meU*meU*meU*meC*meU*meA*meG*meA*meU*meC | 14 | 75.70 | 93.30 |
| | | | | | | *meG | | | |
| | | RD-13023 | Pyrimidines rich: 11a | L9 | 1371 | meU*meC*meC*meU*meG*meU*meC*meC*meA*meU*meC*meC*meU | 14 | 70.60 | 89.05 |
| | | | | | | *meA | | | |
| | | RD-13024 | Pyrimidines rich: 11b | L9 | 1372 | meU*meU*meC*meU*meA*meU*meG*meU*meG*meC*meU*meC*meC | 14 | 77.95 | 93.40 |
| | | | | | | *meC | | | |
| | | RD-13025 | Pyrimidines rich: 12 | L9 | 1373 | meU*meA*meC*meU*meC*meC*meU*meU*meU*meC*meA*meU*meU | 14 | 73.70 | 91.90 |
| | | | | | | *meC | | | |
| L | Balanced | RD-13026 | Balanced | L9 | 1374 | meG*meG*meC*meU*meA*meU*meU*meA*meU*meC*meC*meA*meU | 14 | 79.60 | 91.80 |
| | | | Purineses/ | | | *meA | | | |
| | | | Pyrimidines1 | | | | | | |
| | | RD-13027 | Balanced | L9 | 1375 | meU*meU*meA*meC*meU*meC*meA*meG*meG*meC*meA*meG*meG | 14 | 83.60 | 96.10 |
| | | | Purineses/ | | | *meA | | | |
| | | | Pyrimidines2 | | | | | | |
| | | RD-13028 | Balanced | L9 | 1376 | meA*meU*meG*meU*meG*meC*meA*meA*meC*meC*meA*meU*MeC | 14 | 82.65 | 92.95 |
| | | | Purineses/ | | | *meG | | | |
| | | | Pyrimidines3 | | | | | | |
| | | RD-13029 | Balanced | L9 | 1377 | meC*meC*meG*meG*meU*meG*meU*meA*meU*meG*meU*meC*meA | 14 | 82.05 | 94.60 |
| | | | Purineses/ | | | *meA | | | |
| | | | Pyrimidines4 | | | | | | |
| | | RD-13030 | Balanced | L9 | 1378 | meU*meU*meU*meG*meU*meC*meG*meG*meU*meA*meC*meC*meA | 14 | 80.20 | 94.65 |
| | | | Purineses/ | | | *meA | | | |
| | | | Pyrimidines5 | | | | | | |
| | | RD-13031 | Balanced | L9 | 1379 | meC*meU*meC*meU*meA*meA*meG*meG*meC*meA*meC*meC*meU | 14 | 77.35 | 97.75 |
| | | | Purineses/ | | | *meG | | | |
| | | | Pyrimidines6 | | | | | | |

spacer-9 linker; C3, spacer-C3 linker; C6, spacer-C6 linker; C12, spacer-C12 linker; UUACA, L7 linker; UUCUU, L8 linker; d
spacer, L12 linker; L14, spacer-L14 linker; L15, spacer-L15 linker; L16, spacer-L16 linker.

TABLE 28
Oligonucleotide sequences and duplex compositions of novel ODV designs

| ODV- | | SEQ | | |
|---|---|---|---|---|
| SIRNA | Target | ID | | Size |
| name | gene | NO | Sequence (5′-3′) | (bp) |
| RD-12556 | SOD1 | 1196 | mA*fG*mGfUmGfGmAfAfAfUfGfAmAfGmAfAmAfG*mU*fA | 20 |
| | | 57 | VPmU*fA*mCfUmUfUmCfUmUfCmAfUmUfUmCfCmAfCmCfU*mU*fU | 22 |
| RD-12559 | SOD1 | 1197 | mA*fG*mGfUmGfGmAfAfAfUfGfAmAfGmAfAmAfG*mU*fA-S9- | 34 |
| | | | meU*meU*meG*meU*meA*meU*meU*meC*meU*meA*meU*meG*meU*meU | |
| | | 57 | VPmU*fA*mCfUmUfUmCfUmUfCmAfUmUfUmCfCmAfCmCfU*mU*fU | 22 |
| RD-12941 | SOD1 | 1198 | mA*fG*mGfUmGfGmAfAfAfUfGfAmAfGmAfAmAfG*mU*fA-S18- | 34 |
| | | | meU*meU*meG*meU*meA*meU*meU*meC*meU*meA*meU*meG*meU*meU | |
| | | 57 | VPmU*fA*mCfUmUfUmCfUmUfCmAfUmUfUmCfCmAfCmCfU*mU*fU | 22 |
| RD-12942 | SOD1 | 1199 | mA*fG*mGfUmGfGmAfAfAfUfGfAmAfGmAfAmAfG*mU*fA-C6- | 34 |
| | | | meU*meU*meG*meU*meA*meU*meU*meC*meU*meA*meU*meG*meU*meU | |
| | | 57 | VPmU*fA*mCfUmUfUmCfUmUfCmAfUmUfUmCfCmAfCmCfU*mU*fU | 22 |
| RD-12943 | SOD1 | 1200 | mA*fG*mGfUmGfGmAfAfAfUfGfAmAfGmAfAmAfG*mU*fA-(UUACA)- | 39 |
| | | | meU*meU*meG*meU*meA*meU*meU*meC*meU*meA*meU*meG*meU*meU | |
| | | 57 | VPmU*fA*mCfUmUfUmCfUmUfCmAfUmUfUmCfCmAfCmCfU*mU*fU | 22 |
| RD-12944 | SOD1 | 1201 | mA*fG*mGfUmGfGmAfAfAfUfGfAmAfGmAfAmAfG*mU*[A-(UUCUU)- | 39 |
| | | | meU*meU*meG*meU*meA*meU*meU*meC*meU*meA*meU*meG*meU*meU | |
| | | 57 | VPmU*fA*mCfUmUfUmCfUmUfCmAfUmUfUmCfCmAfCmCfU*mU*fU | 22 |
| RD-12945 | SOD1 | 1202 | mA*fG*mGfUmGfGmAfAfAfUfGfAmAfGmAfAmAfG*mU*fA-C3- | 34 |
| | | | meU*meU*meG*meU*meA*meU*meU*meC*meU*meA*meU*meG*meU*meU | |
| | | 57 | VPmU*fA*mCfUmUfUmCfUmUfCmAfUmUfUmCfCmAfCmCfU*mU*fU | 22 |
| RD-12947 | SOD1 | 1204 | mA*fG*mGfUmGfGmAfAfAfUfGfAmAfGmAfAmAfG*mU*fA-d spacer- | 34 |
| | | | meU*meU*meG*meU*meA*meU*meU*meC*meU*meA*meU*meG*meU*meU | |
| | | 57 | VPmU*fA*mCfUmUfUmCfUmUfCmAfUmUfUmCfCmAfCmCfU*mU*fU | 22 |
| RD-12948 | SOD1 | 1205 | mA*fG*mGfUmGfGmAfAfAfUfGfAmAfGmAfAmAfG*mU*fA-C12- | 34 |
| | | | meU*meU*meG*meU*meA*meU*meU*meC*meU*meA*meU*meG*meU*meU | |
| | | 57 | VPmU*fA*mCfUmUfUmCfUmUfCmAfUmUfUmCfCmAfCmCfU*mU*fU | 22 |
| RD-12949 | SOD1 | 1206 | mA*fG*mGfUmGfGmAfAfAfUfGfAmAfGmAfAmAfG*mU*fA-L14- | 34 |
| | | | meU*meU*meG*meU*meA*meU*meU*meC*meU*meA*meU*meG*meU*meU | |
| | | 57 | VPmU*fA*mCfUmUfUmCfUmUfCmAfUmUfUmCfCmAfCmCfU*mU*fU | 22 |
| RD-12950 | SOD1 | 1207 | mA*fG*mGfUmGfGmAfAfAfUfGfAmAfGmAfAmAfG*mU*fA-L15- | 34 |
| | | | meU*meU*meG*meU*meA*meU*meU*meC*meU*meA*meU*meG*meU*meU | |
| | | 57 | VPmU*fA*mCfUmUfUmCfUmUfCmAfUmUfUmCfCmAfCmCfU*mU*fU | 22 |
| RD-12951 | SOD1 | 1208 | mA*fG*mGfUmGfGmAfAfAfUfGfAmAfGmAfAmAfG*mU*fA-L16- | 34 |
| | | | meU*meU*meG*meU*meA*meU*meU*meC*meU*meA*meU*meG*meU*meU | |
| | | 57 | VPmU*fA*mCfUmUfUmCfUmUfCmAfUmUfUmCfCmAfCmCfU*mU*fU | 22 |
| RD-12952 | SOD1 | 1209 | mA*fG*mGfUmGfGmAfAfAfUfGfAmAfGmAfAmAfG*mU*fA-L9- | 34 |
| | | | meU*meU*meG*meU*meA*meU*meU*meC*meU*meU*meG*meU*meA*meU | |
| | | 57 | VPmU*fA*mCfUmUfUmCfUmUfCmAfUmUfUmCfCmAfCmCfU*mU*fU | 22 |
| RD-12953 | SOD1 | 1210 | mA*fG*mGfUmGfGmAfAfAfUfGfAmAfGmAfAmAfG*mU*fA-L9- | 34 |
| | | | meU*meA*meU*meG*meU*meU*meU*meC*meU*meA*meU*meG*meU*meU | |
| | | 57 | VPmU*fA*mCfUmUfUmCfUmUfCmAfUmUfUmCfCmAfCmCfU*mU*fU | 22 |
| RD-12954 | SOD1 | 1211 | mA*fG*mGfUmGfGmAfAfAfUfGfAmAfGmAfAmAfG*mU*fA-L9- | 34 |
| | | | meU*meU*meG*meU*meA*meU*meC*meU*meU*meA*meU*meG*meU*meU | |
| | | 57 | VPmU*fA*mCfUmUfUmCfUmUfCmAfUmUfUmCfCmAfCmCfU*mU*fU | 22 |
| RD-12955 | SOD1 | 1212 | mA*fG*mGfUmGfGmAfAfAfUfGfAmAfGmAfAmAfG*mU*fA-L9- | 32 |
| | | | meU*meU*meG*meU*meA*meU*meU*meA*meU*meG*meU*meU | |
| | | 57 | VPmU*fA*mCfUmUfUmCfUmUfCmAfUmUfUmCfCmAfCmCfU*mU*fU | 22 |
| RD-12956 | SOD1 | 1213 | mA*fG*mGfUmGfGmAfAfAfUfGfAmAfGmAfAmAfG*mU*fA-L9- | 34 |
| | | | meU*meU*meG*meU*meA*meU*meC*meC*meU*meA*meU*meG*meU*meU | |
| | | 57 | VPmU*fA*mCfUmUfUmCfUmUfCmAfUmUfUmCfCmAfCmCfU*mU*fU | 22 |
| RD-12957 | SOD1 | 1214 | mA*fG*mGfUmGfGmAfAfAfUfGfAmAfGmAfAmAfG*mU*fA-L9- | 26 |
| | | | meU*meU*meG*meU*meA*meU | |
| | | 57 | VPmU*fA*mCfUmUfUmCfUmUfCmAfUmUfUmCfCmAfCmCfU*mU*fU | 22 |
| RD-12958 | SOD1 | 1215 | mA*fG*mGfUmGfGmAfAfAfUfGfAmAfGmAfAmAfG*mU*fA-L9- | 28 |
| | | | meU*meU*meG*meU*meA*meU*meU*meC | |
| | | 57 | VPmU*fA*mCfUmUfUmCfUmUfCmAfUmUfUmCfCmAfCmCfU*mU*fU | 22 |
| RD-12959 | SOD1 | 1216 | mA*fG*mGfUmGfGmAfAfAfUfGfAmAfGmAfAmAfG*mU*fA-L9- | 31 |
| | | | meU*meU*meG*meU*meA*meU*meU*meC*meU*meA*meU | |
| | | 57 | VPmU*fA*mCfUmUfUmCfUmUfCmAfUmUfUmCfCmAfCmCfU*mU*fU | 22 |
| RD-12960 | SOD1 | 1217 | mA*fG*mGfUmGfGmAfAfAfUfGfAmAfGmAfAmAfG*mU*fA-L9- | 34 |
| | | | meU*meU*meG*meU*meA*meU*meA*meG*meU*meA*meU*meG*meL*meU | |
| | | 57 | VPmU*fA*mCfUmUfUmCfUmUfCmAfUmUfUmCfCmAfCmCfU*mU*fU | 22 |
| RD-12961 | SOD1 | 1218 | mA*fG*mGfUmGfGmAfAfAfUfGfAmAfGmAfAmAfG*mU*fA-L9- | 34 |
| | | | meU*meA*meG*meU*meA*meU*meU*meC*meU*meA*meU*meG*meA*meU | |
| | | 57 | VPmU*fA*mCfUmUfUmCfUmUfCmAfUmUfUmCfCmAfCmCfU*mU*fU | 22 |
| RD-12962 | SOD1 | 1219 | mA*fG*mGfUmGfGmAfAfAfUfGfAmAfGmAfAmAfG*mU*fA-L9- | 34 |
| | | | meU*meA*meC*meU*meA*meU*meU*meC*meU*meA*meU*meC*meA*meU | |
| | | 57 | VPmU*fA*mCfUmUfUmCfUmUfCmAfUmUfUmCfCmAfCmCfU*mU*fU | 22 |
| RD-12963 | SOD1 | 1220 | mA*fG*mGfUmGfGmAfAfAfUfGfAmAfGmAfAmAfG*mU*fA-L9- | 34 |
| | | | meU*meA*meC*meA*meU*meA*meU*meC*meA*meU*meA*meC*meA*meU | |
| | | 57 | VPmU*fA*mCfUmUfUmCfUmUfCmAfUmUfUmCfCmAfCmCfU*mU*fU | 22 |
| RD-12964 | SOD1 | 1221 | mA*fG*mGfUmGfGmAfAfAfUfGfAmAfGmAfAmAfG*mU*fA-L9- | 34 |
| | | | meU*meU*meA*meU*meA*meU*meU*meC*meU*meA*meU*meA*meU*meU | |
| | | 57 | VPmU*fA*mCfUmUfUmCfUmUfCmAfUmUfUmCfCmAfCmCfU*mU*fU | 22 |
| RD-12965 | SOD1 | 1222 | mA*fG*mGfUmGfGmAfAfAfUfGfAmAfGmAfAmAfG*mU*fA-L9- | 34 |
| | | | meU*meU*meA*meU*meA*meU*meU*meC*meU*meA*meU*meG*meU*meU | 22 |
| | | 57 | VPmU*fA*mCfUmUfUmCfUmUfCmAfUmUfUmCfCmAfCmCfU*mU*fU | |
| RD-12966 | SOD1 | 1223 | mA*fG*mGfUmGfGmAfAfAfUfGfAmAfGmAfAmAfG*mU*fA-L9- | 34 |
| | | | meU*meA*meG*meU*meA*meU*meU*meC*meU*meA*meU*meG*meU*meU | |
| | | 57 | VPmU*fA*mCfUmUfUmCfUmUfCmAfUmUfUmCfCmAfCmCfU*mU*fU | 22 |
| RD-12967 | SOD1 | 1224 | mA*fG*mGfUmGfGmAfAfAfUfGfAmAfGmAfAmAfG*mU*fA-L9- | 34 |
| | | | meU*meUmeGmeUmeAmeUmeUmeCmeUmeAmeUmeGmeU*meU | |
| | | 57 | VPmU*fA*mCfUmUfUmCfUmUfCmAfUmUfUmCfCmAfCmCfU*mU*fU | 22 |
| RD-12968 | SOD1 | 1225 | mA*fG*mGfUmGfGmAfAfAfUfGfAmAfGmAfAmAfG*mU*fA-L9- | 34 |
| | | | meU*meU*meGmeUmeAmeUmeUmeCmeUmeAmeUmeG*meU*meU | |
| | | 57 | VPmU*fA*mCfUmUfUmCfUmUfCmAfUmUfUmCfCmAfCmCfU*mU*fU | 22 |
| RD-12969 | SOD1 | 1226 | mA*fG*mGfUmGfGmAfAfAfUfGfAmAfGmAfAmAfG*mU*fA-L9- | 34 |
| | | | meU*meU*meG*meUmeAmeUmeUmeCmeUmeU*meG*meU*meU | |
| | | 57 | VPmU*fA*mCfUmUfUmCfUmUfCmAfUmUfUmCfCmAfCmCfU*mU*fU | 22 |
| RD-12970 | SOD1 | 1227 | mA*fG*mGfUmGfGmAfAfAfUfGfAmAfGmAfAmAfG*mU*fA-L9- | 34 |
| | | | meU*meU*meG*meU*meAmeUmeUmeCmeUmeA*meU*meG*meU*meU | |
| | | 57 | VPmU*fA*mCfUmUfUmCfUmUfCmAfUmUfUmCfCmAfCmCfU*mU*fU | 22 |
| RD-12971 | SOD1 | 1228 | mA*fG*mGfUmGfGmAfAfAfUfGfAmAfGmAfAmAfG*mU*fA-L9- | 34 |
| | | | meU*meU*meG*meU*meA*meUmeUmeCmeU*meA*meU*meG*meU*meU | |
| | | 57 | VPmU*fA*mCfUmUfUmCfUmUfCmAfUmUfUmCfCmAfCmCfU*mU*fU | 22 |
| RD-12972 | SOD1 | 1229 | mA*fG*mGfUmGfGmAfAfAfUfGfAmAfGmAfAmAfG*mU*fA-L9- | 34 |
| | | | meU*meU*meG*meU*meA*meU*meUmeC*meU*meA*meU*meG*meU*meU | |
| | | 57 | VPmU*fA*mCfUmUfUmCfUmUfCmAfUmUfUmCfCmAfCmCfU*mU*fU | 22 |
| RD-12973 | SOD1 | 1230 | mA*fG*mGfUmGfGmAfAfAfUfGfAmAfGmAfAmAfG*mU*fA-L9- | 34 |
| | | | meU*meU*meG*meU*meA*meU*mU*mC*meU*meA*meU*meG*meU*meU | |
| | | 57 | VPmU*fA*mCfUmUfUmCfUmUfCmAfUmUfUmCfCmAfCmCfU*mU*fU | 22 |
| RD-12974 | SOD1 | 1231 | mA*fG*mGfUmGfGmAfAfAfUfGfAmAfGmAfAmAfG*mU*fA-L9- | 34 |
| | | | meU*meU*meG*meU*meA*mU*mU*mC*mU*meA*meU*meG*meU*meU | |
| | | 57 | VPmU*fA*mCfUmUfUmCfUmUfCmAfUmUfUmCfCmAfCmCfU*mU*fU | 22 |
| RD-12975 | SOD1 | 1232 | mA*fG*mGfUmGfGmAfAfAfUfGfAmAfGmAfAmAfG*mU*fA-L9- | 34 |
| | | | meU*meU*meG*meU*mA*mU*mU*mC*mU*mA*meU*meG*meU*meU | |
| | | 57 | VPmU*fA*mCfUmUfUmCfUmUfCmAfUmUfUmCfCmAfCmCfU*mU*fU | 22 |
| RD-12976 | SOD1 | 1233 | mA*fG*mGfUmGfGmAfAfAfUfGfAmAfGmAfAmAfG*mU*fA-L9- | 34 |
| | | | meU*meU*meG*mU*mA*mU*mU*mC*mU*mA*mU*meG*meU*meU | |
| | | 57 | VPmU*fA*mCfUmUfUmCfUmUfCmAfUmUfUmCfCmAfCmCfU*mU*fU | 22 |
| RD-12977 | SOD1 | 1234 | mA*fG*mGfUmGfGmAfAfAfUfGfAmAfGmAfAmAfG*mU*fA-L9- | 34 |
| | | | meU*meU*mG*mU*mA*mU*mU*mC*mU*mA*mU*mG*meU*meU | |
| | | 57 | VPmU*fA*mCfUmUfUmCfUmUfCmAfUmUfUmCfCmAfCmCfU*mU*fU | 22 |
| RD-12978 | SOD1 | 1235 | mA*fG*mGfUmGfGmAfAfAfUfGfAmAfGmAfAmAfG*mU*fA-L9- | 34 |
| | | | meU*mU*mG*mU*mA*mU*mU*mC*mU*mA*mU*mG*mU*meU | |
| | | 57 | VPmU*fA*mCfUmUfUmCfUmUfCmAfUmUfUmCfCmAfCmCfU*mU*fU | 22 |
| RD-12979 | SOD1 | 1236 | mA*fG*mGfUmGfGmAfAfAfUfGfAmAfGmAfAmAfG*mU*fA-L9- | 34 |
| | | | mU*mU*mG*mU*mA*mU*mU*mC*mU*mA*mU*mG*mU*mU | |
| | | 57 | VPmU*fA*mCfUmUfUmCfUmUfCmAfUmUfUmCfCmAfCmCfU*mU*fU | 22 |
| RD-12980 | SOD1 | 1237 | mA*fG*mGfUmGfGmAfAfAfUfGfAmAfGmAfAmAfG*mU*fA-L9- | 34 |
| | | | meU*meU*meG*meU*meA*meU*meU*meC*meU*meA*meU*meG*meU | |
| | | 57 | VPmU*fA*mCfUmUfUmCfUmUfCmAfUmUfUmCfCmAfCmCfU*mU*fU | 22 |
| RD-12981 | SOD1 | 1238 | mA*fG*mGfUmGfGmAfAfAfUfGfAmAfGmAfAmAfG*mU*fA-L9- | 32 |
| | | | meU*meU*meG*meU*meA*meU*meU*meC*meU*meA*meU*meG | |
| | | 57 | VPmU*fA*mCfUmUfUmCfUmUfCmAfUmUfUmCfCmAfCmCfU*mU*fU | 22 |
| RD-12982 | SOD1 | 1239 | mA*fG*mGfUmGfGmAfAfAfUfGfAmAfGmAfAmAfG*mU*fA-L9- | 31 |
| | | | meU*meU*meG*meU*meA*meU*meU*meC*meU*meA*meU | |
| | | 57 | VPmU*fA*mCfUmUfUmCfUmUfCmAfUmUfUmCfCmAfCmCfU*mU*fU | 22 |
| RD-12983 | SOD1 | 1240 | mA*fG*mGfUmGfGmAfAfAfUfGfAmAfGmAfAmAfG*mU*fA-L9- | 30 |
| | | | meU*meU*meG*meU*meA*meU*meU*meC*meU*meA | |
| | | 57 | VPmU*fA*mCfUmUfUmCfUmUfCmAfUmUfUmCfCmAfCmCIU*mU*fU | 22 |
| RD-12984 | SOD1 | 1241 | mA*fG*mGfUmGfGmAfAfAfUfGfAmAfGmAfAmAfG*mU*fA-L9- | 29 |
| | | | meU*meU*meG*meU*meA*meU*meU*meC*meU | |
| | | 57 | VPmU*fA*mCfUmUfUmCfUmUfCmAfUmUfUmCfCmAfCmCfU*mU*fU | 22 |
| RD-12985 | SOD1 | 1242 | mA*fG*mGfUmGfGmAfAfAfUfGfAmAfGmAfAmAfG*mU*fA-L9- | 28 |
| | | | meU*meU*meG*meU*meA*meU*meU*meC | |
| | | 57 | VPmU*fA*mCfUmUfUmCfUmUfCmAfUmUfUmCfCmAfCmCfU*mU*fU | 22 |
| RD-12986 | SOD1 | 1243 | mA*fG*mGfUmGfGmAfAfAfUfGfAmAfGmAfAmAfG*mU*fA-L9- | 27 |
| | | | meU*meU*meG*meU*meA*meU*meU | |
| | | 57 | VPmU*fA*mCfUmUfUmCfUmUfCmAfUmUfUmCfCmAfCmCfU*mU*fU | 22 |
| RD-12987 | SOD1 | 1244 | mA*fG*mGfUmGfGmAfAfAfUfGfAmAfGmAfAmAfG*mU*fA-L9- | 26 |
| | | | meU*meU*meG*meU*meA*meU | |
| | | 57 | VPmU*fA*mCfUmUfUmCfUmUfCmAfUmUfUmCfCmAfCmCfU*mU*fU | 22 |
| RD-12988 | SOD1 | 1245 | mA*fG*mGfUmGfGmAfAfAfUfGfAmAfGmAfAmAfG*mU*fA-L9- | 34 |
| | | | meC*meG*meG*meA*meA*meG*meU*meA*meC*meC*meC*meA*meA*meC | |
| | | 57 | VPmU*fA*mCfUmUfUmCfUmUfCmAfUmUfUmCfCmAfCmCfU*mU*fU | 22 |
| RD-12989 | SOD1 | 1246 | mA*fG*mGfUmGfGmAfAfAfUfGfAmAfGmAfAmAfG*mU*fA-L9- | 34 |
| | | | meA*meA*meU*meA*meA*meC*meU*meA*meC*meG*meU*meG*meU*meA | |
| | | 57 | VPmU*fA*mCfUmUfUmCfUmUfCmAfUmUfUmCfCmAfCmCfU*mU*fU | 22 |
| RD-12990 | SOD1 | 1247 | mA*fG*mGfUmGfGmAfAfAfUfGfAmAfGmAfAmAfG*mU*fA-L9- | 34 |
| | | | meA*meA*meU*meA*meC*meG*meA*meA*meA*meC*meC*meU*meG*meA | |
| | | 57 | VPmU*fA*mCfUmUfUmCfUmUfCmAfUmUfUmCfCmAfCmCfU*mU*fU | 22 |
| RD-12991 | SOD1 | 1248 | mA*fG*mGfUmGfGmAfAfAfUfGfAmAfGmAfAmAfG*mU*fA-L9- | 34 |
| | | | meA*meC*meG*meA*meC*meA*meU*meC*meA*meA*meA*meU*meA*meA | |
| | | 57 | VPmU*fA*mCfUmUfUmCfUmUfCmAfUmUfUmCfCmAfCmCfU*mU*fU | 22 |
| RD-12992 | SOD1 | 1249 | mA*fG*mGfUmGfGmAfAfAfUfGfAmAfGmAfAmAfG*mU*fA-L9- | 34 |
| | | | meA*meA*meA*meU*meA*meA*meU*meA*meA*meG*meA*meA*meC*meG | |
| | | 57 | VPmU*fA*mCfUmUfUmCfUmUfCmAfUmUfUmCfCmAfCmCfU*mU*fU | 22 |
| RD-12993 | SOD1 | 1250 | mA*fG*mGfUmGfGmAfAfAfUfGfAmAfGmAfAmAfG*mU*fA-L9- | 34 |
| | | | meC*meC*meA*meA*meC*meC*meA*meU*meC*meG*meG*meA*meU*meU | |
| | | 57 | VPmU*fA*mCfUmUfUmCfUmUfCmAfUmUfUmCfCmAfCmCfU*mU*fU | 22 |
| RD-12994 | SOD1 | 1251 | mA*fG*mGfUmGfGmAfAfAfUfGfAmAfGmAfAmAfG*mU*fA-L9- | 34 |
| | | | meC*meG*meU*meC*meC*meC*meU*meC*meG*meG*meG*meG*meA*meC | |
| | | 57 | VPmU*fA*mCfUmUfUmCfUmUfCmAfUmUfUmCfCmAfCmCfU*mU*fU | 22 |
| RD-12995 | SOD1 | 1252 | mA*fG*mGfUmGfGmAfAfAfUfGfAmAfGmAfAmAfG*mU*fA-L9- | 34 |
| | | | meC*meC*meA*meA*meA*meC*meC*meG*meC*meA*meG*meC*meU*meC | |
| | | 57 | VPmU*fA*mCfUmUfUmCfUmUfCmAfUmUfUmCfCmAfCmCfU*mU*fU | 22 |
| RD-12996 | SOD1 | 1253 | mA*fG*mGfUmGfGmAfAfAfUfGfAmAfGmAfAmAfG*mU*fA-L9- | 34 |
| | | | meC*meG*meC*meC*meA*meC*meC*meC*meU*meC*meU*meG*meG*meC | |
| | | 57 | VPmU*fA*mCfUmUfUmCfUmUfCmAfUmUfUmCfCmAfCmCfU*mU*fU | 22 |
| RD-12997 | SOD1 | 1254 | mA*fG*mGfUmGfGmAfAfAfUfGfAmAfGmAfAmAfG*mU*fA-L9- | 34 |
| | | | meC*meA*meC*meC*meC*meC*meC*meC*meA*meC*meA*meU*meC*meG | |
| | | 57 | VPmU*fA*mCfUmUfUmCfUmUfCmAfUmUfUmCfCmAfCmCfU*mU*fU | 22 |
| RD-12998 | SOD1 | 1255 | mA*fG*mGfUmGfGmAfAfAfUfGfAmAfGmAfAmAfG*mU*fA-L9- | 34 |
| | | | meC*meC*meG*meC*meU*meC*meC*meC*meC*meC*meC*meU*meA*meC | |
| | | 57 | VPmU*fA*mCfUmUfUmCfUmUfCmAfUmUfUmCfCmAfCmCfU*mU*fU | 22 |
| RD-12999 | SOD1 | 1256 | mA*fG*mGfUmGfGmAfAfAfUfGfAmAfGmAfAmAfG*mU*fA-L9- | 34 |
| | | | meG*meG*meC*meA*meC*meA*meA*meG*meG*meA*meU*meU*meC*meG | |
| | | 57 | VPmU*fA*mCfUmUfUmCfUmUfCmAfUmUfUmCfCmAfCmCfU*mU*fU | 22 |
| RD-13000 | SOD1 | 1257 | mA*fG*mGfUmGfGmAfAfAfUfGfAmAfGmAfAmAfG*mU*fA-L9- | 34 |
| | | | meG*meG*meU*meG*meU*meG*meG*meA*meA*meG*meC*meC*meU*meA | |
| | | 57 | VPmU*fA*mCfUmUfUmCfUmUfCmAfUmUfUmCfCmAfCmCfU*mU*fU | 22 |
| RD-13001 | SOD1 | 1258 | mA*fG*mGfUmGfGmAfAfAfUfGfAmAfGmAfAmAfG*mU*fA-L9- | 34 |
| | | | meG*meA*meG*meU*meU*meC*meG*meG*meA*meU*meG*meG*meG*meC | |
| | | 57 | VPmU*fA*mCfUmUfUmCfUmUfCmAfUmUfUmCfCmAfCmCfU*mU*fU | 22 |
| RD-13002 | SOD1 | 1259 | mA*fG*mGfUmGfGmAfAfAfUfGfAmAfGmAfAmAfG*mU*fA-L9- | 34 |
| | | | meG*meA*meG*meC*meG*meG*meG*meG*meU*meG*meC*meG*meC*meC | |
| | | 57 | VPmU*fA*mCfUmUfUmCfUmUfCmAfUmUfUmCfCmAfCmCfU*mU*fU | 22 |
| RD-13003 | SOD1 | 1260 | mA*fG*mGfUmGfGmAfAfAfUfGfAmAfGmAfAmAfG*mU*fA-L9- | 34 |
| | | | meG*meG*meU*meG*meG*meG*meC*meG*meU*meC*meG*meG*meU*meG | |
| | | 57 | VPmU*fA*mCfUmUfUmCfUmUfCmAfUmUfUmCfCmAfCmCfU*mU*fU | 22 |
| RD-13004 | SOD1 | 1261 | mA*fG*mGfUmGfGmAfAfAfUfGfAmAfGmAfAmAfG*mU*fA-L9- | 34 |
| | | | meU*meG*meG*meG*meU*meA*meG*meG*meU*meC*meU*meC*meG*meU | |
| | | 57 | VPmU*fA*mCfUmUfUmCfUmUfCmAfUmUfUmCfCmAfCmCfU*mU*fU | 22 |
| RD-13005 | SOD1 | 1262 | mA*fG*mGfUmGfGmAfAfAfUfGfAmAfGmAfAmAfG*mU*fA-L9- | 34 |
| | | | meU*meU*meG*meU*meC*meC*meG*meU*meU*meC*meG*meC*meU*meA | |
| | | 57 | VPmU*fA*mCfUmUfUmCfUmUfCmAfUmUfUmCfCmAfCmCfU*mU*fU | 22 |
| RD-13006 | SOD1 | 1263 | mA*fG*mGfUmGfGmAfAfAfUfGfAmAfGmAfAmAfG*mU*fA-L9- | 34 |
| | | | meU*meA*meU*meG*meU*meC*meG*meU*meA*meU*meU*meG*meU*meA | |
| | | 57 | VPmU*fA*mCfUmUfUmCfUmUfCmAfUmUfUmCfCmAfCmCfU*mU*fU | 22 |
| RD-13007 | SOD1 | 1264 | mA*fG*mGfUmGfGmAfAfAfUfGfAmAfGmAfAmAfG*mU*fA-L9- | 34 |
| | | | meU*meA*meU*meU*meG*meC*meU*meC*meG*meC*meU*meU*meU*meU | |
| | | 57 | VPmU*A*mCfUmUfUmCfUmUfCmAfUmUfUmCfCmAfCmCfU*mU*fU | 22 |
| RD-13008 | SOD1 | 1265 | mA*fG*mGfUmGfGmAfAfAfUfGfAmAfGmAfAmAfG*mU*fA-L9- | 34 |
| | | | meU*meU*meU*meC*meG*meU*meU*meU*meG*meC*meU*meG*meU*meU | |
| | | 57 | VPmU*A*mCfUmUfUmCfUmUfCmAfUmUfUmCfCmAfCmCfU*mU*fU | 22 |
| RD-13009 | SOD1 | 1266 | mA*fG*mGfUmGfGmAfAfAfUfGfAmAfGmAfAmAfG*mU*fA-L9- | 34 |
| | | | meU*meG*meU*meU*meA*meC*meG*meU*meU*meU*meU*meU*meU*meU | |
| | | 57 | VPmU*fA*mCfUmUfUmCfUmUfCmAfUmUfUmCfCmAfCmCfU*mU*fU | 22 |
| RD-13010 | SOD1 | 1267 | mA*fG*mGfUmGfGmAfAfAfUfGfAmAfGmAfAmAfG*mU*fA-L9- | 34 |
| | | | meG*meG*meG*meU*meU*meA*meA*meA*meA*meC*meC*meC*meG*meG | |
| | | 57 | VPmU*fA*mCfUmUfUmCfUmUfCmAfUmUfUmCfCmAfCmCfU*mU*fU | 22 |
| RD-13011 | SOD1 | 1268 | mA*fG*mGfUmGfGmAfAfAfUfGfAmAfGmAfAmAfG*mU*fA-L9- | 34 |
| | | | meA*meA*meC*meG*meA*meA*meC*meC*meG*meA*meG*meU*meG*meU | |
| | | 57 | VPmU*fA*mCfUmUfUmCfUmUfCmAfUmUfUmCfCmAfCmCfU*mU*fU | 22 |
| RD-13012 | SOD1 | 1269 | mA*fG*mGfUmGfGmAfAfAfUfGfAmAfGmAfAmAfG*mU*fA-L9- | 34 |
| | | | meU*meG*meC*meA*meU*meC*meG*meG*meA*meU*meG*meA*meA*meA | |
| | | 57 | VPmU*fA*mCfUmUfUmCfUmUfCmAfUmUfUmCfCmAfCmCU*mU*fU | 22 |
| RD-13013 | SOD1 | 1270 | mA*fG*mGfUmGfGmAfAfAfUfGfAmAfGmAfAmAfG*mU*fA-L9- | 34 |
| | | | meA*meC*meG*meG*meA*meA*meG*meA*meG*meG*meC*meG*meC*meC | |
| | | 57 | VPmU*fA*mCfUmUfUmCfUmUfCmAfUmUfUmCfCmAfCmCfU*mU*fU | 22 |
| RD-13014 | SOD1 | 1271 | mA*fG*mGfUmGfGmAfAfAfUfGfAmAfGmAfAmAfG*mU*fA-L9- | 34 |
| | | | meG*meU*meA*meA*meG*meU*meA*meC*meG*meC*meA*meG*meG*meG | |
| | | 57 | VPmU*fA*mCfUmUfUmCfUmUfCmAfUmUfUmCfCmAfCmCfU*mU*fU | 22 |
| RD-13015 | SOD1 | 1272 | mA*fG*mGfUmGfGmAfAfAfUfGfAmAfGmAfAmAfG*mU*fA-L9- | 34 |
| | | | meG*meU*meA*meA*meA*meG*meU*meG*meA*meG*meC*meA*meU*meA | |
| | | 57 | VPmU*fA*mCfUmUfUmCfUmUfCmAfUmUfUmCfCmAfCmCfU*mU*fU | 22 |
| RD-13016 | SOD1 | 1273 | mA*fG*mGfUmGfGmAfAfAfUfGfAmAfGmAfAmAfG*mU*fA-L9- | 34 |
| | | | meA*meA*meU*meA*meG*meA*meG*meC*meG*meA*meG*meG*meA*meU | |
| | | 57 | VPmU*fA*mCfUmUfUmCfUmUfCmAfUmUfUmCfCmAfCmCfU*mU*fU | 22 |
| RD-13017 | SOD1 | 1274 | mA*fG*mGfUmGfGmAfAfAfUfGfAmAfGmAfAmAfG*mU*fA-L9- | 34 |
| | | | meC*meG*meC*meA*meG*meA*meG*meA*meA*meU*meG*meA*meA*meA | |
| | | 57 | VPmU*fA*mCfUmUfUmCfUmUfCmAfUmUfUmCfCmAfCmCfU*mU*fU | 22 |
| RD-13018 | SOD1 | 1275 | mA*fG*mGfUmGfGmAfAfAfUfGfAmAfGmAfAmAfG*mU*fA-L9- | 34 |
| | | | meU*meG*meC*meG*meU*meC*meG*meA*meA*meU*meU*meC*meU*meC | |
| | | 57 | VPmU*fA*mCfUmUfUmCfUmUfCmAfUmUfUmCfCmAfCmCfU*mU*fU | 22 |
| RD-13019 | SOD1 | 1276 | mA*G*mGfUmGfGmAfAfAfUfGfAmAfGmAfAmAfG*mU*fA-L9- | 34 |
| | | | meU*meC*meA*meC*meU*meU*meA*meU*meG*meC*meA*meC*meG*meC | |
| | | 57 | VPmU*fA*mCfUmUfUmCfUmUfCmAfUmUfUmCfCmAfCmCfU*mU*fU | 22 |
| RD-13020 | SOD1 | 1277 | mA*fG*mGfUmGfGmAfAfAfUfGfAmAfGmAfAmAfG*mU*fA-L9- | 34 |
| | | | meU*meC*meU*meU*meA*meU*meU*meA*meC*meA*meG*meA*meU*meC | |
| | | 57 | VPmU*fA*mCfUmUfUmCfUmUfCmAfUmUfUmCfCmAfCmCfU*mU*fU | 22 |
| RD-13021 | SOD1 | 1278 | mA*fG*mGfUmGfGmAfAfAfUfGfAmAfGmAfAmAfG*mU*fA-L9- | 34 |
| | | | meU*meU*meC*meC*meC*meC*meU*meU*meG*meA*meU*meA*meA*meC | |
| | | 57 | VPmU*fA*mCfUmUfUmCfUmUfCmAfUmUfUmCfCmAfCmCfU*mU*fU | 22 |
| RD-13022 | SOD1 | 1279 | mA*fG*mGfUmGfGmAfAfAfUfGfAmAfGmAfAmAfG*mU*fA-L9- | 34 |
| | | | meC*meC*meU*meU*meU*meU*meC*meU*meA*meG*meA*meU*meC*meG | |
| | | 57 | VPmU*fA*mCfUmUfUmCfUmUfCmAfUmUfUmCfCmAfCmCfU*mU*fU | 22 |
| RD-13023 | SOD1 | 1280 | mA*fG*mGfUmGfGmAfAfAfUfGfAmAfGmAfAmAfG*mU*fA-L9- | 34 |
| | | | meU*meC*meC*meU*meG*meU*meC*meC*meA*meU*meC*meC*meU*meA | |
| | | 57 | VPmU*fA*mCfUmUfUmCfUmUfCmAfUmUfUmCfCmAfCmCfU*mU*fU | 22 |
| RD-13024 | SOD1 | 1281 | mA*fG*mGfUmGfGmAfAfAfUfGfAmAfGmAfAmAfG*mU*fA-L9- | 34 |
| | | | meU*meU*meC*meU*meA*meU*meG*meU*meG*meC*meU*meC*meC*meC | |
| | | 57 | VPmU*fA*mCfUmUfUmCfUmUfCmAfUmUfUmCfCmAfCmCfU*mU*fU | 2.2 |
| RD-13025 | SOD1 | 1282 | mA*fG*mGfUmGfGmAfAfAfUfGfAmAfGmAfAmAfG*mU*fA-L9- | 34 |
| | | | meU*meA*meC*meU*meC*meC*meU*meU*meU*meC*meA*meU*meU*meC | |
| | | 57 | VPmU*fA*mCfUmUfUmCfUmUfCmAfUmUfUmCfCmAfCmCfU*mU*fU | 22 |
| RD-13026 | SOD1 | 1283 | mA*fG*mGfUmGfGmAfAfAfUfGfAmAfGmAfAmAfG*mU*fA-L9- | 34 |
| | | | meG*meG*meC*meU*meA*meU*meU*meA*meU*meC*meC*meA*meU*meA | |
| | | 57 | VPmU*fA*mCfUmUfUmCfUmUfCmAfUmUfUmCfCmAfCmCfU*mU*fU | 22 |
| RD-13027 | SOD1 | 1284 | mA*fG*mGfUmGfGmAfAfAfUfGfAmAfGmAfAmAfG*mU*fA-L9- | 34 |
| | | | meU*meU*meA*meC*meU*meC*meA*meG*meG*meC*meA*meG*meG*meA | |
| | | 57 | VPmU*fA*mCfUmUfUmCfUmUfCmAfUmUfUmCfCmAfCmCfU*mU*fU | 22 |
| RD-13028 | SOD1 | 1285 | mA*fG*mGfUmGfGmAfAfAfUfGfAmAfGmAfAmAfG*mU*fA-L9- | 34 |
| | | | meA*meU*meG*meU*meG*meC*meA*meA*meC*meC*meA*meU*meC*meG | |
| | | 57 | VPmU*fA*mCfUmUfUmCfUmUfCmAfUmUfUmCfCmAfCmCU*mU*fU | 22 |
| RD-13029 | SOD1 | 1286 | mA*fG*mGfUmGfGmAfAfAfUfGfAmAfGmAfAmAfG*mU*fA-L9- | 34 |
| | | | meC*meC*meG*meG*meU*meG*meU*meA*meU*meG*meU*meC*meA*meA | |
| | | 57 | VPmU*fA*mCfUmUfUmCfUmUfCmAfUmUfUmCfCmAfCmCfU*mU*fU | 22 |
| RD-13030 | SOD1 | 1287 | mA*fG*mGfUmGfGmAfAfAfUfGfAmAfGmAfAmAfG*mU*fA-L9- | 34 |
| | | | meU*meU*meU*meG*meU*meC*meG*meG*meU*meA*meC*meC*meA*meA | |
| | | 57 | VPmU*fA*mCfUmUfUmCfUmUfCmAfUmUfUmCfCmAfCmCfU*mU*fU | 22 |
| RD-13031 | SOD1 | 1288 | mA*fG*mGfUmGfGmAfAfAfUfGfAmAfGmAfAmAfG*mU*fA-L9- | 34 |
| | | | meC*meU*meC*meU*meA*meA*meG*meG*meC*meA*meC*meC*meU*meG | |
| | | 57 | VPmU*fA*mCfUmUfUmCfUmUfCmAfUmUfUmCfCmAfCmCfU*mU*fU | 22 |

¶n.b.: upper case, RNA; *, phosphorothioate (PS) backbone modification; f, 2′-fluoro; m, 2′-O-methyl (2′-OMe); me, 2′-O-methoxyethyl (2′MOE); VP, 5′-(E)-vinylphosphonate; C, 5′-methyl cytosine; U, 5′-methyl uracil; S18, spacer-18 linker; S9, spacer-9 linker; L9, spacer-9 linker; C3, spacer-C3 linker; C6, spacer-C6 linker; C12, spacer-C12 linker; UUACA, L7 linker; UUCUU, L8 linker; d spacer, L12 linker; L14, spacer-L 14 linker; L15, spacer-L15 linker; L16, spacer-L16 linker.

TABLE 29
Oligonucleotide strand sequences and duplex compositions

| ODV- | | SEQ | | |
|---|---|---|---|---|
| siRNA | Target | ID | | Size |
| name | gene | NO | Sequence (5′-3′) | (bp) |
| RD-12404 | SOD1 | 947 | fU*mA*fCmUfUmUfUmGfUfGfUmAfGmUfAmCfA*mA*fA-S9- | 33 |
| | | | meU*meU*meG*meU*meA*meU*meU*meC*meU*meA*meU*meG*meU*meU | |
| | | 48 | mU*fU*mUfGmUfAmCfUmAfCmAfCmAfAmAfAmGfUmA*fC*mU | 21 |
| RD-12401 | SOD1 | 948 | GGUGGAAAUGAAGAAAGUA | 19 |
| | | 949 | UACUUUCUUCAUUUCCACCUU | 21 |
| RD-12402 | SOD1 | 46 | fG*mG*fUmGfGmAfAmAfUfGfAmAfGmAfAmAf*GmU*fA | 19 |
| | | 47 | mU*fA*mCfUmUfUmCfUmUfCmAfUmUfUmCfCmAfCmC*fU*mU | 21 |
| RD-12403 | SOD1 | 45 | fG*mG*fUmGfGmAfAmAfUfGfAmAfGmAfAmAf*GmU*fA-S9- | 33 |
| | | | meU*meU*meG*meU*meA*meU*meU*meC*meU*meA*meU*meG*meU*meU | |
| | | 47 | mU*A*mCfUmUfUmCfUmUfCmAfUmUfUmCfCmAfCmC*fU*mU | 21 |
| RD-12557 | SOD1 | 45 | fG*mG*fUmGfGmAfAmAfUfGfAmAfGmAfAmAf*GmU*fA-S9- | 33 |
| | | | meU*meU*meG*meU*meA*meU*meU*meC*meU*meA*meU*meG*meU*meU | |
| | | 57 | VPmU*fA*mCfUmUfUmCfUmUfCmAfUmUfUmCfCmAfCmC*U*mU | 22 |
| RD-12556 | SOD1 | 1196 | mA*fG*mGfUmGfGmAfAfAfUfGfAmAfGmAfAmAfG*mU*fA | 20 |
| | | 57 | VPmU*fA*mCfUmUfUmCfUmUfCmAfUmUfUmCfCmAfCmCfU*mU*fU | 22 |
| RD-12559 | SOD1 | 1197 | mA*fG*mGfUmGfGmAfAfAfUfGfAmAfGmAfAmAfG*mU*fA-S9- | 34 |
| | | | meU*meU*meG*meU*meA*meU*meU*meC*meU*meA*meU*meG*meU*meU | |
| | | 57 | VPmU*fA*mCfUmUfUmCfUmUfCmAfUmUfUmCfCmAfCmCfU*mU*fU | 22 |
| RD-12929 | SOD1 | 1197 | mA*fG*mGfUmGfGmAfAfAfUfGfAmAfGmAfAmAfG*mU*fA-S9- | 34 |
| | | | meU*meU*meG*meU*meA*meU*meU*meC*meU*meA*meU*meG*meU*meU | |
| | | 47 | mU*fA*mCfUmUfUmCfUmUfCmAfUmUfUmCfCmAfCmCfU*mU*fU | 21 |

¶n.b.: upper case, RNA; *, phosphorothioate (PS) backbone modification; f, 2′-fluoro; m, 2′-O-methyl (2′-OMe); me, 2′-O-methoxyethyl (2′MOE); VP, 5′-(E)-vinylphosphonate; C, 5′-methyl cytosine; U, 5′-methyl uracil; S9, spacer-9 linker;

TABLE 30
Oligonucleotide strand sequences and duplex compositions

| ODV- | | SEQ | | |
|---|---|---|---|---|
| siRNA | Target | ID | | Size |
| name | gene | NO | Sequence (5′-3′) | (bp) |
| DS17- | SOD1 | 1289 | AGGUGGAAAUGAAGAAAGUA | 20 |
| 29N3 | | 1290 | UACUUUCUUCAUUUCCACCUUU | 22 |
| RD-12559 | SOD1 | 1197 | mA*fG*mGfUmGfGmAfAfAfUfGfAmAfGmAfAmAfG*mU*fA-S9- | 34 |
| | | | meU*meU*meG*meU*meA*meU*meU*meC*meU*meA*meU*meG*meU*meU | |
| | | 57 | VPmU*fA*mCfUmUfUmCfUmUfCmAfUmUfUmCfCmAfCmCfU*mU*fU | 22 |
| RD-12952 | SOD1 | 1209 | mA*fG*mGfUmGfGmAfAfAfUfGfAmAfGmAfAmAfG*mU*fA-L9- | 34 |
| | | | meU*meU*meG*meU*meA*meU*meU*meC*meU*meU*meG*meU*meA*meU | |
| | | 57 | VPmU*fA*mCfUmUfUmCfUmUfCmAfUmUfUmCfCmAfCmCfU*mU*fU | 22 |
| RD-12982 | SOD1 | 1239 | mA*fG*mGfUmGfGmAfAfAfUfGfAmAfGmAfAmAfG*mU*fA-L9- | 31 |
| | | | meU*meU*meG*meU*meA*meU*meU*meC*meU*meA*meU | |
| | | 57 | VPmU*fA*mCfUmUfUmCfUmUfCmAfUmUfUmCfCmAfCmCfU*mU*fU | 22 |
| RD-12983 | SOD1 | 1240 | mA*fG*mGfUmGfGmAfAfAfUfGfAmAfGmAfAmAfG*mU*fA-L9- | 30 |
| | | | meU*meU*meG*meU*meA*meU*meU*meC*meU*meA | |
| | | 57 | VPmU*fA*mCfUmUfUmCfUmUfCmAfUmUfUmCfCmAfCmCfU*mU*fU | 22 |
| RD-12979 | SOD1 | 1236 | mA*fG*mGfUmGfGmAfAfAfUfGfAmAfGmAfAmAfG*mU*fA-L9- | 34 |
| | | | mU*mU*mG*mU*mA*mU*mU*mC*mU*mA*mU*mG*mU*mU | |
| | | 57 | VPmU*fA*mCfUmUfUmCfUmUfCmAfUmUfUmCfCmAfCmCfU*mU*fU | 22 |
| RD-13015 | SOD1 | 1272 | mA*fG*mGfUmGfGmAfAfAfUfGfAmAfGmAfAmAfG*mU*fA-L9- | 34 |
| | | | meG*meU*meA*meA*meA*meG*meU*meG*meA*meG*meC*meA*meU*meA | |
| | | 57 | VPmU*fA*mCfUmUfUmCfUmUfCmAfUmUfUmCfCmAfCmCfU*mU*fU | 22 |
| RD-13006 | SOD1 | 1263 | mA*fG*mGfUmGfGmAfAfAfUfGfAmAfGmAfAmAfG*mU*fA-L9- | 34 |
| | | | meU*meA*meU*meG*meU*meC*meG*meU*meA*meU*meU*meG*meU*meA | |
| | | 57 | VPmU*fA*mCfUmUfUmCfUmUfCmAfUmUfUmCfCmAfCmCfU*mU*fU | 22 |
| RD-12998 | SOD1 | 1255 | mA*fG*mGfUmGfGmAfAfAfUfGfAmAfGmAfAmAfG*mU*fA-L9- | 34 |
| | | | meC*meC*meG*meC*meU*meC*meC*meC*meC*meC*meC*meU*meA*meC | |
| | | 57 | VPmU*fA*mCfUmUfUmCfUmUfCmAfUmUfUmCfCmAfCmCfU*mU*fU | 22 |
| RD-13150 | SOD1 | 1291 | mA*fG*mGfUmG-C6x1-mAfAfAfUfGfAmAfGmAfAmAfG*mU*fA | 19 |
| | | 57 | VPmU*fA*mCfUmUfUmCfUmUfCmAfUmUfUmCfCmAfCmCfU*mU*fU | 22 |
| RD-13151 | SOD1 | 1292 | mA*fG*mGfUmG-C6x2-mAfAfAfUfGfAmAfGmAfAmAfG*mU*fA | 19 |
| | | 57 | VPmU*fA*mCfUmUlUmCfUmUfCmAfUmUfUmCfCmAfCmCfU*mU*fU | 22 |
| RD-13356 | SOD1 | 1293 | mA*fG*mGfUmG-C6x5-mAfAfAfUfGfAmAfGmAfAmAfG*mU*fA | 19 |
| | | 57 | VPmU*fA*mCfUmUfUmCfUmUfCmAfUmUfUmCfCmAfCmCfU*mU*fU | 22 |
| RD-13351 | SOD1 | 1294 | mA*fG*mGfUmG-(C6x7- | 33 |
| | | | meU*meU*meG*meU*meA*meU*meU*meC*meU*meA*meU*meG*meU*meU)- | |
| | | | mAfAfAfUfGfAmAfGmAfAmAfG*mU*fA | |
| | | 57 | VPmU*fA*mCfUmUfUmCfUmUfCmAfUmUfUmCfCmAfCmCfU*mU*fU | 22 |
| RD-13180 | SOD1 | 1295 | mA*fG*mGfUmGfGmAfAfAfUfGfAmAfGmAfAmAfG*mU*fA*S9*meU*meU*meG | 34 |
| | | | *meU*meA*meU*meU*meC*meU*meA*meU*meG*meU*meU | |
| | | 57 | VPmU*fA*mCfUmUfUmCfUmUfCmAfUmUfUmCfCmAfCmCfU*mU*fU | 22 |
| RD-12941 | SOD1 | 1296 | mA*fG*mGfUmGfGmAfAfAfUfGfAmAfGmAfAmAfG*mU*fA-S18- | 34 |
| | | | meU*meU*meG*meU*meA*meU*meU*meC*meU*meA*meU*meG*meU*meU | |
| | | 57 | VPmU*fA*mCfUmUfUmCfUmUfCmAfUmUfUmCfCmAfCmCfU*mU*fU | 22 |
| RD-12942 | SOD1 | 1297 | mA*fG*mGfUmGfGmAfAfAfUfGfAmAfGmAfAmAfG*mU*fA-C6- | 34 |
| | | | meU*meU*meG*meU*meA*meU*meU*meC*meU*meA*meU*meG*meU*meU | |
| | | 57 | VPmU*fA*mCfUmUfUmCfUmUfCmAfUmUfUmCfCmAfCmCfU*mU*fU | |
| RD-12967 | SOD1 | 1298 | mA*fG*mGfUmGfGmAfAfAfUfGfAmAfGmAfAmAfG*mU*fA-L9- | 34 |
| | | | meU*meUmeGmeUmeAmeUmeUmeCmeUmeAmeUmeGmeU*meU | |
| | | 57 | VPmU*fA*mCfUmUfUmCfUmUfCmAfUmUfUmCfCmAfCmCfU*mU*fU | 22 |

¶n.b.: upper case, RNA; *, phosphorothioate (PS) backbone modification; f, 2′-fluoro; m, 2′-O-methyl (2′-OMe); me, 2′-O-methoxyethyl (2′MOE); VP, 5′-(E)-vinylphosphonate; C, 5′-methyl cytosine; U, 5′-methyl uracil; S9, spacer-9 linker; C6x1, C6x1 linker; C6x2, C6x2 linker; C6x5, C6x5 linker; C6x7, C6x7 linker,

### EQUIVALENTS AND INCORPORATION BY REFERENCE

All references cited herein are incorporated by reference to the same extent as if each individual publication, database entry (e.g., Genbank sequences or GeneID entries), patent application, or patent, was specifically and individually indicated incorporated by reference in its entirety, for all purposes. This statement of incorporation by reference is intended by Applicants, pursuant to 37 C.F.R. § 1.57(b)(1), to relate to each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

While the application has been particularly shown and described with reference to a preferred embodiment and various alternate embodiments, it is understood by persons skilled in the relevant art that various changes in form and details can be made therein without departing from the spirit and scope of the application.

1. An oligonucleotide agent comprising a non-targeting single-stranded oligonucleotide, wherein the single-stranded oligonucleotide is at least 6 nucleotides in length, wherein the single-stranded oligonucleotide is capable of facilitating delivery of a double-stranded oligonucleotide, wherein at least one phosphodiester bond between two adjacent nucleotides in the single-stranded oligonucleotide sequence is substituted by a phosphorothioate (PS), mesyl phosphoramidate or boranophosphate bond; and wherein the double-stranded oligonucleotide comprises a sense strand and an antisense strand.

2. (canceled)

3. (canceled)

4. The oligonucleotide agent according to claim 1, wherein the single-stranded oligonucleotide and the double-stranded oligonucleotide is conjugated with zero, one or more linking components.

5. The oligonucleotide agent according to claim 1, wherein all nucleotides of the single-stranded oligonucleotide are non-chemically modified nucleotides, or at least one nucleotide is a chemically modified nucleotide; and/or wherein all nucleotides of the double-stranded oligonucleotide are non-chemically modified nucleotides, or at least one nucleotide is a chemically modified nucleotide, or at least one phosphodiester bond between two adjacent nucleotides in the nucleotide sequence is substituted by a phosphorothioate, mesyl phosphoramidate or boranophosphate bond.

6. (canceled)

7. The oligonucleotide agent according to claim 5, wherein the chemically modified nucleotide comprises one or more of the following modifications:
a. modification of 2′-OH of the ribose in the nucleotide;
b. modification or absence of a base moiety on the nucleoside ring in the nucleotide;
c. a nucleotide being a locked or bridged nucleic acid, and
d. a nucleotide being a deoxyribonucleotide (DNA); and/or
wherein the chemically modified nucleotide has a 2′-OH ribose modification selected from: a 2′-fluoro-2′-deoxynucleoside (2′-F) modification, a 2′-O-methyl (2′-O-Me) modification, and a 2′-O-(2-methoxyethyl) (2′-O-MOE) modification; and/or
wherein the single-stranded oligonucleotide is composed of one or more nucleotides selected from the group of RNA, DNA, bridged nucleic acid (BNA), locked nucleic acid (LNA) and peptide nucleic acid (PNA).

8. (canceled)

9. (canceled)

10. The oligonucleotide agent according to claim 1, wherein the single-stranded oligonucleotide comprises at least one phosphorothioate (PS) backbone substitution.

11. (canceled)

12. (canceled)

13. The oligonucleotide agent according to claim 5, wherein the at least one chemically modified nucleotide is a nucleotide having an addition of a 5′-phosophate moiety at the 5′ end of the nucleotide sequence, or a nucleotide having an addition of a 5-methyl cytosine moiety at the 5′ end of the nucleotide sequence.

14. The oligonucleotide agent according to claim 13, wherein at least one chemically modified nucleotide is a nucleotide having an addition of a 5′-(E)-vinylphosphonate moiety.

15. (canceled)

16. The oligonucleotide agent according to claim 1, wherein the single-stranded oligonucleotide is 6-22 nucleotides in length.

17. (canceled)

18. (canceled)

19. The oligonucleotide agent according to claim 1, wherein the single strand oligonucleotide comprises:
1) a nucleotide sequence that is at least 90% identical to the nucleotide sequence selected from the group consisting of SEQ ID NOs: 1-22;
2) a chemically modified nucleotide sequence that is at least 80% identical to a nucleotide sequence selected from the group of SEO ID NOs: 1299-1379; or
3) a chemically modified nucleotide sequence having 0, 1, 2 or 3 different chemical modifications than a nucleotide sequence selected from the group consisting of SEO ID NOs: 1299-1379.

20. The oligonucleotide agent according to claim 1, wherein the nucleotide sequence of the single-stranded oligonucleotide comprises 35-65% adenines; and/or
wherein the nucleotide sequence of the single-stranded oligonucleotide comprises 35-72% cytosines; and/or
wherein the nucleotide sequence of the single-stranded oligonucleotide comprises 35-65% guanosines; and/or
wherein the nucleotide sequence of the single-stranded oligonucleotide comprises 35-72% uracil; and/or
wherein the nucleotide sequence of the single-stranded oligonucleotide comprises 64-78% purines; and/or
wherein the nucleotide sequence of the single-stranded oligonucleotide comprises 64-86% pyrimidines; and/or
wherein the nucleotide sequence of the single-stranded oligonucleotide comprises 42-58% purines and 42-58% pyrimidines.

21. (canceled)

22. (canceled)

23. (canceled)

24. (canceled)

25. (canceled)

26. (canceled)

27. The oligonucleotide agent according to claim 1, wherein the nucleotide sequence of the single-stranded oligonucleotide comprises at least about 14%, at least about 28%, at least about 42%, at least about 57%, at least about 71%, at least about 85%, at least about 92%, or about 100% of the nucleotides having a 2′Ome modification.

28. The oligonucleotide agent according to claim 1, wherein the nucleotide sequence of the single-stranded oligonucleotide is a palindrome sequence.

29. (canceled)

30. (canceled)

31. The oligonucleotide agent according to claim 1, wherein one or more of the double-stranded oligonucleotide and one or more of the single-stranded oligonucleotide are covalently conjugated by a linking component.

32. (canceled)

33. (canceled)

34. (canceled)

35. (canceled)

36. (canceled)

37. (canceled)

38. (canceled)

39. (canceled)

40. (canceled)

41. (canceled)

42. The oligonucleotide agent according to claim 31, wherein the linking component conjugated with the nucleotide in the single-stranded oligonucleotide or the double-stranded oligonucleotide, or both the single-stranded oligonucleotide and the double-stranded oligonucleotide through a phosphorothioate (PS) bond.

43. (canceled)

44. (canceled)

45. The oligonucleotide agent according to claim 4, wherein the linking component comprises a direct bond, or an oxygen or sulfur atom, or a unit selected from the following group: NR1, C(O), C(O)O, C(O)NR1, SO, SO2, and SO2NH; where R1 is hydrogen, acyl, aliphatic or substituted aliphatic; or the linking component is selected from the group consisting of: substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, wherein one or more methylenes are interrupted or terminated by O, S, S(O), SO2, N(R′)2, C(O), cleavable linking group, substituted or unsubstituted aryl, substituted or unsubstituted heteroaryl, and substituted or unsubstituted heterocyclic; or
the linking component is selected from one or more of an ethylene glycol chain, an alkyl chain, an alkenyl chain, an alkynyl chain, a peptide, carbohydrates, thiol linkage, a phosphodiester, a phosphorothioate, a phosphoramidate, an amide, a carbamate, a tetrazole linkage, and a benzimidazole linkage.

46. (canceled)

47. (canceled)

48. The oligonucleotide agent according to claim 4, wherein the linking component is selected from the group consisting of:
a) L1 or S18 (spacer-18 linker) (1,1-bis(4-methoxyphenyl)-1-phenyl-2,5,8,11,14,17-hexaoxanonadecan-19-yl (2-cyanoethyl) diisopropylphosphoramidite);
b) L4 or C6 (spacer-C6 linker) (6-(bis(4-methoxyphenyl)(phenyl)methoxy)hexyl (2-cyanoethyl) diisopropylphosphoramidite);
c) L6 (1,1-bis(4-methoxyphenyl)-1-phenyl-2,5,8,11,14-pentaoxahexadecan-16-yl (2-cyanoethyl) diisopropylphosphoramidite);
d) L9 or S9 (spacer-9 linker) (2-(2-(2-(bis(4-methoxyphenyl)(phenyl)methoxy)ethoxy)ethoxy)ethyl (2-cyanoethyl) diisopropylphosphoramidite);
e) L10 or C3 (spacer-C3 linker) (3-(bis(4-methoxyphenyl)(phenyl)methoxy)propyl (2-cyanoethyl) diisopropylphosphoramidite);
f) L12 (d spacer) ((2R,3S)-2-((bis(4-methoxyphenyl)(phenyl)methoxy)methyl)tetrahydrofuran-3-yl (2-cyanoethyl) diisopropylphosphoramidite);
g) L13 or C12 (spacer-C12 linker) (12-(bis(4-methoxyphenyl)(phenyl)methoxy)dodecyl (2-cyanoethyl) diisopropylphosphoramidite);
h) L14 (spacer-L14 linker) (((1r,4r)-4-((bis(4-methoxyphenyl)(phenyl)methoxy)methyl)cyclohexyl)methyl (2-cyanoethyl) diisopropylphosphoramidite);
i) L15 (spacer-L15 linker) (4-(2-(bis(4-methoxyphenyl)(phenyl)methoxy)ethyl)phenethyl (2-cyanoethyl) diisopropylphosphoramidite);
j) L16 (spacer-L16 linker) (2-(1-(2-(bis(4-methoxyphenyl)(phenyl)methoxy)ethyl)cyclohexyl)ethyl(2-cyanoethyl) diisopropylphosphoramidite);
k) C6x1 ((2S,3S,4S,5S)-2-((bis(4-methoxyphenyl)(phenyl)methoxy)methyl)-5-methoxy-4-(pent-4-yn-1-yloxy)tetrahydrofuran-3-yl (2-cyanoethyl) diisopropylphosphoramidite);
l) C6x2 ((2S,3S,4S,5S)-5-((bis(4-methoxyphenyl)(phenyl)methoxy)methyl)-2-methoxy-4-(pent-4-yn-1-yloxy)tetrahydrofuran-3-yl (2-cyanoethyl) diisopropylphosphoramidite);
m) C6x5 (2-((2-(bis(4-methoxyphenyl)(phenyl)methoxy)ethyl)(pent-4-yn-1-yl)amino)ethyl (2-cyanoethyl) diisopropylphosphoramidite); and
n) C6x7 ((9H-fluoren-9-yl)methyl (4-((2S,4R)-2-((bis(4-methoxyphenyl)(phenyl)methoxy)methyl)-4-((bis(diisopropylamino)phosphanyl)oxy)pyrrolidin-1-yl)-4-oxobutyl)carbamate).

49. The oligonucleotide agent of claim 4, wherein the oligonucleotide agent comprises a nucleotide sequence that is at least 90% identical to the nucleotide sequences selected from the group consisting of:
1) siSOD1M2-AC2 (N22)-S1V3v-Qu5 (SEQ ID NO: 58) and an antisense strand having a nucleotide sequence of SEQ ID NO: 57 that has partial complementarity with the sense strand of siSOD1M2-AC2 (N22)-S1V3v-Qu5 (SEQ ID NO: 58);
2) siSOD1M2-AC2 (N15)-S1V3v-Qu5 (SEQ ID NO: 60) and an antisense strand having a nucleotide sequence of SEQ ID NO: 57 that has partial complementarity with the sense strand of siSOD1M2-AC2 (N15)-S1V3v-Qu5 (SEQ ID NO: 60);
3) siSOD1M2-AC2 (N12)-S1V3v-Qu5 (SEQ ID NO: 62) and an antisense strand having a nucleotide sequence of SEQ ID NO: 57 that has partial complementarity with the sense strand of siSOD1M2-AC2 (N12)-S1V3v-Qu5 (SEQ ID NO: 62); and
4) siSOD1M2-AC2 (N6)-S1V3v-Qu5 (SEQ ID NO: 64) and an antisense strand having a nucleotide sequence of SEQ ID NO: 57 that has partial complementarity with the sense strand of siSOD1M2-AC2 (N6)-S1V3v-Qu5 (SEQ ID NO: 64);
5) siHTT-AC2-S1L1 (SEQ ID NO: 28) and an antisense strand having a nucleotide sequence of SEQ ID NO: 27 that has partial complementarity with the sense strand of siHTT-AC2-S1L1 (SEQ ID NO: 28);
6) siApp-8-AC2 (N18)-S1L1V3v (SEQ ID NO: 32) and an antisense strand having a nucleotide sequence of SEQ ID NO: 31 that has partial complementarity with the sense strand of siApp-8-AC2 (N18)-S1L1V3v (SEQ ID NO: 32);
7) siApp-8-AC2 (N15)-S1L1V3v (SEQ ID NO: 34) and an antisense strand having a nucleotide sequence of SEQ ID NO: 31 that has partial complementarity with the sense strand of siApp-8-AC2 (N15)-S1L1V3v (SEQ ID NO: 34);
8) siApp-8-AC2 (N12)-S1L1V3v (SEQ ID NO: 36) and an antisense strand having a nucleotide sequence of SEQ ID NO: 31 that has partial complementarity with the sense strand of siApp-8-AC2 (N12)-S1L1V3v (SEQ ID NO: 36);
9) R6-04M1-AC2 (18)-S1L1V3v (SEQ ID NO: 68) and an antisense strand having a nucleotide sequence of SEQ ID NO: 67 that has partial complementarity with the sense strand of R6-04M1-AC2 (18)-S1L1V3v (SEQ ID NO: 68);
10) R6-04M1-AC2 (16)-S1L1V3v (SEQ ID NO: 70) and an antisense strand having a nucleotide sequence of SEQ ID NO: 67 that has partial complementarity with the sense strand of R6-04M1-AC2 (16)-S1L1V3v (SEQ ID NO: 70);
11) R6-04M1-AC2 (15)-S1L1V3v (SEQ ID NO: 72) and an antisense strand having a nucleotide sequence of SEQ ID NO: 67 that has partial complementarity with the sense strand of R6-04M1-AC2 (15)-S1L1V3v (SEQ ID NO: 72);
12) R6-04M1-AC2 (14)-S1L1V3v (SEQ ID NO: 74) and an antisense strand having a nucleotide sequence of SEQ ID NO: 67 that has partial complementarity with the sense strand of R6-04M1-AC2 (14)-S1L1V3v (SEQ ID NO: 74);
13) R6-04M1-AC2 (13)-S1L1V3v (SEQ ID NO: 76) and an antisense strand having a nucleotide sequence of SEQ ID NO: 67 that has partial complementarity with the sense strand of R6-04M1-AC2 (13)-S1L1V3v (SEQ ID NO: 76);
14) R6-04M1-AC2 (12)-S1L1V3v (SEQ ID NO: 78) and an antisense strand having a nucleotide sequence of SEQ ID NO: 67 that has partial complementarity with the sense strand of R6-04M1-AC2 (12)-S1L1V3v (SEQ ID NO: 78);
15) R6-04M1-AC2 (11)-S1L1V3v (SEQ ID NO: 80) and an antisense strand having a nucleotide sequence of SEQ ID NO: 67 that has partial complementarity with the sense strand of R6-04M1-AC2 (11)-S1L1V3v (SEQ ID NO: 80);
16) R6-04M1-AC2 (10)-S1L1V3v (SEQ ID NO: 82) and an antisense strand having a nucleotide sequence of SEQ ID NO: 67 that has partial complementarity with the sense strand of R6-04M1-AC2 (10)-S1L1V3v (SEQ ID NO: 82);
17) R6-04M1-AC2 (9)-S1L1V3v (SEQ ID NO: 84) and an antisense strand having a nucleotide sequence of SEQ ID NO: 67 that has partial complementarity with the sense strand of R6-04M1-AC2 (9)-S1L1V3v (SEQ ID NO: 84); and
18) R6-04M1-AC2 (8)-S1L1V3v (SEQ ID NO: 86) and an antisense strand having a nucleotide sequence of SEQ ID NO: 67 that has partial complementarity with the sense strand of R6-04M1-AC2 (8)-S1L1V3v (SEQ ID NO: 86).

50. (canceled)

51. (canceled)

52. (canceled)

53. (canceled)

54. The oligonucleotide agent according to claim 1, wherein the single-stranded oligonucleotide or the double-stranded oligonucleotide is conjugated to one or more conjugation groups.

55. (canceled)

56. (canceled)

57. (canceled)

58. (canceled)

59. The oligonucleotide agent according to claim 1, wherein the double-stranded oligonucleotide is a small interfering RNA (siRNA) or a small activating RNA (saRNA).

60. The oligonucleotide agent according to claim 1, wherein the sense or antisense strand of the double-stranded oligonucleotide has a nucleotide sequence that is at least 90% identical to the nucleotide sequence siApp-8-S1V1 (SEQ ID NO: 28) or siApp-8-S1V1 (SEQ ID NO: 27); or the sense or antisense strand of the double-stranded oligonucleotide has a nucleotide sequence that is at least 90% identical to the nucleotide sequence R6-04 (20)-S1V1v (CM-4) (SEQ ID NO: 66) or R6-04 (20)-S1V1v (CM-4) (SEQ ID NO: 67).

61. The oligonucleotide agent according to claim 1, wherein the oligonucleotide agent comprises a small interfering RNA (siRNA), wherein the siRNA comprises a sense strand and an antisense strand to form a duplex structure, wherein the antisense strand comprises a nucleotide sequence comprising at least 10 contiguous nucleotides, with 0, 1, 2 or 3 mismatches, and having at least 85% nucleotide sequence complementarity or homology to a portion of the nucleotide sequence of SEQ ID NO: 895, SEQ ID NO: 59, SEQ ID NO: 61, SEQ ID NO:61, or any one of SEQ ID NO: 1068-1113; wherein the oligonucleotide agent is capable of inhibiting expression of superoxide dismutase 1 (SOD1) in a cell.

62. The oligonucleotide agent according to claim 61, wherein the sense strand of the double-stranded oligonucleotide has a nucleotide sequence that is at least 90% identical to the nucleotide sequence selected from the group consisting of: siSOD1-5 (SEQ ID NO: 357), siSOD1-8 (SEQ ID NO: 358), siSOD1-10 (SEQ ID NO:
359), siSOD1-11 (SEQ ID NO: 360), siSOD-17 (SEQ ID NO: 357), siSOD1-35 (SEQ ID NO: 362), and siSOD1-37 through siSOD1-447 (SEQ ID NOs: 363-624), siSOD1-547 through siSOD1-694 (SEQ ID NOs: 976-1021), siSOD1-231-E (SEQ ID NO: 38), siSOD1-231-TT (SEQ ID NO: 40), siSOD1-231-M1 (SEQ ID NO: 42), siSOD1-231-S2 (SEQ ID NO: 44), siSOD1-388-E (SEQ ID NO: 46), siSOD1-388-TT (SEQ ID NO: 48), siSOD1-388-M1 (SEQ ID NO: 50), siSOD1-388-S2 (SEQ ID NO: 52), siSODIM2-L1 (SEQ ID NO: 54), and siSODIM2-S1V5 (SEQ ID NO: 56).

63. The oligonucleotide agent according to claim 61, wherein the antisense strand of the double-stranded oligonucleotide has a nucleotide sequence that is at least 90% identical to the nucleotide sequence selected from the group consisting of: siSOD1-5 (SEQ ID NO: 626), siSOD1-8 (SEQ ID NO: 627), siSOD1-10 (SEQ ID NO: 628), siSOD1-11 (SEQ ID NO: 629), siSOD-17 (SEQ ID NO: 630), siSOD1-35 (SEQ ID NO: 631), and siSOD1-37-through siSOD1-447 (SEQ ID NOs: 632-893), siSOD1-547 through siSOD1-694 (SEQ ID NOs: 1022-1067), siSOD1-231-E (SEQ ID NO: 39), siSOD1-231-TT (SEQ ID NO: 41), siSOD1-231-M1 (SEQ ID NO: 43), siSOD1-231-S2 (SEQ ID NO: 45), siSOD1-388-E (SEQ ID NO: 47), siSOD1-388-TT (SEQ ID NO: 49), siSOD1-388-M1 (SEQ ID NO: 51), siSOD1-388-S2 (SEQ ID NO: 53), siSODIM2-L1 (SEQ ID NO: 47); and siSOD1M2-S1V1v-Qu5 (SEQ ID NO: 57).

64. (canceled)

65. (canceled)

66. The oligonucleotide agent according to claim 6160, wherein the sense strand of the siRNA has a nucleotide sequence that has at least 85% homology to the nucleotide sequence selected from the group consisting of: DS17-0001 (SEQ ID NO: 384), DS17-0002 (SEQ ID NO: 372), DS17-0003 (SEQ ID NO: 409), DS17-0004 (SEQ ID NO: 357), DS17-0005 (SEQ ID NO: 486), DS17-0029 (SEQ ID NO: 588), DS17-01N3 (SEQ ID NO: 912), DS17-02N3 (SEQ ID NO: 914), DS17-03N3 (SEQ ID NO: 916), DS17-04N3 (SEQ ID NO: 918), DS17-05N3 (SEQ ID NO: 920) and SEQ ID NOs: 976-1021.

67. The oligonucleotide agent according to claim 61, wherein the antisense strand of the siRNA has a nucleotide sequence that has at least 85% homology to the nucleotide sequence selected from the group consisting of: DS17-0001 (SEQ ID NO: 653), DS17-0002 (SEQ ID NO: 641), DS17-0003 (SEQ ID NO: 678), DS17-0004 (SEQ ID NO: 626), DS17-0005 (SEQ ID NO: 755), DS17-0029 (SEQ ID NO: 857), DS17-01N3 (SEQ ID NO: 913), DS17-02N3 (SEQ ID NO: 915), DS17-03N3 (SEQ ID NO: 917), DS17-04N3 (SEQ ID NO: 919), DS17-05N3 (SEQ ID NO: 921), and SEQ ID NOs: 1022-1067.

68. The oligonucleotide agent according to claim 61, wherein the sense strand and the antisense strand of the siRNA have nucleotide sequences that are independently at least 85% homologous to the nucleotide sequence pairs selected from the following groups:
(a)
(SEQ ID NO: 384 and SEQ ID NO: 653)
DS17-0001,
(b)
(SEQ ID NO: 372 and SEQ ID NO: 641)
DS17-0002,
(c)
(SEQ ID NO: 409 and SEQ ID NO: 678)
DS17-0003,
(d)
(SEQ ID NO: 357 and SEQ ID NO: 626)
DS17-0004,
(e)
(SEQ ID NO: 486 and SEQ ID NO: 755)
DS17-0005,
(f)
(SEQ ID NO: 588 and SEQ ID NO: 857)
DS17-0029,
(g)
(SEQ ID NO: 912 and SEQ ID NO: 913)
DS17-01N3,
(h)
(SEQ ID NO: 914 and SEQ ID NO: 915)
DS17-02N3,
(i)
(SEQ ID NO: 916 and SEQ ID NO: 917)
DS17-03N3,
(j)
(SEQ ID NO: 918 and SEQ ID NO: 919)
DS17-04N3,
(k)
(SEQ ID NO: 920 and SEQ ID NO: 921)
DS17-05N3,
(l)
(SEQ ID NO: 922 and SEQ ID NO: 923)
DS17-01M3,
(m)
(SEQ ID NO: 924 and SEQ ID NO: 925)
DS17-02M3,
(n)
(SEQ ID NO: 926 and SEQ ID NO: 927)
DS17-03M3,
(o)
(SEQ ID NO: 928 and SEQ ID NO: 929)
DS17-04M3,
and
(p)
(SEQ ID NO: 930 and SEQ ID NO: 931)
DS17-05M3.

69. (canceled)

70. The oligonucleotide agent according to claim 1, wherein the oligonucleotide agent comprises an siRNA and a non-targeting ACO, wherein the siRNA comprises a sense strand and an antisense strand, wherein the ACO comprises a nucleotide sequence that is at least 90%, at least 95%, or 100% identical to SEQ ID NO: 954, and the oligonucleotide agent is capable of inhibiting the expression of superoxide dismutase 1 (SOD1) in a cell.

71. The oligonucleotide agent according to claim 70, wherein the sense strand and the antisense strand of the siRNA have nucleotide sequences that independently are at least 85% homologous to the nucleotide sequence pairs selected from the following groups:
a.
(SEQ ID NO: 922 and SEQ ID NO: 923)
DS17-01M3,
b.
(SEQ ID NO: 924 and SEQ ID NO: 925)
DS17-02M3,
c.
(SEQ ID NO: 926 and SEQ ID NO: 927)
DS17-03M3,
d.
(SEQ ID NO: 928 and SEQ ID NO: 929)
DS17-04M3,
e.
(SEQ ID NO: 930 and SEQ ID NO: 931)
DS17-05M3,
f.
(SEQ ID NO: 932 and SEQ ID NO: 933)
DS17-01M3-AC1 (me14)-L9V3,
g.
(SEQ ID NO: 934 and SEQ ID NO: 935)
DS17-02M3-AC1 (me14)-L9V3,
h.
(SEQ ID NO: 936 and SEQ ID NO: 937)
DS17-03M3-AC1 (me14)-L9V3,
i.
(SEQ ID NO: 938 and SEQ ID NO: 939)
DS17-04M3-AC1 (me14)-L9V3,
j.
(SEQ ID NO: 940 and SEQ ID NO: 941)
DS17-05M3-AC1 (me14)-L9V3,
k.
(SEQ ID NO: 942 and SEQ ID NO: 47)
DS17-29M2-AC1 (me14)-L9V3,
l.
(SEQ ID NO: 932 and SEQ ID NO: 47)
DS17-01M3v-AC1 (me14)-L9V3,
m.
(SEQ ID NO: 934 and SEQ ID NO: 943)
DS17-02M3v-AC1 (me14)-L9V3,
n.
(SEQ ID NO: 936 and SEQ ID NO: 944)
DS17-03M3v-AC1 (me14)-L9V3,
o.
(SEQ ID NO: 938 and SEQ ID NO: 950)
DS17-04M3v-AC1 (me14)-L9V3,
p.
(SEQ ID NO: 940 and SEQ ID NO: 951)
DS17-05M3v-AC1 (me14)-L9V3,
and
q.
(SEQ ID NO: 952 and SEQ ID NO: 939)
DS17-04M3-asSOD1-1-L9V3.

72. The oligonucleotide agent according to claim 1, wherein the oligonucleotide agent comprises a non-targeting ACO conjugated sense strand of a siRNA and an antisense strand of the siRNA, wherein the non-targeting ACO conjugated sense strand comprises a linking component covalently conjugating the ACO and the sense strand, wherein the antisense strand comprises a nucleotide sequence that is at least 90%, at least 95% homology, or 100% identical to SEQ ID NO: 57.

73. The oligonucleotide agent according to the claim 72, wherein the non-targeting ACO conjugated sense strand comprises a nucleotide sequence that is at least 90%, at least 95%, or 100% identical to a nucleotide sequence selected from the group consisting of SEQ ID NOs: 1197-1288 and SEQ ID NOs: 1291-1298.

74. The oligonucleotide agent according to claim 72, wherein the linking component is selected from the linking component group listed in SEQ ID NOs: 1197-1288 in Table 28 and SEQ ID NOs: 1291-1298 in Table 30.

75-84. (canceled)

85. A pharmaceutical composition, comprising the oligonucleotide agent of claim 1 and optionally at least one pharmaceutically acceptable carrier selected from an aqueous carrier, liposome or LNP, polymer, micelle, colloid, metal nanoparticle, non-metallic nanoparticle, bioconjugates, and polypeptide.

86-111. (canceled)

112. A method of treatment comprising administering sufficient amount of the oligonucleotide agent of claim 1 to a subject in need thereof, wherein the treatment is one selected from the group consisting of:
1) silencing the SOD1 gene expression or decreases the SOD1 protein;
2) treating or delaying the onset or progression of Amyotrophic lateral sclerosis (ALS);
3) decreasing or inhibiting the expression of a HTT gene or huntingtin protein;
4) treating or delaying the onset or progression of Huntington's disease (HD);
5) increasing or activating the expression of an App gene or amyloid precursor protein (APP);
6) treating or delaying the onset or progression of APP associated diseases including Cerebral Amyloid Angiopathy App-related (CAA-APP) and Alzheimer Disease (AD);
7) increasing or activating the expression of an SMN2 gene or SMN2 protein; and
8) treating or delaying the onset or progression of spinal muscular atrophy (SMA).
